(19) United States
(12) Patent Application Publication
(10) Pub. No.: US 2026/0337217 A1
(43) Pub. Date: Oct. 29, 2026

(54) GALNAC COMPOUND, CONJUGATE, COMPOSITION, AND USE THEREOF

(71) Applicant: RIGERNA THERAPEUTICS (BEIJING) CO., LTD., Beijing (CN)

(72) Inventors: Yuanyu HUANG, Beijing (CN); Haitao LI, Beijing (CN); Yongxin GAO, Beijing (CN); Zhichao LIU, Beijing (CN); Xiaofeng HAN, Beijing (CN)

(73) Assignee: RIGERNA THERAPEUTICS (BEIJING) CO., LTD., Beijing (CN)

(21) Appl. No.: 19/483,056

(22) PCT Filed: May 20, 2024
(86) PCT No.: PCT/CN2024/094102

§ 371 Date: Nov. 10, 2025

(30) Foreign Application Priority Data
May 24, 2023 (CN) 202310596547.1
May 24, 2023 (CN) 202310597844.8

(51) Int. Cl. C07H 15/26; A61K 31/7056; A61K 31/7105

(52) U.S. Cl. CPC C07H 15/26; A61K 31/7056; A61K 31/7105

(57) ABSTRACT

The present invention relates to the field of drugs, and in particular, to a compound, a conjugate, a composition, and use thereof. The oligonucleotide conjugate provided is synthesized by means of the provided compound. The oligonucleotide conjugate can target parenchymal hepatic cells with high efficiency and can effectively inhibit target gene expression in the parenchymal hepatic cells. The oligonucleotide conjugate can be used for treating and/or preventing liver-derived diseases and has the advantages of high in-vivo activity, stable and lasting pharmacodynamic action, excellent safety, and low animal-level toxicity.

This application claims the priority of Chinese Patent Application No. 2023105965471, filed with the China National Intellectual Property Administration on May 24, 2023, and titled with “COMPOUND, CONJUGATE, COMPOSITION AND USE THEREOF”, the disclosure of which is hereby incorporated by reference in its entirety.

This application claims the priority of Chinese Patent Application No. 2023105978448, filed with the China National Intellectual Property Administration on May 24, 2023, and titled with “COMPOUND AND PREPARATION METHOD AND USE THEREOF”, the disclosure of which is hereby incorporated by reference in its entirety.

### FIELD

The present disclosure relates to the field of nucleic acid drugs, in particular to a GalNAc compound, conjugate, composition and use thereof.

### BACKGROUND

Asialoglycoprotein receptor (ASGPR), also known as liver lectin, is a transmembrane protein that is highly and specifically expressed on the surface of hepatic sinusoidal and basolateral cells. ASGPR is primarily composed of two subunits, H1 and H2, which together exhibit endocytic activity. A normal mature hepatocyte contains approximately 500,000 receptor molecules, with about 10% of ASGPR expressed on the cell membrane. ASGPR specifically recognizes, binds to, and endocytoses certain glycoproteins in blood circulation that has galactose or N-acetylgalactosamine residues at terminals, facilitating their metabolism in hepatocytes. By conjugating a targeting ligand with oligonucleotides, the liver-targeted delivery of oligonucleotides can be achieved through the link of the targeting ligand to ASGPR.

### SUMMARY

In view of this, the present disclosure provides a compound, conjugate, composition and use thereof.

The oligonucleotide conjugate provided by the present disclosure can highly effectively target the liver and effectively inhibit the expression of a target gene in liver. The oligonucleotide conjugate can be used in the treatment and/or prevention of a hepatogenic disease, and has higher in vivo activity and more stable and lasting efficiency than those of control carrier.

In order to achieve the above purposes, the present disclosure provides the following technical solutions.

In a first aspect of the present disclosure, the present disclosure provides a compound having a structure represented by formula (Ia), or a stereoisomer, a pharmaceutically acceptable salt or a prodrug thereof,

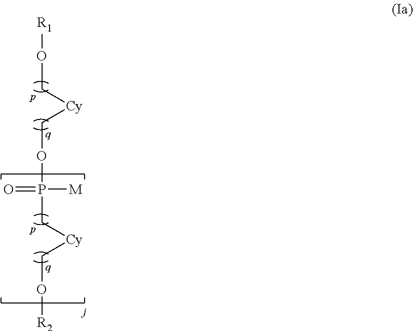

in formula (Ia), R₁ is selected from the group consisting of H, a hydroxyl protecting group and

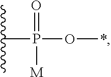

wherein * represents a site for linking an active pharmaceutical molecule;
R₂ is selected from the group consisting of H, a reactive phosphorus group and

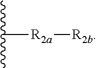

wherein R_{2b} is a solid phase support containing an amino functional group, and R₂ₐ is a covalent linking group linked to the amino functional group;
j is selected from the group consisting of 0, 1, 2 and 3;
each M is independently selected from the group consisting of hydroxyl and mercapto;
each p is independently selected from the group consisting of 0, 1, 2, 3 and 4;
each q is independently selected from the group consisting of 0, 1, 2, 3 and 4;
each Cy is independently

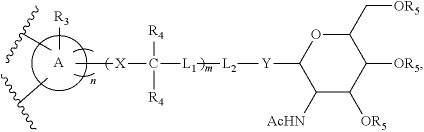

wherein A is a 5-6 membered oxygen-containing heterocycle, R₃ is selected from the group consisting of H and C₁-C₆ alkoxy, n is selected from the group consisting of 1 and 2, each X is independently selected from the group consisting of O, S and NH, each R₄ is independently selected from the group consisting of H, C₁-C₆ alkyl, and C₁-C₆ alkoxy, each L₁ is independently selected from the group consisting of a substituted or unsubstituted triazolyl and a substituted or unsubstituted amido, m is selected from the group consisting of 1 and 2, and each L₂ is independently selected from the group consisting of a substituted or unsubstituted C₁-C₃₀ alkylidene and

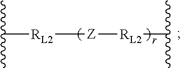

wherein each r is independently selected from the group consisting of 1, 2, 3, 4, 5, 6, 7, 8, 9, and 10, each Z is independently selected from the group consisting of O, S and NH, each R_{L2} is independently a substituted or unsubstituted C₁-C₁₀ alkylidene, each Y is independently selected from the group consisting of O, S and NH, and each R₅ is independently selected from the group consisting of H, a substituted or unsubstituted C₁-C₄ alkyacyl and a substituted or unsubstituted C₆-C₁₂ arylacyl.

In some optional embodiments of the present disclosure, A is a 5 to 6-membered saturated oxygen-containing heterocycle.

In some optional embodiments of the present disclosure, A is a 5 to 6-membered saturated heterocycle containing one oxygen atom.

In some optional embodiments of the present disclosure, A is selected from the group consisting of

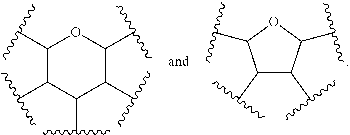 [SMILES: *C1OC(*)C(*)C(*)C1* ; *C1OC(*)C(*)C1*]

In some specific embodiments of the present disclosure, A is

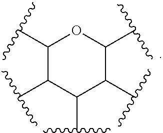 [SMILES: *C1OC(*)C(*)C(*)C1*]

In some specific embodiments of the present disclosure, A is

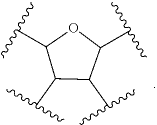 [SMILES: *C1OC(*)C(*)C1*]

In some specific embodiments of the present disclosure, m is 1.

In some specific embodiments of the present disclosure, X is O.

In some specific embodiments of the present disclosure, each R₄ is H.

In some optional embodiments of the present disclosure, each L₁ is independently selected from the group consisting of:

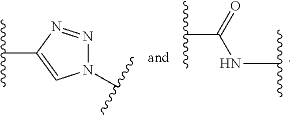 [SMILES: *c1cn(*)nn1 ; *NC(*)=O]

In some specific embodiments of the present disclosure, each L₁ is

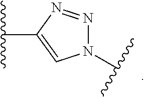 [SMILES: *c1cn(*)nn1]

In some specific embodiments of the present disclosure, each L₁ is

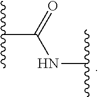 [SMILES: *NC(*)=O]

In some optional embodiments of the present disclosure, R₃ is selected from the group consisting of H and C₁-C₃ alkoxy.

In some specific embodiments of the present disclosure, R₃ is selected from the group consisting of H and methoxy.

In some specific embodiments of the present disclosure, R₃ is H.

In some specific embodiments of the present disclosure, R₃ is methoxy.

In some optional embodiments of the present disclosure, each L₂ is independently

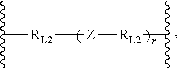

wherein each r is independently selected from the group consisting of 1, 2, 3, 4, 5, 6, 7, 8, 9 and 10, each Z is independently selected from the group consisting of O, S and NH, and each R_{L2} is independently a substituted or unsubstituted C₁-C₁₀ alkylidene.

In some optional embodiments of the present disclosure, each L_{L2} is independently a substituted or unsubstituted C₁-C₅ alkylidene.

In some optional embodiments of the present disclosure, each L_{L2} is independently a substituted or unsubstituted C₁-C₃ alkylidene.

In some optional embodiments of the present disclosure, each L_{L2} is independently a substituted or unsubstituted C₂ alkylidene.

In some optional embodiments of the present disclosure, each r is independently selected from the group consisting of 2 and 3.

In some optional embodiments of the present disclosure, each L₂ is independently

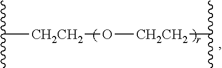

wherein r is selected from the group consisting of 1, 2, 3, 4, 5, 6, 7, 8, 9 and 10.

In some specific embodiments of the present disclosure, each L₂ is independently

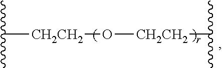

wherein r is selected from the group consisting of 2 and 3.

In some specific embodiments of the present disclosure, each L₂ is

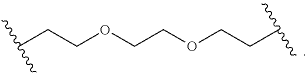 [SMILES: *CCOCCOCC*]

In some specific embodiments of the present disclosure, each L₂ is

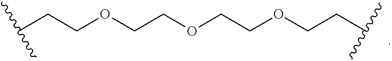 [SMILES: *CCOCCOCCOCC*]

In some specific embodiments of the present disclosure, each Y is O.

In some embodiments of the present disclosure, each R₅ is independently selected from the group consisting of H,

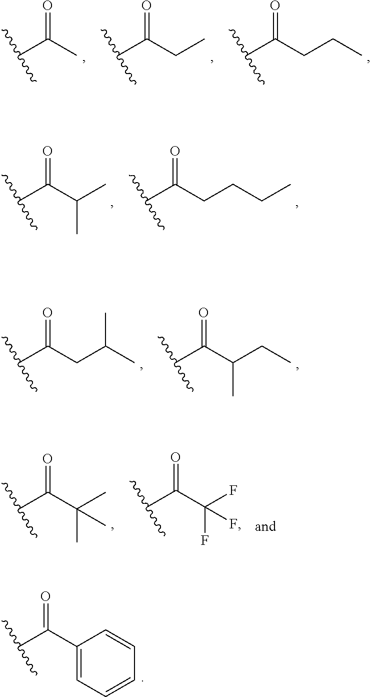

In some specific embodiments of the present disclosure, each R₅ is

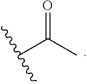 [SMILES: *C(C)=O]

In some specific embodiments of the present disclosure, each R₅ is H.

In some embodiments of the present disclosure, p and q are each independently selected from the group consisting of 0 and 1.

In some specific embodiments of the present disclosure, p=1 and q=1.

In some specific embodiments of the present disclosure, p=1 and q=0.

In some specific embodiments of the present disclosure, p=0 and q=1.

In some specific embodiments of the present disclosure, p=0 and q=0.

In the present disclosure, the hydroxyl protecting group may be any hydroxyl protecting group, provided that it can protect hydroxyl, and the specific type is not limited. In some embodiments, the hydroxyl protecting group is stable under an alkaline condition but can be removed under an acidic condition.

In some optional embodiments of the present disclosure, the hydroxyl protecting group can be used in the present disclosure includes, but is not limited to monomethoxytrityl, dimethoxytrityl, trimethoxytrityl, 9-phenylxanthine-9-yl (Pixyl), 9-(p-methoxyphenyl)xanthine-9-yl (Mox), trityl (Tr), 4-methoxytrityl (MMTr), 4,4′-dimethoxytrityl (DMTr) and 4,4′,4″-trimethoxytrityl (TMTr).

In some optional embodiments of the present disclosure, the hydroxyl protecting group is selected from the group consisting of trityl (Tr), 4-methoxytrityl (MMTr), 4,4′-dimethoxytrityl (DMTr) and 4,4′,4″-trimethoxytrityl (TMTr).

In some specific embodiments of the present disclosure, the hydroxyl protecting group is 4,4′-dimethoxytrityl (DMTr).

In the present disclosure, the term “reactive phosphorous group” refers to a phosphorous-containing group that can be removed by reacting with other compounds.

In some optional embodiments of the present disclosure, the reactive phosphorus group is selected from the group consisting of

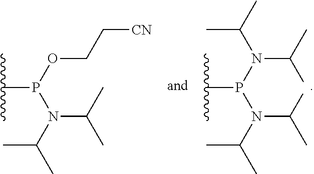 [SMILES: *P(OCCC#N)N(C(C)C)C(C)C ; *P(N(C(C)C)C(C)C)N(C(C)C)C(C)C]

In some specific embodiments of the present disclosure, the reactive phosphorus group is

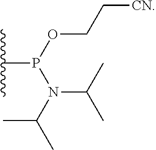 [SMILES: *P(OCCC#N)N(C(C)C)C(C)C]

In some optional embodiments of the present disclosure, the active pharmaceutical molecule is selected from the group consisting of a small molecule drug, an antibody, and an oligonucleotide.

In some optional embodiments of the present disclosure, the oligonucleotide is selected from the group consisting of a single-stranded oligonucleotide and a double-stranded oligonucleotide.

In some optional embodiments of the present disclosure, the active pharmaceutical molecule is a single-stranded oligonucleotide.

In some optional embodiments of the present disclosure, the single-stranded oligonucleotide is an antisense oligonucleotide (ASO).

In some specific embodiments of the present disclosure, the active pharmaceutical molecule is a double-stranded oligonucleotide.

In some optional embodiments of the present disclosure, the double-stranded oligonucleotide is a small interfering RNA (siRNA).

In some specific embodiments of the present disclosure, the active pharmaceutical molecule is siRNA.

In some specific embodiments of the present disclosure, the siRNA is conjugated with one of the above compounds, and the compound is conjugated to the 3′end of the siRNA sense strand.

In some optional embodiments of the present disclosure, the siRNA is conjugated with one of the above compounds, and the compound is conjugated to the 5′end of the siRNA sense strand.

In some specific embodiments of the present disclosure, the siRNA is conjugated with two of the above compounds, and the two compounds are conjugated to the 3′end and 5′end of the siRNA sense strand, respectively.

In some optional embodiments of the present disclosure, the solid phase support is selected from the group consisting of resin containing an amino functional group and controlled pore glass bead containing an amino functional group.

In some optional embodiments of the present disclosure, R_{2b} is

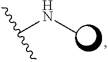

wherein [symbol] represents resin (e.g., polystyrene (PS) resin) or controlled pore glass bead (CPG).

In some specific embodiments of the present disclosure, R₂ₐ is

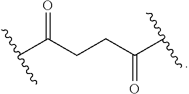 [SMILES: *C(=O)CCC(*)=O]

In some specific embodiments of the present disclosure,

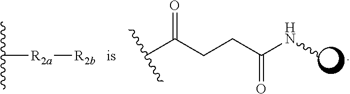

In some optional embodiments of the present disclosure, j is selected from the group consisting of 0, 1 and 2.

In some specific embodiments of the present disclosure, j is 0.

In some specific embodiments of the present disclosure, j is 1.

In some specific embodiments of the present disclosure, j is 2.

In some optional embodiments of the present disclosure, in formula (Ia), each Cy independently has a structure selected from the group consisting of:

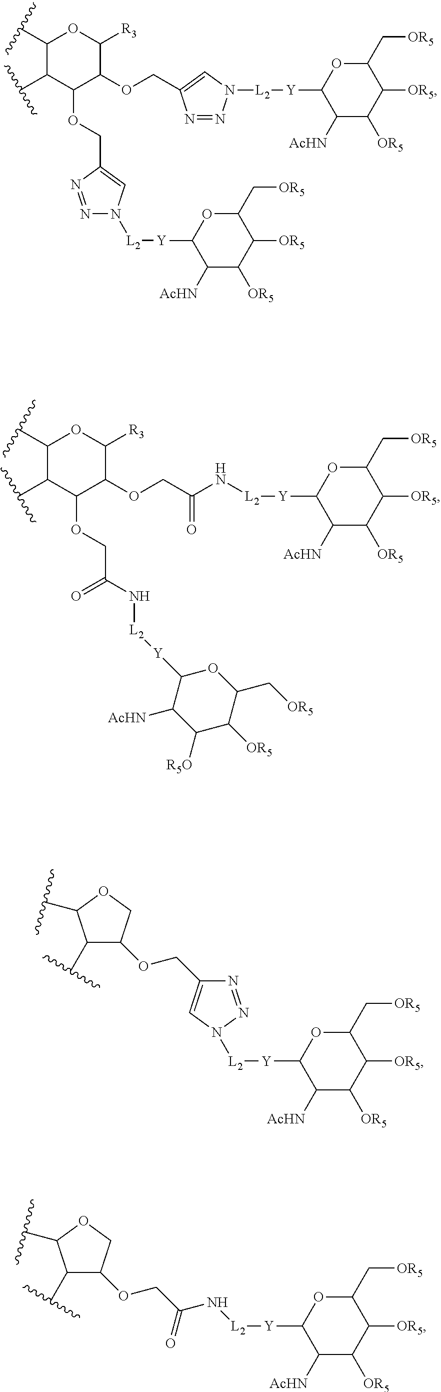

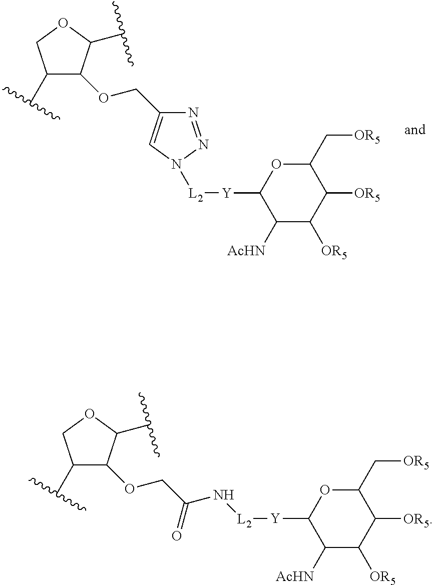

In some specific embodiments of the present disclosure, the compound has a structure selected from the group consisting of

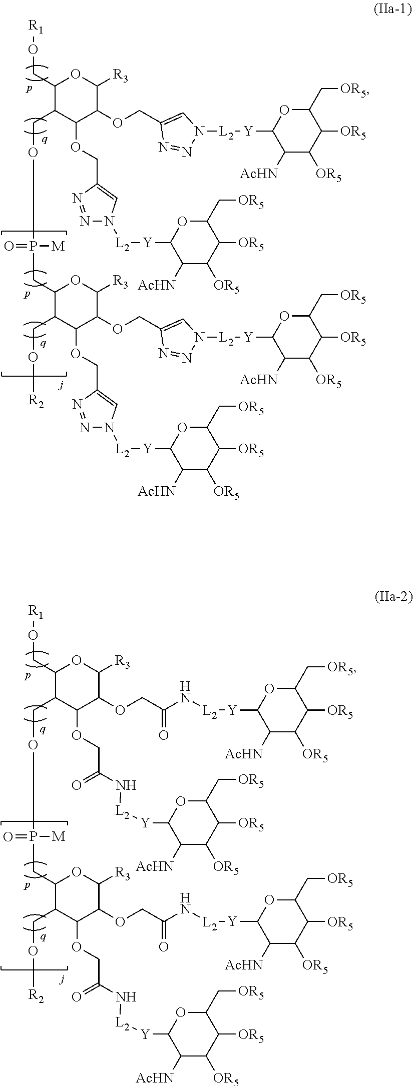

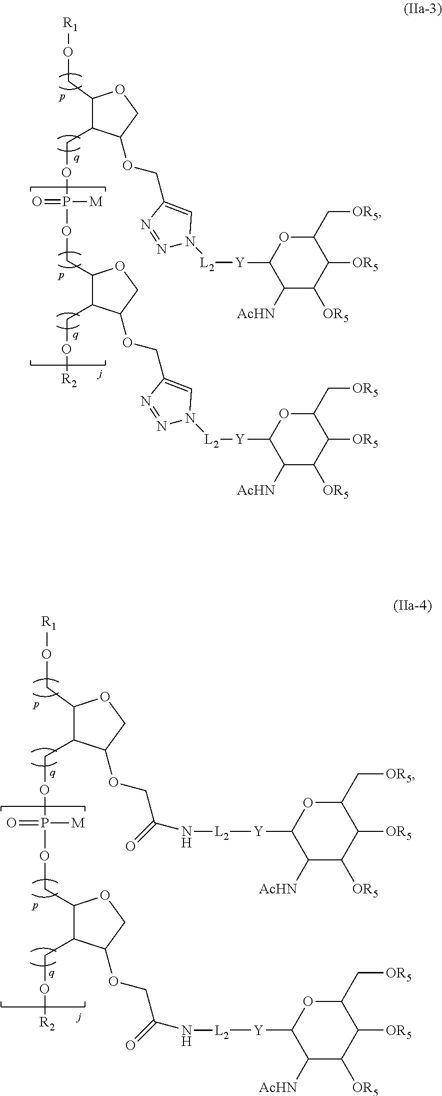

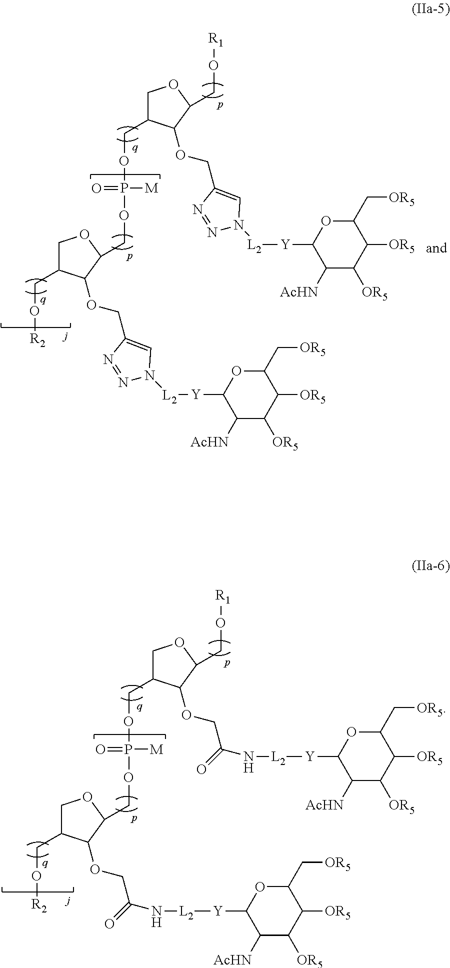

In some specific embodiments of the present disclosure, the compound is selected from the group consisting of:

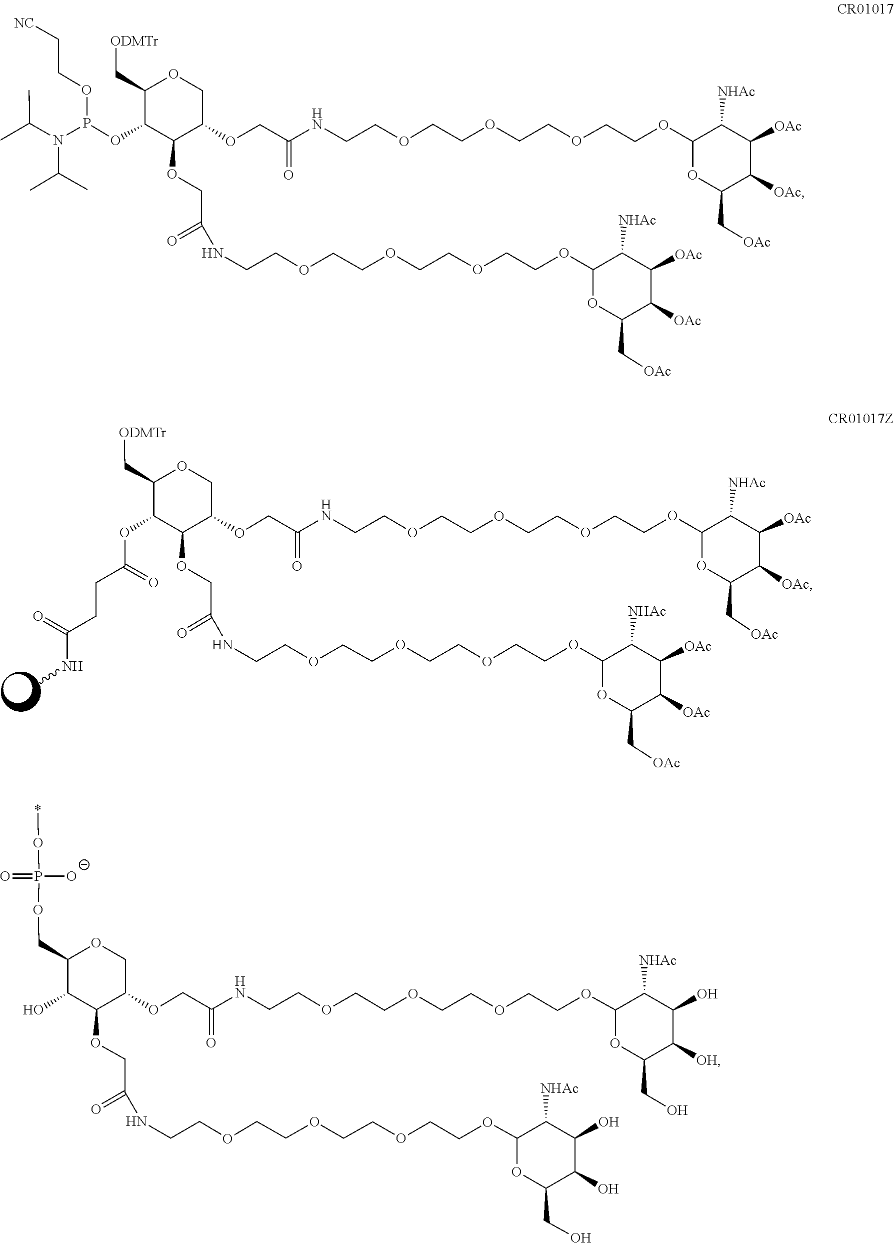

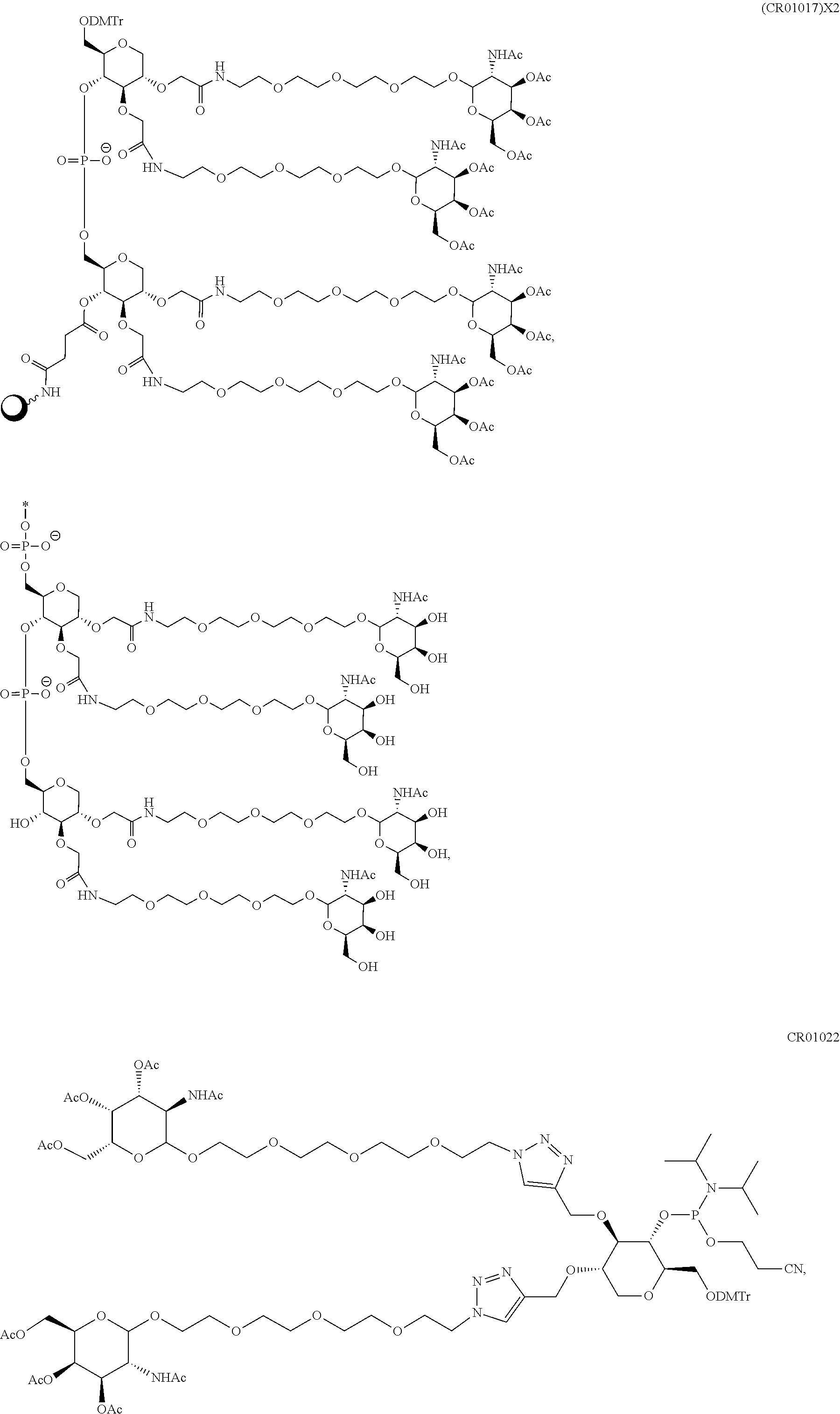

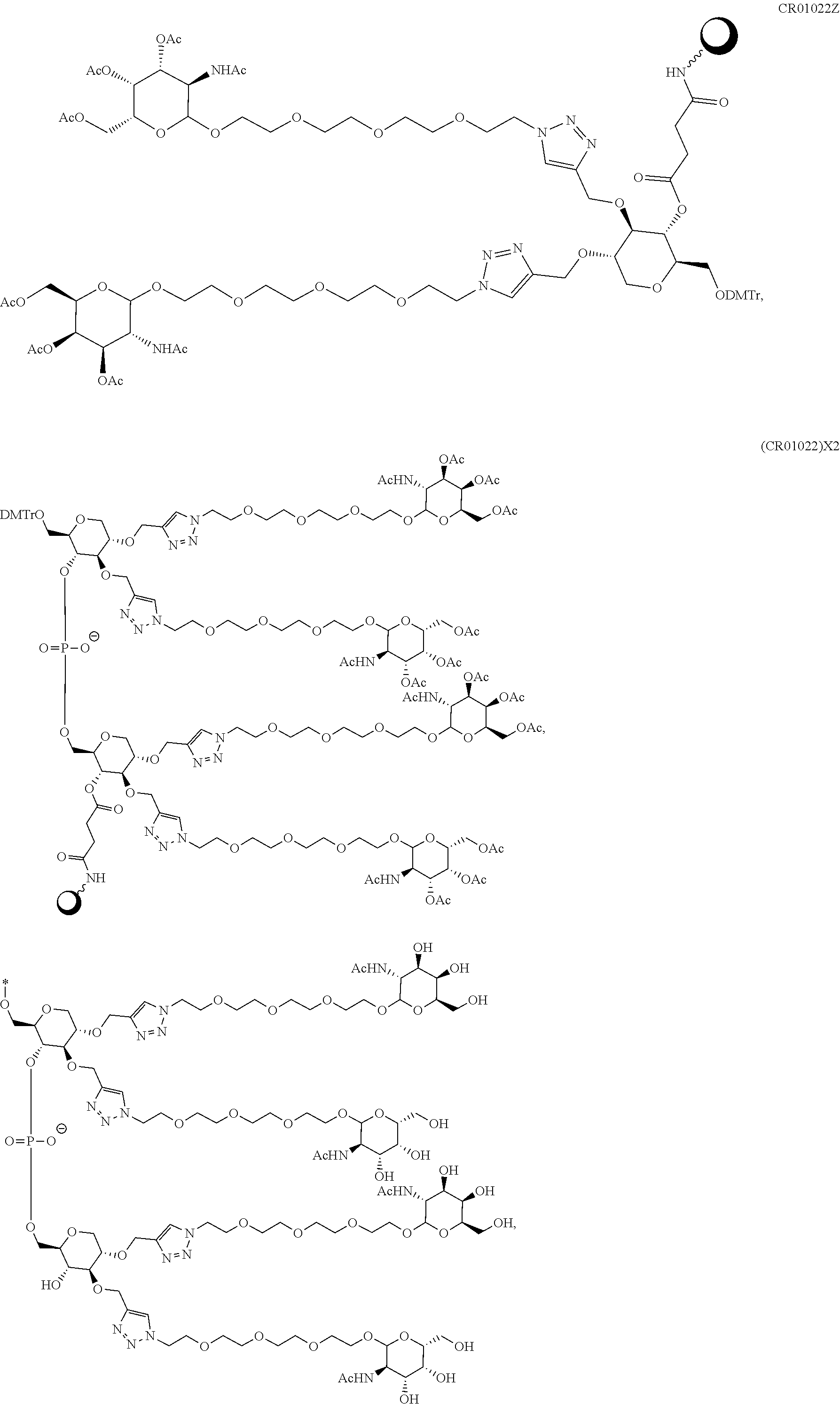

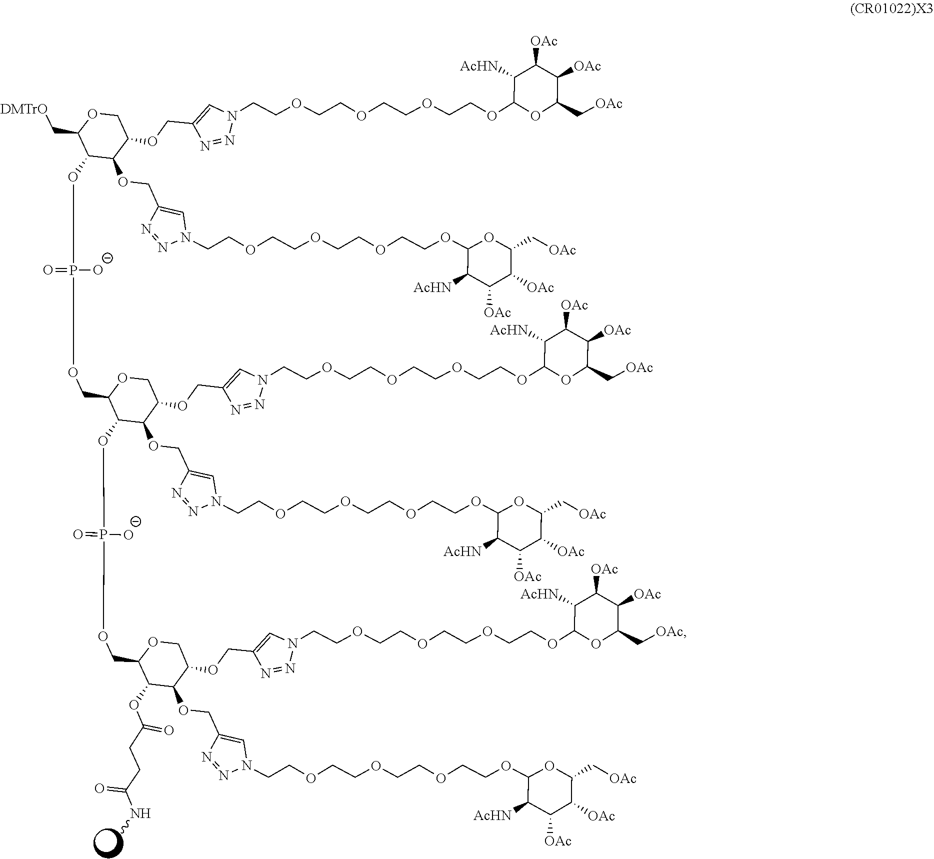

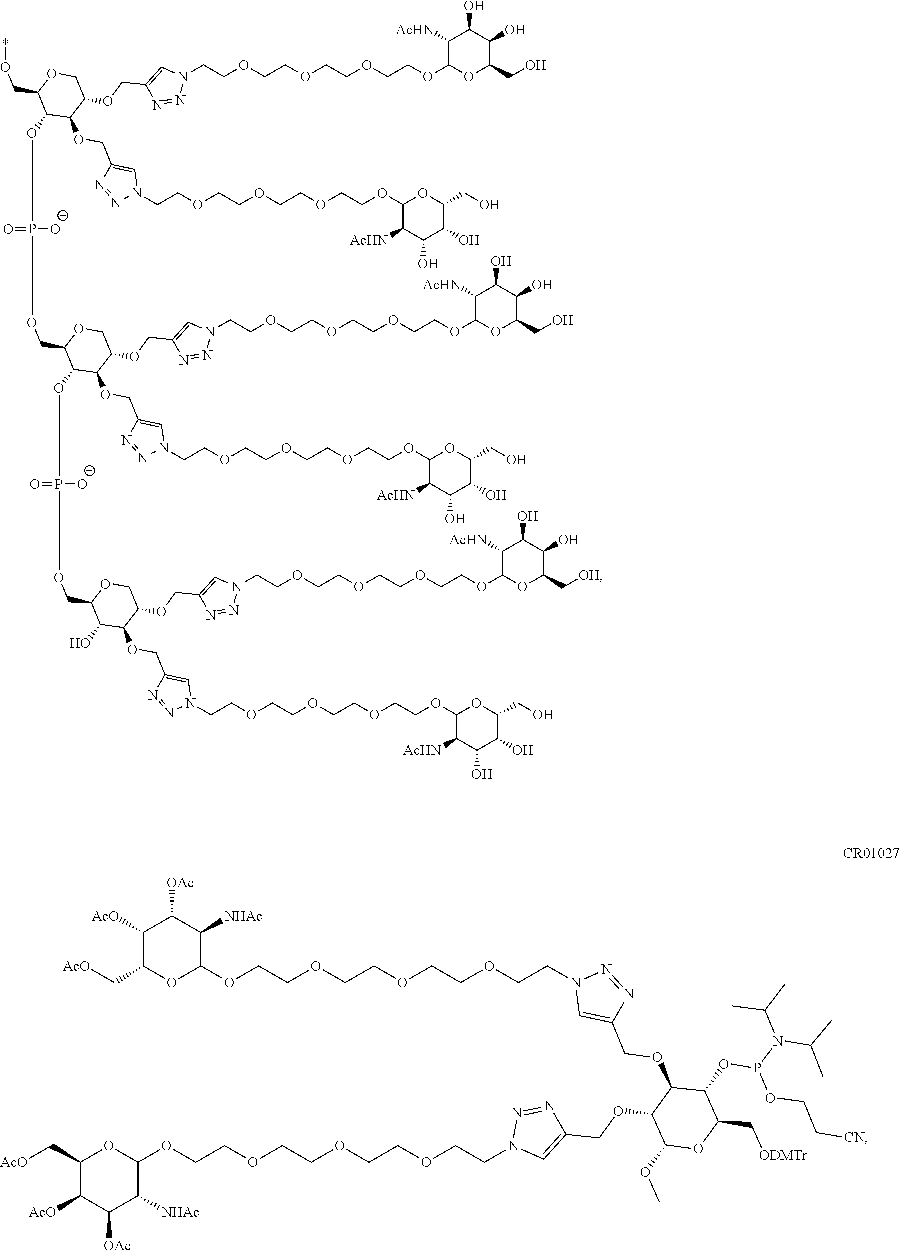

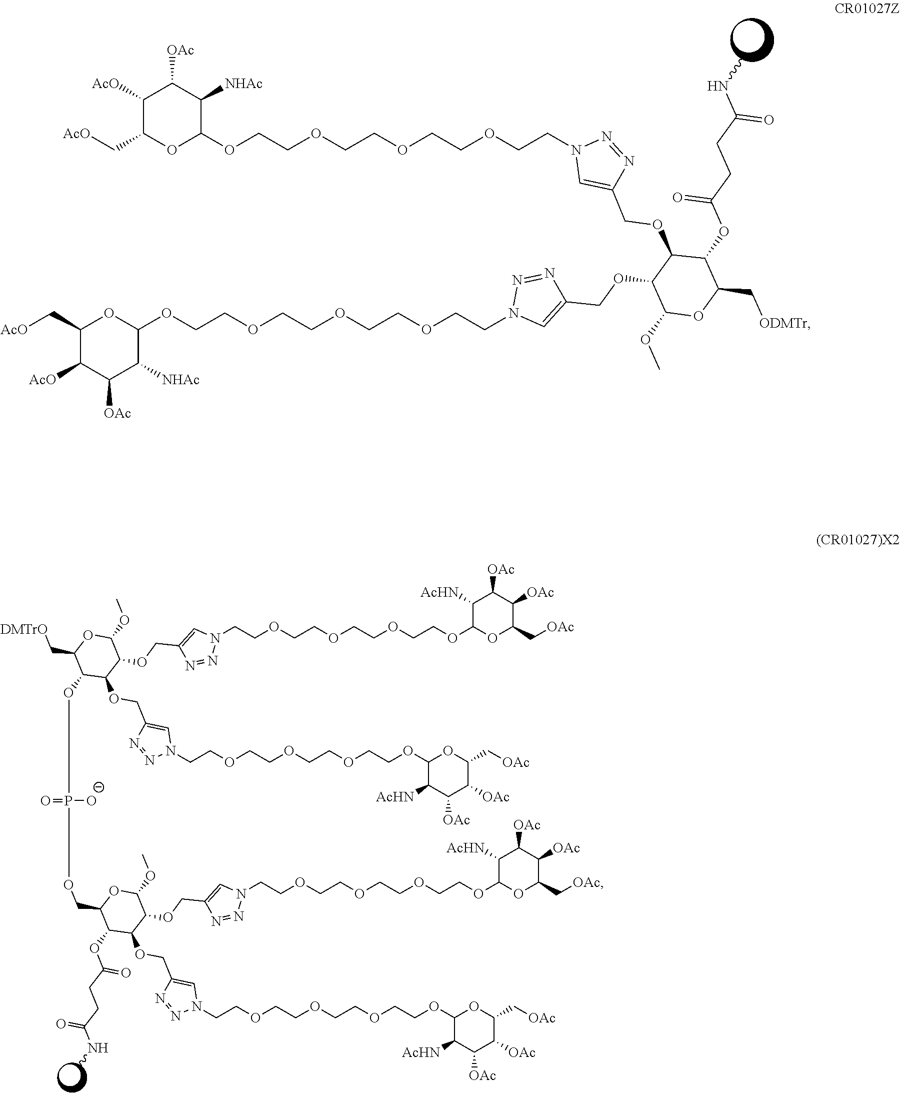

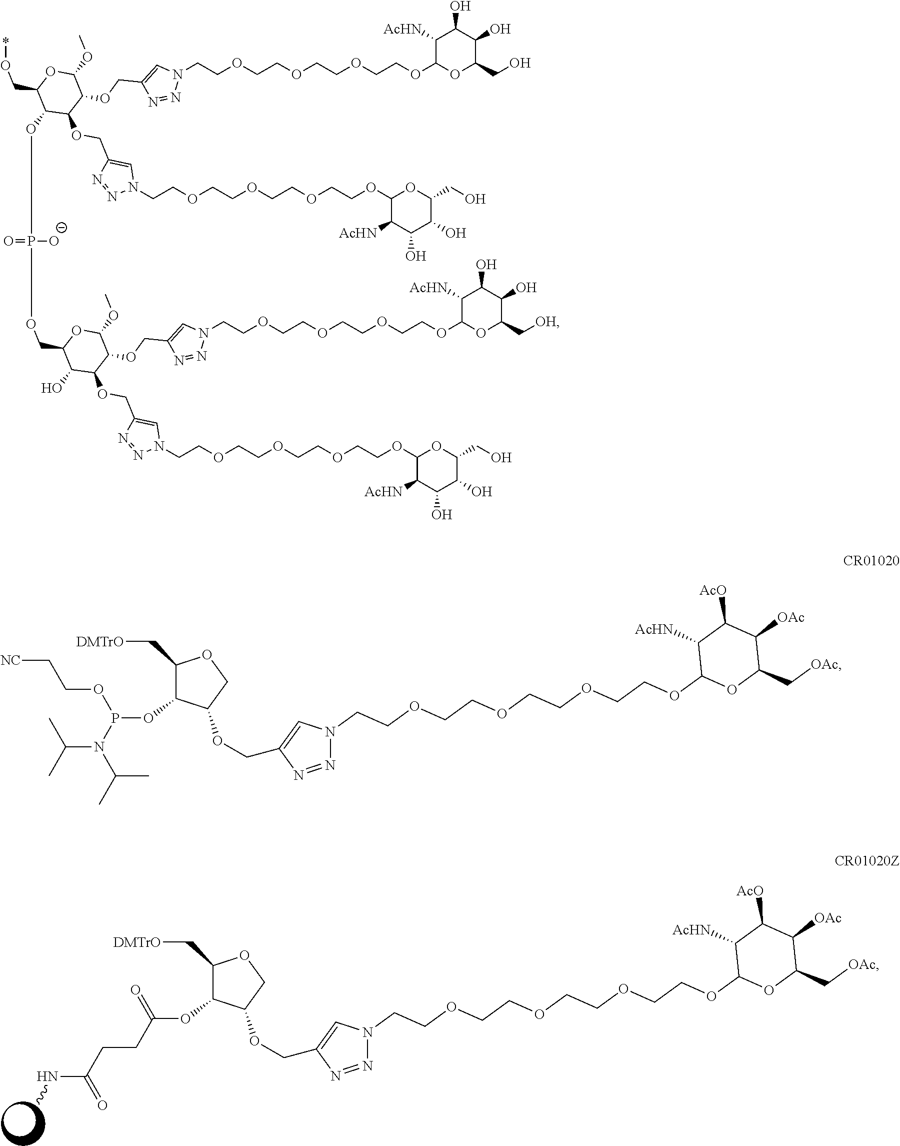

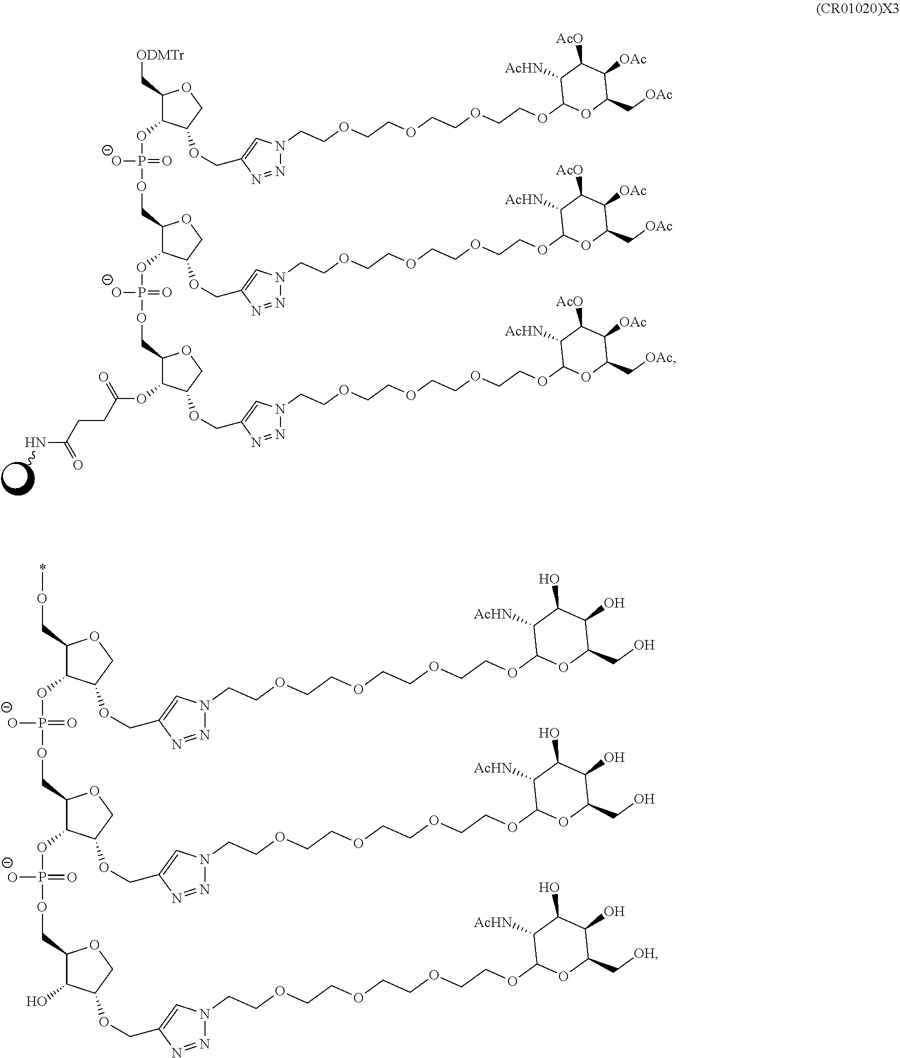

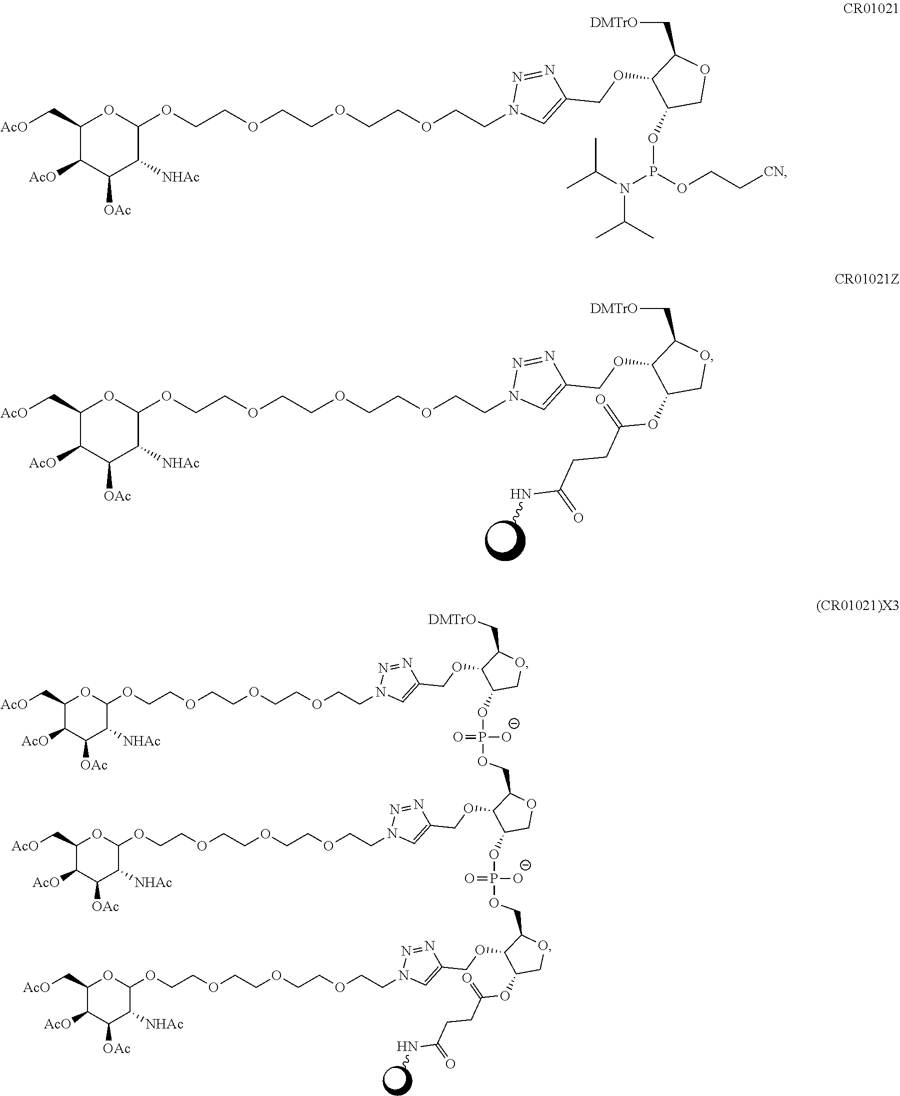

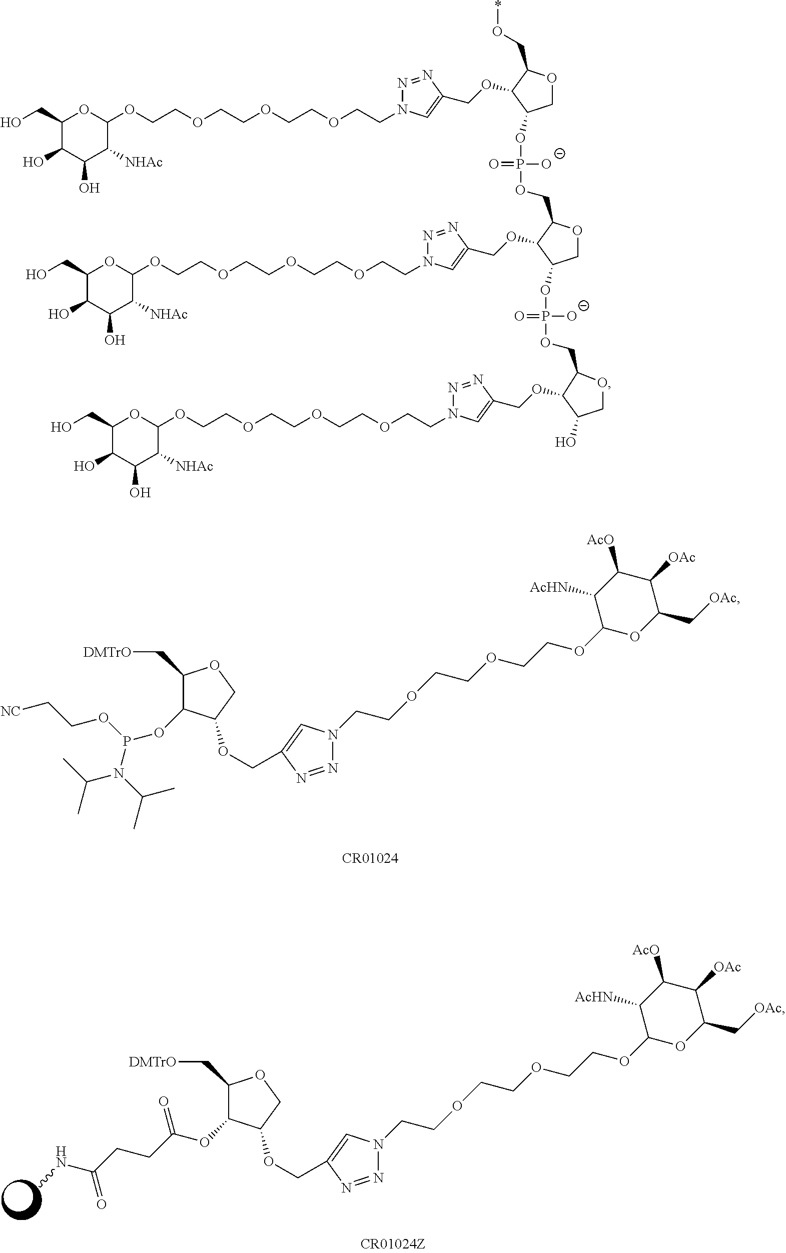

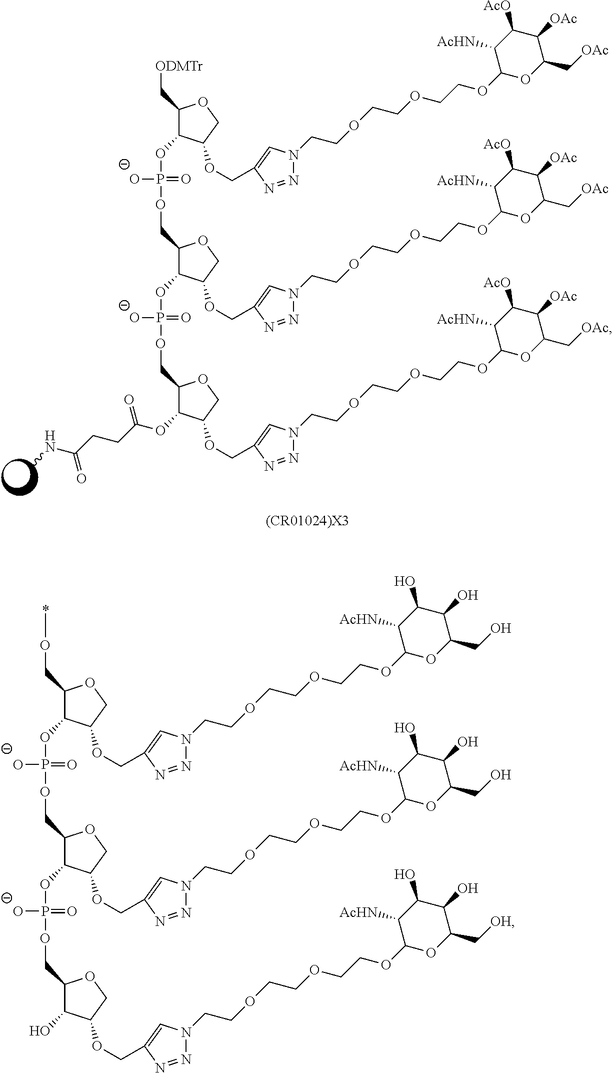

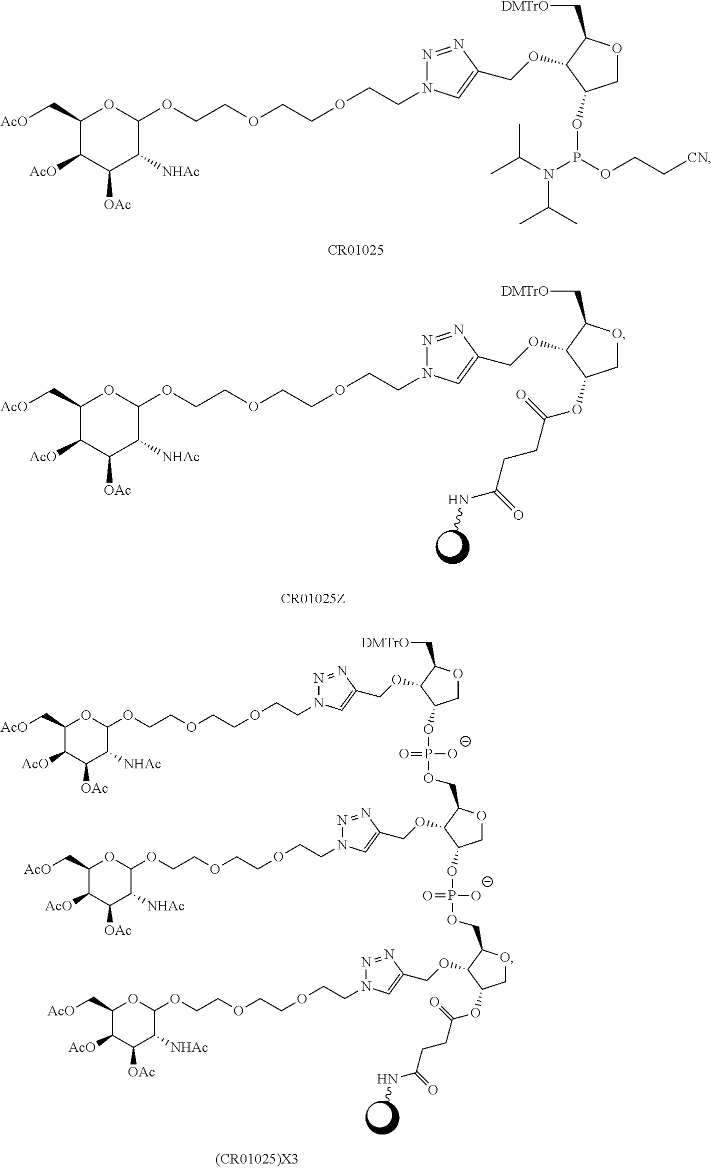

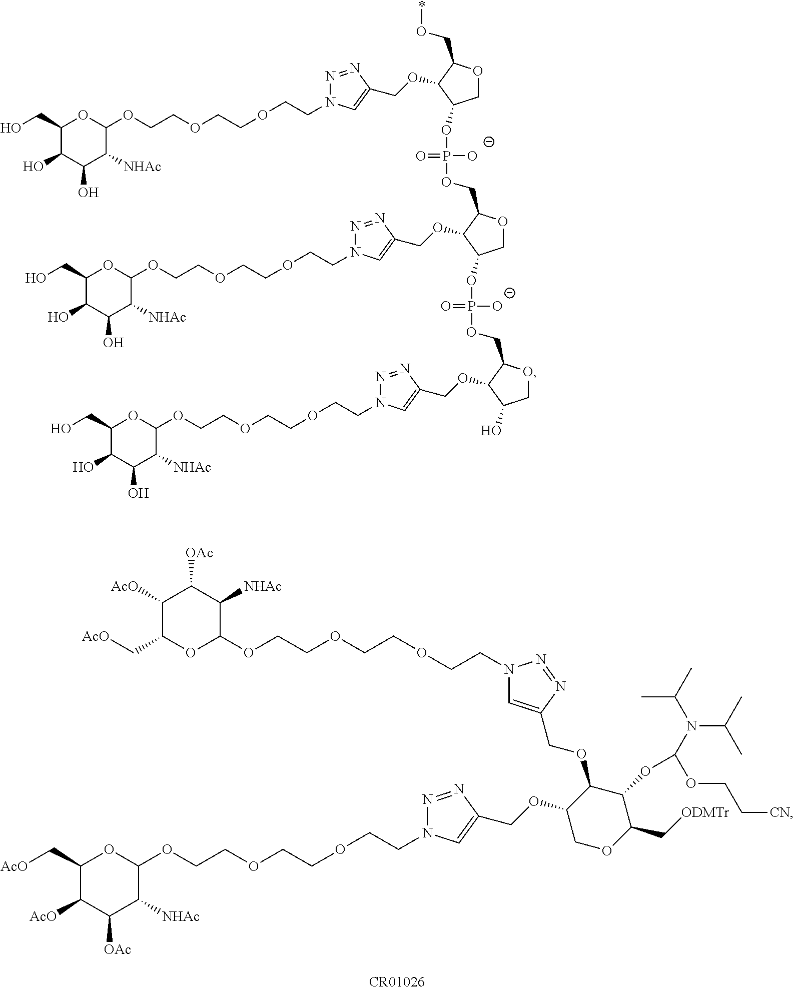

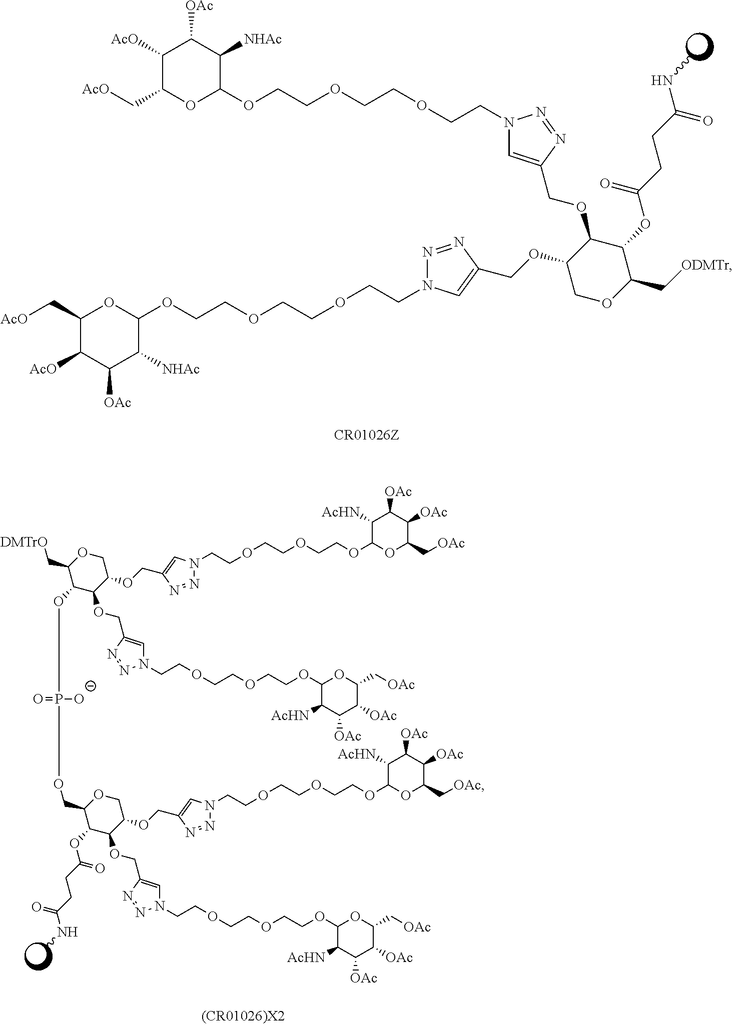

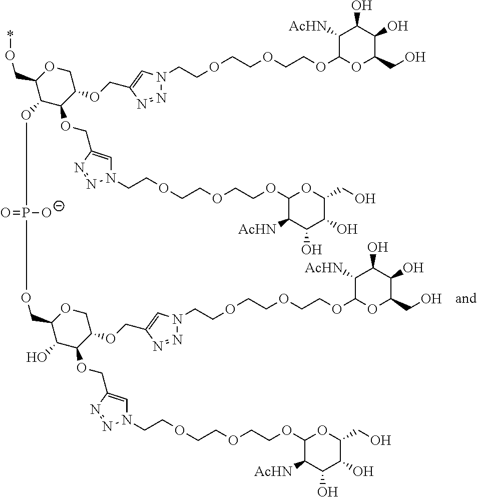 [SMILES: *OC[C@H]1OC[C@H](OCc2cn(CCOCCOCCOC3O[C@H](CO)[C@H](O)[C@H](O)[C@H]3NC(C)=O)nn2)[C@@H](OCc2cn(CCOCCOCCOC3O[C@H](CO)[C@H](O)[C@H](O)[C@H]3NC(C)=O)nn2)[C@@H]1OP(=O)([O-])OC[C@H]1OC[C@H](OCc2cn(CCOCCOCCOC3O[C@H](CO)[C@H](O)[C@H](O)[C@H]3NC(C)=O)nn2)[C@@H](OCc2cn(CCOCCOCCOC3O[C@H](CO)[C@H](O)[C@H](O)[C@H]3NC(C)=O)nn2)[C@@H]1O]

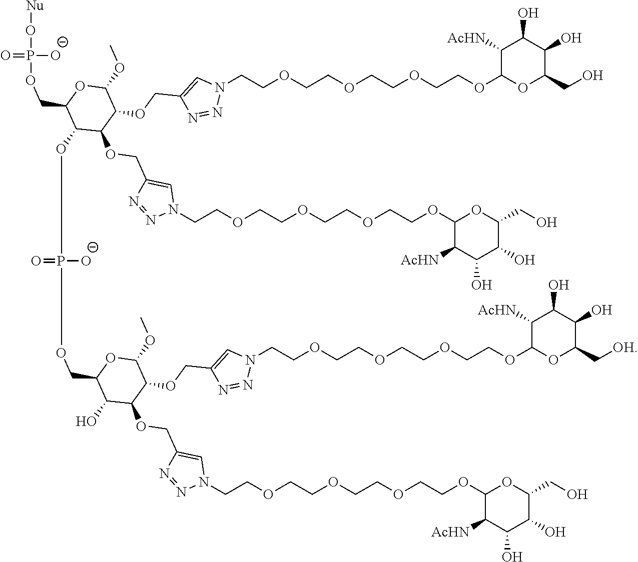

In a second aspect of the present disclosure, the present disclosure further provides a conjugate having a structure represented by formula (Ib), or a stereoisomer, a pharmaceutically acceptable salt or a prodrug thereof:

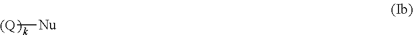

in formula (Ib), Nu represents an oligonucleotide;
k is selected from the group consisting of 1 and 2;
Q has a structure of

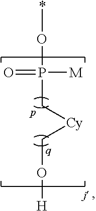

wherein j′ is selected from the group consisting of 1, 2, 3 and 4, and
*, M, p, q and Cy are as defined in any above-mentioned description.

Wherein,

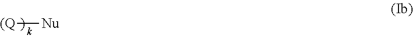

means that k Qs are attached to Nu. When k is 2, k Qs are attached to different sites of Nu, respectively.

In some optional embodiments of the present disclosure, j′ is selected from the group consisting of 1, 2 and 3.

In some specific embodiments of the present disclosure, j′ is 1.

In some specific embodiments of the present disclosure, j′ is 2.

In some specific embodiments of the present disclosure, j′ is 3.

In some specific embodiments of the present disclosure, k is 1.

In some specific embodiments of the present disclosure, k is 2.

In some optional embodiments of the present disclosure, the oligonucleotide is selected from the group consisting of a single-stranded oligonucleotide and a double-stranded oligonucleotide.

In some optional embodiments of the present disclosure, the oligonucleotide is a single-stranded oligonucleotide. The single-stranded oligonucleotide includes, but is not limited to, antisense oligonucleotide (ASO), aptamer, ribozyme, deoxyribozyme and circular RNA.

In some optional embodiments of the present disclosure, the single-stranded oligonucleotide is ASO.

In some specific embodiments of the present disclosure, the oligonucleotide is a double-stranded oligonucleotide. The double-stranded oligonucleotide includes, but is not limited to, small interfering RNA (siRNA), double-stranded RNA (dsRNA), microRNA (miRNA), (small guide RNA (sgRNA), small activating RNA (saRNA) and short hairpin RNA (shRNA).

In some specific embodiments of the present disclosure, the double-stranded oligonucleotide is siRNA.

In some specific embodiments of the present disclosure, Nu represents a double-stranded oligonucleotide.

In some specific embodiments of the present disclosure, Nu represents siRNA.

In some specific embodiments of the present disclosure, k is 1, and one Q is conjugated to the 3′ end of the siRNA sense strand.

In some optional embodiments of the present disclosure, k is 1, and one Q is conjugated to the 5′ end of the siRNA sense strand.

In some specific embodiments of the present disclosure, k is 2, and two Q are respectively conjugated to the 3′ end and 5′ end of the siRNA sense strand.

In some optional embodiments of the present disclosure, the conjugate has a structure selected from the group consisting of

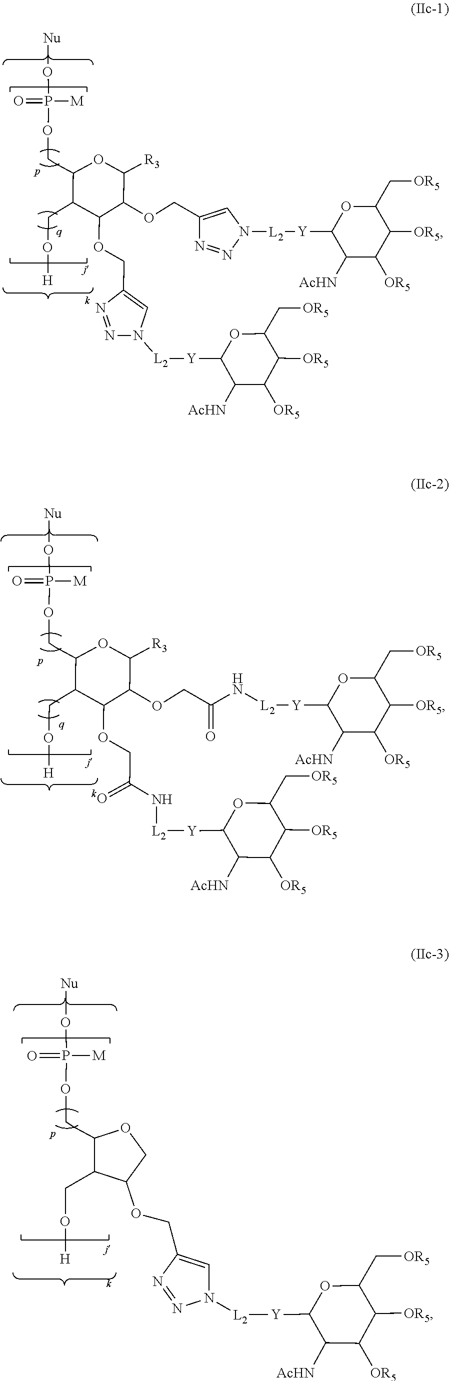

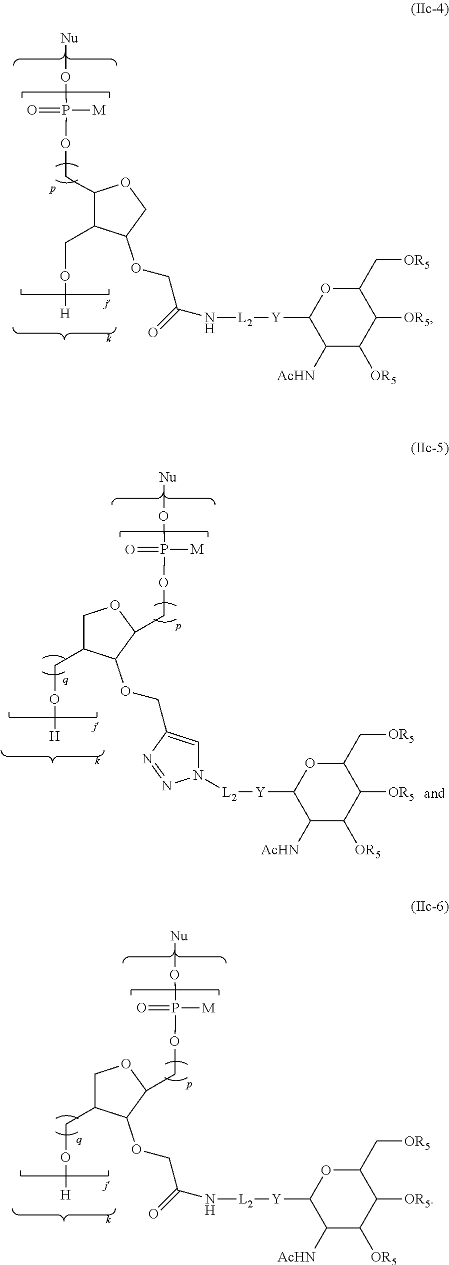

In some specific embodiments of the present disclosure, the conjugate is selected from the group consisting of

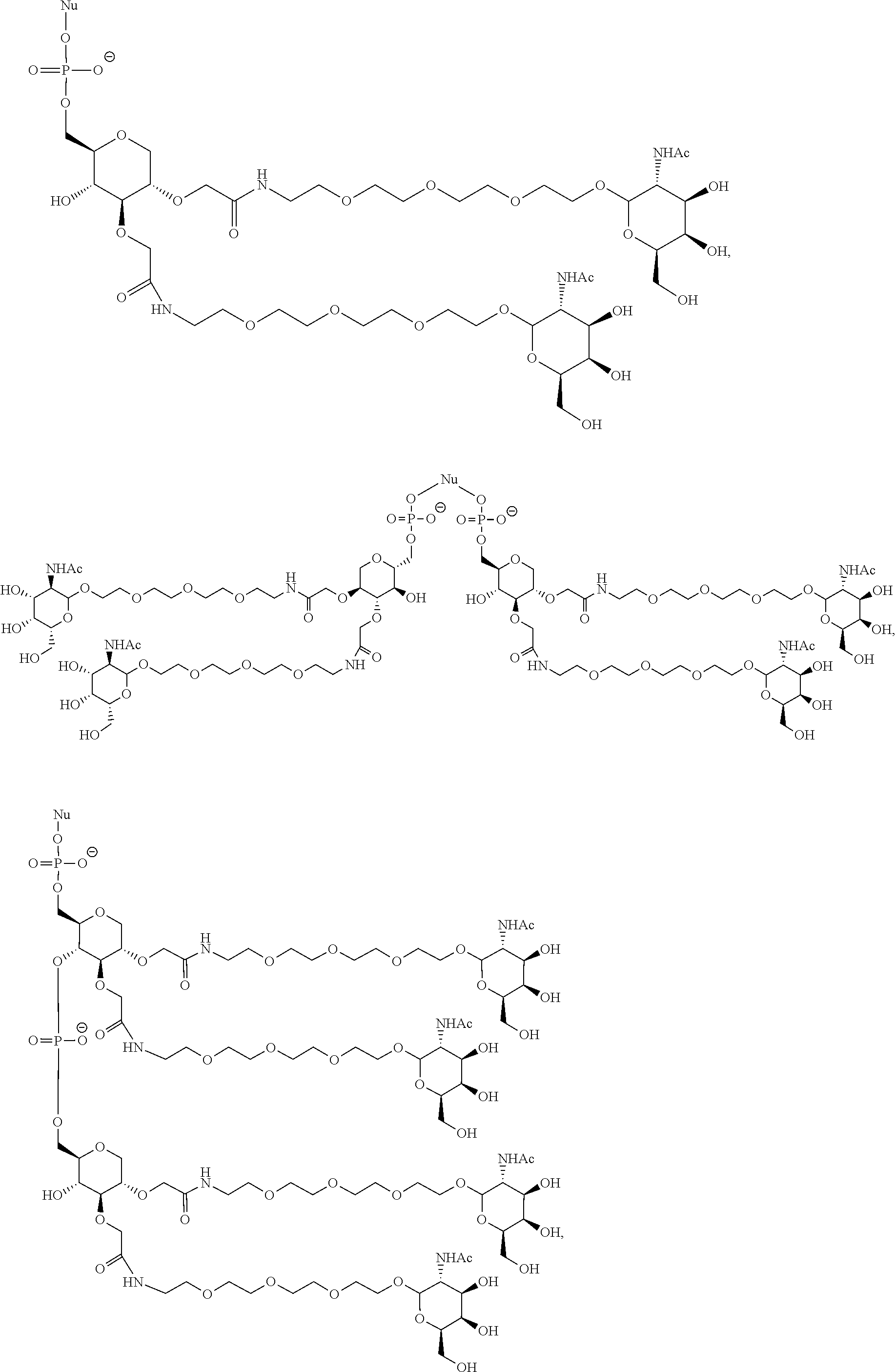

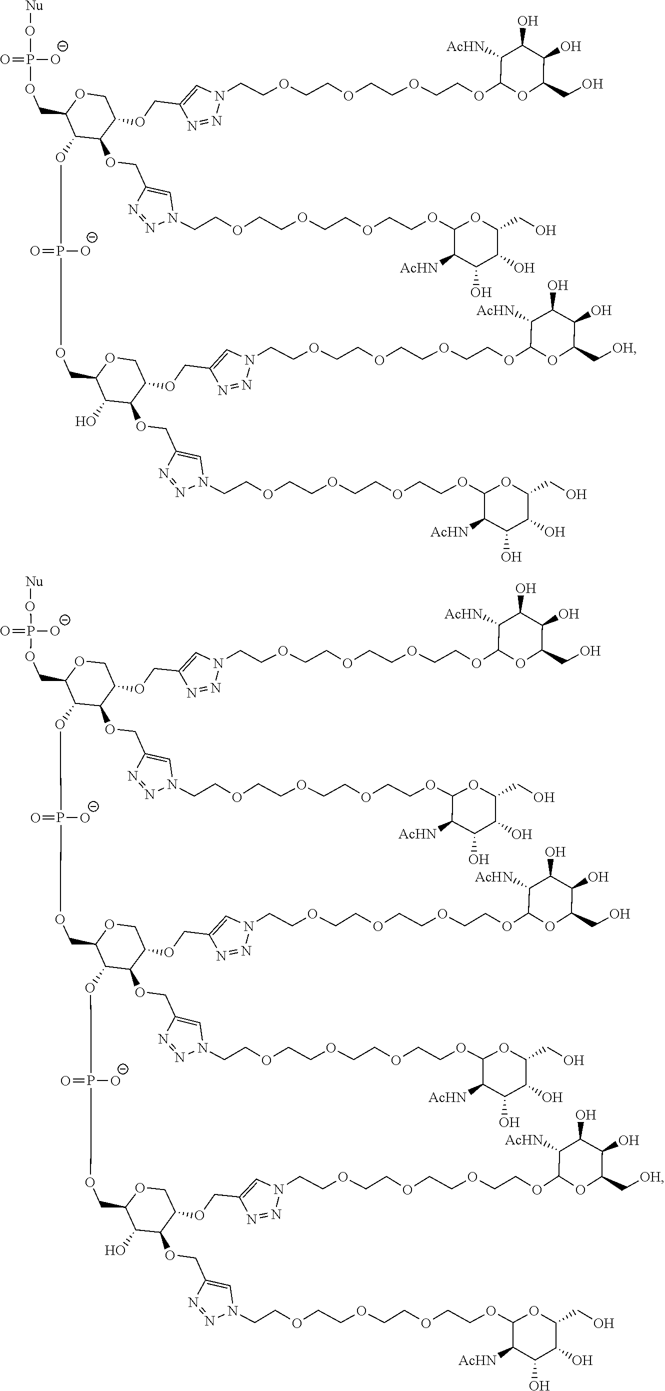

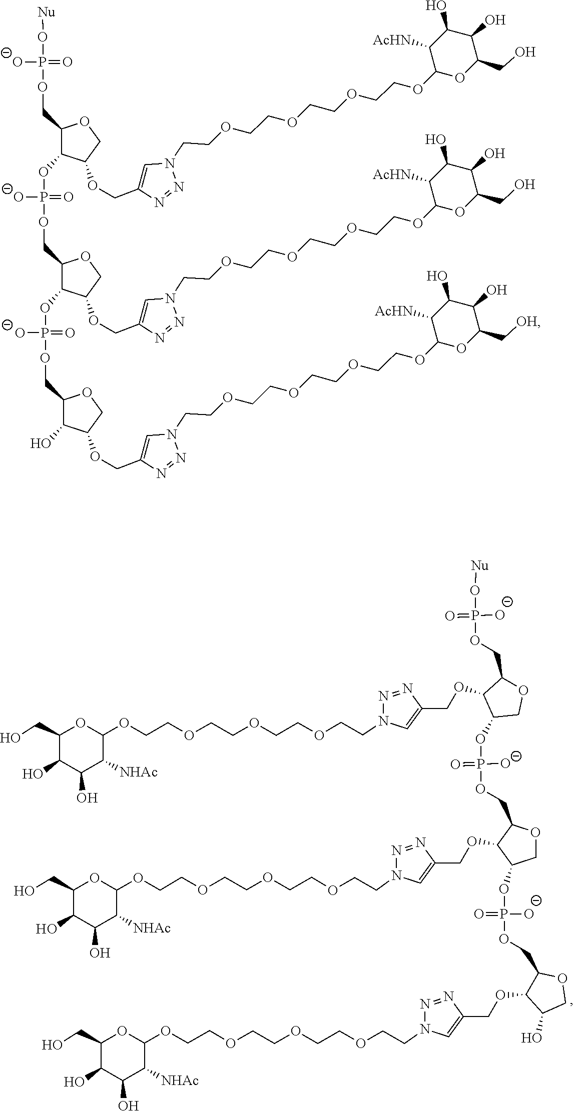

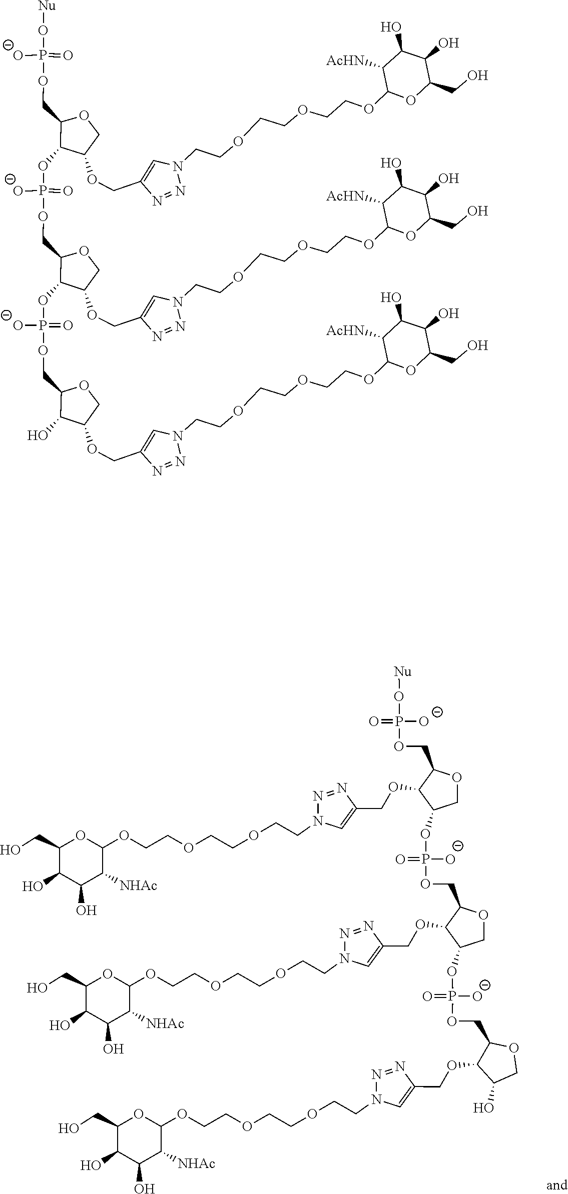

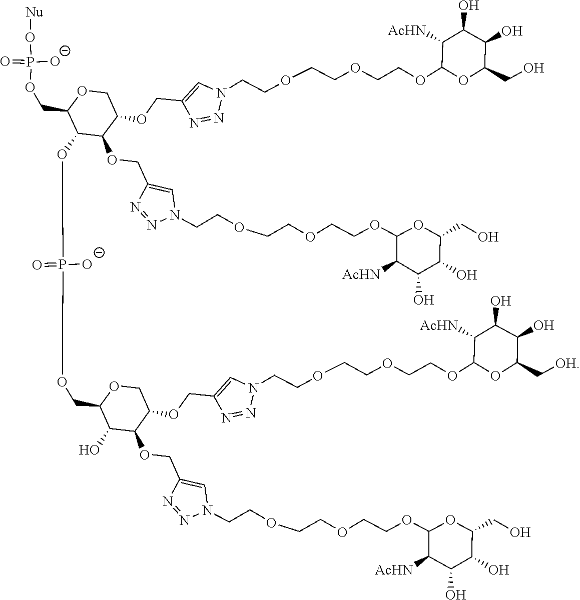

In a third aspect of the present disclosure, the present disclosure further provides a composition comprising the conjugate, or the stereoisomer, the pharmaceutically acceptable salt or the prodrug thereof according to the second aspect.

In some optional embodiments of the present disclosure, the composition further comprises optionally one or more pharmaceutically acceptable carriers or excipients.

In a fourth aspect of the present disclosure, the present disclosure further provides use of (I), (II) and/or (III) in the manufacture of a medicament for preventing and/or treating a disease:
(I) the compound, or the stereoisomer, the pharmaceutically acceptable salt or the prodrug thereof according to the first aspect,
(II) the conjugate, or the stereoisomer, the pharmaceutically acceptable salt or the prodrug thereof according to the second aspect, and/or
(III) the composition according to the third aspect.

In some embodiments of the present disclosure, the disease is a pathological condition or disease caused by abnormal expression of a target gene in target cells.

In some embodiments of the present disclosure, the disease is a hepatogenic disease.

In a fifth aspect of the present disclosure, the present disclosure further provides use of (I), (II) and/or (III) in the manufacture of a medicament for reducing expression or activity of a target gene:
(I) the compound, or the stereoisomer, the pharmaceutically acceptable salt or the prodrug thereof according to the first aspect,
(II) the conjugate, or the stereoisomer, the pharmaceutically acceptable salt or the prodrug thereof according to the second aspect, and/or
(III) the composition according to the third aspect.

In a sixth aspect of the present disclosure, the present disclosure further provides a medicament comprising (I), (II) and/or (III), and a pharmaceutically acceptable excipient or adjuvant:
(I) the compound, or the stereoisomer, the pharmaceutically acceptable salt or the prodrug thereof according to the first aspect,
(II) the conjugate, or the stereoisomer, the pharmaceutically acceptable salt or the prodrug thereof according to the second aspect, and/or
(III) the composition according to the third aspect.

In a seventh aspect of the present disclosure, the present disclosure further provides a pharmaceutical combination comprising the medicament according to the sixth aspect and any additional active ingredient.

In an eighth aspect of the present disclosure, the present disclosure further provides a method for treating and/or preventing a disease in a subject in need thereof, comprising administering to the subject a pharmaceutically acceptable amount of:
(I) the conjugate, or the stereoisomer, the pharmaceutically acceptable salt or the prodrug thereof according to the second aspect, and/or
(II) the composition according to the third aspect, and/or
(III) the medicament according to the sixth aspect, and/or
(IV) the pharmaceutical combination according to the seventh aspect.

In some specific embodiments of the present disclosure, the disease is a pathological condition or disease caused by abnormal expression of a target gene in target cells.

In a ninth aspect of the present disclosure, the present disclosure further provides a method for reducing expression or activity of a target gene, comprising contacting (I), (II) (III) and/or (IV) with a cell:
(I) the conjugate, or the stereoisomer, the pharmaceutically acceptable salt or the prodrug thereof according to the second aspect, and/or
(II) the composition according to the third aspect, and/or
(III) the medicament according to the sixth aspect, and/or
(IV) the pharmaceutical combination according to the seventh aspect.

In a tenth aspect of the present disclosure, the present disclosure further provides a compound represented by formula (Ic), or a stereoisomer thereof.

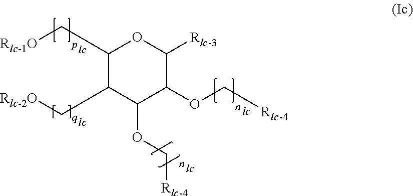

in formula (Ic), p_{Ic} and q_{Ic} are each independently selected from the group consisting of 0, 1, 2 and 3;
each n_{Ic} is independently selected from the group consisting of 1, 2, 3, 4 and 5;
R_{Ic-1} is selected from the group consisting of H and R_{Ic-1}′, and R_{Ic-1}′ is a hydroxyl protecting group;
R_{Ic-2} is selected from the group consisting of H and R_{Ic-2}′, and R_{Ic-2}′ is

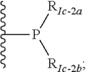

wherein R_{Ic-2a} is a secondary amino group, and R_{Ic-2b} is a secondary amino group or a C₁-C₃ alkoxy substituted with a cyano;
R_{Ic-3} is selected from the group consisting of H and C₁-C₃ alkoxy; and
each R_{Ic-4} is independently selected from the group consisting of

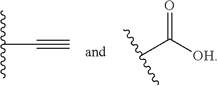

In some embodiments of the present disclosure, each R_{Ic-4} is

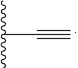 [SMILES: *C#C]

In some embodiments of the present disclosure, each R_{Ic-4} is

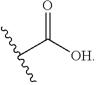 [SMILES: *C(=O)O]

In some embodiments of the present disclosure, p_{Ic} and q_{Ic} are each independently selected from the group consisting of 0 and 1, for example p_{Ic}=1 and q_{Ic}=1, p_{Ic}=1 and q_{Ic}=0, p_{Ic}=0 and q_{Ic}=1, or p_{Ic}=0 and q_{Ic}=0.

In some embodiments of the present disclosure, p_{Ic}=1 and q_{Ic}=0.

In some embodiments of the present disclosure, each n is 1, each n is 2, each n is 3, each n is 4, or each n is 5.

In some embodiments of the present disclosure, each n_{Ic} is 1.

Exemplarily, when p=1, q=0 and n=1, the compound represented by formula (Ic) has a structure of

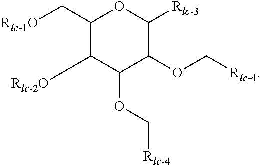

In some embodiments of the present disclosure, the hydroxyl protecting group has a structure of

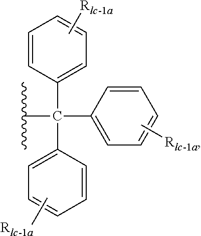

wherein each R_{Ic-1a} is independently selected from the group consisting of H, C₁-C₃ alkyl and C₁-C₃ alkoxy.

In some embodiments of the present disclosure, each R_{Ic-1a} is independently selected from the group consisting of H and C₁-C₃ alkoxy.

In some embodiments of the present disclosure, each R_{Ic-1a} is independently selected from the group consisting of H and methoxy.

In some embodiments of the present disclosure, the hydroxyl protecting group is selected from the group consisting of trityl (Tr), 4-methoxytrityl (MMTr), 4,4′-dimethoxytrityl (DMTr) and 4,4′,4″-trimethoxytrityl (TMTr).

In some embodiments of the present disclosure, the hydroxyl protecting group is 4,4′-dimethoxytrityl (DMTr).

In some embodiments of the present disclosure, R_{Ic-2a} is

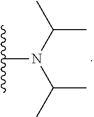 [SMILES: *N(C(C)C)C(C)C]

In some embodiments of the present disclosure, R_{Ic-2b} is selected from the group consisting of

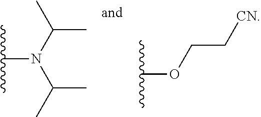

In some embodiments of the present disclosure, R_{Ic-2b} is

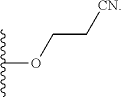 [SMILES: *OCCC#N]

In some embodiments of the present disclosure,

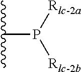

is selected from the group consisting of

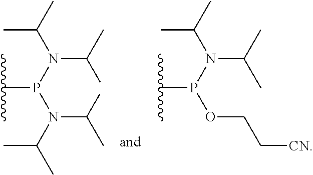 [SMILES: *P(N(C(C)C)C(C)C)N(C(C)C)C(C)C ; *P(OCCC#N)N(C(C)C)C(C)C]

In some embodiments of the present disclosure,

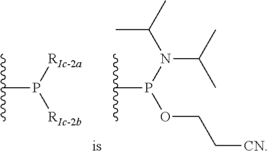

In some embodiments of the present disclosure, R_{Ic-2} is selected from the group consisting of H,

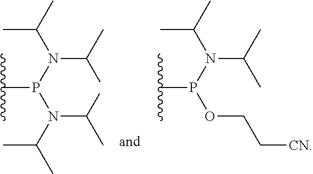 [SMILES: *P(N(C(C)C)C(C)C)N(C(C)C)C(C)C ; *P(OCCC#N)N(C(C)C)C(C)C]

In some embodiments of the present disclosure, R_{Ic-2} is selected from the group consisting of H and

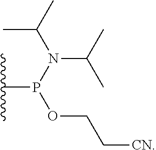 [SMILES: *P(OCCC#N)N(C(C)C)C(C)C]

In some embodiments of the present disclosure, R_{Ic-2} is H.

In some embodiments of the present disclosure, R_{Ic-2} is

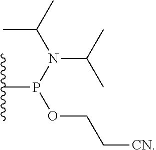 [SMILES: *P(OCCC#N)N(C(C)C)C(C)C]

In some embodiments of the present disclosure, the compound has a structure selected from the group consisting of:

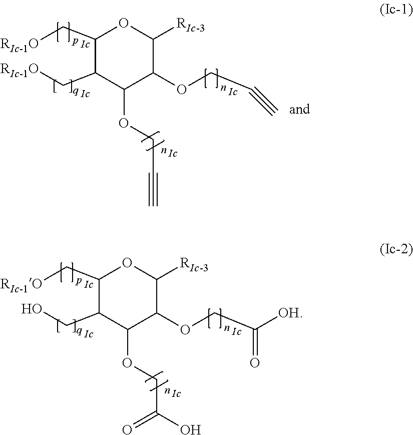

In some embodiments of the present disclosure, the compound has a structure selected from the group consisting of:

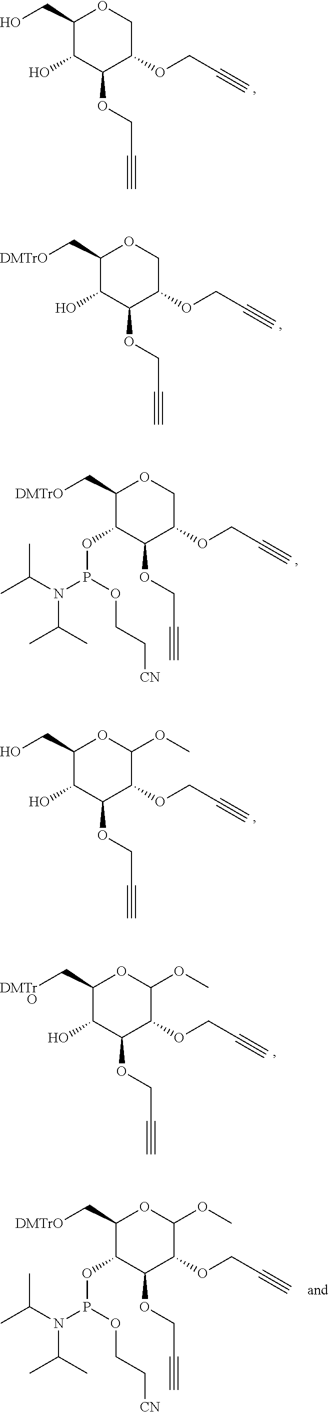

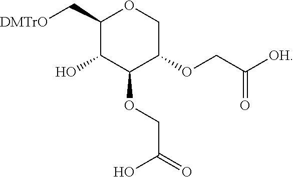

In an eleventh aspect of the present disclosure, the present disclosure further provides use of the compound or the stereoisomer thereof according to the tenth aspect of the present disclosure in the manufacture of a targeting ligand capable of binding to a cell surface receptor.

In some embodiments of the present disclosure, the cell surface receptor is an asialoglycoprotein receptor.

Exemplarily, the targeting ligand is selected from the group consisting of

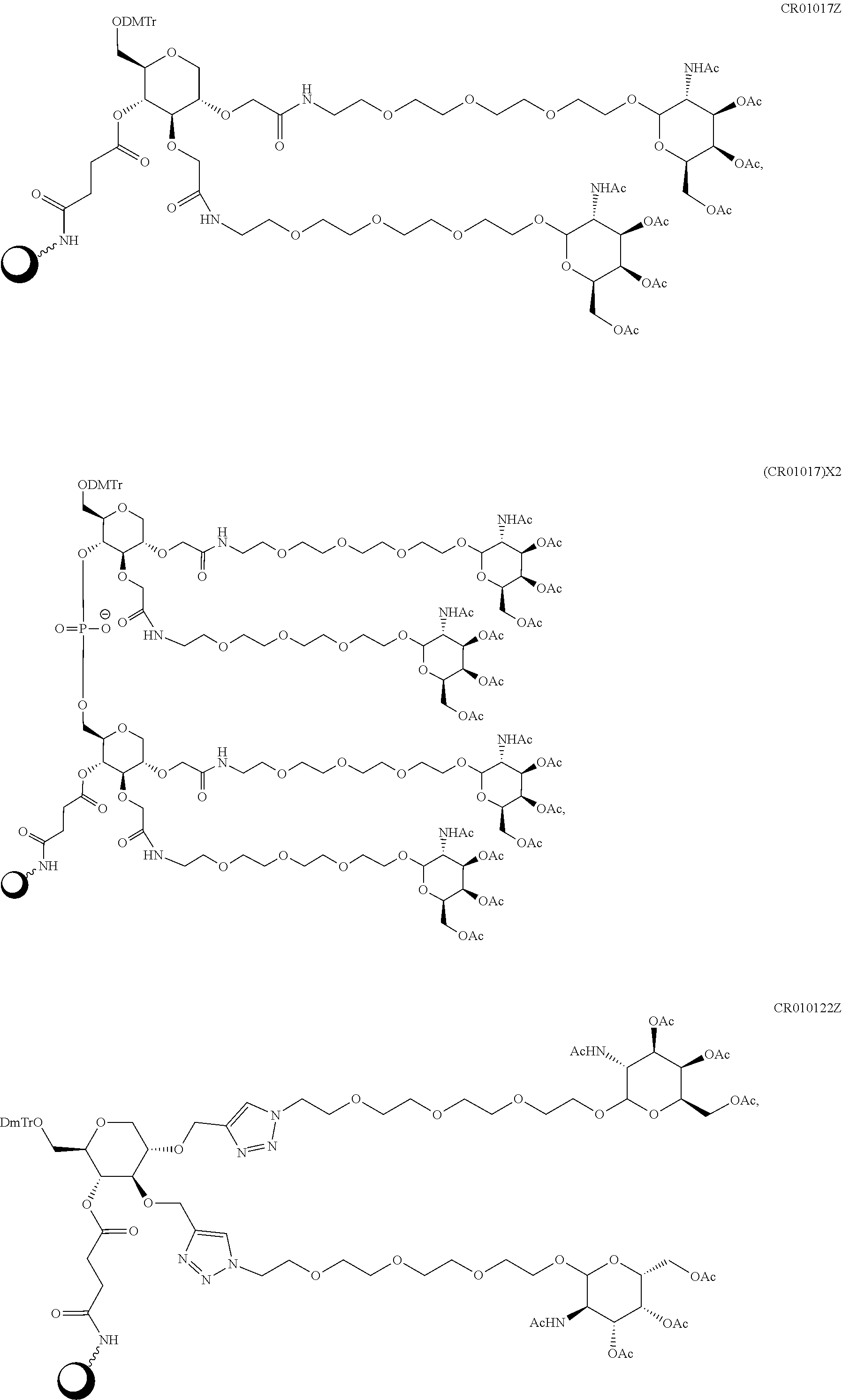

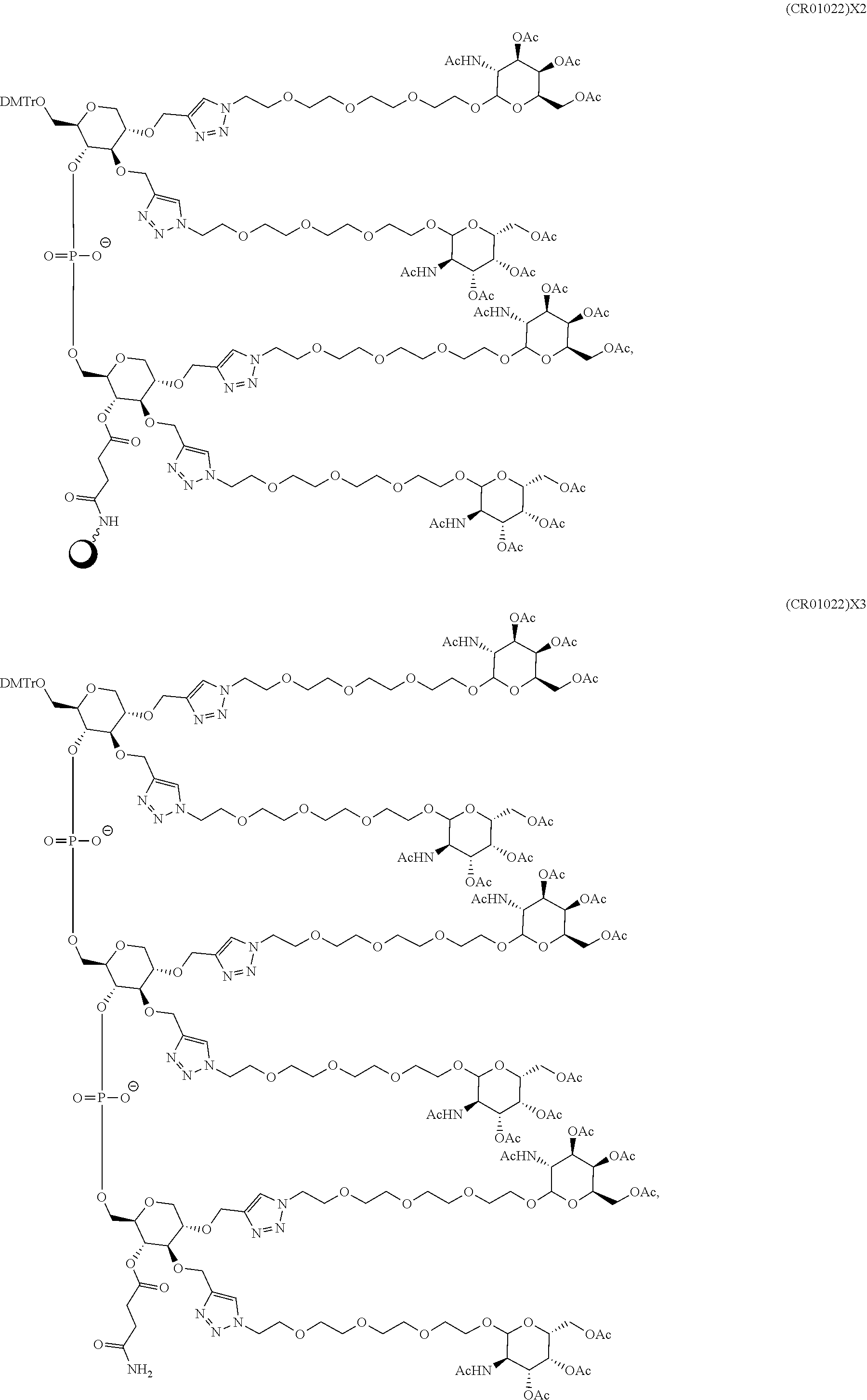

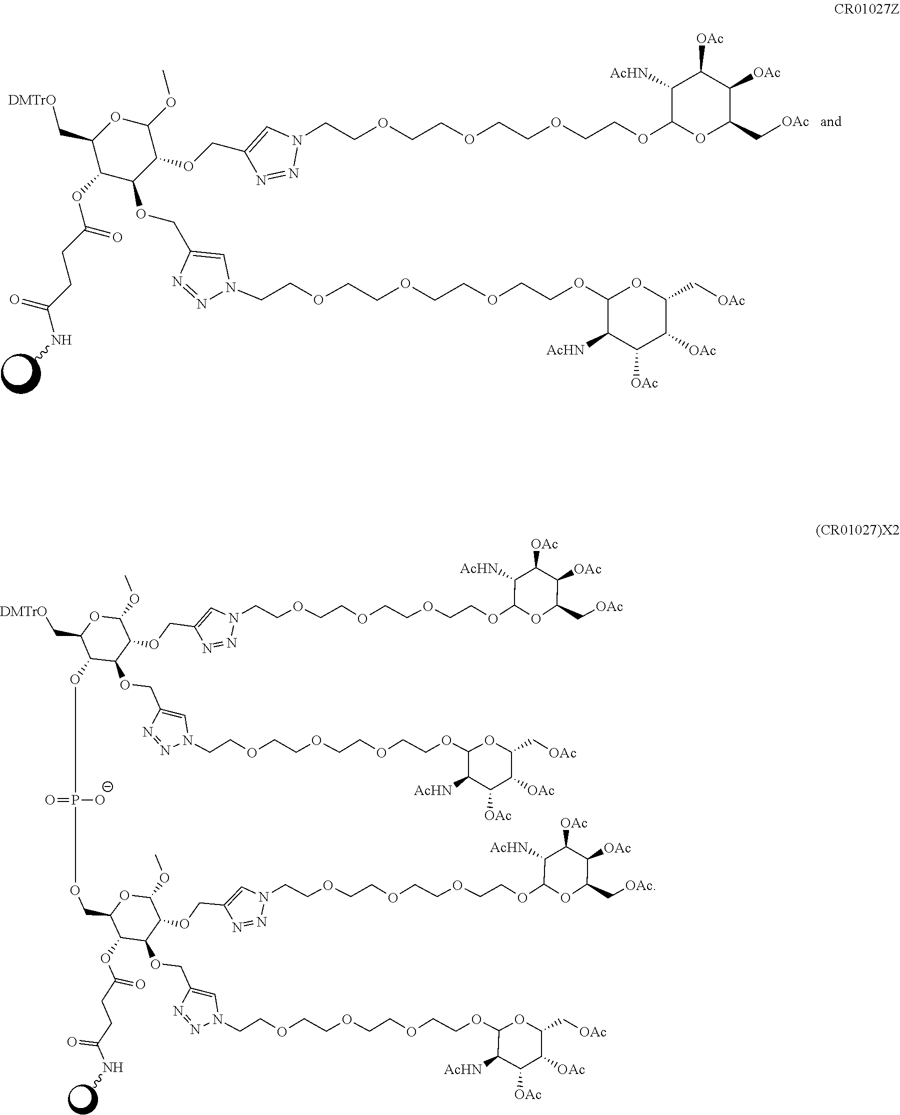

In a twelfth aspect of the present disclosure, the present disclosure further provides use of the compound or the stereoisomer thereof according to the tenth aspect of the present disclosure in the manufacture of a medicament for reducing expression or activity of a target gene in a target cell.

In some embodiments of the present disclosure, the target cell is a hepatocyte.

In some embodiments of the present disclosure, the medicament is a RNAi drug.

In some embodiments of the present disclosure, the medicament is a siRNA drug.

Exemplarily, the siRNA drug is selected from the group consisting of

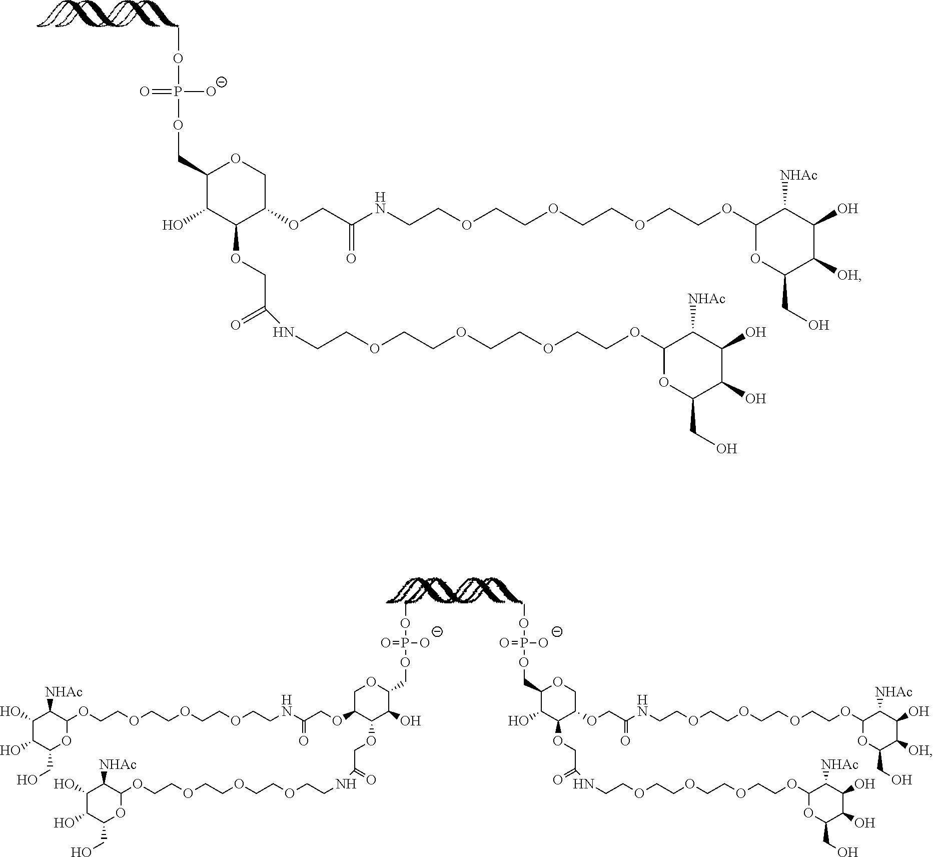

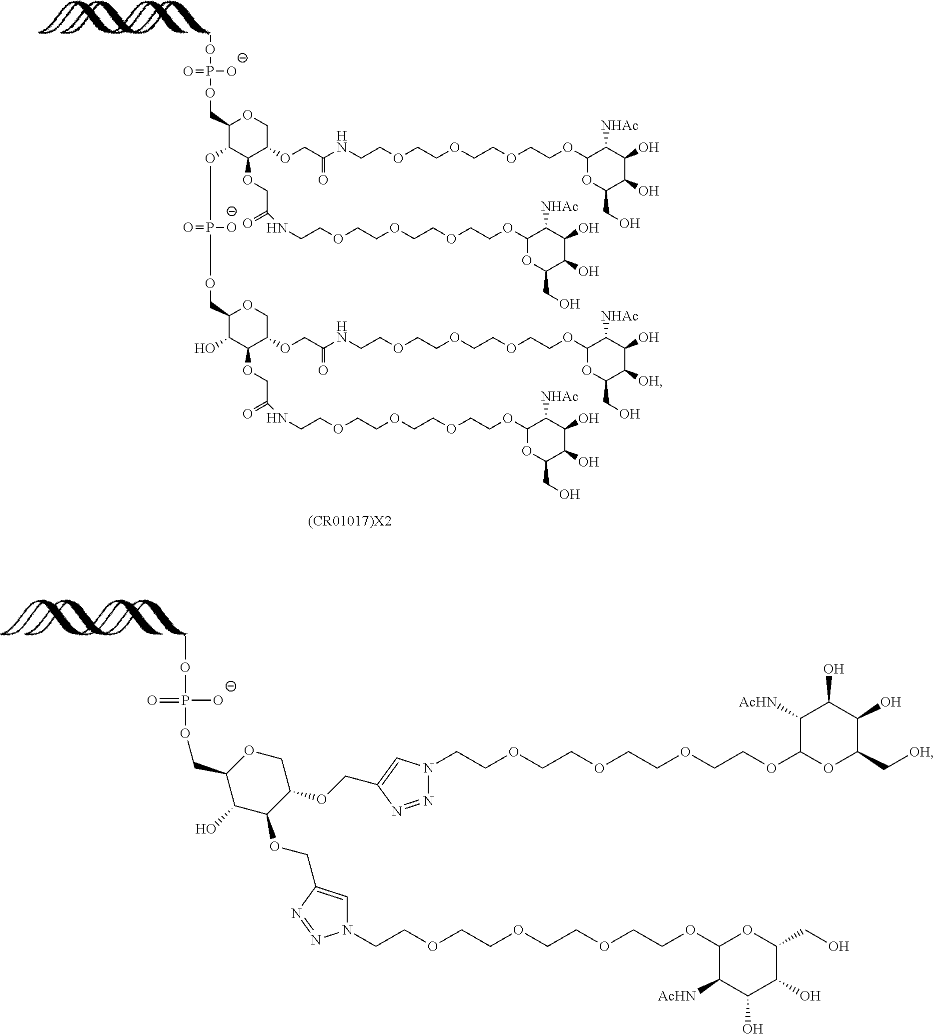

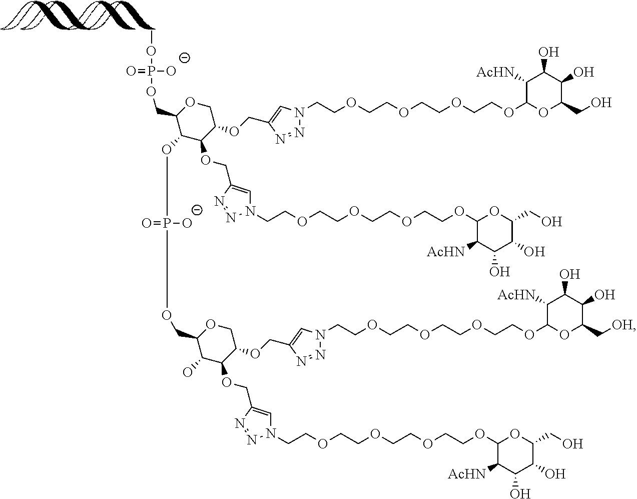

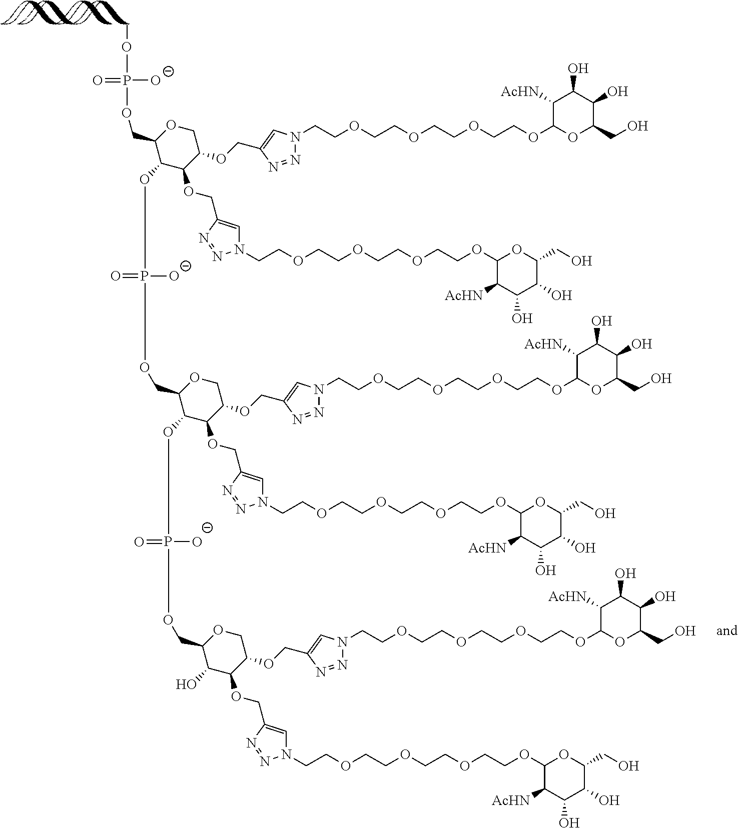

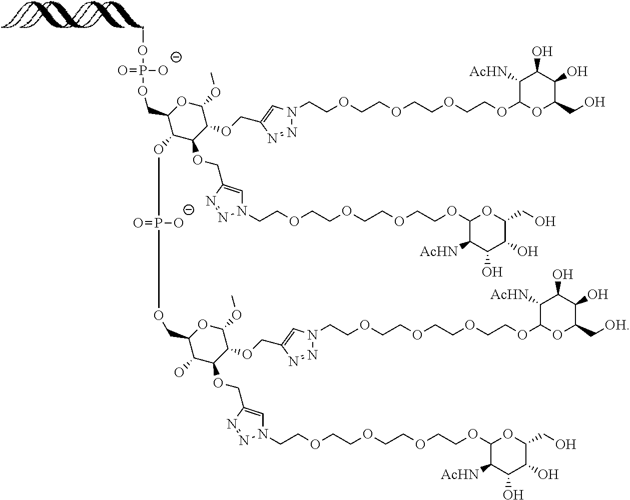

Wherein, [symbol] represents siRNA.

Wherein, when the siRNA drug comprises one targeting ligand, the targeting ligand is conjugated to the 3′ end of the sense strand. When the siRNA drug comprises two targeting ligands, the two targeting ligands are conjugated to the 3′ end and 5′ end of the sense strand, respectively.

In the thirteenth aspect of the present disclosure, the present disclosure further provides a method for producing a compound represented by formula (Ic-1a), formula (Ic-1b) or formula (Ic-1c), or a stereoisomer thereof:

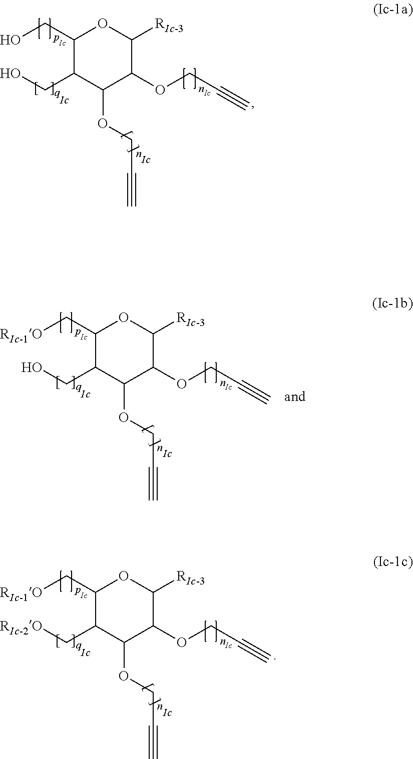

Wherein, R_{Ic-1}′, R_{Ic-2}′, R_{Ic-3}, p_{Ic}, q_{Ic}, and n_{Ic} are as defined in the tenth aspect of the present disclosure.

The method comprises the following steps:

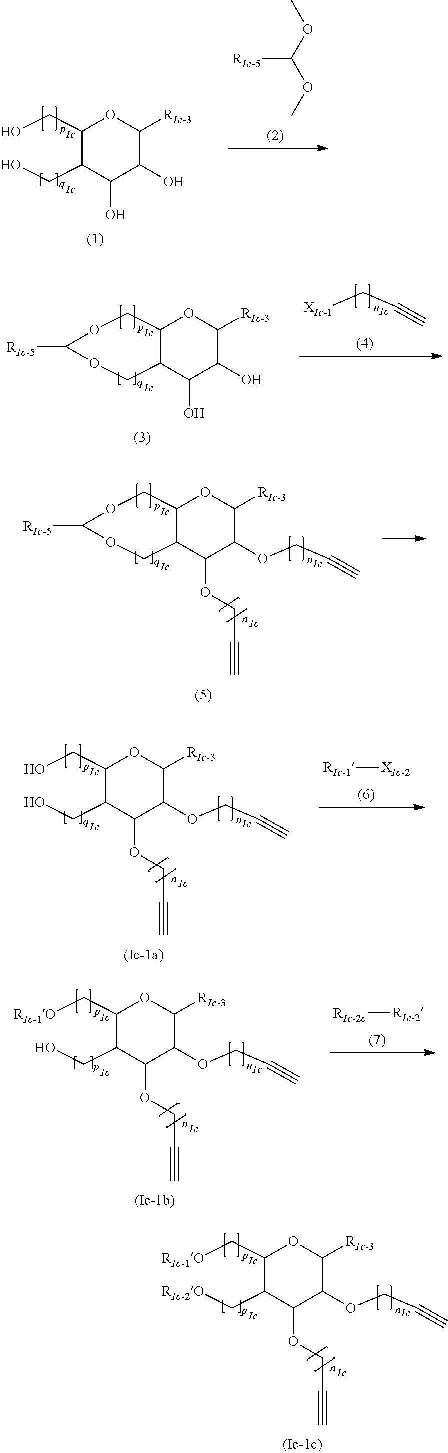

S1-1: subjecting a compound represented by formula (1) and a compound represented by formula (2) to an aldolization reaction in the presence of an acidic catalyst to obtain a compound represented by formula (3), wherein R_{Ic-5} is a phenyl (Ph group);
S1-2: subjecting the compound represented by formula (3), a hydrogenated salt and a compound represented by formula (4) to an electrophilic substitution reaction to obtain a compound represented by formula (5), wherein X_{Ic-1} represents a halogen;
S1-3: subjecting the compound represented by formula (5) to a deprotection of hydroxyl reaction under an acidic condition to obtain a compound represented by formula (Ic-1a);
S1-4: subjecting the compound represented by formula (Ic-1a) and a compound represented by formula (6) to a nucleophilic substitution reaction to obtain a compound represented by formula (Ic-1b), wherein X_{Ic-2} represents a halogen; and
S1-5: subjecting the compound represented by formula (Ic-1b) and a compound represented by formula (7) to a condensation reaction to obtain a compound of formula (Ic-1c), wherein R_{Ic-2c} is selected from the group consisting of a halogen and a secondary amino group.

In some embodiments of the present disclosure, in step S1-1, the molar ratio of the compound represented by formula (1): the compound represented by formula (2): the acidic catalyst is 1:(2-4):(0.01-0.2), for example 1:(2.5-3.5):(0.01-0.2), 1:(2.8-3.2):(0.01-0.2), 1:3:(0.01-0.2), 1:(2-4):(0.05-0.15), 1:(2-4):(0.08-0.12), 1:(2-4):0.1 or 1:3:0.1.

In some embodiments of the present disclosure, in step S1-1, the molar ratio of the compound represented by formula (1): the compound represented by formula (2): the acidic catalyst is 1:3:0.1.

In some embodiments of the present disclosure, in step S1-1, the acidic catalyst is a sulfonic acid catalyst.

In some embodiments of the present disclosure, in step S1-1, the acidic catalyst is DL-10-camphorsulfonic acid (CAS No. 5872-08-2).

In some embodiments of the present disclosure, in step S1-1, the aldolization reaction is carried out at a temperature ranging from 20 °C to 40 °C, for example, 25 °C, 30 °C or 35 °C.

In some embodiments of the present disclosure, in step S1-1, the aldolization reaction is carried out for 4-10 h, for example 4.5 h, 5 h, 5.5 h, 6 h, 6.5 h, 7 h, 7.5 h, 8 h, 8.5 h, 9 h or 9.5 h.

In some embodiments of the present disclosure, in step S1-1, a reaction solvent used in the aldolization reaction is a polar aprotic solvent, for example acetonitrile.

In some embodiments of the present disclosure, in step S1-1, R_{Ic-5} is phenyl (Ph group).

In some embodiments of the present disclosure, in step S1-2, the molar ratio of the compound represented by formula (3): the compound represented by formula (4): the catalyst is 1:(3-5):(3-5), for example 1:(3.5-4.5):(3-5), 1:(3.8-4.2):(3-5), 1:4:(3-5), 1:(3-5):(3.5-4.5), 1:(3-5):(3.8-4.2), 1:(3-5):4 or 1:4:4.

In some embodiments of the present disclosure, in step S1-2, the molar ratio of the compound represented by formula (3): the compound represented by formula (4): the catalyst is 1:4:4.

In some embodiments of the present disclosure, in step S1-2, the hydrogenated salt is selected from the group consisting of sodium hydride, potassium hydride and magnesium hydride.

In some embodiments of the present disclosure, in step S1-2, the hydrogenated salt is sodium hydride.

In some embodiments of the present disclosure, in step S1-2, the electrophilic substitution reaction is carried out by reacting the compound represented by formula (3) and the hydrogenated salt at −10 °C to 0 °C (for example −9 °C, −8 °C, −7 °C, −6 °C, −5 °C, −4 °C, −3 °C, −2 °C or −1 °C) for 25-35 min (for example 30 min), adding the compound represented by formula (4), and performing a reaction at 20-40 °C (for example 25 °C, 30 °C or 35 °C) for 1-5 h (for example 1.5 h, 2 h, 2.5 h, 3 h, 3.5 h, 4 h or 4.5 h).

In some embodiments of the present disclosure, in step S1-2, a reaction solvent used in the electrophilic substitution reaction is a polar aprotic solvent, for example N,N-dimethylformamide (DMF).

In some embodiments of the present disclosure, in step S1-2, X_{Ic-1} is Br.

In some embodiments of the present disclosure, in step S1-3, the acidic condition is provided by an organic acid. Wherein the organic acid may be selected from the group consisting of formic acid (CAS No. 64-18-6), acetic acid (CAS No. 64-19-7), propionic acid (CAS No. 79-09-4), butyric acid (CAS No. 107-92-6), and valeric acid (CAS No. 109-52-4).

In some embodiments of the present disclosure, in step S1-3, the acidic condition is provided by acetic acid. Specifically, it is 50%-80% by mass acetic acid aqueous solution, and more specifically, it is 70% by mass acetic acid aqueous solution.

In some embodiments of the present disclosure, in step S1-3, the deprotection of hydroxyl reaction is carried out at a temperature ranging from 60 °C to 80 °C, for example 65 °C, 70 °C or 75 °C.

In some embodiments of the present disclosure, in step S1-3, a reaction solvent used in the deprotection of hydroxyl reaction is a polar aprotic solvent, for example dichloromethane (DCM).

In some embodiments of the present disclosure, in step S1-4, the molar ratio of the compound represented by formula (I-1a) to the compound represented by formula (6) is 1:(1.1-1.5), for example 1:1.15, 1:1.2, 1:1.25, 1:1.3, 1:1.35, 1:1.4 or 1:1.45.

In some embodiments of the present disclosure, in step S1-4, the compound represented by formula (6) is selected from the group consisting of trityl chloride (also known as triphenyl chloromethane, abbreviated as Trt-Cl, CAS No. 76-83-5), trityl bromide (CAS No. 596-43-0), 4-methyltrityl chloride (also known as 4-methyltriphenyl chloromethane, CAS No. 23429-44-9), 4-methoxytrityl chloride (also known as 4-methoxytriphenyl chloromethane (MMT-Cl), CAS No. 14470-28-1), 4,4′-dimethoxytrityl chloride (also known as 4,4′-dimethoxytriphenyl chloromethane, abbreviated as DMT-Cl, CAS No. 40615-36-9) and 4,4′,4″-trimethoxytrityl chloride (CAS No. 49757-42-8).

In some embodiments of the present disclosure, in step S1-4, the compound represented by formula (6) is 4,4′-dimethoxytrityl chloride.

In some embodiments of the present disclosure, in step S1-4, the nucleophilic substitution reaction is carried out in an inert atmosphere, for example nitrogen or helium.

In some embodiments of the present disclosure, in step S1-4, the nucleophilic substitution reaction is carried out at a temperature ranging from 20 °C to 40 °C, for example 25 °C, 30 °C or 35 °C.

In some embodiments of the present disclosure, in step S1-4, the nucleophilic substitution reaction is carried out for 2-5 h, for example 2.5 h, 3 h, 3.5 h, 4 h or 4.5 h.

In some embodiments of the present disclosure, in step S1-4, X_{Ic-2} is Br.

In some embodiments of the present disclosure, in step S1-5, the molar ratio of the compound represented by formula (I-1b) to the compound represented by formula (7) is 1:(0.6-0.9), for example 1:0.65, 1:0.7, 1:0.71, 1:0.72, 1:0.73, 1:0.74, 1:0.75, 1:0.76, 1:0.77, 1:0.78, 1:0.79, 1:0.8 or 1:0.85.

In some embodiments of the present disclosure, in step S1-5, the molar ratio of the compound represented by formula (I-1b) to the compound represented by formula (7) is 1.5:1.1 (approximately 1:0.73).

In some embodiments of the present disclosure, in step S1-5, the compound represented by formula (7) is selected from the group consisting of 2-cyanoethyl-N,N-diisopropylchlorophosphoramidite (CAS No. 89992-70-1), bis (diisopropylamino) chlorophosphine and bis(diisopropylamino)(2-cyanoethoxy)phosphine (CAS No. 102691-36-1).

In some embodiments of the present disclosure, in step S1-5, the compound represented by formula (7) is 2-cyanoethyl-N,N-diisopropylchlorophosphoramidite.

In some embodiments of the present disclosure, in step S1-5, R_{Ic-2c} is halogen.

In some embodiments of the present disclosure, in step S1-5, R_{Ic-2c} is chlorine (Cl).

In some embodiments of the present disclosure, in step S1-5, the condensation reaction is carried out in an inert atmosphere.

In some embodiments of the present disclosure, in step S1-5, a reaction solvent used in the condensation reaction is a polar aprotic solvent, for example dichloromethane.

When R_{Ic-3} is H, the compound represented by formula (1) has a structure of

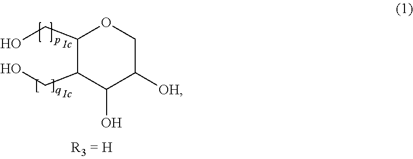

wherein the compound represented by formula (1) with H as R_{Ic-3} is commercially available but relatively expensive. To reduce costs, the compound represented by formula (1) with H as R_{Ic-3} can be prepared by the following method, which comprises the following steps:

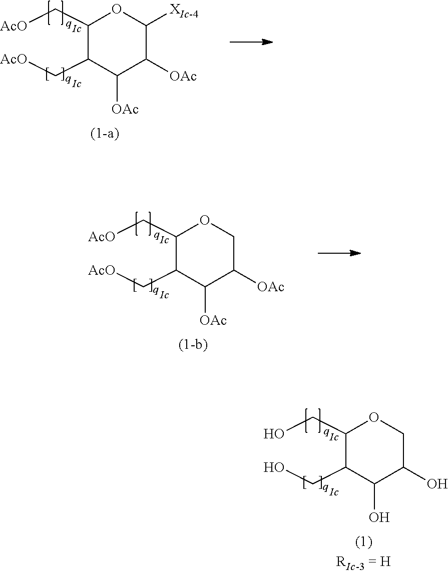

step 1: subjecting a compound represented by formula (1-a) to a hydro-dehalogenation reaction in the presence of a reducing agent and a free radical initiator to obtain a compound represented by formula (1-b), wherein X_{Ic-4} represents a halogen (e.g., F, Cl, Br or I); and
step 2: subjecting the compound represented by formula (1-b) to a hydrolysis reaction to obtain a compound represented by formula (1), wherein R_{Ic-3} is H.

In some embodiments of the present disclosure, in step 1, the molar ratio of the compound represented by formula (1-a): the reducing agent: the free radical initiator is 1:(0.9-1.2):(0.01-0.2), for example 1:(0.95-1.2):(0.01-0.2), 1:(0.95-1.1):(0.01-0.2), 1:(0.98-1.05):(0.01-0.2), 1:(0.98-1.02):(0.01-0.2), 1:1:(0.01-0.2), 1:(0.9-1.2):(0.05-0.15), 1:(0.9-1.2):(0.08-0.12), 1:(0.9-1.2):0.1 or 1:1:0.1.

In some embodiments of the present disclosure, in step 1, the reducing agent is tributyltin hydride.

In some embodiments of the present disclosure, in step 1, the free radical initiator is an azo-initiator. Wherein the azo-initiator may be azobisisobutyronitrile.

In some embodiments of the present disclosure, in step 1, the hydro-dehalogenation reaction is carried out at a temperature ranging from 100 °C to 120 °C, for example 105 °C, 110 °C or 115 °C.

In some embodiments of the present disclosure, in step 1, a reaction solvent used in the hydro-dehalogenation reaction is an aprotic solvent, for example methylbenzene.

In some embodiments of the present disclosure, in step 1, the hydro-dehalogenation reaction is carried out for 1-5 h, for example 1.5 h, 2 h, 2.5 h, 3 h, 3.5 h, 4 h or 4.5 h.

In some embodiments of the present disclosure, in step 2, the hydrolysis reaction is alkaline hydrolysis reaction.

In some embodiments of the present disclosure, in step 2, the alkaline hydrolysis reaction is carried out using a C₁-C₃ alkoxide and C₁-C₃ alcohol.

In some embodiments of the present disclosure, the C₁-C₃ alkoxide is selected from the group consisting of lithium methoxide, sodium methoxide, potassium methoxide, rubidium methoxide, cesium methoxide, lithium ethoxide, sodium ethoxide, potassium ethoxide, sodium n-propoxide, potassium n-propoxide, sodium isopropoxide and potassium isopropoxide.

In some embodiments of the present disclosure, the C₁-C₃ alkoxide is lithium methoxide.

In some embodiments of the present disclosure, the C₁-C₃ alcohol is selected from the group consisting of methanol, ethanol, n-propanol and isopropanol.

In some embodiments of the present disclosure, the C₁-C₃ alcohol is methanol.

In some embodiments of the present disclosure, in step 2, the molar ratio of the compound represented by formula (1-b) to the alkoxide is 1:(0.05-0.2), for example 1:0.06, 1:0.07, 1:0.08, 1:0.09, 1:0.1, 1:0.11, 1:0.12, 1:0.13, 1:0.14, 1:0.15, 1:0.16, 1:0.17, 1:0.18 or 1:0.19.

In a fourteenth aspect of the present disclosure, the present disclosure further provides a method for producing a compound represented by formula (Ic-2) or a stereoisomer thereof,

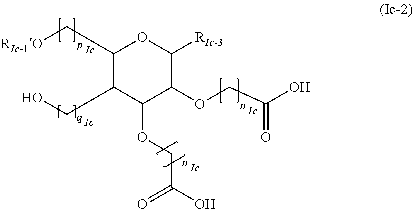

wherein, R_{Ic-1}′, R_{Ic-3}, p_{Ic}, q_{Ic}, and n_{Ic} are as defined in the tenth aspect of the present disclosure.

The method comprises the following steps:

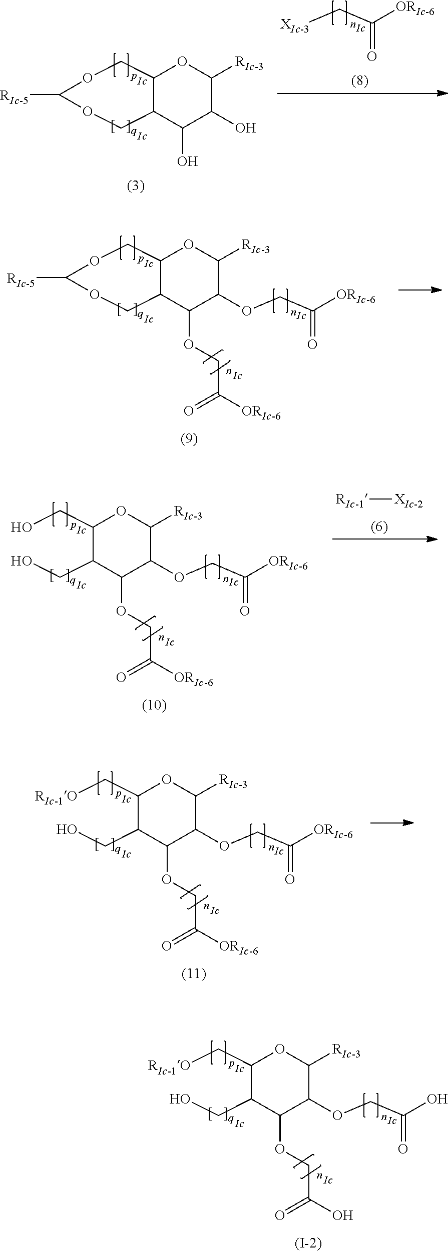

S2-1: subjecting a compound represented by formula (3), a hydrogenated salt, a compound represented by formula (8) and an iodized salt to an electrophilic substitution reaction to obtain a compound represented by formula (9), wherein R₅ is a substituted or unsubstituted C₆-C₁₂ aryl, X_{Ic-3} represents a halogen, and R_{Ic-6} is a substituted or unsubstituted C₁-C₅ alkyl;
S2-2: subjecting the compound represented by formula (9) to a deprotection of hydroxyl reaction under an acidic condition to obtain a compound represented by formula (10);
S2-3: subjecting the compound represented by formula (10) and a compound represented by formula (6) to a nucleophilic substitution reaction to obtain a compound represented by formula (11); and
S2-4: subjecting the compound represented by formula (11) to a hydrolysis reaction to obtain a compound represented by formula (Ic-2).

In some embodiments of the present disclosure, in step S2-1, the molar ratio of the compound represented by formula (3): the hydrogenated salt: the compound represented by formula (8): iodized salt is 1:(3-5):(1.5-2.5):(1-2), for example 1:(3.5-4.5):(1.5-2.5):(1-2), 1:(3.8-4.2):(1.5-2.5):(1-2), 1:4:(1.5-2.5):(1-2), 1:(3-5):(1.8-2.2):(1-2), 1:(3-5):(1.9-2.1):(1-2), 1:(3-5):2:(1-2), 1:(3-5):(1.5-2.5):(1.3-1.7), 1:(3-5):(1.5-2.5):(1.4-1.6), 1:(3-5):(1.5-2.5):1.5 or 1:4:2:1.5.

In some embodiments of the present disclosure, in step S2-1, the molar ratio of the compound represented by formula (3): the hydrogenated salt: the compound represented by formula (8): the iodized salt is 1:4:2:1.5.

In some embodiments of the present disclosure, in step S2-1, the electrophilic substitution reaction is carried out by reacting the compound represented by formula (3) with the hydrogenated salt at −10 °C to 0 °C (for example −9 °C, −8 °C, −7 °C, −6 °C, −5 °C, −4 °C, −3 °C, −2 °C or −1 °C) for 0.5-1.5 h (for example 0.75 h, 1 h or 1.25 h), and adding the compound represented by formula (8) and the iodized salt for 3-5 h (for example 3.5 h, 4 h or 4.5 h) of reaction at 20 °C-40 °C (for example 25 °C, 30 °C or 35 °C).

In some embodiments of the present disclosure, in step S2-1, R_{Ic-5} is phenyl (Ph group).

In some embodiments of the present disclosure, in step S2-1, R_{Ic-6} is a substituted or unsubstituted C₁-C₃ alkyl.

In some embodiments of the present disclosure, in step S2-1, R_{Ic-6} is ethyl.

In some embodiments of the present disclosure, in step S2-2, the acidic condition is provided by an organic acid. Wherein the organic acid may be selected from the group consisting of formic acid (CAS No. 64-18-6), acetic acid (CAS No. 64-19-7), propionic acid (CAS No. 79-09-4), butyric acid (CAS No. 107-92-6), and valeric acid (CAS No. 109-52-4).

In some embodiments of the present disclosure, in step S2-2, the acidic condition is provided by acetic acid. Specifically, it is 50%-80% by mass acetic acid aqueous solution, and more specifically, it is 70% by mass acetic acid aqueous solution.

In some embodiments of the present disclosure, in step 52-2, the deprotection of hydroxyl reaction is carried out at a temperature ranging from 40 °C to 60 °C, for example 45 °C, 50 °C or 55 °C.

In some embodiments of the present disclosure, in step S2-3, the molar ratio of the compound represented by formula (10) to the compound represented by formula (6) is 1:(1.1-1.5), for example 1:1.15, 1:1.2, 1:1.25, 1:1.3, 1:1.35, 1:1.4 or 1:1.45.

In some embodiments of the present disclosure, in step S2-3, the molar ratio of the compound represented by formula (10) to the compound represented by formula (6) is 1:1.3.

In some embodiments of the present disclosure, in step S2-3, the nucleophilic substitution reaction is carried out at a temperature ranging from 20 °C-40 °C, for example 25 °C, 30 °C or 35 °C.

In some embodiments of the present disclosure, in step S2-4, the hydrolysis reaction is an alkaline hydrolysis reaction.

In some embodiments of the present disclosure, in step S2-4, the hydrolysis reaction is carried out using 0.5-1.5 mol/L NaOH aqueous solution, for example 0.8 mol/L NaOH aqueous solution, 1 mol/L NaOH aqueous solution or 1.2 mol/L NaOH aqueous solution.

In some embodiments of the present disclosure, in step S2-4, the hydrolysis reaction is carried out at a temperature ranging from 20 °C-40 °C, for example 25 °C, 30 °C or 35 °C.

The beneficial effects of the present disclosure are as follows.

The present disclosure provides a compound, conjugate, composition, and use thereof. The present disclosure provides an oligonucleotide conjugate produced by conjugating the compound provided by the present disclosure to a specific oligonucleotide (e.g., siRNA). This compound can achieve a highly effectively targeted delivery of the oligonucleotide conjugate to liver cells, and effectively inhibit the expression of a target gene in liver cells. It can be used for the treatment and/or prevention of pathological conditions or diseases caused by abnormal expression of a target gene in liver cells, exhibiting higher in vivo activity and more stable and longer lasting efficiency than those of control carrier. It is supposed to have good safety profile and low toxicity at animal level.

### BRIEF DESCRIPTION OF DRAWINGS

In order to more clearly illustrate the technical solutions in the embodiments of the present disclosure or the prior art, the drawings required for describing the embodiments of the present disclosure or the prior art will be briefly introduced below.

FIG. 1 shows the relative expression levels of the target gene in mice after administration of the siRNA conjugate of Example 1.

FIG. 2 shows the relative expression levels of the target gene in mice after administration of the siRNA conjugate of Example 2.

FIG. 3 shows the relative expression levels of the target gene in mice after administration of the siRNA conjugate of Example 3.

FIG. 4 shows the relative expression levels of the target gene in mice after administration of the siRNA conjugate of Example 4.

FIG. 5 shows the relative expression levels of the target gene in mice after administration of the siRNA conjugate of Example 5.

FIG. 6 shows the relative expression levels of the target gene in mice after administration of the siRNA conjugate of Example 6.

FIG. 7 shows the relative expression levels of the target gene in mice after administration of the siRNA conjugate of Example 7.

FIG. 8 shows the relative expression levels of the target gene in mice after administration of the siRNA conjugate of Example 8.

FIG. 9 shows the relative expression levels of the target gene in mice after administration of the siRNA conjugate of Example 9.

FIG. 10 shows the relative expression levels of the target gene in mice after administration of the siRNA conjugate of Example 10.

FIG. 11 shows the relative expression levels of the target gene in mice after administration of the siRNA conjugate of Example 11.

FIG. 12 shows the relative expression levels of the target gene in mice after administration of the siRNA conjugate of Example 12.

FIG. 13 shows the relative expression levels of the target gene in mice after administration of the siRNA conjugate of Example 13.

FIG. 14 shows the relative expression levels of the target gene in mice after administration of the siRNA conjugate of Example 14.

FIG. 15 shows the relative expression levels of the target gene in mice after administration of the siRNA conjugate of Example 15.

FIG. 16a shows the results of measurement of AST in blood of animals in each group after administration of the siRNA conjugate of Example 16.

FIG. 16b shows the results of measurement of ALT in blood of animals in each group after administration of the siRNA conjugate of Example 16.

FIG. 16c shows the results of measurement of TBIL in blood of animals in each group after administration of the siRNA conjugate of Example 16.

FIG. 16d shows the results of measurement of Urea in blood of animals in each group after administration of the siRNA conjugate of Example 16.

FIG. 16e shows the results of measurement of Crea in blood of animals in each group after administration of the siRNA conjugate of Example 16.

FIG. 16f shows the results of measurement of TC in blood of animals in each group after administration of the siRNA conjugate of Example 16.

FIG. 16g shows the results of measurement of TG in blood of animals in each group after administration of the siRNA conjugate of Example 16.

FIG. 16h shows the results of measurement of WBC in blood of animals in each group after administration of the siRNA conjugate of Example 16.

FIG. 16i shows the results of measurement of NEUT in blood of animals in each group after administration of the siRNA conjugate of Example 16.

FIG. 16j shows the results of measurement of LYMPH in blood of animals in each group after administration of the siRNA conjugate of Example 16.

FIG. 16k shows the results of histopathological examination on liver tissues of animals in each group after administration of the siRNA conjugate of Example 16.

### DETAILED DESCRIPTION

The present disclosure discloses a compound, conjugate, composition, and use thereof, which can be achieved by those skilled in the art through appropriately improving the process parameters in light of the present disclosure. It should be particularly noted that all similar substitutions and modifications are obvious to those skilled in the art, and they are deemed to be included in the present disclosure. The method and the application of the present disclosure have been described through the preferred embodiments. It is obvious that those skilled in the art can change or appropriately modify and combine the method and application described herein without departing from the content, spirit and scope of the present disclosure to realize and apply the technology of the present disclosure.

The affinity of ASGPR for ligands is primarily influenced by the following factors:
a. Type of terminal sugar of the ligand. ASGPR can recognize ligands with terminal Gal or GalNAc. GalNAc exhibits 10- to 50-fold higher affinity for ASGPR compared to Gal. GalNAc ligands are more likely to evade from recognition by Kupffer cells, thereby targeting hepatocytes more.
b. Position of substitution in sugar molecule: 1-OH, 2-OH (or 2-amino), 3-OH, 4-OH, and 5-CH₂— in Gal (or GalNAc) all participate in binding to the ASGPR receptor. When the 5-CH₂— is connected with a negatively charged group or when the 4-OH, 3-OH, 2-OH (or 2-amino) are substituted, the affinity for receptor is reduced sharply. When 1-OH undergoes glycosylation substitution, the affinity for receptor is reduced in a case that the glycosidic bond is in α-configuration, and the affinity for receptor is not affected in a case that the glycosidic bond is in β-configuration and the substituent has a linear structure. Only 6-OH does not participate in binding to receptor and points to the solvent region, making it suitable as a site for linking carriers and drugs.
c. Spatial distance between the terminal sugar residue and the carrier. In a case that the sugar residue is separated from the carrier by at least 6 CH₂ units, it can be effectively recognized by ASGPR.

Additionally, ligands with different molecular cluster structures and their distinct modes of conjugation to oligonucleotides significantly influence oligonucleotide activity in vivo. Higher activity suggests better therapeutic efficacy or lower dosage requirements. If the therapeutic efficacy is equivalent, a lower dosage also suggests lower toxicity.

Based on the affinity characteristics for the ASGPR receptor, the present disclosure provides a ligand with high affinity for ASGPR to achieve targeted delivery of drugs or genes to liver, thereby regulating the expression of target genes.

### INTERPRETATION OF TERMS

The term “aliphatic ring” means a cyclic carbon skeleton structure. The term “5- to 8-membered aliphatic ring” refers to a single ring structure with 5 to 8 carbon atoms in the cyclic carbon skeleton. The term “saturated aliphatic ring” refers to a cyclic carbon skeleton composed entirely of carbon-carbon single bonds.

The term “5- to 8-membered aliphatic heterocycle” refers to an aliphatic ring in which one or more carbon atoms are replaced by heteroatoms. The term “saturated aliphatic heterocycle” refers to a saturated aliphatic ring in which one or more carbon atoms are replaced by heteroatoms.

The term “chain-like alkyl” refers to a chain-like hydrocarbon group represented by a general formula of

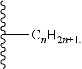

The chain-like alkyl may be straight-chain or branched-chain alkyl. Wherein, the term “C₁-C₆ chain-like alkyl” refers to a chain-like alkyl containing 1 to 6 carbon atoms.

The term “chain-like alkylidene” refers to a chain-like alkylene represented by a general formula of

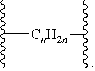

The chain-like alkylidene may be straight-chain or branched-chain alkylidene.

The term “CH₂CH₂” refers to a C₂ straight-chain alkyl (i.e., ethylidene), with a structural formula of

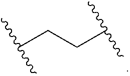 [SMILES: *CC*]

The term “chain-like alkyloxy” refers to

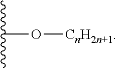

The term “chain-like alkyacyl” refers to

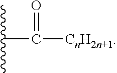

For example, the “C₁-C₆ chain-like alkyacyl” may be a formyl, acetyl, propionyl, n-butanoyl, or iso-butanoyl.

The term “NH” refers to imino, with a structural formula of

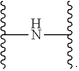 [SMILES: *N*]

The term “CO” refers to carbonyl, with a structural formula of

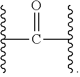 [SMILES: *C(*)=O]

The term “CN” refers to cyano, with a structural formula of

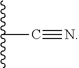 [SMILES: *C#N]

The term “triazole” has a structural formula of

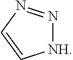

In the context of this disclosure, the term “pharmaceutically acceptable salt” include pharmaceutically acceptable acid addition salts and pharmaceutically acceptable base addition salts.

In the context of this disclosure, the term “pharmaceutically acceptable acid addition salt” means a salt formed with an inorganic or organic acid which is capable of maintaining the bioavailability of the free base without bringing other side effects. Inorganic acid salts include, but are not limited to, hydrochlorides, hydrobromides, sulfates, nitrates, phosphates, and the like. Organic acid salts include, but are not limited to, formate, acetate, 2,2-dichloroacetate, trifluoroacetate, propionate, hexanoate, octoate, decanoate, undecylenate, glycolate, gluconate, lactate, sebacate, adipates, glutaric acid salts, malonates, oxalates, maleates, succinates, fumarates, tartrates, citrates, palmitates, stearates, oleates, cinnamate, laurate, malate, glutamate, pyroglutamate, aspartate, benzoate, methanesulfonate, besylate, p-toluenesulfonate, alginate, ascorbate, salicylate, 4-aminosalicylate, naphthalene disulfonate, and the like. These salts can be prepared by methods known in the art.

In the context of this disclosure, the term “pharmaceutically acceptable base addition salt” means a salt formed with an inorganic or organic base which is capable of maintaining the bioavailability of the free acid without bringing other side effects. Salts derived from inorganic bases include, but are not limited to, sodium salts, potassium salts, lithium salts, ammonium salts, calcium salts, magnesium salts, iron salts, zinc salts, copper salts, manganese salts, aluminum salts, and the like. Preferred inorganic salts are ammonium, sodium, potassium, calcium and magnesium salts, preferably sodium salt. Salts derived from organic bases include, but are not limited to, the following salts: primary amines, secondary amines and tertiary amines, substituted amines, including naturally substituted amines, cyclic amines, and basic ion exchange resins. For example, ammonia, isopropylamine, trimethylamine, diethylamine, triethylamine, tripropylamine, ethanolamine, diethanolamine, triethanolamine, dimethylethanolamine, 2-dimethylaminoethanol, 2-diethylaminoethanol, bicyclo hexylamine, lysine, arginine, histidine, caffeine, procaine, choline, betaine, ethylenediamine, glucosamine, methylglucosamine, theobromine, purine, piperazine, piperidine, N-ethylpiperidine, polyamine resin, and the like. Preferred organic bases include isopropylamine, diethylamine, ethanolamine, trimethylamine, dicyclohexylamine, choline, and caffeine. These salts can be prepared by methods known in the art.

In the context of this disclosure, the term “oligonucleotide” refers to deoxyribonucleic acid (DNA) or ribonucleic acid (RNA), generally consisting of 10-50 nucleotides. The oligonucleotide can regulate gene expression through a number of processes including ribonucleic acid interference, ribonuclease-mediated target degradation, splicing regulation, non-coding RNA inhibition, gene activation, and programmed genome editing.

“ [symbol] ” denotes the site of attachment between groups by a covalent bond.

In the structure formula of the compound or the ligand of the present disclosure, a bond “ [symbol] ” represents an unspecified configuration. If chiral isomerism exist in the chemical structure, the bond “ [symbol] ” may be “ [symbol] ” or “ [symbol] ”, or contains both the configurations of “ [symbol] ” and “ [symbol] ”. Although all of the above structure formulas are drawn as certain isomeric forms for the sake of simplicity, the present disclosure may include all isomers, such as tautomers, rotamers, geometric isomers, diastereomers, racemates and enantiomers.

In the structure formula of the compound or the ligand described herein, a bond “ [symbol] ” is not specified with a configuration; that is, if cis-trans isomerization exists in the chemical structure, it may be in a Z configuration or an E configuration, or contains both configurations of E and Z.

The following definitions used herein should be applied unless otherwise noted. For the purposes of the present disclosure, the chemical elements are consistent with the Periodic Table based on CAS number and Handbook of Chemistry and Physics (75th Edition), 1994. In addition, general principles of organic chemistry may refer to Organic Chemistry by Thomas Sorrell (University Science Books, Sausalito: 1999), and March's Advanced Organic Chemistry by Michael B. Smith and Jerry March (John Wiley & Sons, New York: 2007), the entire contents of which are incorporated herein by reference.

Unless stated otherwise or there is an obvious conflict in context, the articles “a,” “an,” and “the” used herein are intended to include “at least one” or “one or more”. Therefore, these articles used herein refer to articles of one or more (i.e., at least one) objects. For example, “a component” means one or more components, that is, more than one component may be considered to be applied or used in an example of the embodiment.

The term “comprise” is an open-ended expression, and it includes the content specified in the present disclosure, but does not exclude other aspects.

The term “stereoisomer” refers to compounds having the same chemical structure, but different arrangement of atoms or groups in space. The stereoisomer includes enantiomer, diastereomer, conformational isomer (rotamer), geometric isomer (cis/trans) isomers, atropisomer, etc.

The term “chiral” refers to molecules which are non-superimposable on their mirror images, while the term “achiral” refers to molecules which are superimposable on their mirror images.

The “enantiomer” refers to two isomers of a compound which are non-superimposable but are mirror images of each other.

The “diastereomer” refers to stereoisomers which have two or more chiral centers and are not mirror images of each other. Diastereomers have different physical properties, such as melting points, boiling points, spectral properties, and reactivity. Diastereomer mixtures can be separated by high resolution analytical operations, for example, electrophoresis and chromatography, such as HPLC.

The stereochemical definitions and rules used in the present disclosure generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994.

Like those described herein, the compounds of the present disclosure may optionally be substituted with one or more substituents, such as the compounds of the general formula above, or the particular examples in the embodiments, subclasses, and classes of compounds encompassed by the present disclosure.

Generally, the term “substituted” means that one or more hydrogen atoms in a given structure are replaced by specific substituents. Unless otherwise indicated, a substituted group may have a substituent at each substitutable position of the group. When more than one position in a given structure formula can be substituted with one or more substituents selected from specific groups, the substituents at each substitutable position may be the same or different.

The term “unsubstituted” means that the specified group has no substituents.

The term “optionally substituted with . . . ” can be used interchangeably with the term “unsubstituted or substituted with . . . ”, i.e., the structure is unsubstituted or substituted with one or more of the substituents described herein. The substituents described herein include, but are not limited to: D, F, Cl, Br, I, N₃, CN, NO₂, OH, SH, NH₂, alkyl, haloalkyl, haloalkoxy, haloalkylamino, alkenyl, alkynyl, alkoxy, alkylamino, cycloalkyl, heterocyclyl, aryl, heteroaryl and the like.

Further, it is noted that, unless otherwise expressly indicated, the expressions “each . . . is independently”, “ . . . each independently is“and” . . . independently is” used in the present disclosure are interchangeable and should be understood in a broad sense, that is, it can mean that the specific options expressed between the same symbols in different groups do not affect each other, or it can mean the specific options expressed between the same symbols in the same group do not affect each other. Taking R₃ as an example, the specific options of R₃ between the structure formula “C₁-C₅₀ alkylidene optionally substituted with R₃” and the structure formula “—C(O)—NH—C₁₋₅₀ alkylidene optionally substituted with R₃” do not affect each other.

The term “small interfering RNA (siRNA)” refers to a double-stranded RNA that comprises a sense and an antisense strands, and each strand is 17 to 30 nucleotides in length. The siRNA mediates the targeted cleavage of RNA transcripts via RNA-induced silencing complex (RISC) pathway through the formation of a RISC. Specifically, the siRNA directs the specific degradation of mRNA sequences through the known process of RNA interference (RNAi), which inhibits the translation of mRNA into amino acids and thereby transformation into proteins.

In the context of the present disclosure, the term “antisense strand (or called as guide strand)” includes a region that is substantially complementary to a target sequence. The term “sense strand (or passenger strand)” refers to an iRNA strand that comprises a region that is substantially complementary to the antisense strand. The term “substantially complementary” means fully complementary or at least partially complementary. For example, the antisense strand is fully complementary or at least partially complementary to a target sequence. In the case of partial complementarity, the mismatch may be present within the internal or terminal region of the molecule, wherein the most tolerated mismatch is present within the terminal region, e.g., within the 5, 4, 3 or 2 nucleotides at the 5′- and/or 3′-end of the iRNA.

It is noted that the antisense strand being “at least partially substantially complementary” to the mRNA means that the antisense strand has a polynucleotide that is substantially complementary to a contiguous portion of the mRNA of interest.

In the context of the present disclosure, an “oligonucleotide” refers to deoxyribonucleic acid (DNA) or ribonucleic acid (RNA), generally consisting of 10 to 50 nucleotides. Oligonucleotides can regulate gene expression through a number of processes including ribonucleic acid interference, ribonuclease-mediated target degradation, splicing regulation, non-coding RNA inhibition, gene activation, and programmed genome editing.

In the context of the present disclosure, “antisense oligonucleotides (ASOs)” are single-stranded oligonucleotide molecules, typically consisting of 10 to 50 nucleotides. After entering the cell, ASO binds to its complementary target mRNA by the base complementary pairing under the action of ribonuclease H1 to inhibit the expression of the target gene.

In the context of the present disclosure, unless otherwise specified, uppercase letters A, U, G, C and T represent the base composition of the nucleotides, lowercase m represents that the nucleotide adjacent to the left side of the letter m is a 2′-methoxy modified nucleotide, lowercase f represents that the nucleotide adjacent to the left side of the letter f is a 2′-fluoro modified nucleotide, and lowercase s represents that the two nucleotides adjacent to both sides of the letter s are linked by a phosphorothioate linkage.

In the context of the present disclosure, the term “pharmaceutically acceptable carrier” includes any solvent, dispersion medium, coating material, surfactant, antioxidant, preservative (such as an antibacterial agent and an antifungal agent), isotonic agent, salt, drug stabilizer, binder, excipient, dispersing agent, lubricant, sweetener, flavoring agent, coloring agent, or combination thereof. These carriers are known to a person skilled in the art (as described in Remington's Pharmaceutical Sciences, 18th Ed. Mack Printing Company, 1990, pp. 1289-1329). Except for any conventional carrier that is incompatible with the active ingredient, the uses thereof in treatment or in a pharmaceutical composition are encompassed.

In the context of the present disclosure, the term “pharmaceutically acceptable excipient” may include any solvent, solid excipient, diluent, other liquid excipient, etc., which is suitable for the specific target dosage form. Except to the extent that any conventional excipient is incompatible with the siRNA of the present disclosure, for example, producing any adverse biological effects or harmful interactions with any other components of a pharmaceutically acceptable composition, the uses thereof are also contemplated by the present disclosure.

In the context of the present disclosure, the term “subject” refers to any animal, e.g., mammal or marsupial. The subject of the present disclosure includes, but is not limited to, human, non-human primate (e.g., monkeys), mouse, pig, horse, donkey, cow, sheep and any kind of poultry.

In the context of the present disclosure, “treatment” “alleviation” or “amelioration” may be used interchangeably herein. These terms refer to an approach for obtaining beneficial or desirable results, including but not limited to therapeutic benefit. “Therapeutic benefit” means eradication or amelioration of potential disorder to be treated. Also, a therapeutic benefit is achieved by eradicating or ameliorating one or more of physiological symptoms associated with the potential disorder such that an improvement is observed in the subject, notwithstanding that the subject may still be afflicted with the potential disorder.

In the context of the present disclosure, “prevention” and “prevent” are used interchangeably. These terms refer to an approach for obtaining beneficial or desirable results, including but not limited to prophylactic benefit. In order to obtain “prophylactic benefit”, a conjugate, an RNAi reagent, or a composition may be administered to a subject at risk of developing a particular disease, or to a subject reported with one or more physical symptoms of a disease, even though the diagnosis of this disease may not have been made yet.

In the context of the present disclosure, unless otherwise specified, the reagent ratios described in the embodiments of this disclosure are calculated based on volume ratio (v/v).

### 2. Preparation of Compounds

Reagents used in the preparation of compounds and their sources in the present disclosure are as follows.
1) Tributyltin hydride, CAS No. 688-73-3, was purchased from Beijing Ouhe Technology Co., Ltd.
2) Benzaldehyde dimethyl acetal, CAS No. 125-88-8, was purchased from Beijing Ouhe Technology Co., Ltd.
3) DL-10-camphorsulfonic acid, CAS No. 5872-08-2, was purchased from Beijing Ouhe Technology Co., Ltd.
4) 3-bromopropyne, CAS No. 106-96-7, was purchased from Beijing Ouhe Technology Co., Ltd.
5) 4,4′-dimethoxytrityl chloride (abbreviated as DMTrCl), CAS No. 40615-36-9, was purchased from Beijing Ouhe Technology Co., Ltd.
6) 4,5-dicyanoimidazole (abbreviated as DCI), CAS No. 1122-28-7, was purchased from Beijing Ouhe Technology Co., Ltd.
7) Bis(diisopropylamino)(2-cyanoethoxy)phosphine, CAS No. 102691-36-1, was purchased from Beijing Ouhe Technology Co., Ltd.
8) 1-O-acetyl-2,3,5-O-tri-benzoyl-BETA-L-ribofuranose, CAS No. 3080-30-6, was purchased from Beijing Ouhe Technology Co., Ltd.
9) Triethylsilane, CAS No. 617-86-7, was purchased from Beijing Ouhe Technology Co., Ltd.
10) Trimethylsilyl trifluoromethanesulfonate (abbreviated as TMSOTf), CAS No. 27607-77-8, was purchased from Beijing Ouhe Technology Co., Ltd.
11) Cesium carbonate, CAS No. 534-17-8, was purchased from Beijing Ouhe Technology Co., Ltd.

12) Silver oxide (Ag₂O), CAS No. 20667-12-3, was purchased from Beijing Ouhe Technology Co., Ltd.
13) Potassium iodide (KI), CAS No. 7681-11-0, was purchased from Shanghai Bide Pharmatech Co., Ltd.
14) p-toluenesulfonyl chloride (TsCl), CAS No. 98-59-9, was purchased from Beijing Ouhe Technology Co., Ltd.
15) Azidotrimethylsilane (TMSN₃), CAS No. 4648-54-8, was purchased from Ouhe Technology Co., Ltd.
16) Potassium fluoride (KF), CAS No. 7789-23-3, was purchased from Beijing Ouhe Technology Co., Ltd.
17) N,N-dimethylformamide (DMF), CAS No. 68-12-2, was purchased from Beijing Ouhe Technology Co., Ltd.
18) D-galactosamine pentaacetate, CAS No. 76375-60-5, was purchased from Beijing Ouhe Technology Co., Ltd.
19) Scandium trifluoromethanesulfonate, CAS No. 144026-79-9, was purchased from Beijing Ouhe Technology Co., Ltd.
20) (S)-(+)-solketal, CAS No. 22323-82-6, was purchased from Beijing Ouhe Technology Co., Ltd.
21) Sodium hydride (NaH), CAS No. 7646-69-7, was purchased from Beijing Ouhe Technology Co., Ltd.
21) Amberlyst-15 (divinylbenzene-styrenesulfonic acid copolymer, also known as amberlyst 15 ion-exchange resin), CAS No. 39389-20-3, was purchased from Beijing Ouhe Technology Co., Ltd.
22) Aminoalkyl-CPG (model: C3006-1000), was purchased from Beijing Ouhe Technology Co., Ltd.
23) Succinic anhydride, CAS No. 108-30-5, was purchased from Beijing Ouhe Technology Co., Ltd.
24) Reference compound L96-PS.

Compound L96-PS was purchased from Kailaiying Pharmaceutical Group (Tianjin) Co., Ltd., with a loading amount of 120±12 μmol/g, as measured by UV/HPLC.

The reference compound L96-PS has a structure of

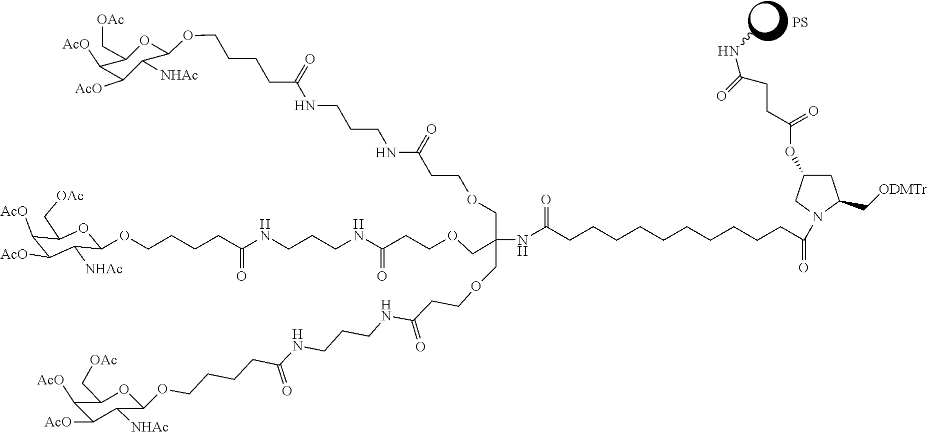

wherein, PS represents polystyrene as a solid phase support.

Unless otherwise specified, the reagents (Table 1) and instruments (Table 2) used herein were all commercially available products from the following manufacturers.

TABLE 1
Main reagents

| Name | Manufacturer |
|---|---|
| 1 × PBS | M&C GENE TECHNOLOGY |
| | (BEIJING) LTD. |
| RNA Extraction Kit (HanWei) | Zhejiang Hanwei Technology Co., Ltd. |
| Reverse Transcription System | Promega Corporation |
| SYBR Select Master Mix | ABI |
| RNA Later | Thermo Fisher Scientific |

TABLE 2
Main instruments

| Name | Manufacturer |
|---|---|
| Fully automatic nucleic acid extraction equipment | Zhejiang Hanwei Technology Co., Ltd. |
| High-speed freezing centrifuge | Eppendorf |
| NANODROP OneC | Thermo Fisher Scientific |
| Gradient PCR Amplifier | Eppendorf |
| Fluorescence quantitative PCR instrument | ABI StepOne Plus |
| Gel imager | Shanghai Tanon Science & Technology Co., Ltd. |
| Electrophoresis equipment | Beijing Liuyi Biotechnology Co., Ltd. |
| Automatic Tissue Homogenizer-Tissuelyser II | Shanghai Jingxin Industrial Development Co., Ltd |

Raw materials and reagents used in the compound, conjugate, composition and use thereof provided by the present disclosure are commercially available.

The present disclosure is further illustrated below in conjunction with examples.

### Preparation Example 1: Synthesis of Compound NM041

In this preparation example, the synthesis process of compound NM041 is as follows:

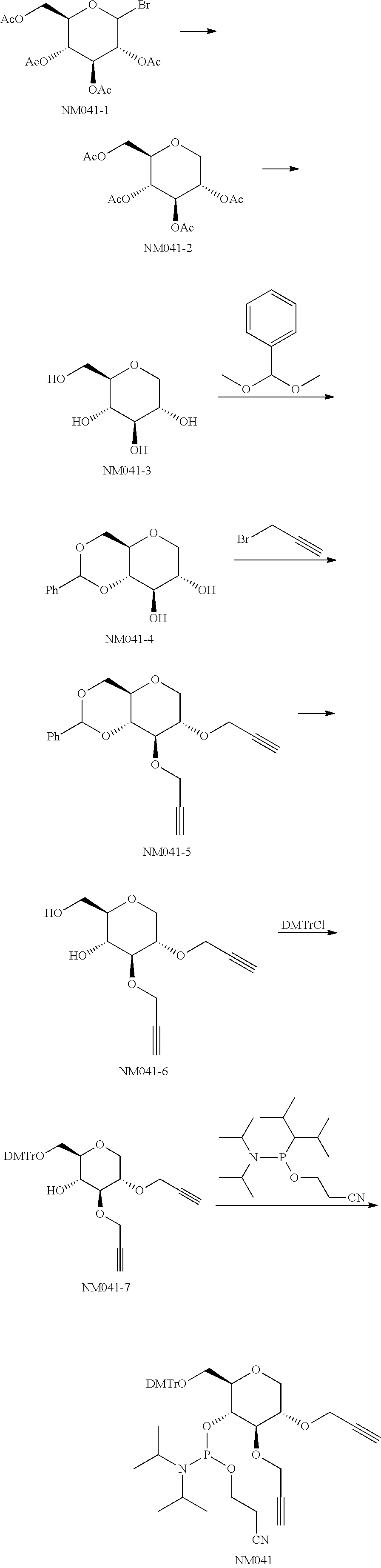

### Synthesis of Compound NM041-2

Compound NM041-1 (25.0 g, 1.0 eq) was dissolved in 250 mL of methylbenzene. The mixture was heated to 110 °C. Tributyltin hydride (17.7 g, 1.0 eq) and azobisisobutyronitrile (1 g, 0.1 eq) were added. The mixture was refluxed at 110 °C for 2 h. After the reaction was completed, the reaction solution was cooled to 25 °C. Ethyl acetate (100 ml) and potassium fluoride (10.59 g in 30 ml water, 3.0 eq) were added, stirred at 25 °C for 2 h and filtered to separate the organic phase. The aqueous phase was washed twice with ethyl acetate (50 ml×2). The organic phases were combined, washed once with 50 ml of saturated aqueous solution of sodium chloride, dried over anhydrous sodium sulfate, suction-filtered, and concentrated under reduced pressure to obtain compound NM041-2 as a light yellow oil (18.7 g 87.9% yield). MS ESI (m/z)=333.1 [M+H]⁺.

### (1-2) Synthesis of Compound NM041-3

Compound NM041-2 (18.7 g, 1 eq) was dissolved in 50 ml of anhydrous methanol, and sodium methoxide (0.288 g, 0.1 eq) was added. The mixture was stirred at 25 °C for 1 h. After the reaction was completed, the reaction solution was adjusted to pH 6-7 with a solution of 4 mol/L hydrogen chloride in 1,4-dioxane in an ice bath. The resulting solution was concentrated, subjected to water removal twice with acetonitrile and dried under vacuum to obtain compound NM041-3 as a white solid (9.4 g, 100% yield).

### (1-3) Synthesis of Compound NM041-4

Compound NM041-3 (9.4 g, 1.0 eq) was dissolved in 150 ml of acetonitrile, and benzaldehyde dimethyl acetal (30 ml, 3 eq) and DL-10-camphorsulfonic acid (1.5 g, 0.1 eq) were separately added. The mixture was stirred at 25 °C for 5 h. 3 ml of triethylamine was added, and the mixture was stirred at 25 °C for another 30 min. After the reaction was completed, the reaction solution was concentrated. 100 ml of water was added, and the reaction solution was extracted twice with ethyl acetate (100 ml×2). The organic phases were combined, dried over anhydrous sodium sulfate, suction-filtered, concentrated under reduced pressure, and then purified by normal phase chromatography (eluent: ethyl acetate/petroleum ether=57/43, v/v) to obtain compound NM041-4 as a white solid (8.6 g 59.5% yield). MS ESI (m/z)=252.2 [M+H]⁺.

### (1-4) Synthesis of Compound NM041-5

Compound NM041-4 (4.3 g, 1.0 eq) was dissolved in 40 ml of N,N-dimethylformamide (DMF), and added with sodium hydride (2.7 g 68 mmol, 4 eq) in an ice bath. The mixture was reacted for 30 min in an ice bath, added with 3-bromopropyne (8.1 g, 4 eq) and stirred at 25 °C for 2 h. 20 ml of water was added for quenching the reaction. After the reaction was completed, the reaction solution was extracted three times with ethyl acetate (50 ml×3). The organic phases were combined and washed five times with saturated sodium chloride solution (20 ml×5), dried over anhydrous sodium sulfate, filtered, and concentrated to obtain compound NM041-5 as a brown oil (100% yield). MS ESI (m/z)=329.2 [M+H]⁺.

### (1-5) Synthesis of Compound NM041-6

Compound NM041-5 (5.6 g, 1.0 eq) was dissolved in 30 ml of dichloromethane (DCM), and 300 ml of 70% by mass acetic acid aqueous solution was added. The mixture was reacted at 70 °C for 1 h. After the reaction was completed, the reaction solution was directly concentrated to obtain compound NM041-6 as a yellow oil (100% yield). MS ESI (m/z)=241 [M+H]⁺.

### (1-6) Synthesis of Compound NM041-7

Compound NM041-6 (5.6 g, 1.0 eq) was dissolved in 50 ml of pyridine. 4,4′-dimethoxytriphenyl chloromethane (10.2 g, 1.3 eq, abbreviated as DMTrCl) was added in an ice bath. The nitrogen replacement was carried out three times. The mixture was stirred at 25 °C for 3 h, and then 50 ml of methanol was added for quenching the reaction. After the reaction was completed, the reaction solution was concentrated. 50 ml of water was added, and the reaction solution was extracted three times with ethyl acetate (50 ml×3). The organic phases were combined, dried over anhydrous sodium sulfate, filtered, concentrated, and then purified by normal phase chromatography (eluent: ethyl acetate/petroleum ether-16/84, v/v) to obtain compound NM041-7 as a pale yellow solid (5 g 39.6% yield). MS ESI (m/z)=543.1 [M+H]⁺.

### (1-7) Synthesis of Compound NM041

Compound NM041-7 (2.0 g, 1.5 eq) was dissolved in 20 ml of anhydrous dichloromethane, and DCI (347.4 mg, 0.8 eq) and bis(diisopropylamino)(2-cyanoethoxy)phosphine (1.22 g, 1.1 eq) were separately added. The nitrogen replacement was carried out three times. The mixture was stirred at 25 °C for 2 h. After the reaction was completed, 20 ml of saturated sodium bicarbonate solution was added, and the reaction solution was extracted three times with dichloromethane (20 ml×3). The organic phases were combined, dried over anhydrous sodium sulfate, filtered, concentrated, purified by reversed-phase chromatography (C18 chromatographic column, eluent: acetonitrile/water-72/28, v/v), and then dried under vacuum for 12 h to obtain compound NM041 as white powders (2 g, 73.09% yield). MS ESI (m/z)=743.2 [M+H]⁺.

¹H NMR (400 MHz, Acetonitrile-d3) δ 7.53-7.47 (m, 2H), 7.36 (tt, J=9.6, 3.4 Hz, 6H), 7.28-7.22 (m, 1H), 6.94-6.86 (m, 4H), 4.45 (d, J=2.4 Hz, 1H), 4.35 (dt, J=6.1, 1.9 Hz, 2H), 4.18 (ddd, J=11.2, 5.4, 2.6 Hz, 1H), 3.81 (s, 7H), 3.79-3.20 (m, 8H), 3.07 (dt, J=10.5, 7.0 Hz, 1H), 2.79 (q, J=2.6 Hz, 1H), 2.72 (dt, J=5.1, 2.4 Hz, 1H), 2.69-2.63 (m, 1H), 2.48-2.34 (m, 1H), 2.17 (d, J=1.0 Hz, 2H), 1.08 (dd, J=6.8, 4.1 Hz, 10H), 0.90 (d, J=6.8 Hz, 2H).

### Preparation Example 2: Synthesis of Compound NM042

In this preparation example, the synthesis process of compound NM042 is as follows:

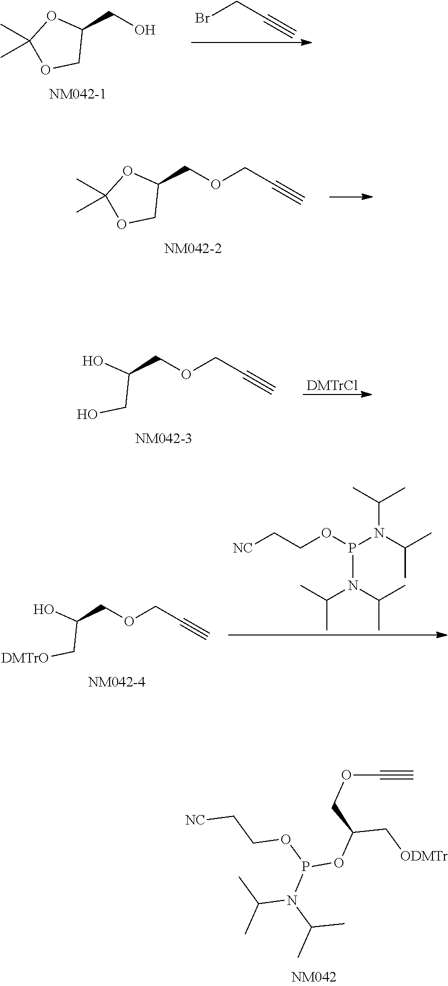

### (2-1) Synthesis of Compound NM042-2

Compound NM042-1 (5.0 g, 1.0 eq.) and sodium hydride (3.78 g, 2.5 eq.) were added to 60 ml of DMF, cooled to 0 °C, and stirred at 0 °C for 0.5 h. Bromopropyne (9.0 g, 2.0 eq.) was slowly added under stirring, and the mixture was stirred at 25 °C for 4 h. After the reaction was completed, 100 ml of water was added, and the reaction solution was extracted three times with ethyl acetate (3×100 ml). The organic phases were combined, washed once with 100 ml of saturated brine, dried over anhydrous sodium sulfate, filtered, and concentrated to obtain compound NM042-2 as an oil (9.0 g). ESI-MS (m/z)=171 [M+]⁺.

### (2-2) Synthesis of Compound NM042-3

Compound NM042-2 (9.0 g, 37.8 mmol) and Amberlyst-15 resin (13.5 g, 150% w/w) were added to 180 ml of methanol. The nitrogen replacement was carried out three times. The mixture was stirred at 25 °C for 6 h. After the reaction was completed, the reaction mixture was filtered and concentrated to obtain compound NM042-3 as an oil (9.1 g). ESI-MS (m/z)=131.2 [M+H]⁺.

### (2-3) Synthesis of Compound NM042-4

Compound NM042-3 (5 g 1.0 eq.) was added to 75 ml of pyridine solution and cooled to 0 °C, and DMTrCl (16.9 g, 1.69 eq.) was added in batches at 0 °C. The mixture was stirred at 25 °C for 3 h. After the reaction was completed, the reaction solution was concentrated and purified by reversed-phase chromatography (C18 chromatographic column, eluent: acetonitrile/water-72/28, v/v) to obtain compound NM042-4 (15 g 90.4% yield). ESI-MS (m/z)=433.2 [M+H]⁺.

### (2-4) Synthesis of Compound NM042

Compound NM042-4 (2.5 g, 1.0 eq.), bis(diisopropylamino)(2-cyanoethoxy)phosphine (3.5 g, 2.0 eq.), and DCI (0.567 g, 0.8 eq.) were separately added to 50 ml of DCM and stirred under the protection of nitrogen for 3 h. After the reaction was completed, the reaction mixture was washed once with 50 ml of saturated sodium bicarbonate solution. The organic phase was separated, washed once with 50 ml of saturated brine, dried over anhydrous sodium sulfate, filtered, concentrated, and then separated by reverse-phase HPLC to obtain compound NM042 as a white oil (2.0 g, 56% yield). ESI-MS (m/z): 619.3 [M+H]⁺.

¹H NMR (400 MHz, DMSO-d6) δ 0.97-1.04 (d, J=6.7 Hz, 3H), 1.06-1.20 (m, 9H), 3.05-3.14 (dd, J=5.5, 10.7 Hz, 2H), 3.46-3.69 (m, 6H), 3.70-3.75 (d, J=2.5 Hz, 7H), 3.98-4.09 (dt, J=4.9, 10.3 Hz, 1H), 4.09-4.17 (m, 2H), 6.81-6.93 (m, 4H), 7.15-7.34 (m, 7H), 7.35-7.46 (m, 2H).

### Preparation Example 3: Synthesis of Compounds NM014 and NM014A

In this preparation example, the synthesis processes of compounds NM014 and NM014A are as follows:

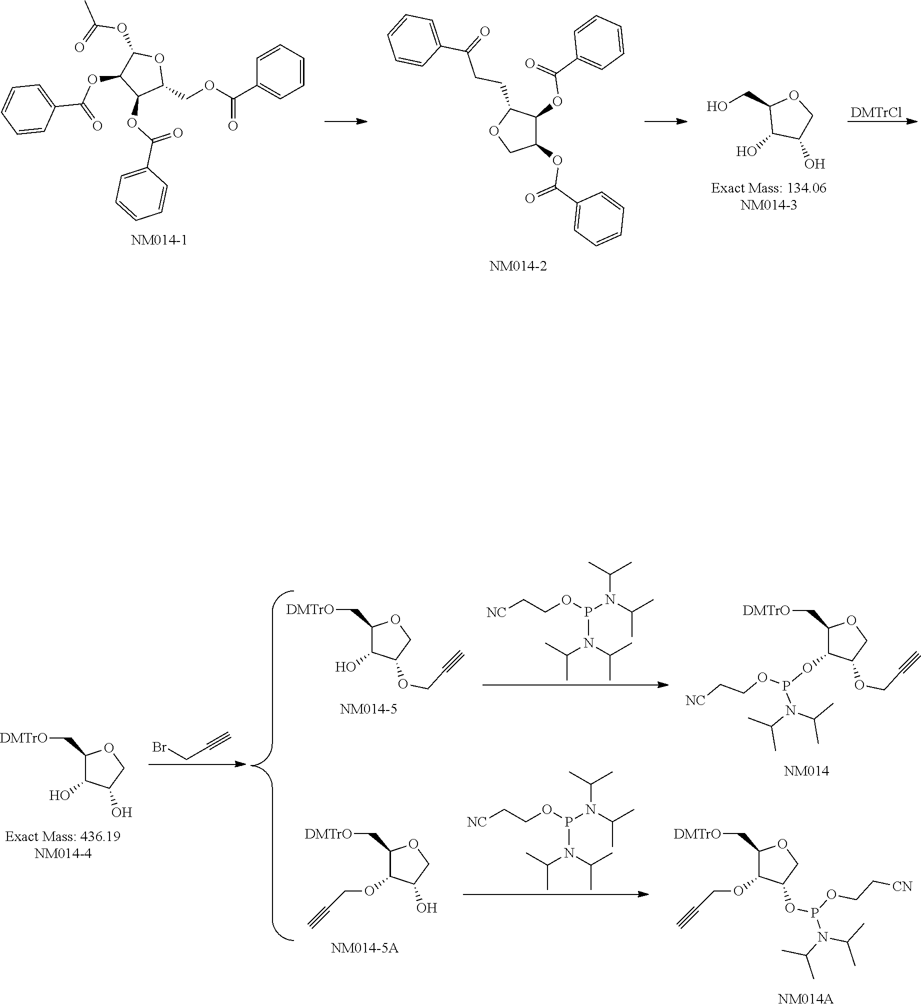

### (3-1) Synthesis of Compound NM014-2

Compound NM014-1 (50 g, 1.0 eq.) was added to 100 ml of ACN. The nitrogen replacement was carried out, and the temperature was lowered to 0 °C. TMSOTf (44.0 g, 2.0 eq.) was added dropwise at 0 °C, followed by triethylsilane (34.5 g, 3.0 eq.). The temperature was slowly raised to 25 °C, and the mixture was reacted at 25 °C for 12 h. After the reaction was completed, 200 ml of purified water was added, and the reaction solution was extracted twice with 200 ml of ethyl acetate. The organic phases were combined, dried over anhydrous sodium sulfate, filtered, concentrated, and then purified by normal phase chromatography (eluent: n-heptane/ethyl acetate=4/1, v/v) to obtain compound NM014-2 (41 g 93.1% yield). ESI-MS (m/z)=445.3 [M+H]⁺.

### (3-2) Synthesis of Compound NM014-3

Compound NM014-2 (41 g, 1.0 eq.) was added to 410 ml of monomethylamine methanol. The mixture was reacted at 25 °C for 12 h. After the reaction was completed, the reaction solution was directly concentrated and purified by normal phase chromatography (eluent: dichloromethane/methanol=10:1 for removal of impurities, followed by dichloromethane/methanol=5:1, v/v for elution) to obtain compound NM014-3 (7.5 g, 60.6% yield). ESI-MS (m/z)=135.2 [M+H]⁺.

### (3-3) Synthesis of Compound NM014-4

Compound NM014-3 (5.16 g, 1.0 eq.) was added to 30 ml of pyridine and cooled to 0 °C, and DMTrCl (14.32 g, 1.1 eq.) was added in batches at 0 °C. The mixture was stirred at 25 °C for 6 h and 10 ml of methanol was then added for quenching the reaction. After the reaction was completed, the reaction solution was directly concentrated, then diluted with 50 ml of ethyl acetate, washed once with 30 ml of saturated sodium bicarbonate solution, and then washed twice with 30 ml of purified water. The organic phase was separated, concentrated, and purified by normal phase chromatography (gradient elution from n-heptane/ethyl acetate=5/1 to n-heptane/ethyl acetate=1/1, v/v) to obtain compound NM014-4 (9.0 g, 53.6% yield). ESI-MS (m/z)=437.3 [M+H]⁺.

### (3-4) Synthesis of Compounds NM014-5 and NM014-5A

Compound NM014-4 (9.0 g, 1.0 eq.) was dissolved in 90 ml of DMF, and cesium carbonate (7.4 g, 1.1 eq.) was added. The mixture was stirred at 25 °C for 10 min and cooled to 0 °C, and bromopropyne (2.5 g, 1.0 eq.) was added dropwise at 0 °C. After the dropwise addition was completed, the mixture was slowly warmed to 25 °C and reacted at 25 °C for 16 h. After the reaction was completed, 100 ml of ethyl acetate was added, and the reaction solution was washed three times with 10 ml of purified water (10 ml×3). The organic phase was separated, dried over anhydrous sodium sulfate, filtered, concentrated, and then purified by normal phase chromatography (eluent: n-heptane/ethyl acetate=5/1, v/v, first removing impurities, then maintaining polarity of eluent) to obtain the first-eluate compound NM014-5 (2.7 g, 27.6% yield) and the later-eluate compound NM014-5A (2.3 g, 23.7% yield). Between the two, the first-eluate compound NM014-5 exhibited ESI-MS (m/z)=475.4 [M+H]⁺, and the later-eluate compound NM014-5A exhibited ESI-MS (m/z)=475.4 [M+H]⁺.

### (3-5) Synthesis of Compound NM014

The first-eluate compound NM014-5 (2.57 g, 1.0 eq.) was added to 26 ml of dichloromethane and continuously stirred. DCI (0.83 g, 1.3 eq.) was added and the nitrogen replacement was carried out. The reaction system was white and turbid and cooled to 0 °C, and bis(diisopropylamino)(2-cyanoethoxy)phosphine (2.44 g, 1.5 eq.) was added dropwise at 0 °C. After the addition was completed, the mixture was slowly warmed to 25 °C, and stirred at 25 °C for 3 h. After the reaction was completed, the reaction system was diluted with 50 ml of dichloromethane. The organic phase was washed once with 20 ml of saturated sodium bicarbonate solution, dried over anhydrous sodium sulfate, filtered, concentrated and then purified by normal phase chromatography (eluent: n-heptane/0.1% triethylamine in ethyl acetate=4/1, v/v) to obtain compound NM014 (907 mg). ESI-MS (m/z)=698.2 [M+Na]⁺.

¹H NMR (400 MHz, DMSO-d6) δ 0.88-0.94 (d, J=6.8 Hz, 4H), 1.05-1.14 (m, 8H), 1.11-1.22 (m, 1H), 1.18-1.26 (m, 1H), 2.52-2.60 (t, J=5.9 Hz, 1H), 2.72-2.80 (m, 1H), 2.89-3.00 (td, J=5.1, 10.7 Hz, 1H), 3.15-3.28 (ddd, J=2.8, 10.2, 23.1 Hz, 1H), 3.41-3.59 (m, 4H), 3.71-3.78 (d, J=2.0 Hz, 6H), 3.75-3.84 (m, 1H), 3.88-3.99 (dq, J=2.7, 18.2 Hz, 1H), 3.99-4.07 (m, 1H), 4.14-4.21 (m, 1H), 4.18-4.30 (m, 1H), 4.27-4.38 (m, 1H), 6.84-6.93 (m, 4H), 7.17-7.35 (m, 7H), 7.36-7.44 (ddd, J=1.3, 4.0, 8.2 Hz, 2H).

### (3-6) Synthesis of Compound NM014A

The later-eluate compound NM014-5A (2.3 g, 1.0 eq.) was added to 23 ml of dichloromethane and continuously stirred. DCI (0.74 g, 1.3 eq.) was added and the nitrogen replacement was carried out. The reaction system was white and turbid, and cooled to 0 °C, and bis(diisopropylamino)(2-cyanoethoxy)phosphine (2.19 g, 1.5 eq.) was added dropwise at 0 °C. After the dropwise addition was completed, the mixture was slowly warmed to 25 °C, and stirred at 25 °C for 3 h. After the reaction was completed, the reaction system was diluted with 50 ml of dichloromethane. The organic phase was washed once with 20 ml of saturated sodium bicarbonate solution, dried over anhydrous sodium sulfate, filtered, concentrated and then purified by normal phase chromatography (eluent: n-heptane/0.1% triethylamine in ethyl acetate=4/1, v/v) to obtain compound NM014A (520 mg). ESI-MS (m/z)=698.2 [M+Na]⁺.

¹H NMR (400 MHz, DMSO-d6) δ 1.10-1.20 (dd, J=6.7, 8.6 Hz, 12H), 2.74-2.82 (m, 2H), 2.96-3.04 (dd, J=4.4, 10.2 Hz, 11H), 3.11-3.19 (dd, J=3.3, 10.2 Hz, 11H), 3.38-3.43 (q, J=2.3 Hz, 1H), 3.55-3.66 (m, 2H), 3.71-3.75 (s, 6H), 3.75-3.85 (m, 3H), 3.85-3.94 (m, 1H), 4.01-4.07 (dd, J=4.4, 9.4 Hz, 1H), 4.09-4.20 (m, 2H), 4.22-4.31 (m, 1H), 4.43-4.52 (dq, J=4.1, 12.0 Hz, 1H), 6.86-6.93 (m, 4H), 7.19-7.35 (m, 7H), 7.38-7.43 (m, 2H).

### Preparation Example 4: Synthesis of Compound NM043

In this preparation example, the synthesis process of compound NM043 is as follows:

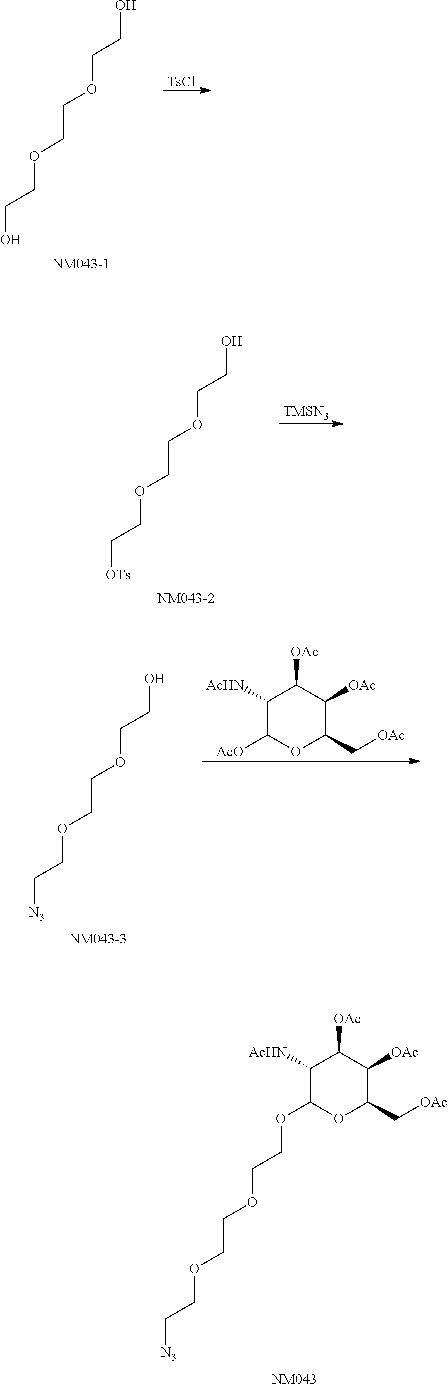

### (4-1) Synthesis of Compound NM043-2

Compound NM043-1 (10 g, 1.0 eq.), silver oxide (23.16 g, 1.5 eq.), and potassium iodide (12.16 g 1.1 eq.) were added to 100 ml of dichloromethane. The mixture was cooled to 0 °C, and a solution of TsCl (12.7 g, 1.0 eq.) in dichloromethane (100 ml) was slowly added. The mixture was stirred at 25 °C for 2 h. After the reaction was completed, the reaction solution was filtered. The organic phase was washed twice with 100 ml of water and then washed once with 100 ml of saturated aqueous solution of sodium chloride, dried over anhydrous sodium sulfate, filtered, and concentrated to obtain compound NM043-2 (12.0 g) as an oil. ESI-MS (m/z)=305.1 [M+H]⁺.

### (4-2) Synthesis of Compound NM043-3

Compound NM043-2 (12.0 g, 1.0 eq), TMSN₃ (azidotrimethylsilane, CAS No. 4648-54-8, 13.62 g, 3.0 eq.), and potassium fluoride (6.9 g 3.0 eq.) were added to 72 ml of DMF. The nitrogen replacement was carried out three times, and the mixture was heated to 60 °C and stirred at 60 °C for 1.5 h. After the reaction was completed, the reaction mixture was cooled to 25 °C, 100 ml of water was added, and the reaction mixture was extracted three times with 60 ml of ethyl acetate. The organic phases were combined, dried over anhydrous sodium sulfate, filtered, concentrated, and purified by reverse-phase chromatography (C18 chromatographic column, eluent: acetonitrile/water=72/28, v/v) to obtain compound NM043-3 (3 g) as an oil. ESI-MS (m/z)=176.3 [M+H]⁺.

### (4-3) Synthesis of Compound NM043

Compound NM043-3 (3.0 g, 1.01 eq.) and D-galactosamine pentaacetate (6.0 g 1.0 eq.) were added to 75 ml of dichloroethane. The mixture was heated to 85 °C. Scandium triflate (0.38 g, 0.05 eq.) was added in batches at 85 °C. The mixture was stirred at 85 °C for 3 h and 16 ml of triethylamine was added for quenching the reaction. After the reaction was completed, the reaction system was cooled to 25 °C. 60 ml of water and 60 ml of dichloromethane were added, and the reaction system was filtered. The organic phase was separated, dried over anhydrous sodium sulfate, filtered, concentrated, and then purified by reverse-phase chromatography (C18 chromatographic column, eluent: acetonitrile/water=72/28, v/v) to obtain compound NM043 (4.5 g) as an oil. ESI-MS (m/z)=505.3 [M+H]⁺.

### Preparation Example 5: Synthesis of Compound NM015

In this preparation example, the synthesis process of compound NM015 is as follows:

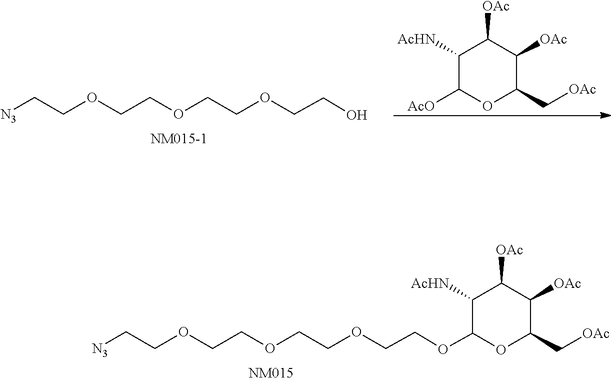 [SMILES: [N-]=[N+]=NCCOCCOCCOCCO ; CC(=O)N[C@H]1C(OC(C)=O)O[C@H](COC(C)=O)[C@H](OC(C)=O)[C@@H]1OC(C)=O ; CC(=O)N[C@H]1C(OCCOCCOCCOCCN=[N+]=[N-])O[C@H](COC(C)=O)[C@H](OC(C)=O)[C@@H]1OC(C)=O]

Compound NM015-1 (5 g, 1.1 eq.) and D-galactosamine pentaacetate (8.07 g, 1.0 eq.) were added to 96 ml of dichloroethane. The mixture was heated to 85 °C. Scandium triflate (0.38 g, 0.77 mmol) was added in batches at 85 °C. The mixture was stirred at 85 °C for 3 h and 16 ml of triethylamine was added for quenching the reaction. After the reaction was completed, the reaction system was cooled to 25 °C. 100 ml of water and 100 ml of dichloromethane were added, and the reaction system was filtered. The organic phase was separated, dried over anhydrous sodium sulfate, filtered, concentrated, and then purified by reverse-phase chromatography (C18 chromatographic column, eluent: acetonitrile/water=72/28, v/v) to obtain compound NM015 (6.6 g) as an oil. ESI-MS (m/z)=549 [M+H]⁺.

### Preparation Example 6: Synthesis of Compound NM064

In this preparation example, the synthesis process of compound NM064 is as follows:

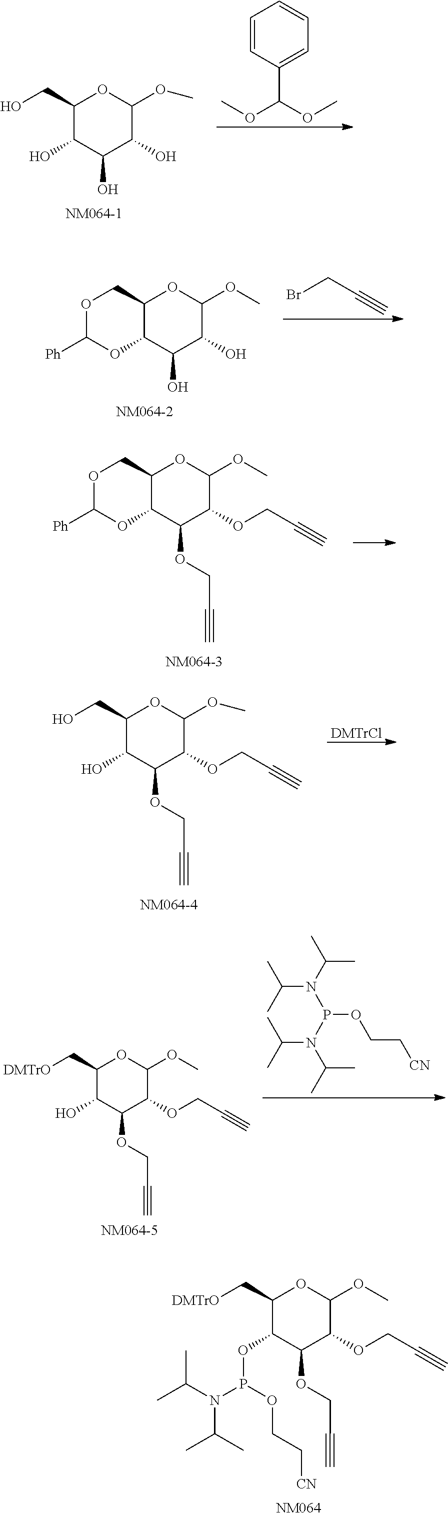

### (6-1) Synthesis of Compound NM064-2

Compound NM064-1 (13 g, 1.0 eq) was dissolved in 150 ml of acetonitrile, and benzaldehyde dimethyl acetal (30 ml, 3 eq) and DL-10-camphorsulfonic acid (1.5 g 0.1 eq) were separately added. The mixture was stirred at 25 °C for 5 h. 3 ml of triethylamine was added, and the mixture was stirred at 25 °C for another 30 min. After the reaction was completed, the reaction solution was concentrated. 100 ml of water was added, and the reaction solution was extracted twice with ethyl acetate (100 ml×2). The organic phases were combined, dried over anhydrous sodium sulfate, filtered, concentrated, and then purified by normal phase chromatography (eluent: ethyl acetate/petroleum ether=57/43, v/v) to obtain compound NM064-2 as a white solid (11.5 g 60.8% yield). MS ESI (m/z)=283 [M+H]⁺.

### (6-2) Synthesis of Compound NM064-3

Compound NM064-2 (5 g, 1.0 eq) was dissolved in 40 ml of DMF. Sodium hydride (2.7 g, 4 eq) was added in an ice bath. The mixture was reacted for 30 min in an ice bath. 3-Bromopropyne (8.1 g, 4 eq) was added and stirred at 25 °C for 2 h. 20 ml of water was added for quenching the reaction. After the reaction was completed, the reaction solution was extracted three times with ethyl acetate (50 ml×3). The organic phases were combined and washed five times with 20 ml of saturated sodium chloride solution (20 ml×5), dried over anhydrous sodium sulfate, filtered, and concentrated to obtain compound NM064-3 as a brown oil (6.3 g, 100% yield). MS ESI (m/z)=359 [M+H]⁺.

### (6-3) Synthesis of Compound NM064-4

Compound NM064-3 (6.3 g, 1.0 eq) was dissolved in 30 ml of DCM, and 300 ml of 70% by mass acetic acid aqueous solution was added. The mixture was reacted at 70 °C for 1 h.

After the reaction was completed, the reaction solution was directly concentrated to obtain compound NM064-4 as a yellow oil (4.78 g, 100% yield). MS ESI (m/z)=271 [M+H]⁺.

### (6-4) Synthesis of Compound NM064-5

Compound NM064-4 (4.78 g, 1.0 eq) was dissolved in 50 ml of pyridine. DMTrCl (7.8 g, 1.3 eq) was added in an ice bath. The nitrogen replacement was carried out three times. The mixture was stirred at 25 °C for 3 h, and then 50 ml of methanol was added for quenching the reaction. After the reaction was completed, the reaction solution was concentrated. 50 ml of water was added, and the reaction solution was extracted three times with 50 ml of ethyl acetate (50 ml×3). The organic phases were combined, dried over anhydrous sodium sulfate, filtered, concentrated, and then purified by normal phase chromatography (eluent: ethyl acetate/petroleum ether=16/84, v/v) to obtain compound NM064-5 as a pale yellow solid (6.7 g, 66.3% yield). MS ESI (m/z)=573 [M+H]⁺.

### (6-5) Synthesis of Compound NM064

Compound NM064-5 (2.0 g, 1.0 eq) was dissolved in 20 ml of anhydrous dichloromethane, and DCI (330.4 mg, 0.8 eq) and bis(diisopropylamino)(2-cyanoethoxy)phosphine (1.16 g, 1.1 eq) were separately added. The nitrogen replacement was carried out three times. The mixture was stirred at 25 °C for 2 h. After the reaction was completed, 20 ml of saturated sodium bicarbonate solution was added, and the reaction solution was extracted three times with 20 ml of dichloromethane (20 ml×3). The organic phases were combined, dried over anhydrous sodium sulfate, filtered, concentrated, purified by reverse-phase chromatography (C18 chromatographic column, eluent: acetonitrile/water=72/28, v/v), and then dried under vacuum for 12 h to obtain compound NM064 as white powders (2 g, 74.07% yield). MS ESI (m/z)=774 [M+H]⁺.

¹H NMR (400 MHz, DMSO-d6) δ 7.45-7.39 (d, J=7.8 Hz, 2H), 7.36-7.18 (tt, J=14.5, 8.5 Hz, 7H), 6.95-6.84 (d, J=7.5 Hz, 4H), 5.01-4.96 (s, 1H), 4.43-4.28 (s, 4H), 3.81-3.70 (s, 8H), 3.65-3.35 (m, 12H), 3.28-3.18 (dt, J=14.5, 7.2 Hz, 1H), 3.06-2.97 (t, J=9.5 Hz, 1H), 2.75-2.68 (m, 1H), 1.07-0.95 (q, J=7.4, 6.8 Hz, 10H), 0.84-0.78 (d, J=6.6 Hz, 2H).

### Preparation Example 7: Synthesis of Compounds CR01022 and CR01022Z (a Monomeric CR01022 is Expressed as (CR01022)×1)

In this preparation example, the synthesis processes of compounds CR01022 and CR01022Z are as follows:

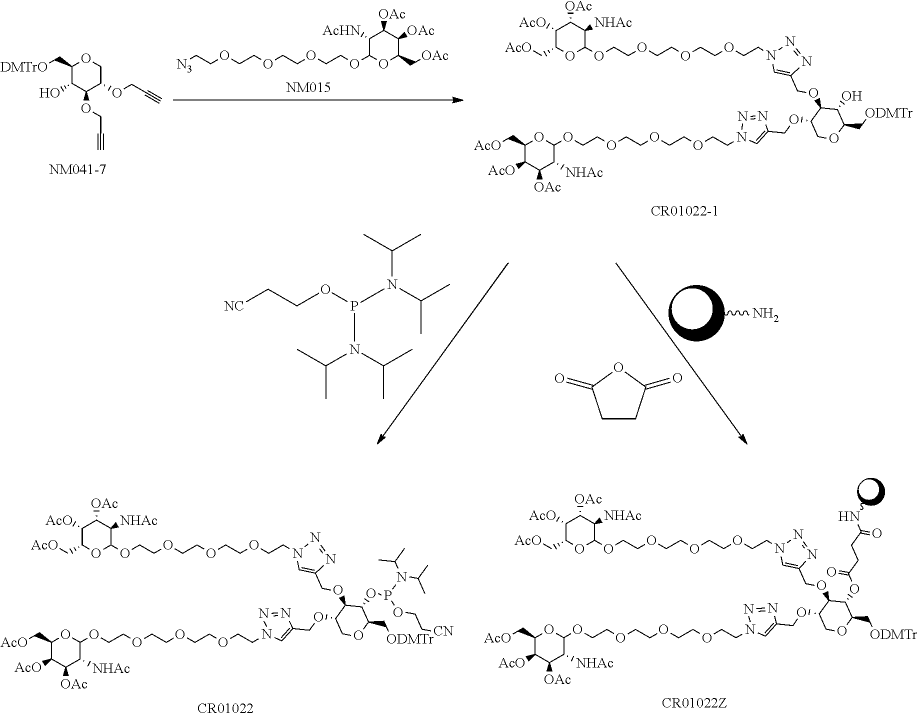

### (7-1) Synthesis of Compound CR01022-1

Compound NM015 (1114 mg, 2.03 mmol, 2.2 eq.), compound NM041-7 (500 mg, 0.922 mmol, 1.0 eq.), cuprous iodide (701 mg, 3.69 mmol, 4.0 eq.), and N,N-diisopropylethylamine (476 mg, 3.69 mmol, 4.0 eq.) were added to 3 mL of N,N-dimethylformamide solution and stirred at 25 °C for 1.5 h. After the reaction was completed, 5 mL of ethyl acetate and 5 mL of water were added, and the reaction solution was filtered. The organic phase was separated, dried over anhydrous sodium sulfate, filtered, concentrated, and purified by reverse-phase chromatography (C18 chromatographic column, eluent: acetonitrile/water=72/28, v/v) to obtain compound CR01022-1 (1 g) as a foam. ESI-MS (m/z)=1639 [M+H]⁺.

### (7-2) Synthesis of Compound CR01022

Compound CR01022-1 (850 mg, 0.523 mmol, 1.0 eq.) was added to 17 ml of dichloromethane. Bis(diisopropylamino)(2-cyanoethoxy)phosphine (313 mg, 1.04 mmol, 2.0 eq.) was added in batches, and 4,5-dicyanoimidazole (49 mg, 0.415 mmol, 0.8 eq.) was added subsequently. The nitrogen replacement was carried out three times. The mixture was stirred at 25 °C for 1 h. After the reaction was completed, the reaction solution was washed once with saturated aqueous solution of sodium bicarbonate. The organic phase was separated, concentrated, and purified by reverse-phase chromatography (C18 chromatographic column, acetonitrile/water=72/28, v/v) to obtain compound CR01022 (560 mg). MS ESI (m/z)=1839 [M+H]⁺.

### (7-3) Synthesis of Compound CR01022Z

To a 25 ml reactor, compound CR01022-1 (160 mg, 0.1 mmol, 1.0 eq.) was added to 3.2 ml of dichloromethane. Succinic anhydride (18 mg, 0.18 mmol, 1.8 eq.), 4-dimethylaminopyridine (1.2 mg, 0.01 mmol, 0.01 eq.), and triethylamine (19.7 mg, 0.2 mmol, 2.0 eq.) were added. The nitrogen replacement was carried out three times. The mixture was stirred at 25 °C for 16 h. After the reaction was completed, the reaction solution was purified by reverse-phase chromatography (C18 chromatographic column, acetonitrile/water=72/28, v/v) to obtain 100 mg of the product.

The above product (100 mg, 0.058 mmol, 1.0 eq.), aminoalkyl-CPG (1.44 g, 0.115 mmol, 80 μmol/g), benzotriazole-N,N,N′,N′-tetramethyluronium hexafluorophosphate (32.67 mg, 0.086 mmol, 1.5 eq.), and N,N-diisopropylethylamine (14.8 mg 0.115 mmol, 2.0 eq.) were mixed and shaken on a shaker for 16 h. The reaction solution was filtered, and the resulting solid was washed once with 10 ml of acetonitrile, and dried under vacuum. The solid, 4-dimethylaminopyridine (2 mg, 0.017 mmol), Cap 1 (15 ml), and Cap 2 (1.5 ml) were mixed and shaken on a shaker for 6 h. The reaction solution was filtered, and the filter cake was washed once with 10 ml of acetonitrile, and dried under vacuum to obtain compound CR01022Z (1.366 g, loading amount: 20-30 μmol/g).

Wherein, Cap1 and Cap2 were capping agents, Cap1 was a solution of 20 vol % N-methylimidazole in a pyridine/acetonitrile mixture, the volume ratio of pyridine to acetonitrile was 3:5, and Cap2 was a solution of 20 vol % acetic anhydride in acetonitrile.

### Preparation Example 8: Synthesis of Compounds CR01027 and CR01027Z (a Monomeric CR01027 is Expressed as (CR01027)×1)

In this preparation example, the synthesis processes of compounds CR01027 and CR01027Z are as follows:

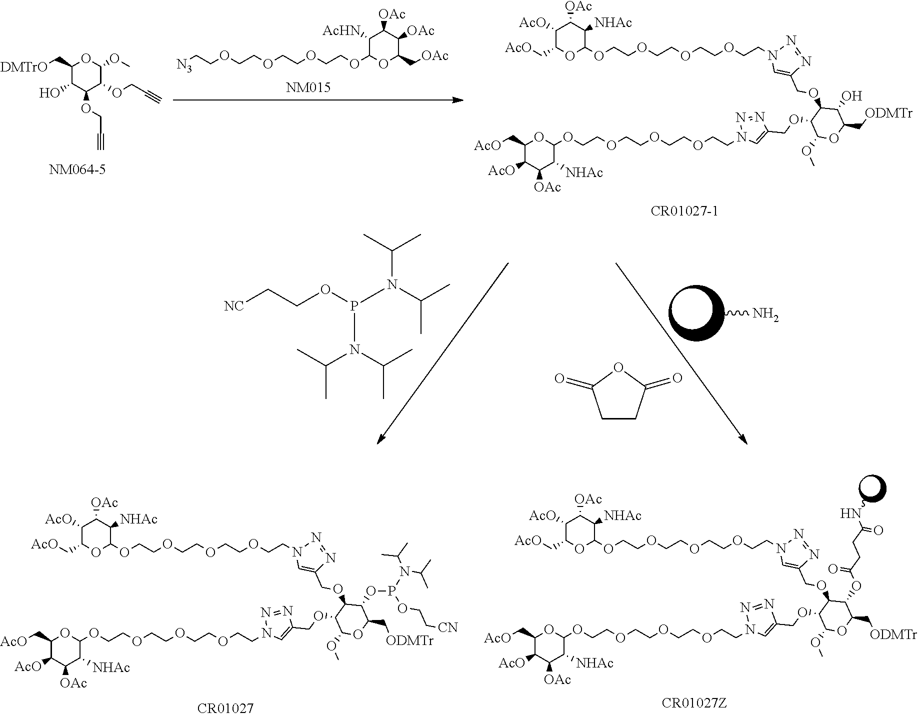

### (8-1) Synthesis of Compound CR01027-1

Compound NM015 (1125 mg, 2.03 mmol, 2.2 eq.), compound NM064-5 (500 mg, 0.922 mmol, 1.0 eq.), cuprous iodide (701 mg, 3.69 mmol, 4.0 eq.), and N,N-diisopropylethylamine (476 mg, 3.69 mmol, 4.0 eq.) were added to 3 mL of N,N-dimethylformamide and stirred at 25 °C for 1.5 h. After the reaction was completed, 5 mL of ethyl acetate and 5 mL of water were added, and the reaction solution was filtered. The organic phase was separated, dried over anhydrous sodium sulfate, concentrated, and purified by reverse-phase chromatography (C18 chromatographic column, acetonitrile/water=72/28, v/v) to obtain compound CR01027-1 (1 g, 68.5% yield) as a foam. ESI-MS (m/z)=1669.7 [M+H]⁺.

### (8-2) Synthesis of Compound CR01027

Compound CR01027-1 (870 mg, 0.530 mmol, 1.0 eq.) was added to 20 ml of dichloromethane. Bis(diisopropylamino)(2-cyanoethoxy)phosphine (330 mg, 1.04 mmol, 2.0 eq.) was added in batches, and 4,5-dicyanoimidazole (52 mg, 0.415 mmol, 0.8 eq.) was added. The nitrogen replacement was carried out three times. The mixture was stirred at 25 °C for 1 h. After the reaction was completed, the reaction solution was washed once with aqueous solution of sodium bicarbonate. The organic phase was separated, concentrated, and purified by reverse-phase chromatography (C18 chromatographic column, acetonitrile/water=72/28, v/v) to obtain compound CR01027 (560 mg, 60.8% yield). MS ESI (m/z)=1869.8 [M+H]⁺.

### (8-3) Synthesis of Compound CR01027Z

Compound CR01027Z was prepared according to the synthesis method of compound CR01022Z described in (7-3) of preparation example 7.

### Preparation Example 9: Synthesis of Compounds CR01020 and CR01020Z

In this preparation example, the synthesis processes of compounds CR01020 and CR01020Z are as follows:

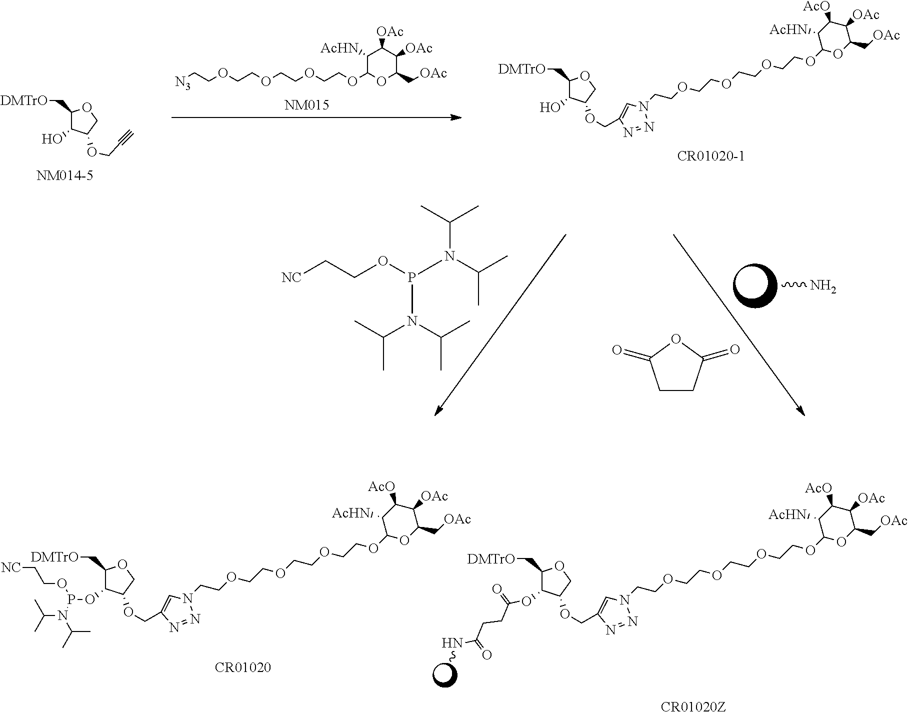

### (9-1) Synthesis of Compound CR01020-1

Compound NM015 (2.55 g 4.64 mmol, 2.2 eq.), compound NM014-5 (1.0 g, 2.11 mmol, 1.0 eq.), cuprous iodide (1.61 g, 8.44 mmol, 4.0 eq.), and N,N-diisopropylethylamine (1.09 g, 8.44 mmol, 4.0 eq.) were added to 10 mL of N,N-dimethylformamide and stirred at 25 °C for 1.5 h. After the reaction was completed, 15 mL of ethyl acetate and 15 mL of water were added, and the reaction solution was filtered. The organic phase was separated, dried over anhydrous sodium sulfate, filtered, concentrated, and purified by reverse-phase chromatography (C18 chromatographic column, acetonitrile/water-72/28, v/v) to obtain compound CR01020-1 (1.6 g 74.1% yield) as a foam. ESI-MS (m/z)=1023.2 [M+]⁺.

### (9-2) Synthesis of Compound CR01020

Compound CR01020-1 (1.0 g, 0.98 mmol, 1.0 eq.) was added to 20 ml of dichloromethane. Bis(diisopropylamino)(2-cyanoethoxy)phosphine (0.59 g, 1.96 mmol, 2.0 eq.) was added in batches, and then 4,5-dicyanoimidazole (92 mg, 0.78 mmol, 0.8 eq.) was added. The nitrogen replacement was carried out three times. The mixture was stirred at room temperature for 1 h. After the reaction was completed, the reaction solution was washed once with aqueous solution of sodium bicarbonate. The organic phase was separated, dried over anhydrous sodium sulfate, filtered, concentrated, and purified by reverse-phase chromatography (C18 chromatographic column, acetonitrile/water=72/28, v/v) to obtain compound CR01020 (780 mg, 65.0% yield). MS ESI (m/z)=1223.5 [M+H]⁺.

### (9-3) Synthesis of Compound CR01020Z

Compound CR01020Z was prepared according to the synthesis method of compound CR01022Z described in (7-3) of preparation example 7.

### Preparation Example 10: Synthesis of Compounds CR01021 and CR01021Z (a Monomeric CR01021 is Expressed as (CR01021)×1)

In this preparation example, the synthesis processes of compounds CR01021 and CR01021Z are as follows:

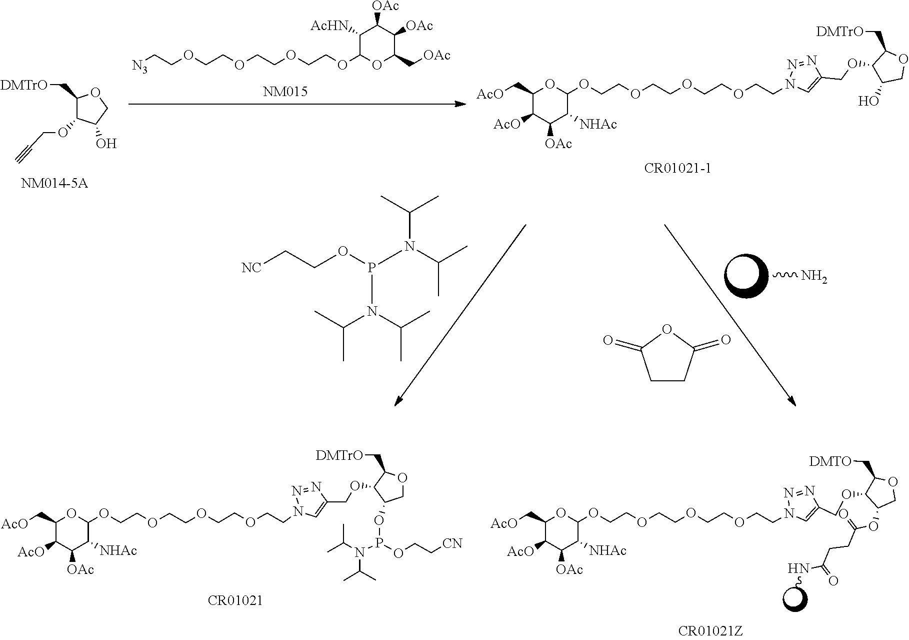

### (10-1) Synthesis of Compound CR01021-1

Compound NM015 (2.55 g, 4.64 mmol, 2.2 eq.), compound NM014-5A (1.0 g, 2.11 mmol, 1.0 eq.), cuprous iodide (1.61 g, 8.44 mmol, 4.0 eq.), and N,N-diisopropylethylamine (1.09 g, 8.44 mmol, 4.0 eq.) were added to 10 mL of N,N-dimethylformamide and stirred at 25 °C for 1.5 h. After the reaction was completed, 15 mL of ethyl acetate and 15 mL of water were added, and the reaction solution was filtered. The organic phase was separated, dried over anhydrous sodium sulfate, filtered, concentrated, and purified by reverse-phase chromatography (C18 chromatographic column, acetonitrile/water=72/28, v/v) to obtain compound CR01021-1 (1.7 g, 78.7% yield) as a foam. ESI-MS (m/z)=1023.2 [M+H]⁺.

### (10-2) Synthesis of Compound CR01021

Compound CR01021-1 (1.0 g, 0.98 mmol, 1.0 eq.) was added to 20 ml of dichloromethane. Bis(diisopropylamino)(2-cyanoethoxy)phosphine (0.59 g, 1.96 mmol, 2.0 eq.) was added in batches, and 4,5-dicyanoimidazole (92 mg, 0.78 mmol, 0.8 eq.) was added. The nitrogen replacement was carried out three times. The mixture was stirred at 25 °C for 1 h. After the reaction was completed, the reaction solution was washed once with aqueous solution of sodium bicarbonate. The organic phase was separated, concentrated, and purified by reverse-phase chromatography (C18 chromatographic column, acetonitrile/water=72/28) to obtain compound CR01021 (880 mg, 73.3% yield). MS ESI (m/z)=1223.5 [M+H]⁺.

### (10-3) Synthesis of Compound CR01021Z

Compound CR01021Z was prepared according to the synthesis method of compound CR01022Z described in (7-3) of preparation example 7.

### Preparation Example 11: Synthesis of Compounds CR01023 and CR01023Z (a Monomeric CR01023 is Expressed as (CR01023)×1)

In this preparation example, the synthesis processes of compounds CR01023 and CR01023Z are as follows:

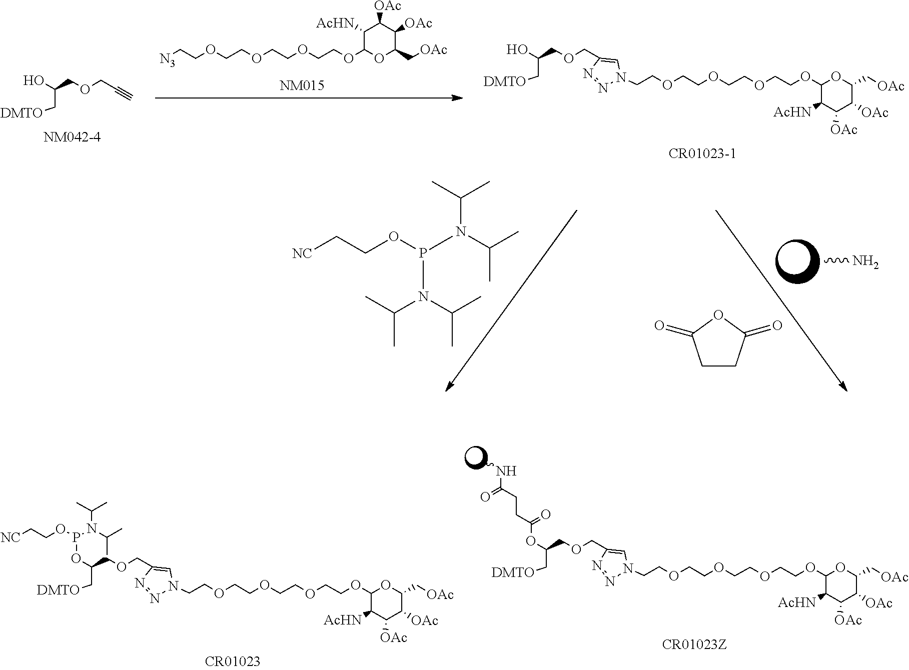

### (11-1) Synthesis of Compound CR01023-1

Compound NM015 (2.79 g 5.08 mmol, 2.2 eq.), compound NM042-4 (1.0 g 2.31 mmol, 1.0 eq.), cuprous iodide (1.76 g, 9.24 mmol, 4.0 eq.), and N,N-diisopropylethylamine (1.19 g, 9.24 mmol, 4.0 eq.) were added to 10 mL of N,N-dimethylformamide and stirred at 25 °C for 1.5 h. Then 15 mL of ethyl acetate and 15 mL of water were added, and the reaction solution was filtered and separated. The organic phase was dried over anhydrous sodium sulfate, filtered, concentrated, and purified by reverse-phase chromatography (C18 chromatographic column, acetonitrile/water=72/28, v/v) to obtain compound CR01023-1 (1.6 g, 70.5% yield) as a foam. ESI-MS (m/z)=980.2 [M+H]⁺.

### (11-2) Synthesis of Compound CR01023

Compound CR01023-1 (1.0 g, 1.02 mmol, 1.0 eq.) was added to 20 ml of dichloromethane. Bis(diisopropylamino)(2-cyanoethoxy)phosphine (0.61 g 2.04 mmol, 2.0 eq.) was added in batches, and 4,5-dicyanoimidazole (96 mg, 0.82 mmol, 0.8 eq.) was added. The nitrogen replacement was carried out three times. The mixture was stirred at 25 °C for 1 h. After the reaction was completed, the reaction solution was washed once with aqueous solution of sodium bicarbonate. The organic phase was separated, concentrated, and purified by reverse-phase chromatography (C18 chromatographic column, acetonitrile/water=72/28, v/v) to obtain compound CR01023 (880 mg, 73.3% yield). MS ESI (m/z)=1181.5 [M+H]⁺.

### (11-3) Synthesis of Compound CR01023Z

Compound CR01023Z was prepared according to the synthesis method of compound CR01022Z described in (7-3) of preparation example 7.

### Preparation Example 12: Synthesis of Compounds CR01024 and CR01024Z (a Monomeric CR01024 is Expressed as (CR01024)×1)

In this preparation example, the synthesis processes of compounds CR01024 and CR01024Z are as follows:

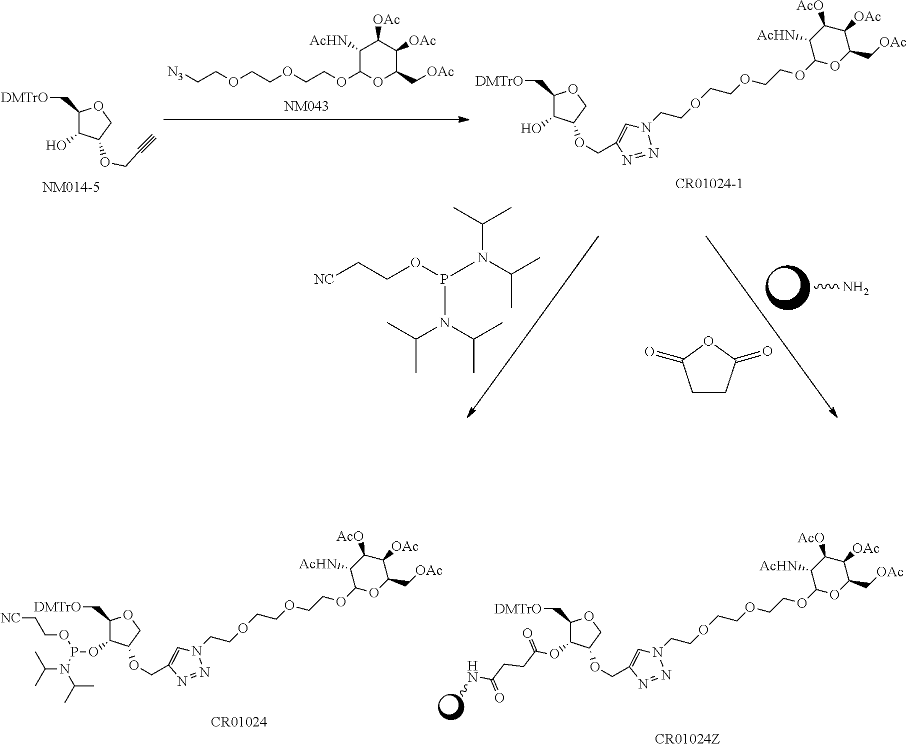

### (12-1) Synthesis of Compound CR01024-1

Compound NM043 (2.34 g, 4.64 mmol, 2.2 eq.), compound NM014-5 (1.0 g, 2.11 mmol, 1.0 eq.), cuprous iodide (1.61 g, 8.44 mmol, 4.0 eq.), and N,N-diisopropylethylamine (1.09 g, 8.44 mmol, 4.0 eq.) were added to 10 mL of N,N-dimethylformamide and stirred at 25 °C for 1.5 h. After the reaction was completed, 15 mL of ethyl acetate and 15 mL of water were added, and the reaction solution was filtered. The organic phase was separated, dried over anhydrous sodium sulfate, filtered, concentrated, and purified by reverse-phase chromatography (C18 chromatographic column, eluent: acetonitrile/water=72/28, v/v) to obtain compound CR01024-1 (1.7 g, 80% yield) as a foam. ESI-MS (m/z)=979.2 [M+]+.

### (12-2) Synthesis of Compound CR01024

To a 25 ml reactor, compound CR01024-1 (1.0 g, 1.02 mmol, 1.0 eq.) and dichloromethane (20 ml) were added. Bis(diisopropylamino)(2-cyanoethoxy)phosphine (0.61 g, 2.04 mmol, 2.0 eq.) was added in batches, and 4,5-dicyanoimidazole (96 mg 0.82 mmol, 0.8 eq.) was added. The nitrogen replacement was carried out three times. The mixture was stirred at room temperature for 1 h. Then the reaction solution was washed once with aqueous solution of sodium bicarbonate and separated. The organic phase was concentrated and purified by reverse-phase chromatography (C18 chromatographic column, acetonitrile/water=72/28, v/v) to obtain compound CR01024 (770 mg, 64.2% yield). MS ESI (m/z)=1179.5 [M+H]⁺.

### (12-3) Synthesis of Compound CR01024Z

Compound CR01024Z was prepared according to the synthesis method of compound CR01022Z described in (7-3) of preparation example 7.

### Preparation Example 13: Synthesis of Compounds CR01025 and CR01025Z (a Monomeric CR01025 is Expressed as (CR01025)×1)

In this preparation example, the synthesis processes of compounds CR01025 and CR01025Z are as follows:

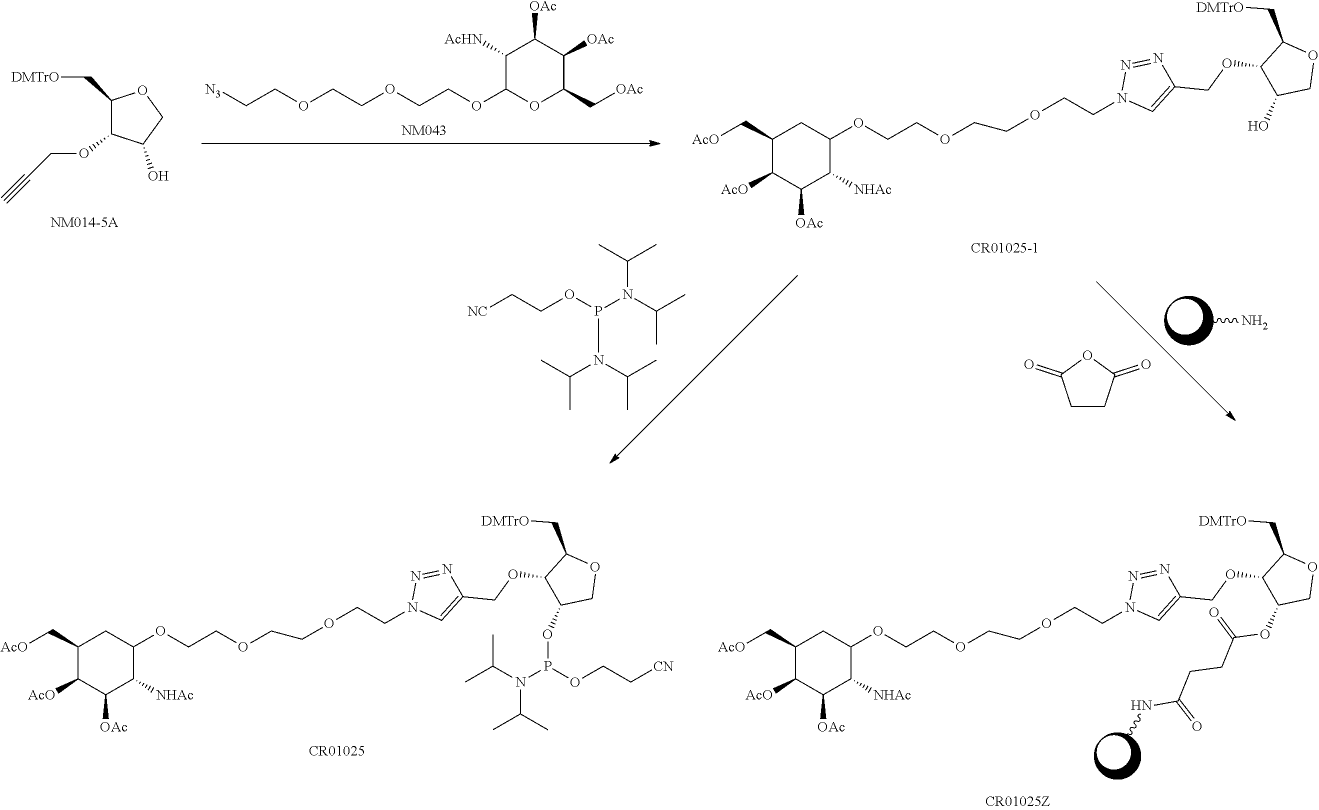

### (13-1) Synthesis of Compound CR01025-1

Compound NM043 (2.34 g, 4.64 mmol, 2.2 eq.), compound NM14-5A (1.0 g, 2.11 mmol, 1.0 eq.), cuprous iodide (1.61 g, 8.44 mmol, 4.0 eq.), and N,N-diisopropylethylamine (1.09 g, 8.44 mmol, 4.0 eq.) were added to 10 mL of N,N-dimethylformamide and stirred at 25 °C for 1.5 h. After the reaction was completed, 15 mL of ethyl acetate and 15 mL of water were added, and the reaction solution was filtered. The organic phase was separated, dried over anhydrous sodium sulfate, filtered, concentrated, and purified by reverse-phase chromatography (C18 chromatographic column, acetonitrile/water=72/28, v/v) to obtain compound CR01025-1 (1.7 g, 80% yield) as a foam. ESI-MS (m/z)=979.2 [M+H].

### (13-2) Synthesis of Compound CR01025

Compound CR01025-1 (1.0 g, 1.02 mmol, 1.0 eq.) was added to 20 ml of dichloromethane. Bis(diisopropylamino)(2-cyanoethoxy)phosphine (0.61 g 2.04 mmol, 2.0 eq.) was added in batches, and 4,5-dicyanoimidazole (96 mg, 0.82 mmol, 0.8 eq.) was added. The nitrogen replacement was carried out three times. The mixture was stirred at room temperature for 1 h. After the reaction was completed, the reaction solution was washed once with aqueous solution of sodium bicarbonate. The organic phase was separated, concentrated and purified by reverse-phase chromatography (C18 chromatographic column, acetonitrile/water=72/28, v/v) to obtain compound CR01025 (775 mg, 64.3% yield). MS ESI (m/z)=1179.5 [M+H]⁺.

### (13-3) Synthesis of Compound CR01025Z

Compound CR01025Z was prepared according to the synthesis method of compound CR01022Z described in (7-3) of preparation example 7.

### Preparation Example 14: Synthesis of Compounds CR01026 and CR01026Z (a Monomeric CR01026 is Expressed as (CR01026)×1)

In this preparation example, the synthesis processes of compounds CR01026 and CR01026Z are as follows:

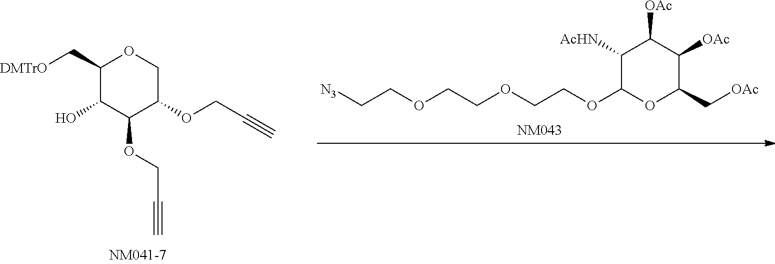

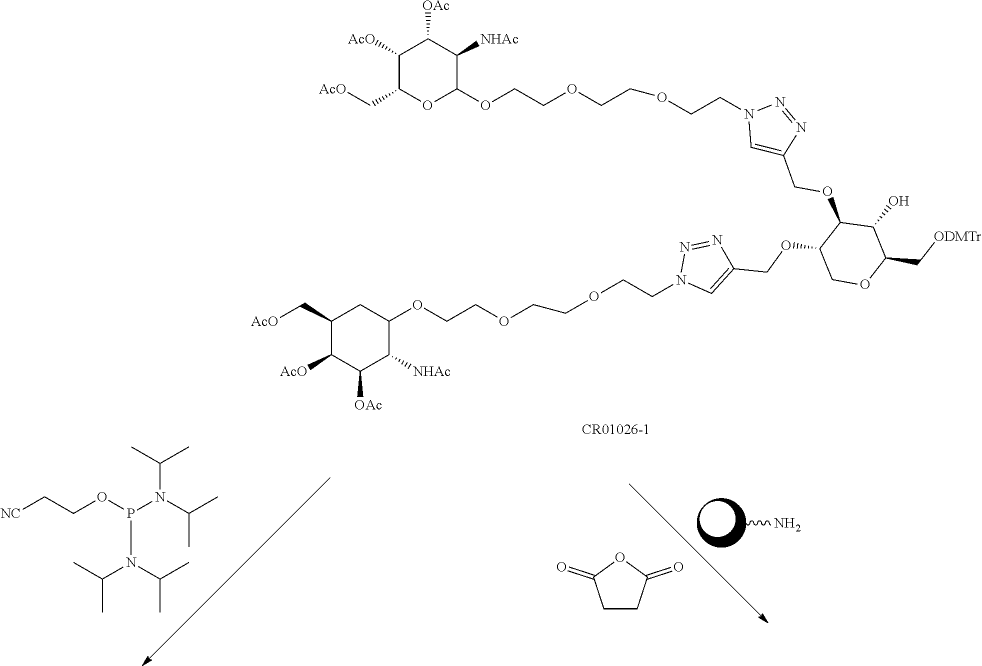

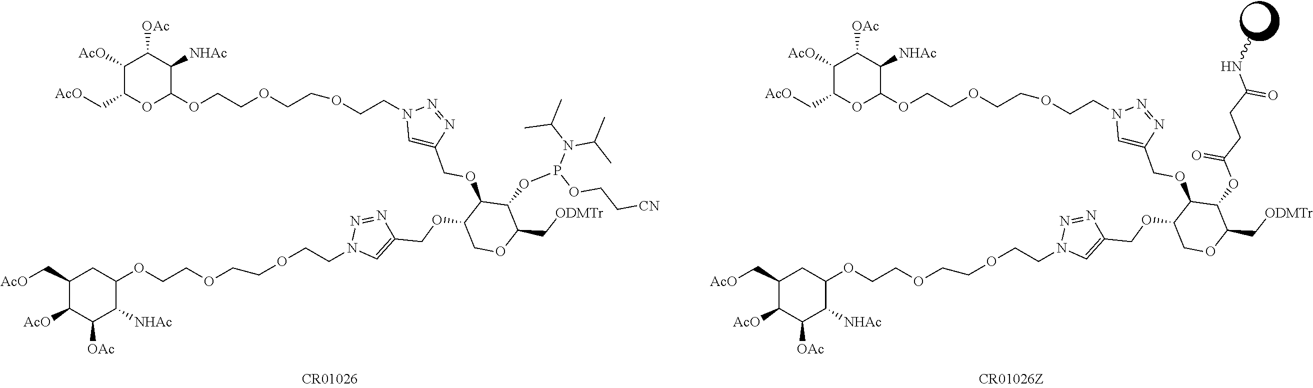

### (14-1) Synthesis of Compound CR01026-1

Compound NM043 (4.08 g, 8.10 mmol, 4.4 eq.), compound NM041-7 (1.0 g 1.84 mmol, 1.0 eq.), cuprous iodide (2.10 g, 11.04 mmol, 6.0 eq.), and N,N-diisopropylethylamine (1.43 g, 11.04 mmol, 6.0 eq.) were added to 20 mL of N,N-dimethylformamide and stirred at 25 °C for 1.5 h. After the reaction was completed, 25 mL of ethyl acetate and 25 mL of water were added, and the reaction solution was filtered. The organic phase was separated, dried over anhydrous sodium sulfate, filtered, concentrated, and purified by reverse-phase chromatography (C18 chromatographic column, acetonitrile/water=72/28, v/v) to obtain compound CR01026-1 (2.5 g, 87.4% yield) as a foam. ESI-MS (m/z)=1551.2 [M+H]⁺.

### (14-2) Synthesis of Compound CR01026

Compound CR01026-1 (1.0 g, 0.64 mmol, 1.0 eq.) was added to 20 ml of dichloromethane. Bis(diisopropylamino)(2-cyanoethoxy)phosphine (0.39 g 1.28 mmol, 2.0 eq.) was added in batches, and 4,5-dicyanoimidazole (60 mg, 0.51 mmol, 0.8 eq.) was then added. The nitrogen replacement was carried out three times. The mixture was stirred at 25 °C for 1 h. After the reaction was completed, the reaction solution was washed once with aqueous solution of sodium bicarbonate. The organic phase was separated, concentrated and purified by reverse-phase chromatography (C18 chromatographic column, acetonitrile/water-72/28, v/v) to obtain compound CR01026 (670 mg, 55.8% yield). MS ESI (m/z)=1751.5 [M+H]⁺.

### (14-3) Synthesis of Compound CR01026Z

Compound CR01026Z was prepared according to the synthesis method of compound CR01022Z described in (7-3) of preparation example 7.

### Preparation Example 15: Synthesis of Compounds CR01017 and CR01017Z (a Monomeric CR01017 is Expressed as (CR01017)×1)

In this preparation example, the synthesis processes of compounds CR01017 and CR01017Z are as follows:

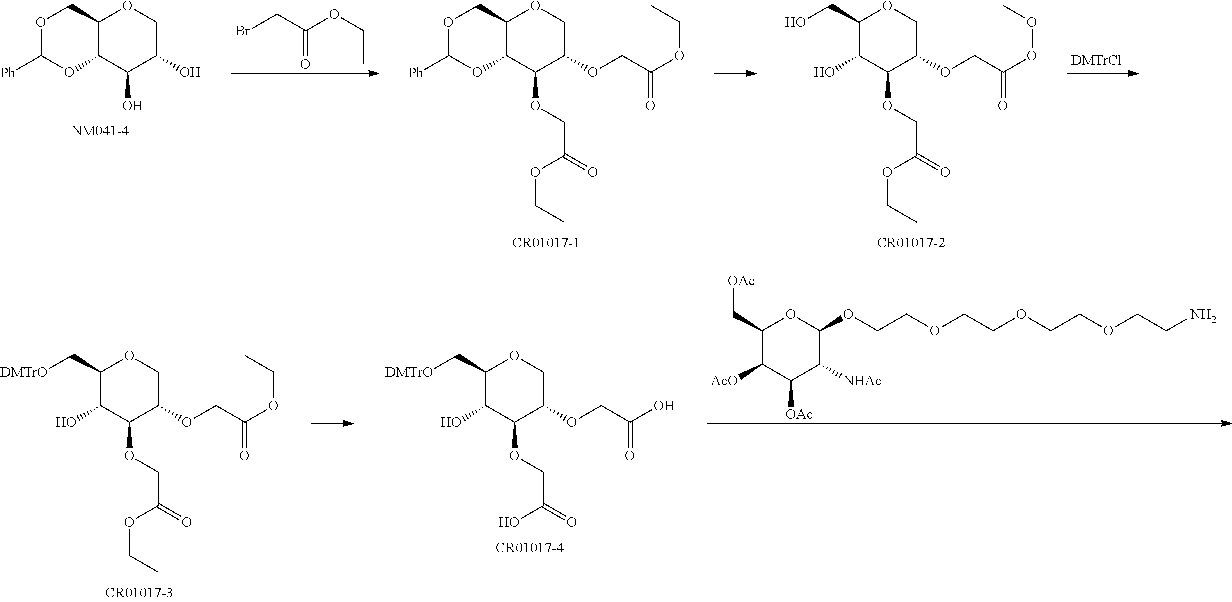

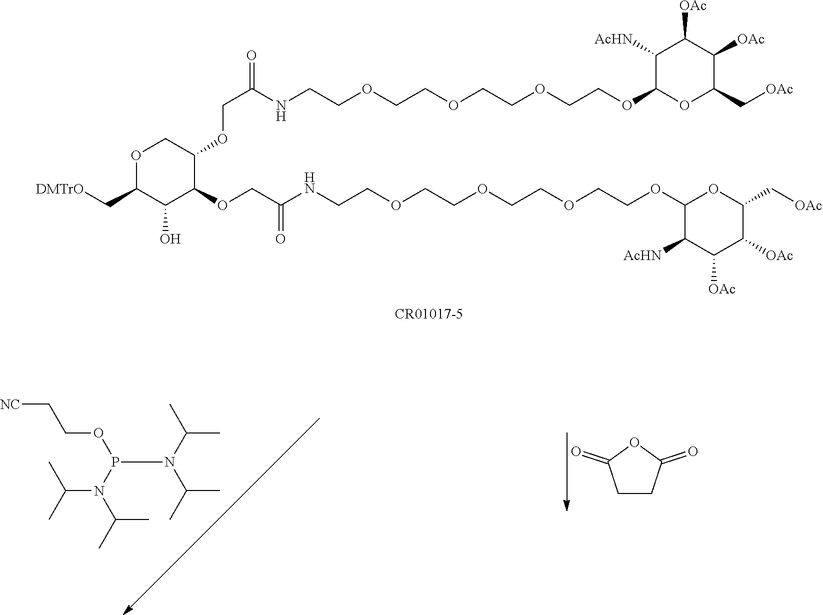

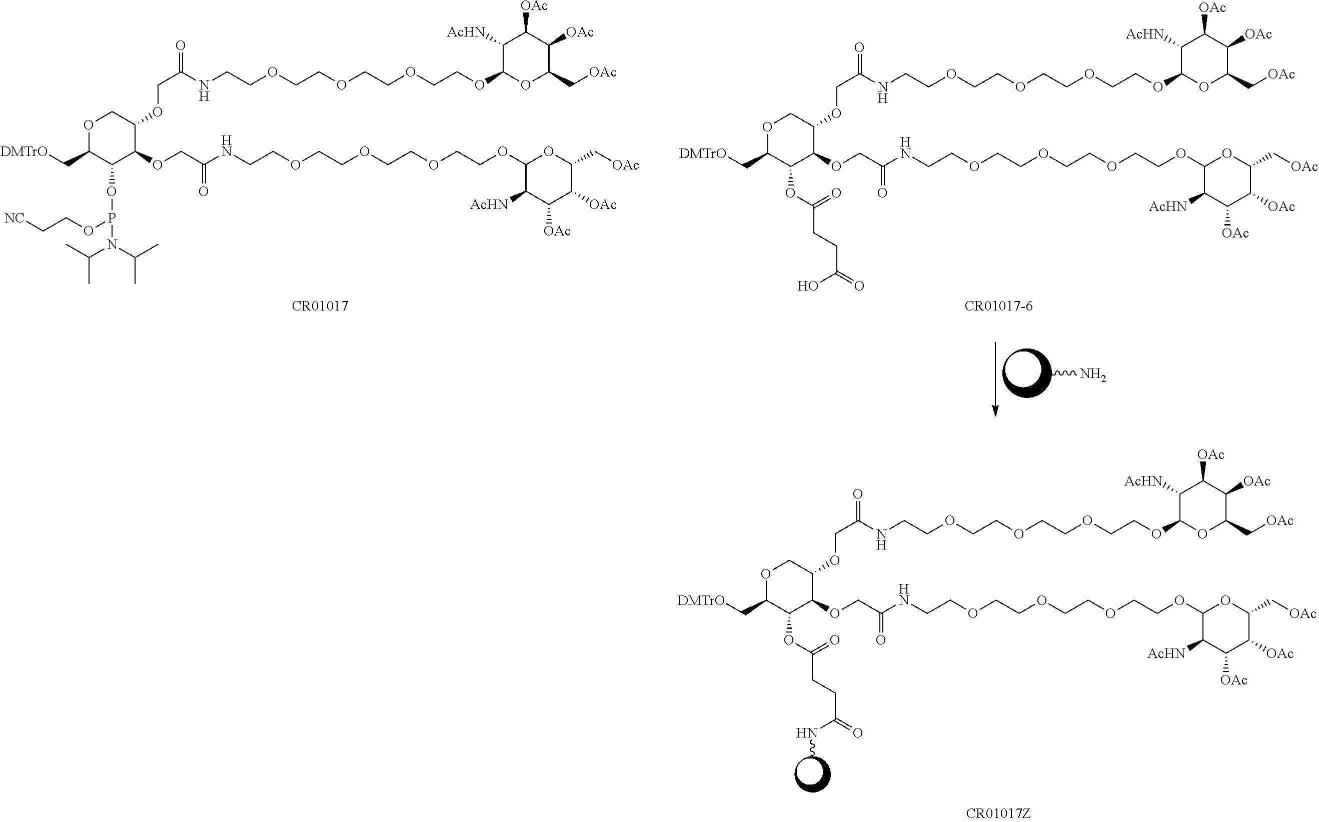

### (15-1) Synthesis of Compound CR01017-1

Compound NM041-4 (4.3 g 17.0 mmol, 1.0 eq) was dissolved in 50 ml of ultra-dry DMF. NaH (2.7 g 68 mmol, 4.0 eq) was added in an ice-bath and stirred for 1 h. Ethyl bromoacetate (5.7 g, 34 mmol, 2.0 eq, CAS No. 105-36-2) and KI (4.3 g 25.9 mmol, 1.5 eq) were added and allowed to react at 25 °C for 3 h. The saturated aqueous solution of ammonium chloride was added for quenching reaction. After the reaction was completed, 20 ml of water was added, and then the reaction solution was extracted three times with 50 ml of ethyl acetate (50 ml×3). The organic phases were combined, washed 10 times with 20 ml of saturated aqueous solution of sodium chloride (20 ml×10), dried over anhydrous sodium sulfate, filtered, and concentrated to obtain compound CR01017-1 as a dark brown oil, which was used directly in the next step. MS ESI (m/z)=425.2 [M+H]⁺.

### (15-2) Synthesis of Compound CR01017-2

Compound CR01017-1 (5 g, 1.0 eq) was dissolved in 30 ml of dichloromethane (DCM), and 300 ml of 70% by mass glacial acetic acid aqueous solution was added. The mixture was reacted at 50 °C for 2 h. After the reaction was completed, the reaction solution was directly concentrated to obtain compound CR01017-2 as a dark brown oil, which was used directly in the next step. MS ESI (m/z)=337 [M+H]⁺.

### (15-3) Synthesis of Compound CR01017-3

Compound CR01017-2 (3.2 g, 9.52 mmol, 1.0 eq) was dissolved in 70 ml of pyridine. DMTrCl (4.19 g, 12.4 mmol, 1.3 eq) was added in an ice bath. The mixture was stirred at 25 °C for 3 h, and then 30 ml of methanol was added for quenching reaction. After the reaction was completed, the reaction solution was concentrated. 20 ml of water was added, and the reaction solution was extracted three times with 50 ml of ethyl acetate (50 ml×3). The organic phases were combined, dried over anhydrous sodium sulfate, filtered, concentrated, and then purified by reverse-phase chromatography (C18 chromatographic column, eluent: acetonitrile/water=52/48, v/v) to obtain compound CR01017-3 as a yellow solid (5 g, 83% yield). MS ESI (m/z)=639 [M+H]⁺.

### (15-4) Synthesis of Compound CR01017-4

Compound CR01017-3 (2 g, 3.16 mmol, 1.0 eq) was dissolved in 10 mL of methanol, and NaOH aqueous solution (6.3 mL, 1 mol/L) was added. The mixture was stirred at 25 °C for 1 h. After the reaction was completed, the pH of the reaction solution was adjusted to neutral with hydrochloric acid. The reaction solution was filtered, and the filtrate was concentrated, and purified by reverse-phase chromatography (C18 chromatographic column, eluent: acetonitrile/water=26/74, v/v) to obtain compound CR01017-4 as a pale yellow solid (920 mg, 51% yield). MS ESI (m/z)=583 [M+H]⁺.

### (15-5) Synthesis of Compound CR01017-5

Compound CR01017-4 (900 mg, 1.55 mmol, 1 eq), (2R,3R,4R,5R,6R)-5-acetylamino-2-(acetoxymethyl)-6-(2-(2-aminoethoxy)ethoxy) (2.02 g, 3.88 mmol, 2.5 eq), and HATU (1.35 g, 3.56 mmol, 2.3 eq) were dissolved in 25 ml of N,N-dimethylformamide. DIEA (800 mg, 6.2 mmol, 4.0 eq) was added in an ice bath. The mixture was stirred at 25 °C for 12 h. After the reaction was completed, the reaction system was purified by reverse-phase chromatography (C18 chromatographic column, eluent: acetonitrile/water=20/80, v/v) to obtain compound CR01017-5 as a white solid (700 mg, 28.6% yield). MS ESI (m/z)=1591.2 [M+H]⁺.

### (15-6) Synthesis of Compound CR01017

Compound CR01017-5 (500 mg, 0.31 mmol, 1.0 eq), 4,5-dicyanoimidazole (30 mg, 0.25 mmol, 0.8 eq), and bis(diisopropylamino)(2-cyanoethoxy)phosphine (138 mg, 0.46 mmol, 1.5 eq) were dissolved in 10 ml of ultra-dry dichloromethane and reacted at 25 °C in a nitrogen atmosphere for 2 h. After the reaction was completed, the reaction solution was washed twice with 10 ml of saturated sodium bicarbonate (10 ml×2). The organic phase was concentrated and purified by reverse-phase chromatography (C18 chromatographic column, eluent: acetonitrile/water=80/20, v/v) to obtain compound CR01017 as a white solid (480 mg, 83.1% yield). MS ESI (m/z)=1791.2 [M+H]⁺.

### (15-7) Synthesis of Compound CR01017-6

Compound CR01017-5 (200 mg, 0.13 mmol, 1.0 eq) was dissolved in 10 ml of dichloromethane. Succinic anhydride (14 mg, 0.14 mmol, 1.1 eq) and triethylamine (26 mg, 0.26 mmol, 2.0 eq) were added. The mixture was stirred at 25 °C for 12 h. After the reaction was completed, the reaction solution was concentrated and purified by reverse-phase chromatography (C18 chromatographic column, eluent: acetonitrile/water=36/64, v/v) to obtain compound CR01017-6 as a white solid (150 mg, 71.4% yield). MS ESI (m/z)=1691.2 [M+H].

### (15-8) Synthesis of Compound CR01017Z

Compound CR01017-6 (70 mg, 0.041 mmol, 1.0 eq), benzotriazole-N,N,N′,N′-tetramethyluronium hexafluorophosphate (abbreviated as HBTU, 22 mg, 0.062 mmol, 1.5 eq), and N,N-diisopropylethylamine (10 mg 0.082 mmol, 2.0 eq) were dissolved in 10 ml of acetonitrile. The mixture was stirred at 25 °C for 5 min. Aminoalkyl-CPG (970 mg, 80 μmol/g) was added. The mixture was reacted on a shaker at 25 °C for 18 h. The reaction solution was filtered and the filter cake was washed twice with 10 ml of dichloromethane (10 ml×2) and then washed twice with 10 ml of acetonitrile (10 ml×2). The filter cake, DMAP (3 mg, 0.024 mmol), Cap1 (10 ml) and Cap2 (1 ml) were mixed and reacted on a shaker at 25 °C for 5 h. The reaction solution was filtered, and the filter cake was rinsed three times with 10 ml of acetonitrile and dried to obtain compound CR01017Z (720 mg).

Wherein, Cap1 and Cap2 were capping agents, Cap1 was a mixed solution of 20 vol % N-methylimidazole in pyridine/acetonitrile, the volume ratio of pyridine to acetonitrile was 3:5, and Cap2 was a solution of 20 vol % acetic anhydride in acetonitrile.

### Preparation Example 16: Synthesis Method 1 of siRNA Conjugate

### (16-1) Synthesis of Sense Strand (SS)

According to the solid-phase nucleic acids synthesis method using phosphoramidite, nucleoside monomers (the compounds CR01017, CR01020, CR01021, CR01022, CR01023, CR01024, CR01025, and CR01026 were each regarded as a single nucleoside monomer) were iteratively linked one by one in the 3′ to 5′ direction according to the nucleotides sequence, starting from the compounds connected to the solid phase support (i.e., CR01017Z, CR01020Z, CR01021Z, CR01022Z, CR01023Z, CR01024Z, CR01025Z, CR01026Z, CR01027Z and L96-PS).

The linking of each nucleoside monomer comprised a four-step reaction of deprotection, coupling capping and oxidation or sulfurization. The synthesis condition was given below.

The nucleoside monomers were prepared to a solution of 0.1 M nucleoside monomer in acetonitrile.

The condition for deprotection reaction in each step was identical, including a temperature of 25 °C, a reaction time of 70 seconds, a solution of dichloroacetic acid in dichloromethane (3 vol %) as a deprotection agent, and a molar ratio of the dichloroacetic acid to the protecting group 4,4′-dimethoxytrityl on the solid phase support of 5:1.

The condition for coupling reaction in each step was identical, including a temperature of 25 °C, a molar ratio of the nucleic acid sequence linked to the solid phase support to the nucleoside monomers of 1:10, a molar ratio of the nucleic acid sequence linked to the solid phase support to a coupling agent of 1:65, a reaction time of 600 seconds, a solution of 0.5 M 5-ethylthio-1H-tetrazole in acetonitrile as a coupling agent, and a mixed solution of 0.2 M xanthane hydride in acetonitrile/pyridine (volume ratio of acetonitrile:pyridine=1:1) as a thio agent.

The condition for capping reaction in each step was identical, including a temperature of 25 °C, a reaction time of 2 min, a mixed solution of Cap1 and Cap2 in a molar ratio of 1:1 as a capping agent, a mixed solution of 20 vol % N-methylimidazole in pyridine/acetonitrile as Cap1, a volume ratio of pyridine to acetonitrile of 3:5, a solution of 20 vol % acetic anhydride in acetonitrile as Cap2, and a molar ratio of N-methylimidazole in the capping agent Cap1:acetic anhydride in the capping agent Cap2:the nucleic acid sequence connected to the solid phase support of 1:1:1.

The condition for oxidation reaction in each step was identical, including a temperature of 25 °C, a reaction time of 3 seconds, 0.05 M iodine water as an oxidation agent, and a molar ratio of iodine to the nucleic acid sequence connected to the solid phase support in the coupling reaction of 30:1. The oxidation reaction was carried out in a mixed solvent of water/pyridine (volume ratio of water:pyridine=1:9). The condition for sulfurization reaction included a temperature of 25 °C, a reaction time of 360 seconds, a solution of 0.2 M xanthane hydride in pyridine as a thio agent, and a molar ratio of the thio agent to the nucleic acid sequence connected to the solid phase support in the coupling reaction of 4:1. The sulfurization reaction was carried out in a mixed solvent of water/pyridine (volume ratio of water:pyridine=1:9).

After the last nucleoside monomer was linked, the nucleic acid sequence connected to the solid phase support was cleaved, deprotected, purified and desalted in turn, and then freeze-dried to obtain the sense strand.

Among them, the conditions for cleavage and deprotection were as follows: the synthesized nucleotide sequence connected to the solid phase support was added into a 25% by mass aqueous ammonia solution for 16 h of reaction at 55 °C, wherein the aqueous ammonia solution was used in an amount of 0.5 ml/μmol. The solvent was removed, and the residue was concentrated under vacuum to dryness. After the treatment by aqueous ammonia solution was completed, the resulting product was dissolved with 0.4 ml/μmol N-methylpyrrolidone relative to the amount of single-stranded nucleic acid, and then added with 0.3 ml/μmol triethylamine and 0.6 ml/μmol triethylamine trihydrofluoride to remove 2′-O-TBDMS protection from the ribose.

The conditions for purification and desalination were as follows: the nucleic acids were purified using a preparative ion chromatography column (Source 15Q) with a gradient elution by NaCl. Specifically, eluting agent 1 was 20 mM sodium phosphate (pH 8.1) in a mixed solvent of water/acetonitrile (volume ratio of water:acetonitrile=9:1); eluting agent 2 was 1.5 M sodium chloride and 20 mM sodium phosphate (pH 8.1) in a mixed solvent of water/acetonitrile (volume ratio of water:acetonitrile=9:1); eluent was a mixture of eluting agent 1 and eluting agent 2 in a ratio of (100:0) to (50:50). The eluate was collected, combined and desalted by using a reverse phase chromatography purification column. The conditions for desalination comprised using a sephadex column (filler: Sephadex-G25) for desalination, and deionized water for elution.

Detection: The purity detection was performed using ion exchange chromatography (IEX-HPLC), and the molecular weight was measured by LC-MS (Liquid ChromatogRLhy-Mass SPlectrometry, Model: LCT Premier, Waters). The measured value of the molecular weight and the theoretical value of the molecular weight were compared. When the measured value was consistent with the theoretical value, it indicated that a sense strand of siRNA conjugated with a compound at 3′ end was obtained.

During the synthesis of the sense chain, the following ligands (carriers) were synthesized respectively.

The dimeric CR01017 (expressed as (CR01017)×2) has the following structural formula:

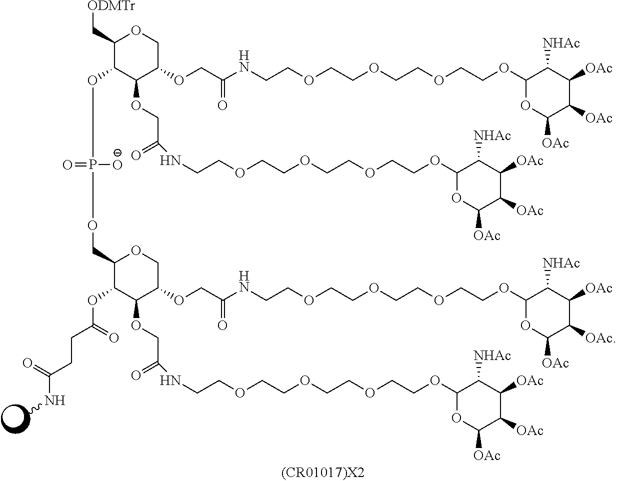

The trimeric CR01020 (expressed as (CR01020)×3) has the following structural formula:

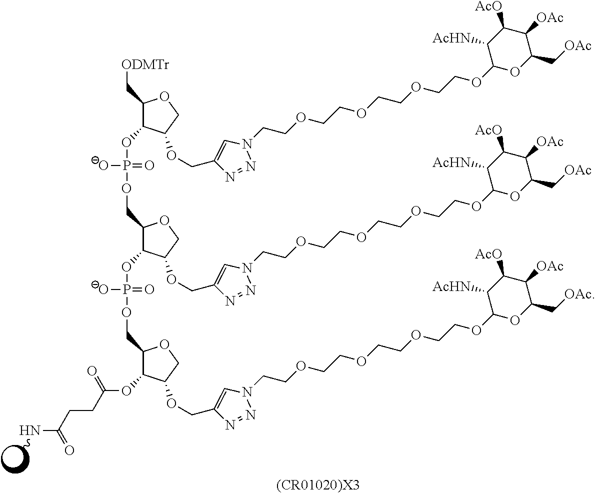

The trimeric CR01021 (expressed as (CR01021)×3) has the following structural formula:

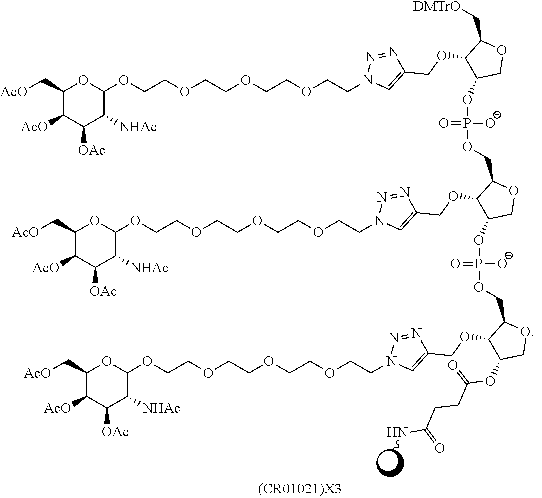

The dimeric CR01022 (expressed as (CR01022)×2) has the following structural formula:

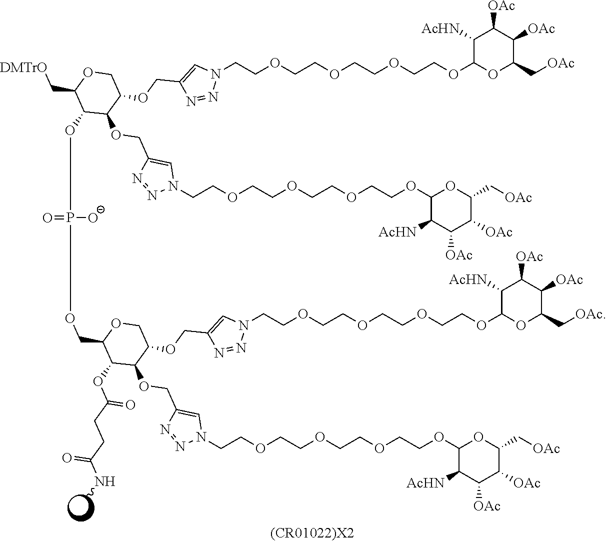

The trimeric CR01022 (expressed as (CR01022)×3) has the following structural formula:

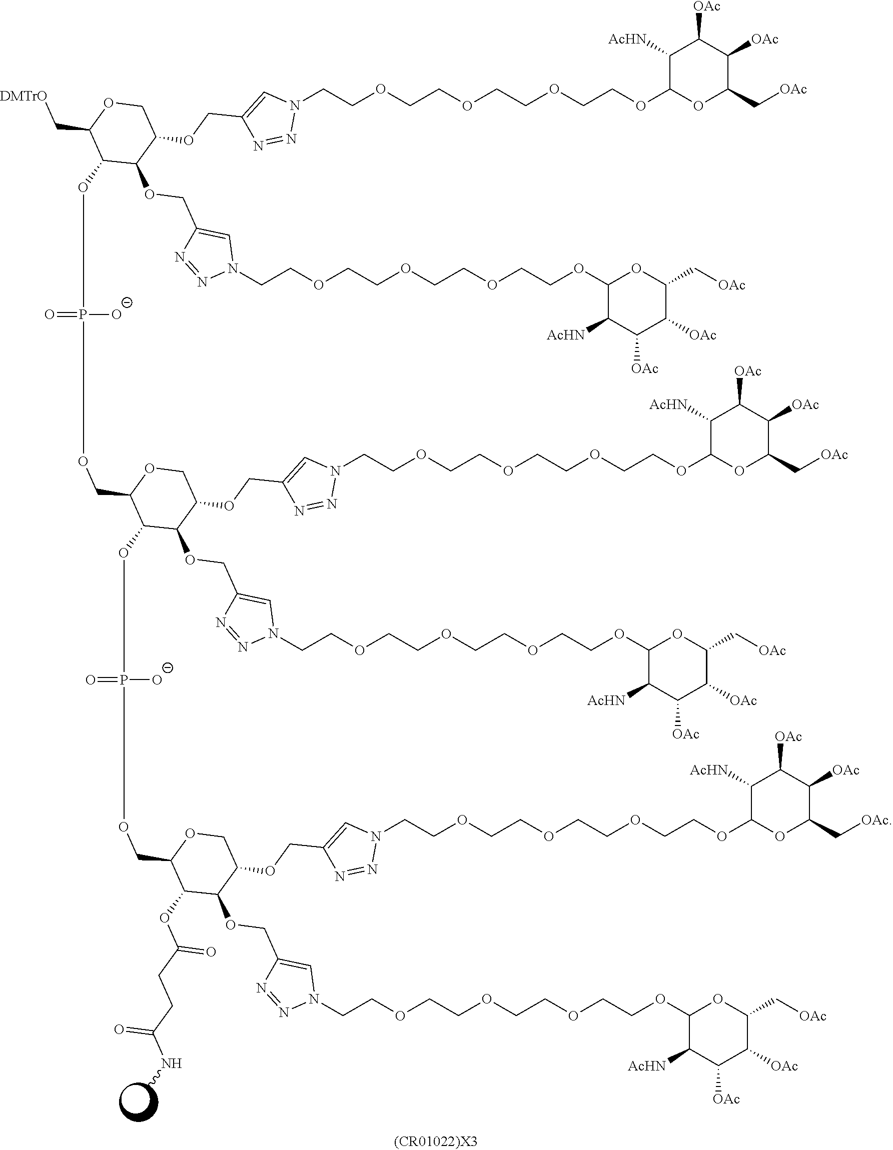

The trimeric CR01023 (expressed as (CR01023)×3) has the following structural formula:

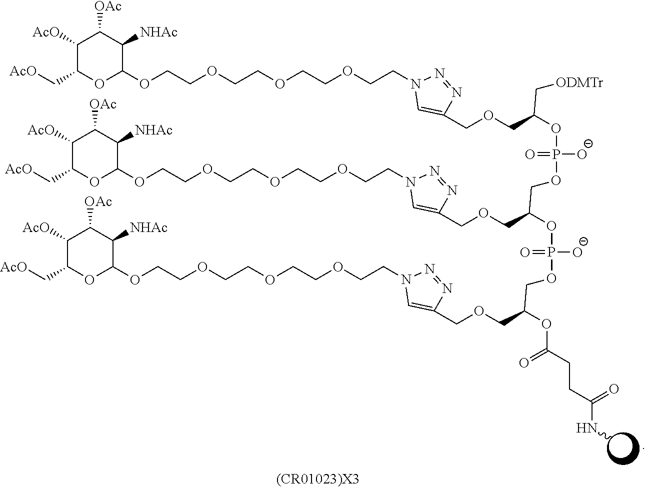

The trimeric CR01024 (expressed as (CR01024)×3) has the following structural formula:

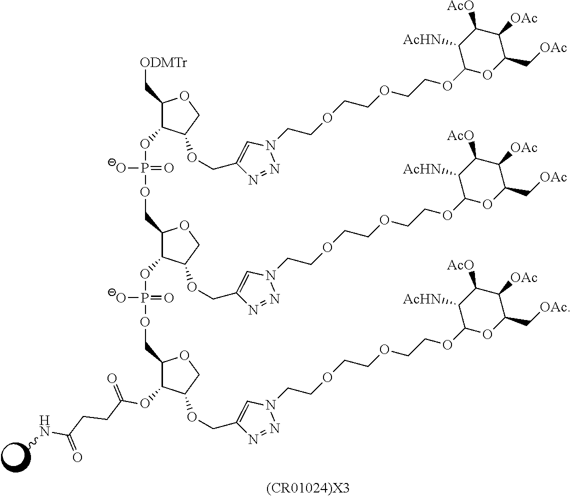

The trimeric CR01025 (expressed as (CR01025)×3) has the following structural formula:

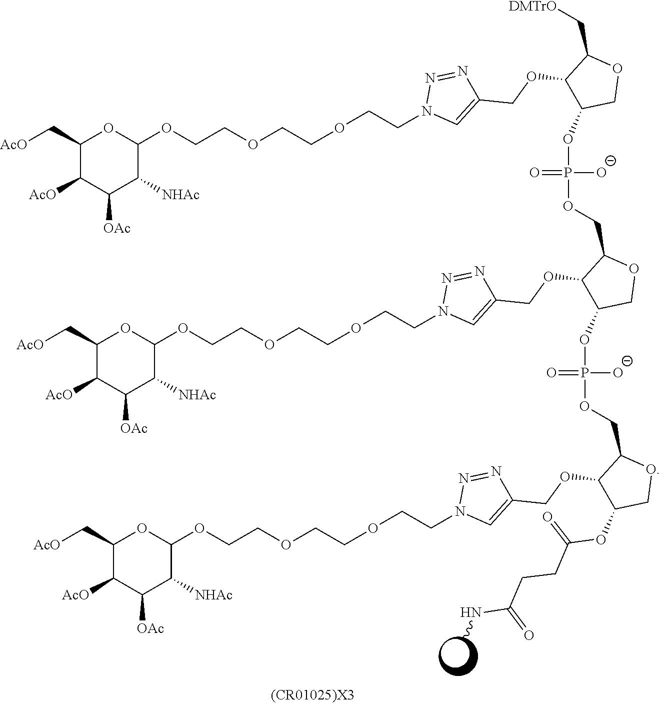

The dimeric CR01026 (expressed as (CR01026)×2) has the following structural formula:

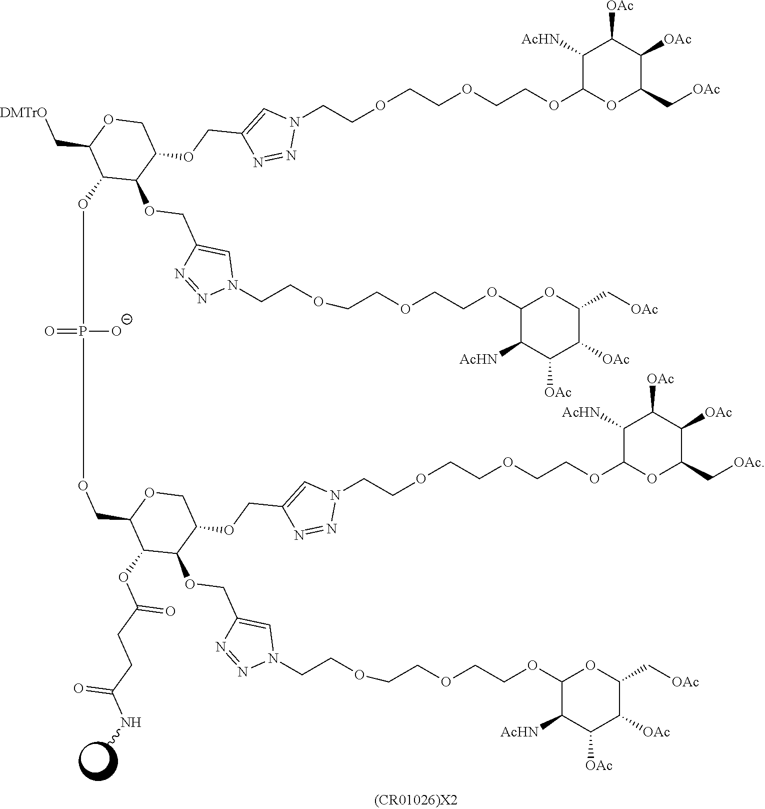

The dimeric CR01027 (expressed as (CR01027)×2) has the following structural formula:

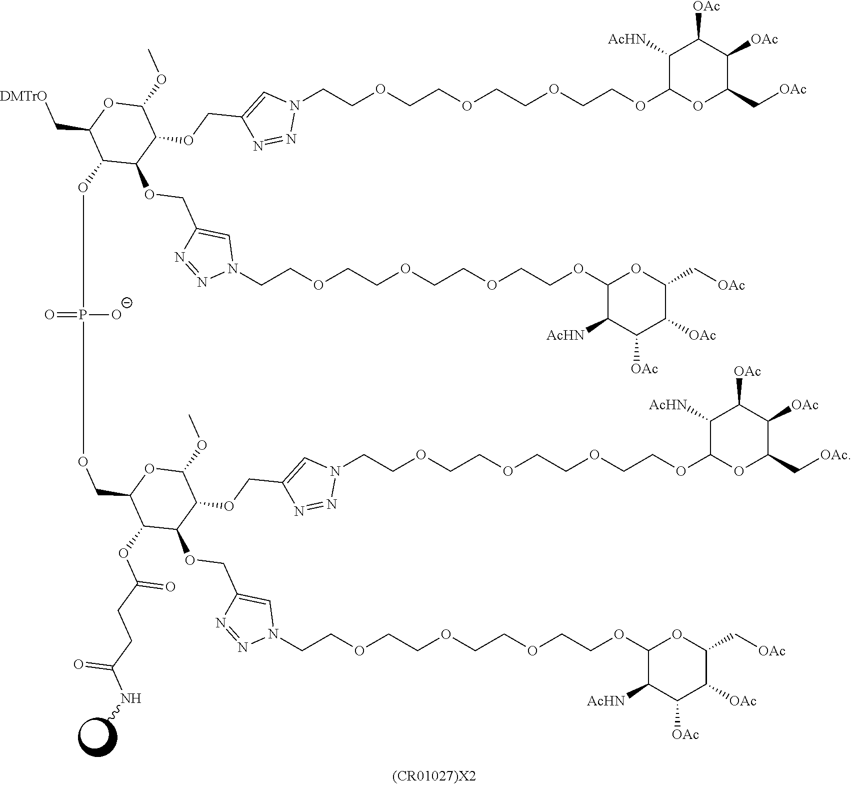

### (16-2) Synthesis of Antisense Strand (AS)

Antisense strands were synthesized using a general solid phase support. The reaction conditions of deprotection, coupling, capping, oxidation or sulfurization, cleavage and deprotection, and purification and desalination in the solid phase synthesis method were the same as those used for the synthesis of the sense strand in step (16-1).

Detection: The purity detection was performed using ion exchange chromatography (IEX-HPLC), and the molecular weight was measured by LC-MS (Liquid ChromatogRLhy-Mass SPlectrometry, Model: LCT Premier, Waters). The measured value of the molecular weight and the theoretical value of the molecular weight were compared. When the measured value was consistent with the theoretical value, it indicated that an antisense strand of siRNA was obtained.

### (16-3) Synthesis of siRNA Conjugate

The sense strand synthesized in step (16-1) and the antisense strand synthesized in step (16-2) were mixed at an equimolar ratio, dissolved in water for injection, heated to 95 °C, slowly cooled to room temperature and left to stand at room temperature for 10 min to allow the sense and antisense strands to form a double-stranded structure by hydrogen bonds, thereby obtaining the target siRNA conjugate.

Detection: Each conjugate was diluted to a concentration of 0.2 mg/mL (calculated based on siRNA) with ultrapure water (produced from Milli-Q ultrapure water system, resistivity: 18.2 MΩ·cm at 25 °C). The molecular weight was measured by LC-MS (Liquid ChromatoRLhy-Mass SPlectrometry, Model: LCT Premier, Waters). When the measured value was consistent with the theoretical value, it indicated that the synthesized conjugates correspond to the designed target double-stranded nucleic acid sequences.

### Preparative Example 17: Synthesis Method 2 of siRNA Conjugate

In this preparative example, the linker connecting the siRNA and the ligand moiety used in the siRNA conjugates provided by the present disclosure is represented by formula A (NM014, NM014A, NM041 and NM042):

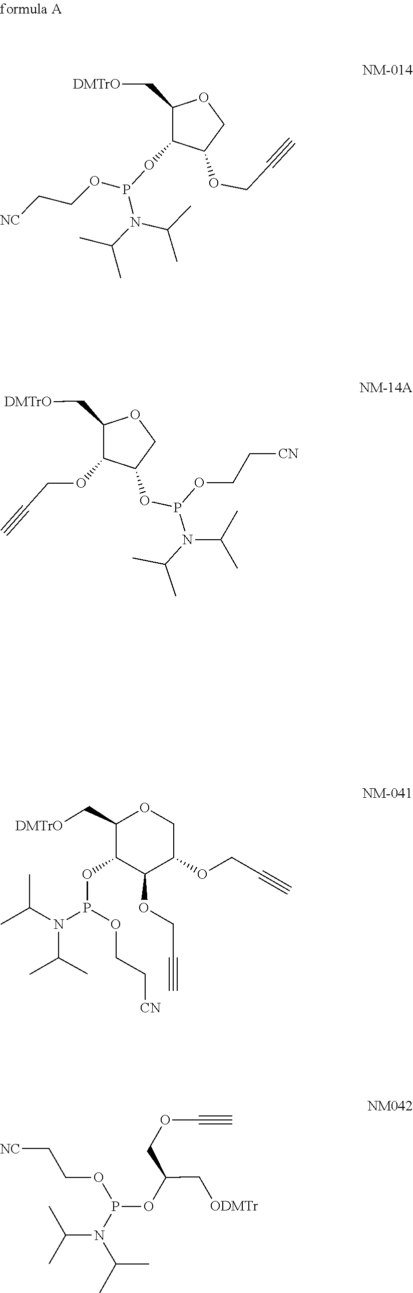

The ligand moiety is represented by formula B (NM015 and NM043)

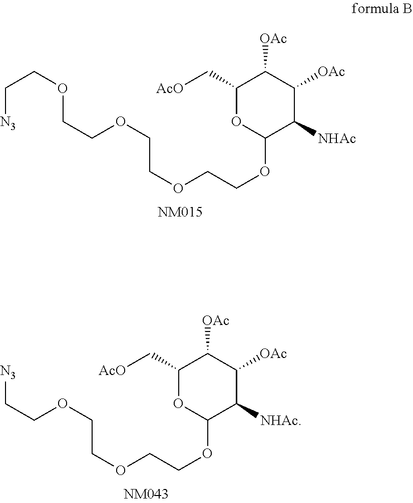 [SMILES: CC(=O)N[C@H]1C(OCCOCCOCCOCCN=[N+]=[N-])O[C@H](COC(C)=O)[C@H](OC(C)=O)[C@@H]1OC(C)=O ; CC(=O)N[C@H]1C(OCCOCCOCCN=[N+]=[N-])O[C@H](COC(C)=O)[C@H](OC(C)=O)[C@@H]1OC(C)=O]

Taking the linker NM041 and the ligand moiety NM015 as examples, the synthesis process of siRNA includes the following steps:

### (17-1) Synthesis of Sense Strand

The synthesis route of the sense strand is shown below:

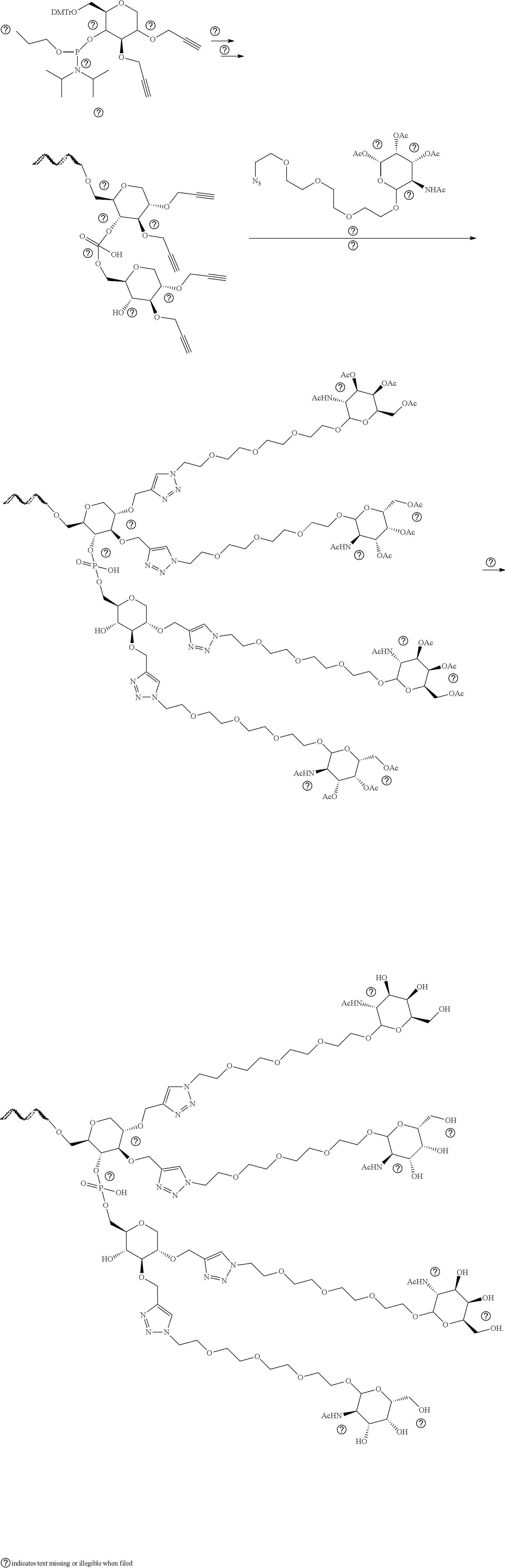

Specifically, the preparation method of the sense strand included the following processes.

S1: Regarding compound NM041 as a nucleoside monomer, NM041 was connected to a solid support by the solid-phase synthesis method using phosphoramidite. Subsequently, following the type and sequence of nucleotides constituting the sense strand of the siRNA above, nucleoside monomers were linked one by one in the direction from 3′ to 5′. The linking of each nucleoside monomer comprised a four-step reaction of deprotection, coupling, capping, and oxidation or sulfurization. The reaction conditions for deprotection, coupling, capping, oxidation and sulfuirization in the synthesis of sense strand were identical to those described in step (16-1).

The partial conjugates containing the sense strand and linker (expressed as SS-(NM041)×2) were separated, where all NM041 linkers were connected to the 3′ end of the sense strand of siRNA.

S2: The terminal alkyne group of SS-(NM041)×2 was conjugated to the ligand NM015 through the “click” reaction to obtain the sense strand conjugate.

S2-1: 150 μL of H₂O, 70 μL of 0.2 mol/L carbonate buffer (pH 9.2), and 70 μL of N,N-dimethylformamide (DMF) were mixed to obtain a mixed solvent. SS-(NM041)×2 was dissolved in the mixed solvent to obtain a SS-(NM041)×2 solution with a concentration of 1.0 eq.

S2-2: 6.0 eq of the ligand compound NM015 was dissolved in 70 μL of DMF to obtain a NM015 solution.

S2-3: The SS-(NM041)×2 solution obtained in 52-1 and the NM015 solution obtained in S2-2 were mixed to obtain a mixed reaction solution.

S2-4: 10.0 eq of tris(3-hydroxypropyltriazolylmethyl)amine (THPTA) and 3.0 eq of CuSO₄·5H₂O were mixed in a volume ratio of THPTA: CuSO₄·5H₂O=10:3, and shaken at 40 °C for 5 min to obtain a mixture. 37 μL of the mixture was added to the mixed reaction solution obtained in S2-3 and subjected to vortex to obtain a mixed solution of intermediate. The pH of the mixed solution of intermediate was measured to be 8.0.

S3: 25.0 eq of sodium ascorbate was rapidly added to the mixed solution of intermediate obtained in S2-4, and subjected to vortex. After 1 h of reaction at 40 °C was completed, a mixed solution of product was obtained.

S4: The purification was as follows. 3 μL of the mixed solution of product obtained in S3 was diluted with a mixed solution of DMF and H₂O at a volume ratio of 1:5 and separated and purified by HPLC. For the HPLC, a C18 column was used as the chromatographic column, an ammonium bicarbonate buffer was used as the mobile phase and gradient elution was adopted. The product purified by HPLC was lyophilized.

### (17-2) Synthesis of Antisense Strand

The preparation was performed according to the synthesis method for the antisense strand (AS) described in preparation example 16 (16-2).

### (17-3) Synthesis of siRNA Conjugate

The preparation was performed according to the synthesis method for the siRNA conjugate described in preparation example 16 (16-3).

For the two methods for synthesizing siRNA conjugates provided in the present disclosure, the method 1 involves synthesizing the carrier first, then the sense strand conjugate, and finally the siRNA conjugate. The method 2 involves synthesizing the partial conjugate of sense strand first, then the sense strand conjugate, and finally the siRNA conjugate.

In the case of synthesizing the same amount of siRNA conjugates, the synthesis method 1 requires fewer raw materials for the synthesis of carriers but takes longer to complete the synthesis of siRNA conjugates compared to the synthesis method 2. In contrast, the synthesis method 2 takes shorter to complete the synthesis of siRNA conjugates, but requires more raw materials for the synthesis of carriers compared to the synthesis method 1.

The siRNA conjugate with a monomeric CR01017 as carrier has a structure of

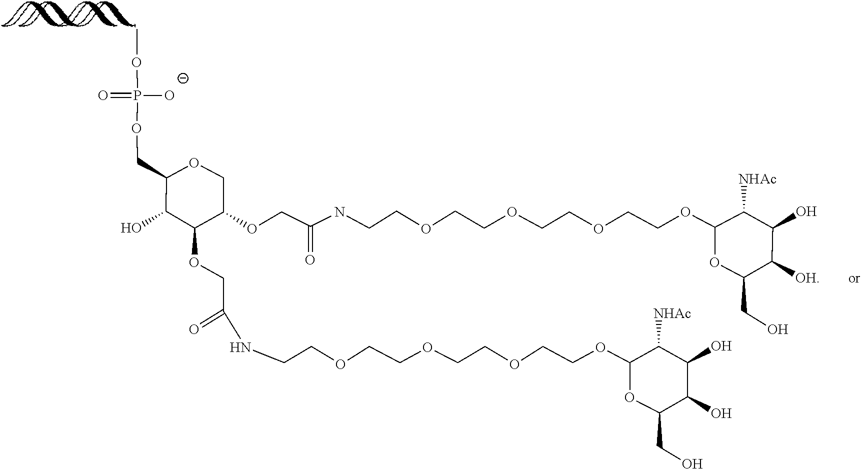

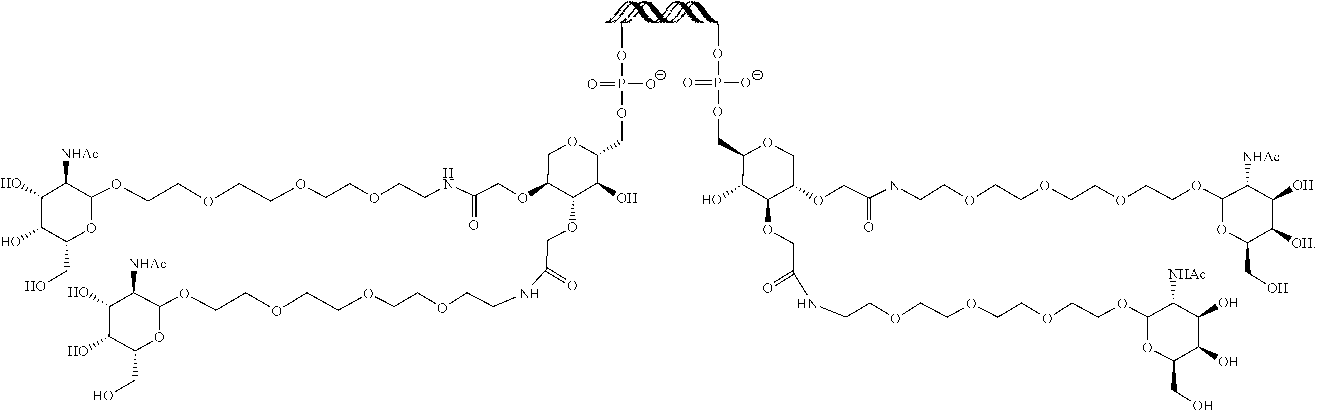

The siRNA conjugate with a dimeric CR01017 as carrier has a structure of

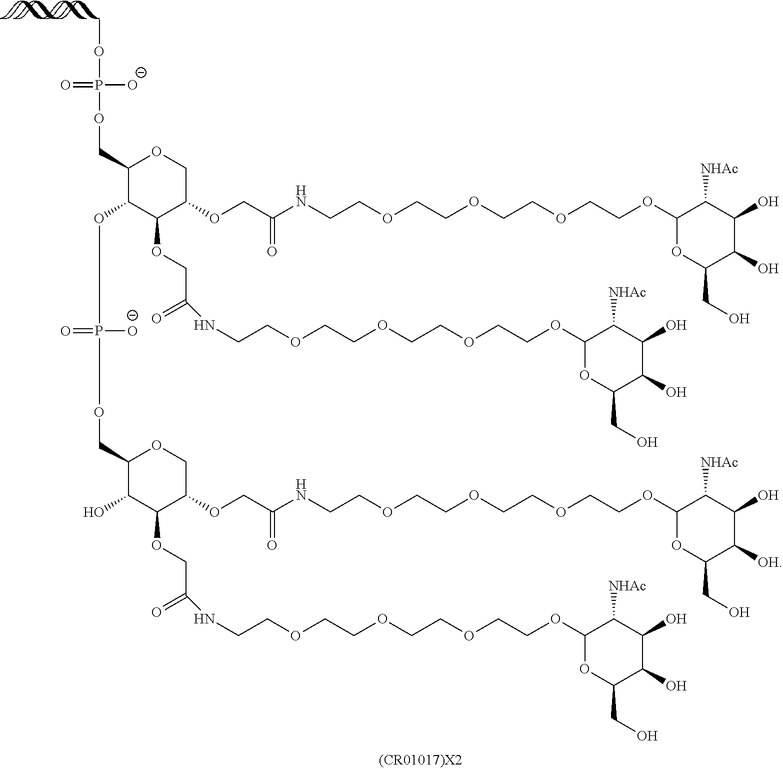 [SMILES: COP(=O)([O-])OC[C@H]1OC[C@H](OCC(=O)NCCOCCOCCOCCOC2O[C@H](CO)[C@H](O)[C@H](O)[C@H]2NC(C)=O)[C@@H](OCC(=O)NCCOCCOCCOCCOC2O[C@H](CO)[C@H](O)[C@H](O)[C@H]2NC(C)=O)[C@@H]1OP(=O)([O-])OC[C@H]1OC[C@H](OCC(=O)NCCOCCOCCOCCOC2O[C@H](CO)[C@H](O)[C@H](O)[C@H]2NC(C)=O)[C@@H](OCC(=O)NCCOCCOCCOCCOC2O[C@H](CO)[C@H](O)[C@H](O)[C@H]2NC(C)=O)[C@@H]1O]

The siRNA conjugate with a trimeric CR01020 as carrier has a structure of

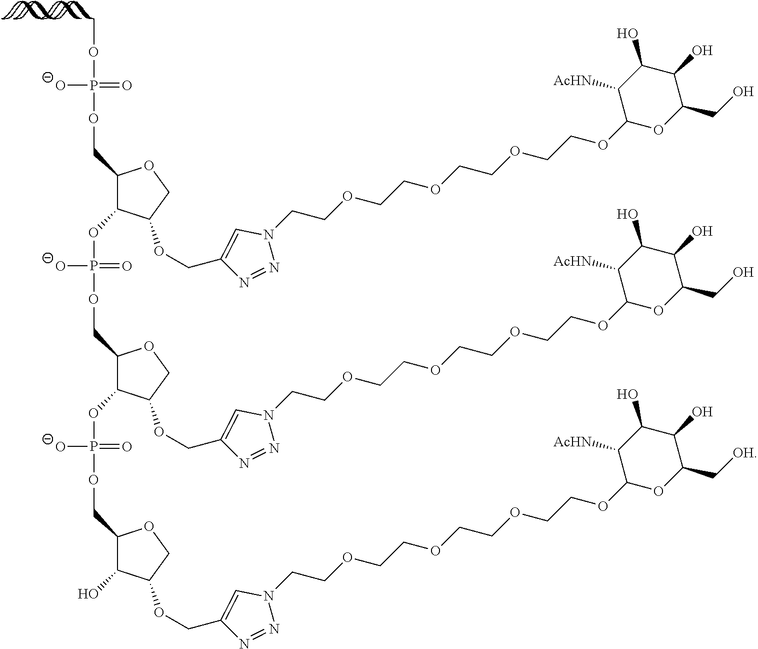 [SMILES: COP(=O)([O-])OC[C@H]1OC[C@H](OCc2cn(CCOCCOCCOCCOC3O[C@H](CO)[C@H](O)[C@H](O)[C@H]3NC(C)=O)nn2)[C@@H]1OP(=O)([O-])OC[C@H]1OC[C@H](OCc2cn(CCOCCOCCOCCOC3O[C@H](CO)[C@H](O)[C@H](O)[C@H]3NC(C)=O)nn2)[C@@H]1OP(=O)([O-])OC[C@H]1OC[C@H](OCc2cn(CCOCCOCCOCCOC3O[C@H](CO)[C@H](O)[C@H](O)[C@H]3NC(C)=O)nn2)[C@@H]1O]

The siRNA conjugate with a trimeric CR01021 as carrier has a structure of

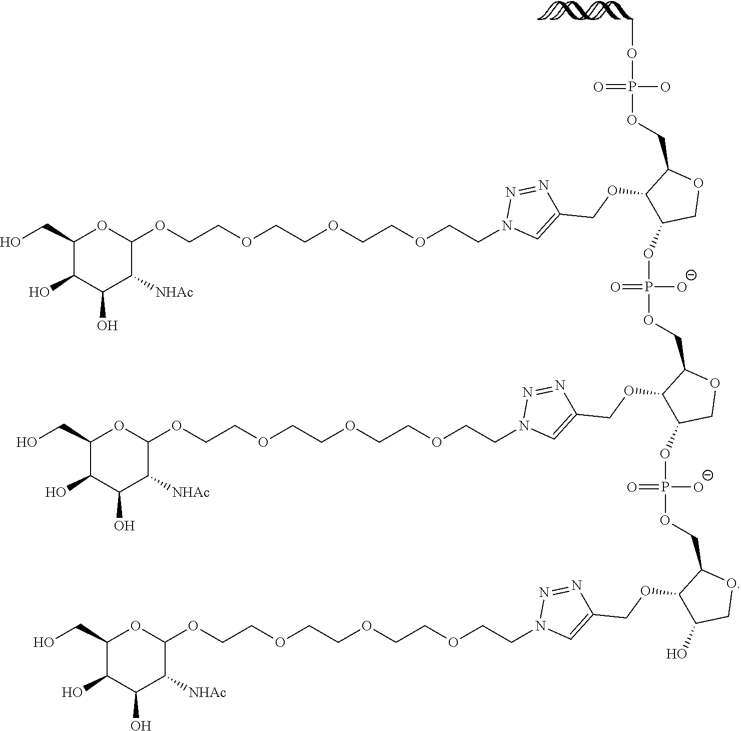 [SMILES: COP(=O)(O)OC[C@H]1OC[C@H](OP(=O)([O-])OC[C@H]2OC[C@H](OP(=O)([O-])OC[C@H]3OC[C@H](O)[C@@H]3OCc3cn(CCOCCOCCOCCOC4O[C@H](CO)[C@H](O)[C@H](O)[C@H]4NC(C)=O)nn3)[C@@H]2OCc2cn(CCOCCOCCOCCOC3O[C@H](CO)[C@H](O)[C@H](O)[C@H]3NC(C)=O)nn2)[C@@H]1OCc1cn(CCOCCOCCOCCOC2O[C@H](CO)[C@H](O)[C@H](O)[C@H]2NC(C)=O)nn1]

The siRNA conjugate with a monomeric CR01022 as carrier has a structure of

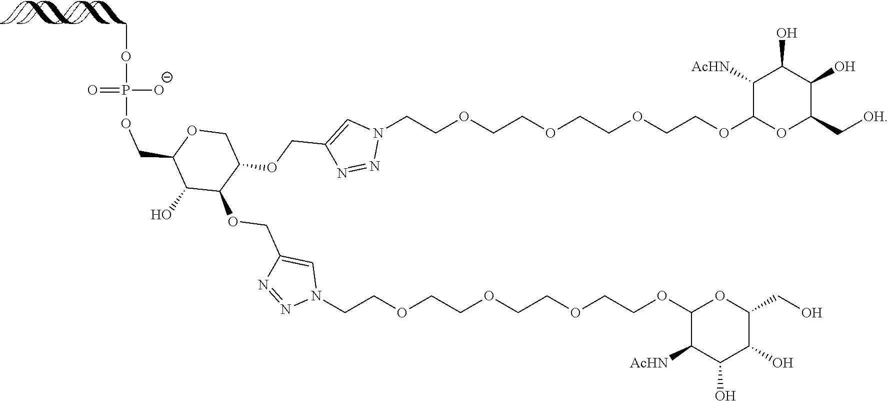 [SMILES: COP(=O)([O-])OC[C@H]1OC[C@H](OCc2cn(CCOCCOCCOCCOC3O[C@H](CO)[C@H](O)[C@H](O)[C@H]3NC(C)=O)nn2)[C@@H](OCc2cn(CCOCCOCCOCCOC3O[C@H](CO)[C@H](O)[C@H](O)[C@H]3NC(C)=O)nn2)[C@@H]1O]

The siRNA conjugate with a dimeric CR01022 as carrier has a structure of

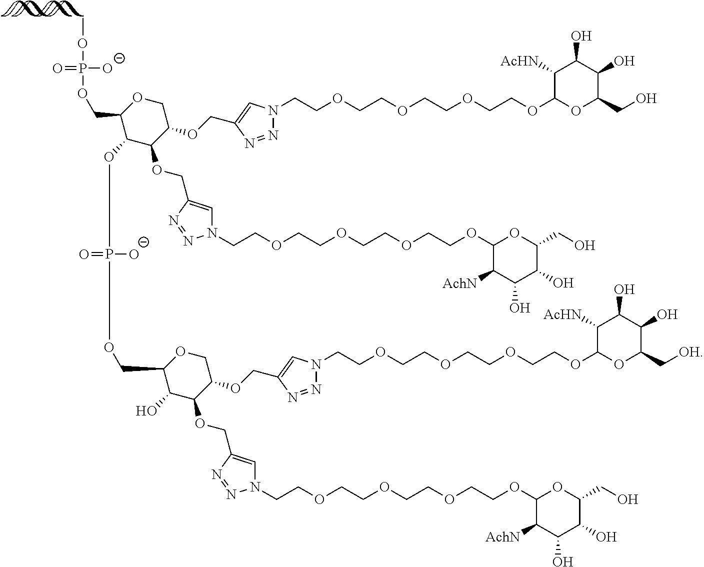

The siRNA conjugate with a trimeric CR01022 as carrier has a structure of

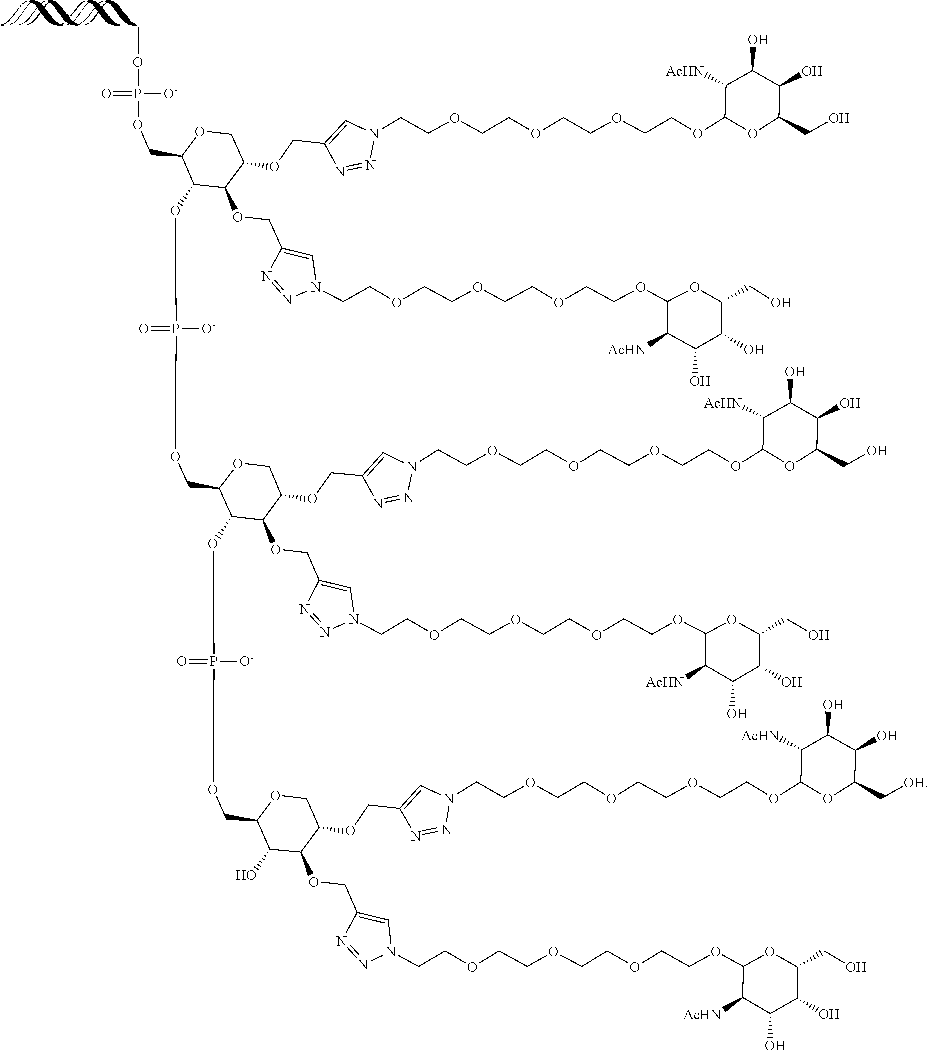

The siRNA conjugate with a trimeric CR01023 as carrier has a structure of

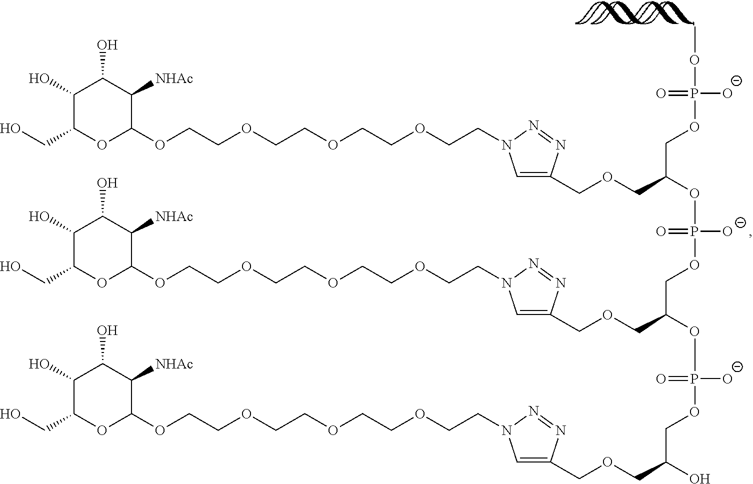 [SMILES: COP(=O)([O-])OC[C@H](COCc1cn(CCOCCOCCOCCOC2O[C@H](CO)[C@H](O)[C@H](O)[C@H]2NC(C)=O)nn1)OP(=O)([O-])OC[C@H](COCc1cn(CCOCCOCCOCCOC2O[C@H](CO)[C@H](O)[C@H](O)[C@H]2NC(C)=O)nn1)OP(=O)([O-])OC[C@@H](O)COCc1cn(CCOCCOCCOCCOC2O[C@H](CO)[C@H](O)[C@H](O)[C@H]2NC(C)=O)nn1]

The siRNA conjugate with a trimeric CR01024 as carrier has a structure of

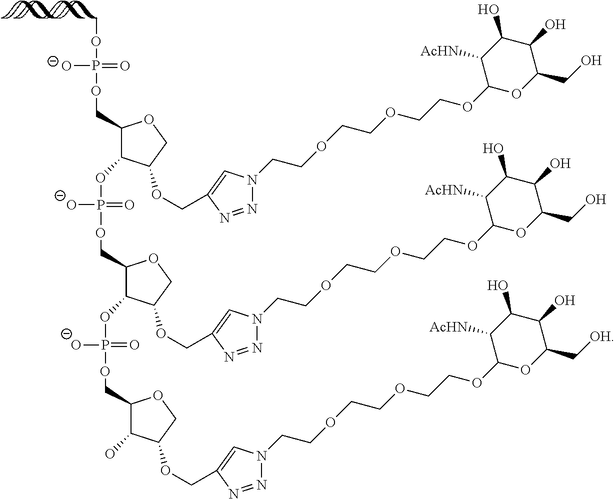 [SMILES: COP(=O)([O-])OC[C@H]1OC[C@H](OCc2cn(CCOCCOCCOC3O[C@H](CO)[C@H](O)[C@H](O)[C@H]3NC(C)=O)nn2)[C@@H]1OP(=O)([O-])OC[C@H]1OC[C@H](OCc2cn(CCOCCOCCOC3O[C@H](CO)[C@H](O)[C@H](O)[C@H]3NC(C)=O)nn2)[C@@H]1OP(=O)([O-])OC[C@H]1OC[C@H](OCc2cn(CCOCCOCCOC3O[C@H](CO)[C@H](O)[C@H](O)[C@H]3NC(C)=O)nn2)[C@@H]1O]

The siRNA conjugate with a trimeric CR01025 as carrier has a structure of

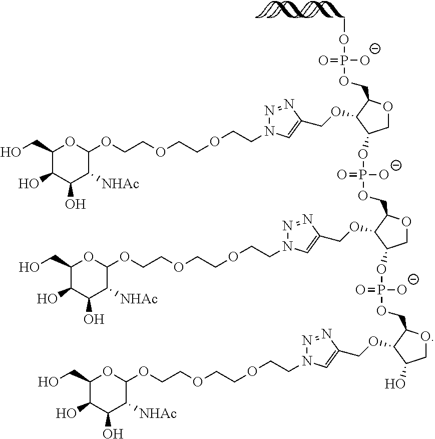 [SMILES: COP(=O)([O-])OC[C@H]1OC[C@H](OP(=O)([O-])OC[C@H]2OC[C@H](OP(=O)([O-])OC[C@H]3OC[C@H](O)[C@@H]3OCc3cn(CCOCCOCCOC4O[C@H](CO)[C@H](O)[C@H](O)[C@H]4NC(C)=O)nn3)[C@@H]2OCc2cn(CCOCCOCCOC3O[C@H](CO)[C@H](O)[C@H](O)[C@H]3NC(C)=O)nn2)[C@@H]1OCc1cn(CCOCCOCCOC2O[C@H](CO)[C@H](O)[C@H](O)[C@H]2NC(C)=O)nn1]

The siRNA conjugate with a dimeric CR01026 as carrier has a structure of

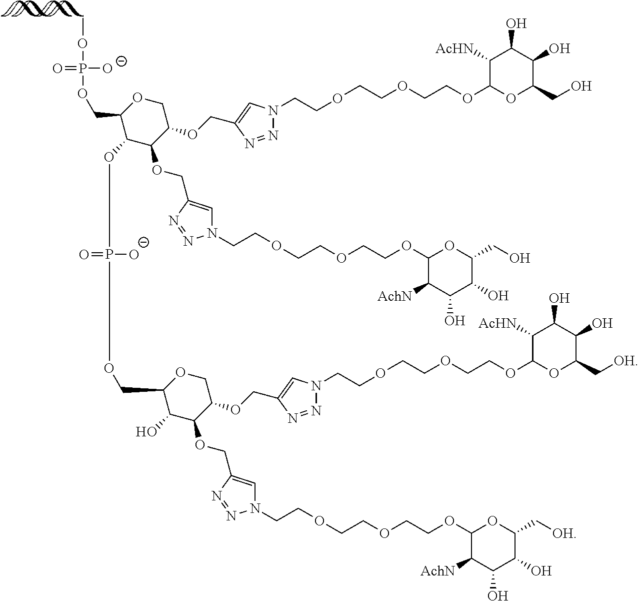

The siRNA conjugate with a dimeric CR01027 as carrier has a structure of:

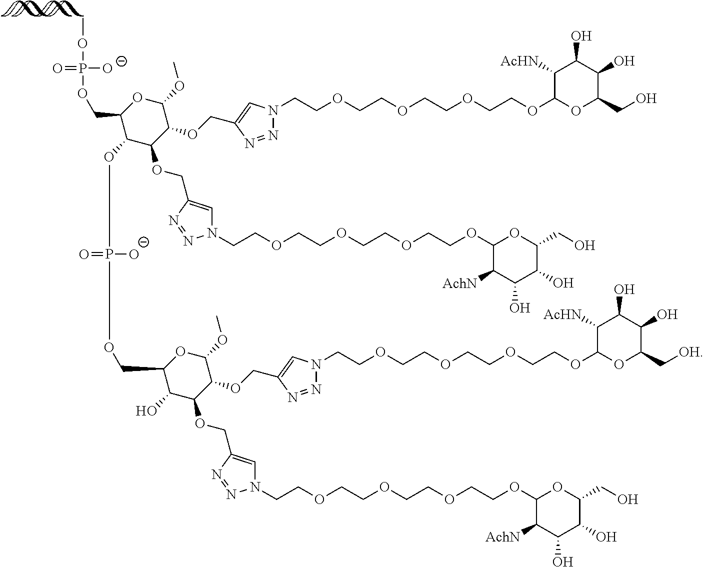

The siRNA conjugate with L96 as carrier has a structure of:

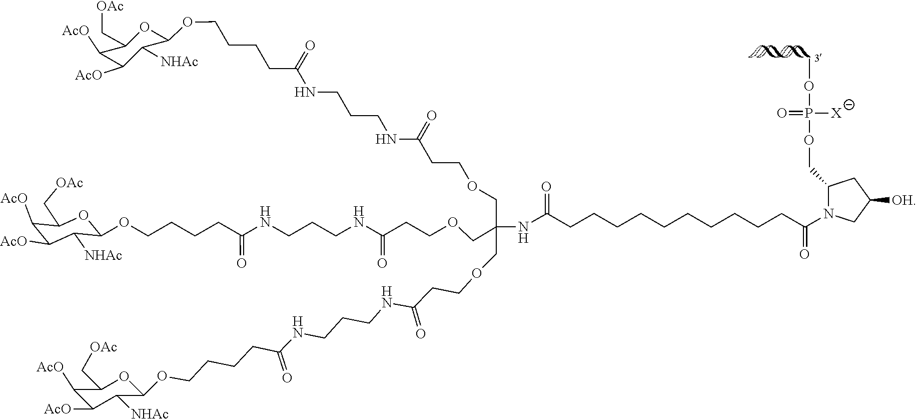

The siRNA conjugates shown in Table 3 were synthesized by the methods described in preparation example 16 and preparation example 17.

In the present disclosure, the conjugates RZ502031, RZ599001, RZ597002, RZ899038, RZ899041, and RZ899042 can only be synthesized by the synthesis method 1 of siRNA conjugate described in preparation example 16.

The conjugates RZ802008, RZ802009, RZ899007, RZ802014, RZ899022, RZ899023, RZ899024, RZ802010, RZ899009, RZ899011, RZ899040, RZ899025, RZ802011, RZ899010, RZ899048, and RZ897013 can be synthesized by either synthesis method 1 of siRNA conjugate described in preparation example 16 or synthesis method 2 of siRNA conjugate described in preparation example 17. Specifically, in the present disclosure, conjugates RZ802010, RZ899009, RZ899011, and RZ899040 were synthesized by the synthesis method 1 of siRNA conjugate described in preparation example 16. Conjugates RZ802008, RZ802009, RZ899007, RZ802014, RZ899022, RZ899023, RZ899024, RZ899025, RZ802011, RZ899010, RZ899048, and RZ897013 were synthesized by the synthesis method 2 of siRNA conjugate described in preparation example 17.

SEQ ID No.1:
UmsUmsUmUmAmAmUfCfCfUmCmAmCmUmCmUmAmAmAm
SEQ ID No.2:
UmsUfsUmAmGmAfGmUmGmAmGmGmAmUfUmAfAmAmAmsUmsGm
SEQ ID No.3:
CmsAmsGmAmCmAmGfAfCfAmAmGmAmCmCmAmUmCm
SEQ ID No.4:
AmsGfsAmUmGmGfUmCmUmUmGmUmCmUfGmUfCmUmGmsGmsAm
SEQ ID No.5:
CmsAmsGmAmCmAmGfAfCfAmAmGmAmCmCmAmUmCmUm
SEQ ID No.6:
CmsCmsAmAmGmAmGfCfAfCmCmAmAmGmAmAmCmUmAm
SEQ ID No.7:
UmsAfsGmUmUmCfUmUmGmGmUmGmCmUfCmUfUmGmGmsUmsUm

TABLE 3
siRNA conjugate

| Number of | | | |
|---|---|---|---|
| siRNA | Number of | | |
| conjugate | carrier | Sequence identifier | Sequence direction 5′-3′ |
| RZ502031 | L96 | SS: SEQ ID No.5_L96 | SS: CmsAmsGmAmCmAmGfAfCfAmAmGm |
| | | | AmCmCmAmUmCmUm_L96 |
| | | AS: SEQ ID No. 4 | AS: AmsGfsAmUmGmGfUmCmUmUmGm |
| | | | UmCmUfGmUfCmUmGmsGmsAm |
| RZ599001 | L96 | SS: SEQ ID No. 1_L96 | SS: UmsUmsUmUmAmAmUfCfCfUmCmAm |
| | | | CmUmCmUmAmAmAm_L96 |
| | | AS: SEQ ID No. 2 | AS: UmsUfsUmAmGmAfGmUmGmAmGm |
| | | | GmAmUfUmAfAmAmAmsUmsGm |
| RZ597002 | L96 | SS: SEQ ID No.6_L96 | CmsCmsAmAmGmAmGfCfAfCmCmAmAm |
| | | | GmAmAmCmUmAm_L96 |
| | | AS: SEQ ID No. 7 | UmsAfsGmUmUmCfUmUmGmGmUmGm |
| | | | CmUfCmUfUmGmGmsUmsUm |
| RZ802008 | (CR01020) × 3 | SS: SEQ ID | SS: CmsAmsGmAmCmAmGfAfCfAmAmGm |
| | | No. 3_(CR01020) × 3 | AmCmCmAmUmCm_(CR01020) × 3 |
| | | AS: SEQ ID No. 4 | AS: AmsGfsAmUmGmGfUmCmUmUmGm |
| | | | UmCmUfGmUfCmUmGmsGmsAm |
| RZ802009 | (CR01020) × 3 | SS: SEQ ID | SS: CmsAmsGmAmCmAmGfAfCfAmAmGm |
| | | No. 5_(CR01020) × 3 | AmCmCmAmUmCmUm_(CR01020) × 3 |
| | | AS: SEQ ID No. 4 | AS: AmsGfsAmUmGmGfUmCmUmUmGm |
| | | | UmCmUfGmUfCmUmGmsGmsAm |
| RZ899007 | (CR01020) × 3 | SS: SEQ ID | SS: UmsUmsUmUmAmAmUfCfCfUmCmAm |
| | | No. 1_(CR01020) × 3 | CmUmCmUmAmAmAm_(CR01020) × 3 |
| | | AS: SEQ ID No. 2 | AS: UmsUfsUmAmGmAfGmUmGmAmGm |
| | | | GmAmUfUmAfAmAmAmsUmsGm |
| RZ802014 | (CR01021) × 3 | SS: SEQ ID | SS: CmsAmsGmAmCmAmGfAfCfAmAmGm |
| | | No. 5_(CR01021) × 3 | AmCmCmAmUmCmUm_(CR01021) × 3 |
| | | AS: SEQ ID No. 4 | AS: AmsGfsAmUmGmGfUmCmUmUmGm |
| | | | UmCmUfGmUfCmUmGmsGmsAm |
| RZ899022 | (CR01021) × 3 | SS: SEQ ID | SS: UmsUmsUmUmAmAmUfCfCfUmCmAm |
| | | No. 1_(CR01021) × 3 | CmUmCmUmAmAmAm_(CR01021) × 3 |
| | | AS: SEQ ID No. 2 | AS: UmsUfsUmAmGmAfGmUmGmAmGm |
| | | | GmAmUfUmAfAmAmAmsUmsGm |
| RZ899023 | (CR01024) × 3 | SS: SEQ ID | SS: UmsUmsUmUmAmAmUfCfCfUmCmAm |
| | | No. 1_(CR01024) × 3 | CmUmCmUmAmAmAm_(CR01024) × 3 |
| | | AS: SEQ ID No. 2 | AS: UmsUfsUmAmGmAfGmUmGmAmGm |
| | | | GmAmUfUmAfAmAmAmsUmsGm |
| RZ899024 | (CR01025) × 3 | SS: SEQ ID | SS: UmsUmsUmUmAmAmUfCfCfUmCmAm |
| | | No. 1_(CR01025) × 3 | CmUmCmUmAmAmAm_(CR01025) × 3 |
| | | AS: SEQ ID No. 2 | AS: UmsUfsUmAmGmAfGmUmGmAmGm |
| | | | GmAmUfUmAfAmAmAmsUmsGm |
| RZ802010 | (CR01022) × 3 | SS: SEQ ID | SS: CmsAmsGmAmCmAmGfAfCfAmAmGm |
| | | No. 5_(CR01022) × 3 | AmCmCmAmUmCmUm_(CR01022) × 3 |
| | | AS: SEQ ID No. 4 | AS: AmsGfsAmUmGmGfUmCmUmUmGm |
| | | | UmCmUfGmUfCmUmGmsGmsAm |
| RZ899009 | (CR01022) × 3 | SS: SEQ ID | SS: UmsUmsUmUmAmAmUfCfCfUmCmAm |
| | | No. 1_(CR01022) × 3 | CmUmCmUmAmAmAm_(CR01022) × 3 |
| | | AS: SEQ ID No. 2 | AS: UmsUfsUmAmGmAfGmUmGmAmGm |
| | | | GmAmUfUmAfAmAmAmsUmsGm |
| RZ899011 | (CR01022) × 2 | SS: SEQ ID | SS: UmsUmsUmUmAmAmUfCfCfUmCmAm |
| | | No. 1_(CR01022) × 2 | CmUmCmUmAmAmAm_(CR01022) × 2 |
| | | AS: SEQ ID No. 2 | AS: UmsUfsUmAmGmAfGmUmGmAmGm |
| | | | GmAmUfUmAfAmAmAmsUmsGm |
| RZ899040 | (CR01022) × 1 | SS: SEQ ID | SS: UmsUmsUmUmAmAmUfCfCfUmCmAm |
| | | No. 1_(CR01022) × 1 | CmUmCmUmAmAmAm_(CR01022) × 1 |
| | | AS: SEQ ID No. 2 | AS: UmsUfsUmAmGmAfGmUmGmAmGm |
| | | | GmAmUfUmAfAmAmAmsUmsGm |
| RZ899025 | (CR01026) × 2 | SS: SEQ ID | SS: UmsUmsUmUmAmAmUfCfCfUmCmAm |
| | | No. 1_(CR01026) × 2 | CmUmCmUmAmAmAm_(CR01026) × 2 |
| | | AS: SEQ ID No. 2 | AS: UmsUfsUmAmGmAfGmUmGmAmGm |
| | | | GmAmUfUmAfAmAmAmsUmsGm |
| RZ802011 | (CR01023) × 3 | SS: SEQ ID | SS: CmsAmsGmAmCmAmGfAfCfAmAmGm |
| | | No. 5_(CR01023) × 3 | AmCmCmAmUmCmUm_(CR01023) × 3 |
| | | AS: SEQ ID No. 4 | AS: AmsGfsAmUmGmGfUmCmUmUmGmUm |
| | | | CmUfGmUfCmUmGmsGmsAm |
| RZ899010 | (CR01023) × 3 | SS: SEQ ID | SS: UmsUmsUmUmAmAmUfCfCfUmCmAm |
| | | No. 1_(CR01023) × 3 | CmUmCmUmAmAmAm_(CR01023) × 3 |
| | | AS: SEQ ID No. 2 | AS: UmsUfsUmAmGmAfGmUmGmAmGm |
| | | | GmAmUfUmAfAmAmAmsUmsGm |
| RZ899048 | (CR01027) × 2 | SS: SEQ ID | SS: UmsUmsUmUmAmAmUfCfCfUmCmAm |
| | | No. 1_(CR01027) × 2 | CmUmCmUmAmAmAm_(CR01027) × 2 |
| | | AS: SEQ ID No. 2 | AS: UmsUfsUmAmGmAfGmUmGmAmGm |
| | | | GmAmUfUmAfAmAmAmsUmsGm |
| RZ897013 | (CR01027) × 2 | SS: SEQ ID | SS: CmsCmsAmAmGmAmGfCfAfCmCmAm |
| | | No. 6_(CR01027) × 2 | AmGmAmAmCmUmAm(CR01027) × 2 |
| | | AS: SEQ ID No. 7 | AS: UmsAfsGmUmUmCfUmUmGmGmUm |
| | | | GmCmUfCmUfUmGmGmsUmsUm |
| RZ899038 | (CR01017) × 2 | SS: SEQ ID | SS: UmsUmsUmUmAmAmUfCfCfUmCmAm |
| | | No. 1_(CR01017) × 2 | CmUmCmUmAmAmAm_(CR01017) × 2 |
| | | AS: SEQ ID No. 2 | AS: UmsUfsUmAmGmAfGmUmGmAmGm |
| | | | GmAmUfUmAfAmAmAmsUmsGm |
| RZ899041 | (CR01017) × 1 | SS: SEQ ID | SS: UmsUmsUmUmAmAmUfCfCfUmCmAm |
| | | No. 1_(CR01017) × 1 | CmUmCmUmAmAmAm_(CR01017) × 1 |
| | | AS: SEQ ID No. 2 | AS: UmsUfsUmAmGmAfGmUmGmAmGm |
| | | | GmAmUfUmAfAmAmAmsUmsGm |
| RZ899042 | (CR01017) × 1 | SS: | SS: (CR01017) × 1_UmsUmsUmUmAmAmUfCf |
| | | (CR01017)× 1_SEQ ID | CfUmCmAmCmUmCmUmAmAmAm_(CR01017) × 1 |
| | | No. 1_(CR01017) × 1 | |
| | | AS: SEQ ID No. 2 | AS: UmsUfsUmAmGmAfGmUmGmAmGm |
| | | | GmAmUfUmAfAmAmAmsUmsGm |

Unless otherwise specified, the meanings of the bases and modifications described in various examples of the present disclosure are as follows: uppercase letters A, U, Q C and T represent the bases of the nucleotides, lowercase letter m represents that the nucleotide represented by its upstream letter is a methoxy-modified nucleotide, lowercase letter f represents that the nucleotide represented by its upstream letter is a fluorine-modified nucleotide, and lowercase letter s represents that the nucleotides represented by two letters adjacent to both sides of the letter s are linked by a phosphorothioate linkage.

Unless otherwise specified, the siRNA sequences used herein were synthesized by Suzhou Biosyntech Co., Ltd, and the PCR primers used herein were synthesized by Tsingke (Beijing) Biotechnology Co., Ltd. The C57BL/6J mice as experimental animals used herein were purchased from SPF (Beijing) Biotechnology Co., Ltd.

TABLE 4
Detection results of siRNA conjugate

| | Sense strand (SS) | | | Antisense strand (AS) | | | siRNA conjugate | |
|---|---|---|---|---|---|---|---|---|
| Number of | Theoretical | Measured | | Theoretical | Measured | | Theoretical | Measured |
| siRNA | molecular | molecular | | molecular | molecular | | molecular | molecular |
| conjugate | weight | weight | Purity | weight | weight | Purity | weight | weight |
| RZ502031 | 8102.29 | 8103.1 | 98.97 | 7017.68 | 7017.5 | 97.95 | 15119.97 | 15120.6 |
| RZ599001 | 7940.1 | 7940.6 | 96.4 | 7096.83 | 7097.3 | 94.61 | 15036.93 | 15037.9 |
| RZ597002 | 8125.33 | 8125.6 | 98.6 | 6932.59 | 6933.4 | 98.7 | 15057.92 | 15059 |
| RZ802008 | 8340.74 | 8342.1 | 99.5 | 7017.68 | 7017.5 | 98 | 15358.42 | 15359.6 |
| RZ802009 | 8660.94 | 8661.9 | 95.8 | 7017.68 | 7017.5 | 98 | 15678.62 | 15679.4 |
| RZ899007 | 8498.75 | 8500.1 | 95.3 | 7096.83 | 7097.3 | 94.6 | 15595.58 | 15597.4 |
| RZ802014 | 8281.89 | 8283.8 | 93.5 | 7017.68 | 7018.2 | 98.7 | 15299.57 | 15302 |
| RZ899022 | 8119.7 | 8121.4 | 96.1 | 7096.83 | 7096.8 | 98.7 | 15216.53 | 15218.2 |
| RZ899023 | 7987.7 | 7988.8 | 98.91 | 7096.83 | 7097.2 | 98.9 | 15084.53 | 15086 |
| RZ899024 | 7987.7 | 7989.1 | 96.2 | 7096.83 | 7097.2 | 98.9 | 15084.53 | 15086.3 |
| RZ802010 | 9750.49 | 9754.9 | 97.1 | 7017.68 | 7017.5 | 98 | 16768.17 | 16772.4 |
| RZ899009 | 9590.3 | 9593 | 94 | 7096.83 | 7097.2 | 92.3 | 16687.13 | 16690.2 |
| RZ899011 | 8443.9 | 8446.8 | 90 | 7096.83 | 7097.2 | 96.6 | 15540.73 | 15544 |
| RZ899040 | 7297.9 | 7298.4 | 94.6 | 7096.83 | 7097.3 | 97.1 | 14394.73 | 14395.7 |
| RZ899025 | 8268.3 | 8269.1 | 90.1 | 7096.83 | 7097.2 | 98.9 | 15365.13 | 15366.3 |
| RZ802011 | 8155.89 | 8535.8 | 93.9 | 7017.68 | 7017.5 | 98 | 15173.57 | 15553.3 |
| RZ899010 | 7993.7 | 8373.5 | 99.5 | 7096.83 | 7097.2 | 92.3 | 15090.53 | 15470.7 |
| RZ899048 | 8505.1 | 8505.6 | 90.5 | 7096.83 | 7097.3 | 92.8 | 15601.93 | 15602.9 |
| RZ897013 | 8690.33 | 8690.4 | 92.7 | 6932.59 | 6933.0 | 91.1 | 15622.92 | 15623.4 |
| RZ899038 | 8347.9 | 8349.3 | 92.7 | 7096.83 | 7097.3 | 97.1 | 15444.73 | 15446.6 |
| RZ899041 | 7249.5 | 7250.7 | 97.8 | 7096.83 | 7097.3 | 97.1 | 14346.33 | 14348 |
| RZ899042 | 8347.9 | 8349.2 | 97.7 | 7096.83 | 7097.3 | 97.1 | 15444.73 | 15446.5 |

As can be seen from the data in Table 4, the sense strand (SS) and antisense strand (AS) exhibited a favorable connection to the ligand and can maintain a high purity.

### General Experiments

### Evaluation of Inhibitory Activity on Target Gene in Mice

6 to 8-week-old female C57BL/6J mice were randomly grouped based on body weight. The administration dose for each mouse was calculated according to the body weight of the mouse in each group, and a single subcutaneous injection in the abdomen was adopted. The siRNA conjugate was prepared with PBS into a solution of the corresponding concentration (calculated based on siRNA) for administration. The administration volume was 5 ml/kg (calculated based on body weight of mouse). The PBS control group was administered with PBS solution without containing the siRNA conjugate with a volume of 5 ml/kg (calculated based on body weight of mouse). The day of administration was noted as Day 1 (D1). At predetermined time points, five mice from each group were sacrificed. The sacrificed mice were subjected to gross dissection, and they liver tissues were collected. The liver tissues were cut into pieces of approximately 2 mm³ and stored in RNA Later.

Liver tissue samples from different experimental groups at different time points were taken from the above RNA later, and homogenized in an automatic tissue homogenizer-Tissuelyser II for 60 seconds. According to the standard procedure for total RNA extraction, the total RNA was extracted using an automatic nucleic acid extractor (purchased from Zhejiang Hanwei Technology Co., Ltd.) and a nucleic acid extraction kit (purchased from Zhejiang Hanwei Technology Co., Ltd.).

A reverse transcription kit (Promega, Reverse Transcription System, A3500) was used, 1 μg of the total RNA was taken, Oligo (dT)₁₅ primers for reverse transcription were used, a 20 μL of reverse transcription system was prepared according to the instructions of the reverse transcription kit, and the reverse transcription reaction was carried out. After the reaction was completed, 80 μL of RNase-Free water was added to the reverse transcription system to obtain the cDNA solution. Next, the mRNA expression of target genes in liver tissues was measured with a real-time fluorescence quantitative PCR kit (ABI, SYBR™ Select Master Mix, Catalog number: 4472908). In the real-time fluorescence quantitative PCR assay, primers for the target gene and primers for the internal reference gene were used to detect the target gene and the internal reference gene, respectively. A 20 μL Real-time PCR reaction system per PCR reaction well was prepared according to the protocol documented in the instructions of the real-time fluorescence quantitative PCR kit, and each reaction system contained 5 μL of cDNA solution obtained by the reverse transcription reaction described above, 10 μL of SYBR™ Select Master Mix, 0.5 μL of 10 μM upstream primer, 0.5 μL of 10 μM downstream primer, and 4 μL of RNase-Free H₂O. The prepared reaction system was placed in a real-time fluorescence quantitative PCR instrument (ABI, StepOnePlus™), and real-time PCR amplification was performed by a three-step method. The amplification procedure comprised pre-denaturing at 95 °C for 10 min, denaturing at 95 °C for 30 seconds, annealing at 60 °C for 30 seconds, and extending at 72 °C for 30 seconds. The above denaturing, annealing and extending processes were repeated for 40 cycles. In this real-time fluorescence quantitative PCR assay, the ΔΔCt method was used to relatively quantify the mRNA expression level of the target gene and the inhibition rate in each test group, which was calculated as follows:

ΔCt(test group)=Ct(target gene in test group)−ΔCt(internal reference gene in test group)

ΔCt(control group)=Ct(target gene in control group)−ΔCt(internal reference gene in control group)

ΔΔCt(test group)=ΔCt(test group)−ΔCt(mean of control group)

ΔΔCt(control group)=ΔCt(control group)−ΔCt(mean of control group)
wherein, ΔCt(mean of control group) is the arithmetic mean of each ΔCt(control group) of five mice sacrificed at the same time point in the control group. Each mouse in both the test and control groups could yield a ΔΔCt value.

The mRNA expression level of the target gene in the test group was normalized using the control group as the baseline, and the mRNA expression level of the target gene in the control group was defined as 100%.

Relative expression level of mRNA of target gene in test group=2−ΔΔCt (test group)×100%.

Inhibition rate of mRNA of target gene in test group=(1−relative expression level of mRNA of target gene in test group)×100%.

Unless otherwise indicated, data for activity assay in vivo are reported as X̅±SD, and the experimental data were plotted and analyzed using software GraphPad prism 8.0.

### Example 1 In Vivo Activity Assay of the Trimeric CR01020 Carrier Conjugated with siRNA Targeting Complement Component 3 (CC3)

According to the method for evaluating inhibitory activity on target gene in mice, in this example, the in vivo inhibitory activity of the siRNA conjugates RZ802008 and RZ802009 conjugated with trimeric CR01020 carrier at 3′ end of sense strand of siRNA targeting CC3, and RZ502031 conjugated with the L96 carrier at 3′ end of sense strand of the same siRNA targeting CC3 on the target gene CC3 in mice was evaluated.

6 to 8-week-old C57BL/6j mice were randomly divided into 4 groups according to their body weight, with 15 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered at a dose of 3 mg/kg (calculated based on siRNA) and a volume of 5 ml/kg. The day of administration was noted as day 1 (D1). Five mice from each group were sacrificed on day 8 (D8), day 15 (D15), and day 29 (D29) after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 5.

TABLE 5
Primer sequence

| Target | Primer | Primer sequence (5′-3′) | SEQ ID No. |
|---|---|---|---|
| CC3 | Upstream primer | CCAGCTCCCCATTAGCTCTG | 8 |
| | Downstream | GCACTTGCCTCTTTAGGAAGTC | 9 |
| | primer | | |
| GAPDH | Upstream primer | TGCACCACCAACTGCTTAG | 10 |
| | Downstream | GGATGCAGGGATGATGTTC | 11 |
| | primer | | |

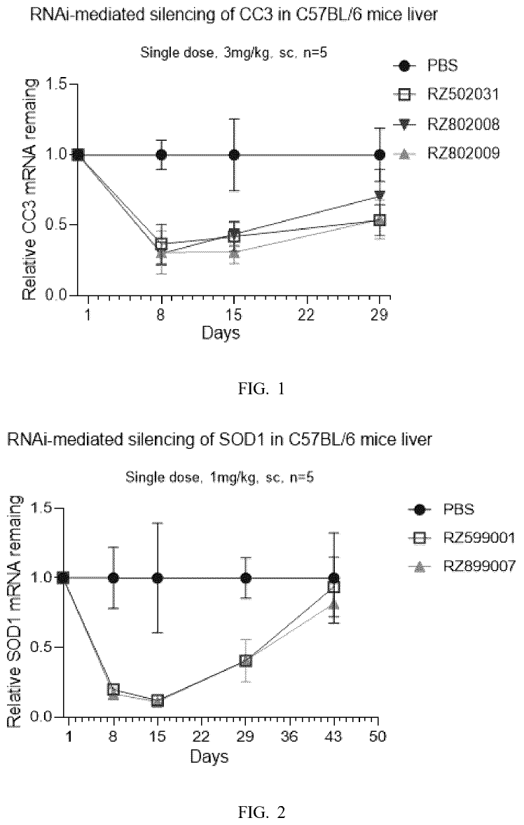

The results of example 1 showed that the conjugate RZ802008 conjugated with trimeric CR01020 at 3′ end of siRNA sense strand exhibited in vivo activity and duration of action basically comparable to those of the conjugate RZ502031 conjugated with L96 at 3′ end of siRNA sense strand. The conjugate RZ802009 conjugated with trimeric CR01020 exhibited superior in vivo activity and comparable duration of action compared to the conjugate RZ502031 conjugated with L96 (FIG. 1 and Table 6).

TABLE 6
Inhibition rate on target gene in mice after administration
of siRNA conjugates in this example

| | D8 | | D15 | | D29 | |
|---|---|---|---|---|---|---|
| | Average | | Average | | Average | |
| | inhibition | ±SD | inhibition | ±SD | inhibition | ±SD |
| Group | rate | value | rate | value | rate | value |
| PBS | 0.00% | 0.1048 | 0.00% | 0.2555 | 0.00% | 0.1892 |
| RZ502031 | 63.27% | 0.1405 | 58.09% | 0.1060 | 46.38% | 0.1120 |
| RZ802008 | 70.16% | 0.0826 | 56.37% | 0.0861 | 29.44% | 0.1896 |
| RZ802009 | 69.29% | 0.1528 | 69.17% | 0.0797 | 45.94% | 0.1397 |

### Example 2 In Vivo Activity Assay of the Trimeric CR01020 Carrier Conjugated with siRNA Targeting Superoxide Dismutase 1 (SOD1)

According to the method for evaluating inhibitory activity on target gene in mice, in this example, the in vivo inhibitory activity of the siRNA conjugates RZ899007 conjugated with trimeric CR01020 carrier at 3′ end of sense strand of siRNA targeting SOD1, and RZ599001 conjugated with the L96 carrier at 3′ end of sense strand of the same siRNA targeting SOD1 on the target gene SOD1 in mice was evaluated.

6 to 8-week-old C57BL/6j mice were randomly divided into 3 groups according to their body weight, with 20 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 1 mg/kg (calculated based on siRNA) and a volume of 5 ml/kg. The day of administration was noted as day 1 (D1). Five mice from each group were sacrificed on day 8 (D8), day 15 (D15), day 29 (D29), and day 43 (D43) after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7.

TABLE 7
Primer sequence

| | | | SEQ ID |
|---|---|---|---|
| Target | Primer | Primer sequence (5′-3′) | No. |
| SOD1 | Upstream | GGGTTCCACGTCCATCAGTA | 12 |
| | primer | | |
| | Downstream | ACACCGTCCTTTCCAGCAGT | 13 |
| | primer | | |
| GAPDH | Upstream | TGCACCACCAACTGCTTAG | 10 |
| | primer | | |
| | Downstream | GGATGCAGGGATGATGTTC | 11 |
| | primer | | |

The results of example 2 showed that the conjugate RZ899007 conjugated with trimeric CR01020 at 3′ end of siRNA sense strand exhibited comparable maximum inhibitory activity and longer duration of action compared to the conjugate RZ599001 conjugated with L96 at 3′ end of siRNA sense strand. (FIG. 2 and Table 8).

TABLE 8
Inhibition rate on target gene in mice after administration of siRNA conjugates in this example

| | D8 | | D15 | | D29 | | D43 | |
|---|---|---|---|---|---|---|---|---|
| | Average | | Average | | Average | | Average | |
| | inhibition | ±SD | inhibition | ±SD | inhibition | ±SD | inhibition | ±SD |
| Group | rate | value | rate | value | rate | value | rate | value |
| PBS | 0.00% | 0.2208 | 0.00% | 0.3939 | 0.00% | 0.1455 | 0.00% | 0.3246 |
| RZ599001 | 80.20% | 0.0367 | 88.03% | 0.0286 | 59.62% | 0.0294 | 6.49% | 0.2161 |
| RZ899007 | 83.37% | 0.0412 | 88.88% | 0.0253 | 59.29% | 0.1513 | 18.34% | 0.1459 |

### Example 3 In Vivo Activity Assay of the Trimeric CR01021 an CR01020 Carriers Conjugated with siRNA Targeting CC3

According to the method for evaluating inhibitory activity on target gene in mice, in this example, the in vivo inhibitory activity of the siRNA conjugates RZ802014 conjugated with trimeric CR01021 carrier at 3′ end of sense strand of siRNA targeting CC3, RZ802009 conjugated with trimeric CR01020 carrier at 3′ end of sense strand of the same siRNA targeting CC3, and RZ502031 conjugated with the L96 carrier at 3′ end of sense strand of the same siRNA targeting CC3 on the target gene CC3 in mice was evaluated. The trimeric CR01021 carrier and trimeric CR01020 carrier differ in their cyclic core structures, which were isomeric.

6 to 8-week-old C57BL/6j mice were randomly divided into 4 groups according to their body weight, with 10 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 3 mg/kg (calculated based on siRNA) and a volume of 5 ml/kg. The day of administration was noted as day 1 (D1). Five mice from each group were sacrificed on day 8 (D8) and day 22 (D22) after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 5 of example 1.

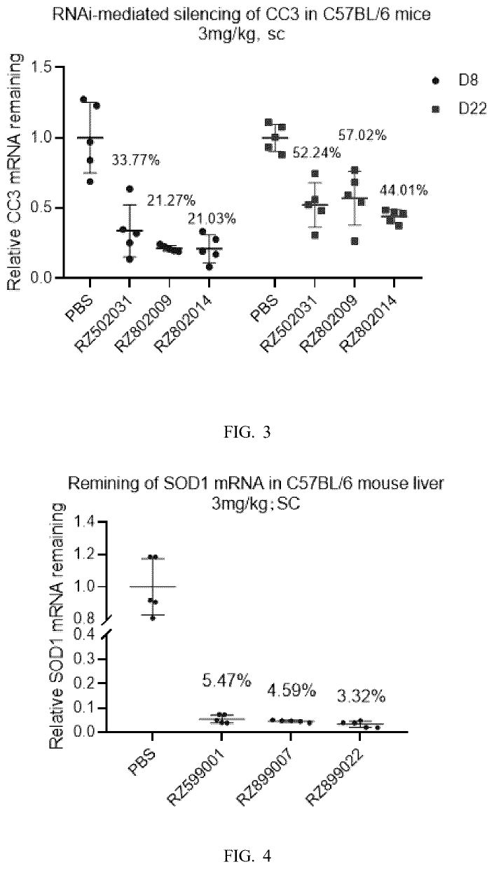

The results of example 3 showed that the conjugate RZ802014 conjugated with trimeric CR01021 exhibited superior in vivo activity compared to the conjugate RZ502031 conjugated with L96, and the conjugate RZ802009 conjugated with trimeric CR01020 exhibited the efficiency basically comparable to that of the conjugate RZ502031 conjugated with L96 (FIG. 3 and Table 9).

TABLE 9
Inhibition rate on target gene in mice after administration
of siRNA conjugates in this example

| | D8 | | D22 | |
|---|---|---|---|---|
| | Average | | Average | |
| Group | inhibition rate | ±SD value | inhibition rate | ±SD value |
| PBS | 0.00% | 0.2503 | 0.00% | 0.0958 |
| RZ502031 | 66.23% | 0.1855 | 47.76% | 0.1580 |
| RZ802009 | 78.73% | 0.0209 | 42.98% | 0.1918 |
| RZ802014 | 78.97% | 0.0979 | 55.99% | 0.0466 |

### Example 4 In Vivo Activity Assay of the Trimeric CR01021 an CR01020 Carriers Conjugated with siRNA Targeting SOD1

According to the method for evaluating inhibitory activity on target gene in mice, in this example, the in vivo inhibitory activity of the siRNA conjugates RZ899022 conjugated with trimeric CR01021 carrier at 3′ end of sense strand of siRNA targeting SOD1, RZ899007 conjugated with trimeric CR01020 carrier at 3′ end of sense strand of the same siRNA targeting SOD1 and RZ599001 conjugated with the L96 carrier at 3′ end of sense strand of the same siRNA targeting SOD1 on the target gene SOD1 in mice was evaluated. The trimeric CR01021 carrier and trimeric CR01020 carrier differed in their cyclic core structures, which were isomeric.

6 to 8-week-old C57BL/6j mice were randomly divided into 4 groups according to their body weight, with 5 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test group was administered with a dose of 3 mg/kg (calculated based on siRNA) and a volume of 5 ml/kg. The day of administration was noted as day 1 (D1). Five mice from each group were sacrificed on day 8 (D8) after the administration. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7 of example 2.

The results of example 4 showed that the siRNA conjugates RZ899022 conjugated with trimeric CR01021 at 3′ end of siRNA sense strand and RZ899007 conjugated with trimeric CR01020 at 3′ end of siRNA sense strand exhibited superior in vivo activity compared to the conjugate RZ599001 conjugated with L96 (FIG. 4 and Table 10).

TABLE 10
Inhibition rate on target gene in mice after administration
of siRNA conjugates in this example

| Group | Average inhibition rate | ±SD value |
|---|---|---|
| PBS | 0.00% | 0.1740 |
| RZ599001 | 94.53% | 0.0172 |
| RZ899007 | 95.41% | 0.0045 |
| RZ899022 | 96.68% | 0.0128 |

### Example 5 In Vivo Activity Assay of the Trimeric CR01024 and CR01025 Carriers Conjugated with siRNA Targeting SOD1

According to the method for evaluating inhibitory activity on target gene in mice, in this example, the in vivo inhibitory activity of the siRNA conjugates RZ899023 conjugated with trimeric CR01024 carrier at 3′ end of sense strand of siRNA targeting SOD1, RZ899024 conjugated with trimeric CR01025 carrier at 3′ end of sense strand of the same siRNA targeting SOD1 and RZ599001 conjugated with L96 at 3′ end of sense strand of the same siRNA targeting SOD1 on the target gene SOD1 in mice was evaluated. Trimeric CR01024 and trimeric CR01020 had the same cyclic core structure, but differed in the length of the linker. CR01025 carrier and CR01021 have the same cyclic core structure, but differed in the length of the linker.

6 to 8-week-old C57BL/6j mice were randomly divided into 4 groups according to their body weight, with 5 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test group was administered with a dose of 3 mg/kg (calculated based on siRNA) and a volume of 5 ml/kg. The day of administration was noted as day 1 (D1). Mice were sacrificed on day 8 (D8) after the administration. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7 of example 2.

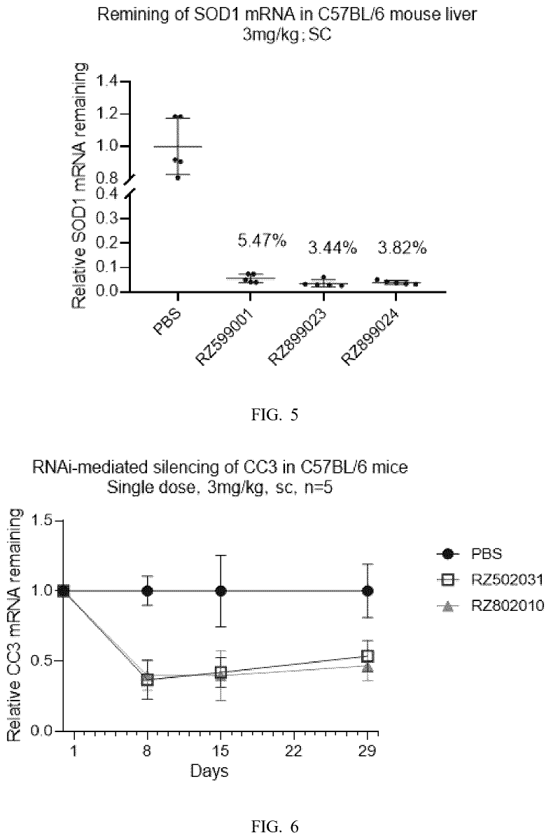

The results of example 5 showed that the siRNA conjugates RZ899023 conjugated with trimeric CR01024 and RZ899024 conjugated with trimeric CR01025 at 3′ end of siRNA sense strand exhibited superior in vivo activity compared to the conjugate RZ599001 conjugated with L96 (FIG. 5 and Table 11).

The results of examples 4 and 5 indicate that the length of linker has little effect on carrier activity.

TABLE 11
Inhibition rate on target gene in mice after administration
of siRNA conjugates in this example

| Group | Average inhibition rate | ±SD value |
|---|---|---|
| PBS | 0.00% | 0.1740 |
| RZ599001 | 94.53% | 0.0172 |
| RZ899023 | 96.56% | 0.0147 |
| RZ899024 | 96.18% | 0.0075 |

### Example 6 In Vivo Activity Assay of the Trimeric CR01022 Carrier Conjugated with siRNA Targeting CC3

According to the method for evaluating inhibitory rate on target gene in mice, in this example, the in vivo inhibitory activity of the siRNA conjugates RZ802010 conjugated with trimeric CR01022 carrier at 3′ end of sense strand of siRNA targeting CC3 and RZ502031 conjugated with L96 at 3′ end of sense strand of the same siRNA targeting CC3 on the target gene CC3 in mice was evaluated.

6 to 8-week-old C57BL/6j mice were randomly divided into 3 groups according to their body weight, with 15 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 3 mg/kg (calculated based on siRNA) and a volume of 5 ml/kg. The day of administration was noted as day 1 (D1). Five mice from each group were sacrificed on day 8 (D8), day 15 (D15), and day 29 (D29) after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 5 of example 1.

The results of example 6 showed that the conjugate RZ802010 conjugated with trimeric CR01022 at 3′ end of siRNA sense strand exhibited comparable maximum inhibitory activity and longer duration of action compared to the conjugate RZ502031 conjugated with L96 (FIG. 6 and Table 12).

TABLE 12
Inhibition rate on target gene in mice after administration of siRNA
conjugates in this example

| | D8 | | D15 | | D29 | |
|---|---|---|---|---|---|---|
| | Average | | Average | | Average | |
| | inhibition | ±SD | inhibition | ±SD | inhibition | ±SD |
| Group | rate | value | rate | value | rate | value |
| PBS | 0.00% | 0.1048 | 0.00% | 0.2555 | 0.00% | 0.1892 |
| RZ502031 | 63.27% | 0.1405 | 58.09% | 0.1060 | 46.38% | 0.1120 |
| RZ802010 | 60.32% | 0.1043 | 60.35% | 0.1778 | 53.33% | 0.1037 |

### Example 7 In Vivo Activity Assay of the Dimeric CR01022 and Trimeric CR01022 Carriers Conjugated with siRNA Targeting SOD1

According to the method for evaluating inhibitory activity on target gene in mice, in this example, the in vivo inhibitory activity of the siRNA sequences RZ899009 conjugated with trimeric CR01022 carrier at 3′ end of sense strand of siRNA targeting SOD1, RZ899011 conjugated with dimeric CR01022 carrier at 3′ end of sense strand of the same siRNA targeting SOD1, and RZ599001 conjugated with L96 at 3′ end of sense strand of the same siRNA targeting SOD1 on the target gene SOD1 in mice was evaluated.

6 to 8-week-old C57BL/6j mice were randomly divided into 4 groups according to their body weight, with 20 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 1 mg/kg (calculated based on siRNA) and a volume of 5 ml/kg. The day of administration was noted as day 1 (D1). Five mice from each group were sacrificed on day 8 (D8), day 15 (D15), day 29 (D29), and day 43 (D43) after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7 of example 2.

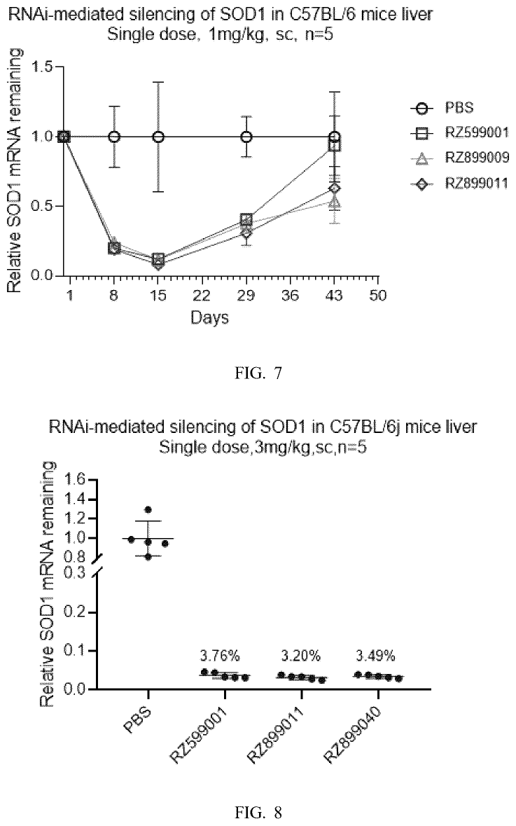

The results of example 7 showed that the maximum inhibitory activity on target gene SOD1 and duration of action of the siRNA sequences RZ899009 conjugated with trimeric CR01022 at 3′ end of siRNA sense strand and RZ899011 conjugated with dimeric CR01022 at 3′ end of siRNA sense strand were basically comparable, which are superior to those of the sequence RZ599001 conjugated with L96 (FIG. 7 and Table 13).

TABLE 13
Inhibition rate on target gene in mice after administration of siRNA conjugates in this example

| | D8 | | D15 | | D29 | | D43 | |
|---|---|---|---|---|---|---|---|---|
| | Average | | Average | | Average | | Average | |
| | inhibition | ±SD | inhibition | ±SD | inhibition | ±SD | inhibition | ±SD |
| Group | rate | value | rate | value | rate | value | rate | value |
| PBS | 0.00% | 0.2208 | 0.00% | 0.3939 | 0.00% | 0.1455 | 0.00% | 0.3246 |
| RZ599001 | 80.20% | 0.0367 | 88.03% | 0.0286 | 59.62% | 0.0294 | 6.49% | 0.2161 |
| RZ899009 | 76.08% | 0.0373 | 88.47% | 0.0438 | 62.49% | 0.0690 | 46.23% | 0.1603 |
| RZ899011 | 81.02% | 0.0330 | 91.97% | 0.0322 | 69.32% | 0.0898 | 37.12% | 0.1585 |

### Example 8 In Vivo Activity Assay of the Monomeric CR01022 and Dimeric CR01022 Carriers Conjugated with siRNA Targeting SOD1

According to the method for evaluating inhibitory activity on target gene in mice, in this example, the in vivo inhibitory activity of the siRNA sequences RZ899011 conjugated with dimeric CR01022 carrier at 3′ end of sense strand of siRNA targeting SOD1, RZ899040 conjugated with monomeric CR01022 carrier at 3′ end of sense strand of the same siRNA targeting SOD1, and RZ599001 conjugated with L96 at 3′ end of sense strand of the same siRNA targeting SOD1 target, on the target gene SOD1 in mice was evaluated.

6 to 8-week-old C57BL/6j mice were randomly divided into 4 groups according to their body weight, with 5 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 1 mg/kg (calculated based on siRNA) and a volume of 5 ml/kg. The day of administration was noted as day 1 (D1). Mice were sacrificed on day 8 (D8) after the administration. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7 of example 2.

The results of example 8 showed that the siRNA sequences RZ899011 conjugated with dimeric CR01022 at 3′ end of siRNA sense strand and RZ899040 conjugated with monomeric CR01022 at 3′ end of siRNA sense strand exhibited highly effective inhibition on the target gene SOD1 on D8. Their inhibitory activities are basically comparable, and both are slightly superior to that of the conjugate RZ599001 conjugated with L96 (FIG. 8 and Table 14).

TABLE 14
Inhibition rate on target gene in mice after administration
of siRNA conjugates in this example

| | D8 | |
|---|---|---|
| Group | Average inhibition rate | ±SD value |
| PBS | 0.00% | 0.1788 |
| RZ599001 | 96.24% | 0.0073 |
| RZ899011 | 96.80% | 0.0057 |
| RZ899040 | 96.51% | 0.0047 |

### Example 9 In Vivo Activity Assay of the Dimeric CR01026 an Dimeric CR01022 Carriers Conjugated with siRNA Targeting SOD1

According to the method for evaluating inhibitory activity on target gene in mice, in this example, the in vivo inhibitory activity of the siRNA sequences RZ899025 conjugated with dimeric CR01026 carrier at 3′ end of sense strand of siRNA targeting SOD1, RZ899011 conjugated with dimeric CR01022 carrier at 3′ end of sense strand of the same siRNA targeting SOD1, and RZ599001 conjugated with L96 at 3′ end of sense strand of the same siRNA targeting SOD1 target, on the target gene SOD1 in mice was evaluated. Dimeric CR01026 and dimeric CR01020 had the same cyclic core structure, but differed in the length of the linker.

6 to 8-week-old C57BL/6j mice were randomly divided into 4 groups according to their body weight, with 20 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 3 mg/kg (calculated based on siRNA) and a volume of 5 ml/kg. The day of administration was noted as day 1 (D1). Five mice from each group were sacrificed on day 8 (D8), day 29 (D29), day 43 (D43), and day 57 (D57) after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7 of example 2.

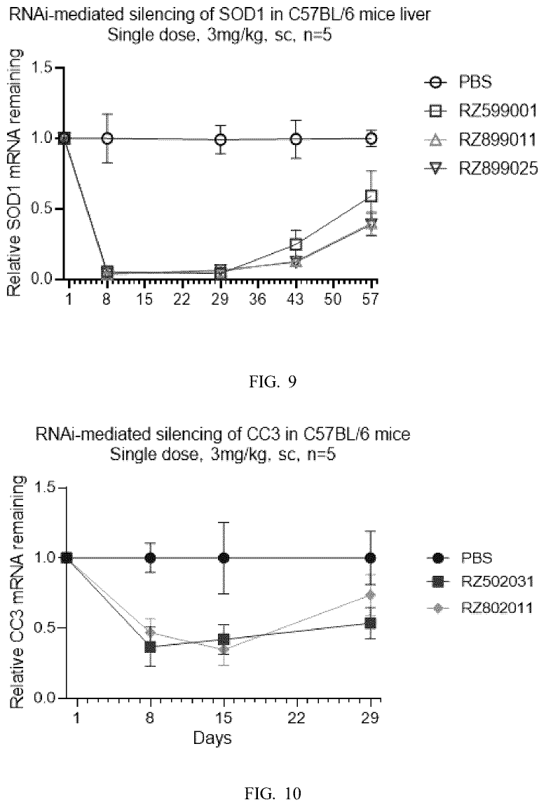

The results of example 8 showed that the siRNA sequences RZ899025 conjugated with dimeric CR01026 and RZ899011 conjugated with dimeric CR01022 at 3′ end of siRNA sense strand exhibited in vivo activity comparable to that of the sequence RZ599001 conjugated with L96 on D8 and D29, and superior to that of the sequence RZ599001 conjugated with L96 on D43, showing that both conjugates RZ899025 conjugated with dimeric CR01026 and RZ899011 conjugated with dimeric CR01022 had longer duration of action compared to the conjugate RZ599001 conjugated with L96, and the length of linker had little effect on the activity of CR01026 and CR01022 carriers (FIG. 9 and Table 15).

TABLE 15
Inhibition rate on target gene in mice after administration of siRNA conjugate in this example

| | D8 | | D29 | | D43 | | D57 | |
|---|---|---|---|---|---|---|---|---|
| | Average | | Average | | Average | | Average | |
| | inhibition | SD | inhibition | SD | inhibition | SD | inhibition | SD |
| Group | rate | value | rate | value | rate | value | rate | value |
| PBS | 0.00% | 0.1740 | 0.00% | 0.1007 | 0.00% | 0.1348 | 0.00% | 0.0582 |
| RZ599001 | 94.53% | 0.0172 | 95.94% | 0.0097 | 75.14% | 0.1032 | 40.78% | 0.1768 |
| RZ899011 | 96.41% | 0.0069 | 94.30% | 0.0123 | 86.89% | 0.0283 | 59.94% | 0.0821 |
| RZ899025 | 96.00% | 0.0100 | 93.60% | 0.0204 | 87.74% | 0.0327 | 61.08% | 0.0798 |

### Example 10 In Vivo Activity Assay of the Trimeric CR01023 Carrier Conjugated with siRNA Targeting CC3

According to the method for evaluating inhibitory activity on target gene in mice, in this example, the in vivo inhibitory activity of the siRNA sequences RZ802011 conjugated with trimeric CR01023 carrier and RZ502031 conjugated with L96 at 3′ end of sense strand of siRNA on the target gene CC3 in mice was evaluated.

6 to 8-week-old C57BL/6j mice were randomly divided into 3 groups according to their body weight, with 15 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 3 mg/kg (calculated based on siRNA) and a volume of 5 ml/kg. The day of administration was noted as day 1 (D1). Five mice from each group were sacrificed on D8, D15, and D29 after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 5 of example 1.

The results of example 10 showed that the two tested conjugates RZ802011 conjugated with trimeric CR01023 and RZ502031 conjugated with L96 exhibited comparable activity overall on D8 and D15. However, the conjugate RZ502031 conjugated with L96 exhibited relatively higher inhibition on target gene on D29 (FIG. 10 and Table 16).

TABLE 16
Inhibition rate on target gene in mice after administration
of siRNA conjugates in this example

| | D8 | | D15 | | D29 | |
|---|---|---|---|---|---|---|
| | Average | | Average | | Average | |
| | inhibition | ±SD | inhibition | ±SD | inhibition | ±SD |
| Group | rate | value | rate | value | rate | value |
| PBS | 0.00% | 0.1048 | 0.00% | 0.2555 | 0.00% | 0.1892 |
| RZ502031 | 63.27% | 0.1405 | 58.09% | 0.1060 | 46.38% | 0.1120 |
| RZ802011 | 53.23% | 0.1005 | 65.30% | 0.1118 | 26.35% | 0.1448 |

### Example 11 In Vivo Activity Assay of the Trimeric CR01023 Carrier Conjugated with siRNA Targeting SOD1

According to the method for evaluating inhibitory activity on target gene in mice, in this example, the in vivo inhibitory activity of the siRNA sequences RZ899010 conjugated with trimeric CR01023 carrier at 3′ end of sense strand of siRNA and RZ599001 conjugated with L96 at 3′ end of sense strand of siRNA, on the target gene SOD1 in mice was evaluated.

6 to 8-week-old C57BL/6j mice were randomly divided into 3 groups according to their body weight, with 15 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 1 mg/kg (calculated based on siRNA) and a volume of 5 ml/kg. The day of administration was noted as day 1 (D1). Five mice from each group were sacrificed on day 8 (D8), day 15 (D15), and day 29 (D29) after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7 of example 2.

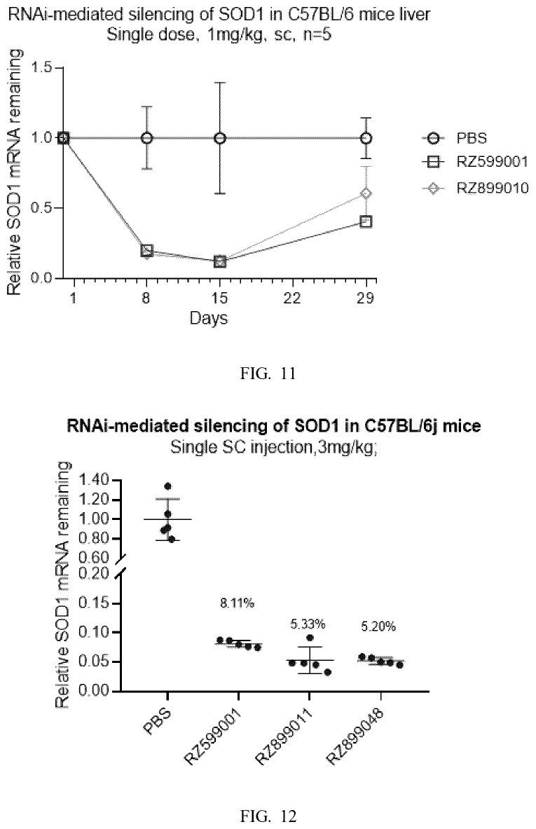

The results of example 11 showed that the two tested conjugates RZ899010 conjugated with trimeric CR01023 and RZ599001 conjugated with L96 exhibited comparable activity overall on D8 and D15. However, the conjugate RZ599001 conjugated with L96 exhibited relatively higher inhibition on target gene on D29 (FIG. 11 and Table 17).

TABLE 17
Inhibition rate on target gene in mice after administration
of siRNA conjugates in this example

| | D8 | | D15 | | D29 | |
|---|---|---|---|---|---|---|
| | Average | | Average | | Average | |
| | inhibition | ±SD | inhibition | ±SD | inhibition | ±SD |
| Group | rate | value | rate | value | rate | value |
| PBS | 0.00% | 0.2208 | 0.00% | 0.3939 | 0.00% | 0.1455 |
| RZ599001 | 80.20% | 0.0367 | 88.03% | 0.0286 | 59.62% | 0.0294 |
| RZ899010 | 82.53% | 0.0309 | 87.56% | 0.0348 | 39.30% | 0.1903 |

### Example 12 In Vivo Activity Assay of the Dimeric CR01027 and Dimeric CR01022 Carriers Conjugated with siRNA Targeting SOD1

According to the method for evaluating inhibitory activity on target gene in mice, in this example, the in vivo inhibitory activity of the siRNA sequences RZ899048 conjugated with dimeric CR01027 carrier at 3′ end of siRNA sense strand, RZ899011 conjugated with dimeric CR01022 carrier at 3′ end of siRNA sense strand and RZ599001 conjugated with L96 at 3′ end of siRNA sense strand, on the target gene SOD1 in mice was evaluated. CR01027 differed from CR01022 in the addition of a methoxy group at position 1 of the core structure.

6 to 8-week-old C57BL/6j mice were randomly divided into 4 groups according to their body weight, with 5 mice in each group. The siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 3 mg/kg and a volume of 5 ml/kg. The day of administration was noted as day 1 (D1). Five mice from each group were sacrificed on day 8 (D8) after administration. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7 of example 2.

The results of example 12 showed that the conjugate RZ899048 conjugated with dimeric CR01027 exhibited higher inhibition on target gene than that of the conjugate RZ599001 conjugated with L96 and comparable to that of the conjugate RZ899011 conjugated with dimeric CR01022 on D8 (FIG. 12 and Table 18).

TABLE 18
Inhibition rate on target gene in mice after administration
of siRNA conjugates in this example

| | D8 | |
|---|---|---|
| Group | Average inhibition rate | SD value |
| PBS | 0.00% | 0.2122 |
| RZ599001 | 91.89% | 0.0058 |
| RZ899011 | 94.67% | 0.0224 |
| RZ899048 | 94.80% | 0.0058 |

### Example 13 In Vivo Activity Assay of the Dimeric CR01027 Carrier Conjugated with siRNA Targeting Angiopoietin-Like 3 (ANGPTL3)

According to the method for evaluating inhibitory activity on target gene in mice, in this example, the in vivo inhibitory activity of the siRNA sequences RZ897013 conjugated with dimeric CR01027 carrier at 3′ end of siRNA sense strand and RZ597002 conjugated with L96 at 3′ end of siRNA sense strand, on the target gene ANGPTL3 in mice was evaluated.

6 to 8-week-old C57BL/6j mice were randomly divided into 4 groups according to their body weight, with 5 mice in each group. The siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 3 mg/kg and a volume of 5 ml/kg. The day of administration was noted as day 1 (D1). Five mice from each group were sacrificed on day 8 (D8) after administration. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 19.

TABLE 19
Primer sequence

| | | | SEQ ID |
|---|---|---|---|
| Target | Primer | Primer sequence (5′-3′) | No. |
| ANGPTL3 | Upstream | GAGGAGCAGCTAACCAACTTAAT | 14 |
| | primer | | |
| | Downstream | TCTGCATGTGCTGTTGACTTAAT | 15 |
| | primer | | |
| GAPDH | Upstream | TGCACCACCAACTGCTTAG | 10 |
| | primer | | |
| | Downstream | GGATGCAGGGATGATGTTC | 11 |
| | primer | | |

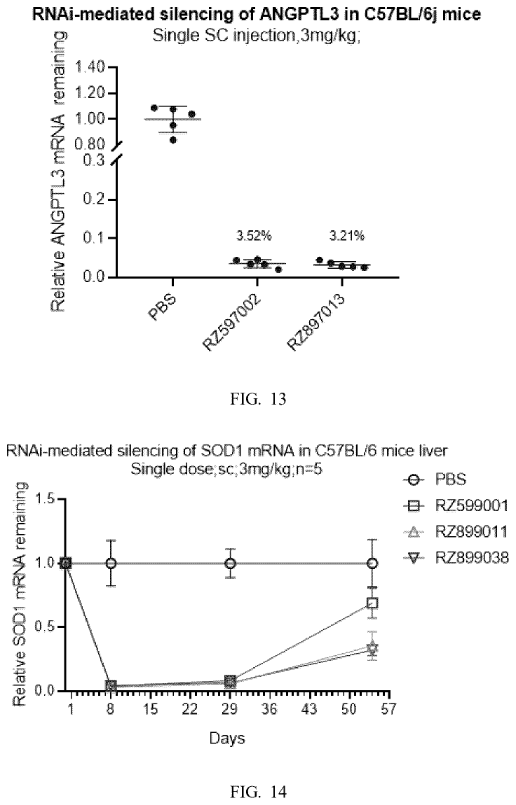

The results of example 13 showed that the conjugate conjugated with dimeric CR01027 exhibited inhibitory effect comparable to that of the conjugate RZ597002 conjugated with L96 (FIG. 13 and Table 20).

TABLE 20
Inhibition rate on target gene in mice after administration
of siRNA conjugates in this example

| | D8 | |
|---|---|---|
| Group | Average inhibition rate | SD value |
| PBS | 0.00% | 0.1039 |
| RZ597002 | 96.48% | 0.0103 |
| RZ897013 | 96.79% | 0.0083 |

### Example 14 In Vivo Activity Assay of the Dimeric CR01027 and Dimeric CR01022 Carriers Conjugated with siRNA Targeting SOD1

According to the method for evaluating inhibitory activity on target gene in mice, in this example, the in vivo inhibitory activity of the siRNA sequences RZ899038 conjugated with dimeric CR01017 carrier at 3′ end of sense strand of siRNA targeting SOD1, RZ899011 conjugated with dimeric CR01022 carrier at 3′ end of sense strand of siRNA targeting SOD1, and RZ599001 conjugated with L96 at 3′ end of sense strand of siRNA targeting SOD1, on the target gene SOD1 in mice was evaluated. The dimeric CR01017 carrier differed from the dimeric CR01022 carrier mainly in that the linker of the CR01017 carrier had an amide bond structure, and the linker of the CR01022 carrier had a triazole structure.

6 to 8-week-old C57BL/6j mice were randomly divided into 4 groups according to their body weight, with 15 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 3 mg/kg (calculated based on siRNA) and a volume of 5 ml/kg. The day of administration was noted as day 1 (D1). Five mice from each group were sacrificed on day 8 (D8), day 29 (D29), and 54 days (D54) after the administration, respectively. Liver tissues of mice were collected, from which RNA was extracted. The reverse transcription reaction and real-time PCR amplification were carried out. The mRNA expression level of the target gene in each test group was relatively quantified by the above-mentioned ΔΔCt method.

Primer sequences used in this example are shown in Table 7 of example 2.

The results of example 14 showed that the siRNA sequences RZ899038 conjugated with dimeric CR01017 carrier at 3′ end of siRNA sense strand and RZ899011 conjugated with dimeric CR01022 carrier at 3′ end of siRNA sense strand exhibited superior in vivo activity compared to the sequence RZ599001 conjugated with L96, showing whether the linker had a triazole structure (CR01022) or structure amide (CR01017) had little effect on activity of carrier (FIG. 14 and Table 21).

TABLE 21
Inhibition rate on target gene in mice after administration
of siRNA conjugates in this example

| | D8 | | D29 | | D54 | |
|---|---|---|---|---|---|---|
| | Average | | Average | | Average | |
| | inhibition | ±SD | inhibition | ±SD | inhibition | ±SD |
| Group | rate | value | rate | value | rate | value |
| PBS | 0.00% | 0.1788 | 0.00% | 0.1118 | 0.00% | 0.1850 |
| RZ599001 | 96.24% | 0.0073 | 91.87% | 0.0144 | 31.07% | 0.1161 |
| RZ899011 | 96.80% | 0.0057 | 94.10% | 0.0124 | 64.50% | 0.1123 |
| RZ899038 | 95.83% | 0.0133 | 93.54% | 0.0111 | 67.79% | 0.0420 |

### Example 15 In Vivo Activity Assay of the CR01027 at Varying Amount and Conjugation Position Conjugated with siRNA Targeting SOD1

According to the method for evaluating inhibitory activity on target gene in mice, in this example, the in vivo inhibitory activity of the siRNA sequences RZ899041 conjugated with a monomeric CR01017 carrier at 3′ end of siRNA sense strand, RZ899042 conjugated with monomeric CR01017 carriers at both the 5′ and 3′ ends of siRNA sense strand, RZ899038 conjugated with a dimeric CR01017 carrier at 3′ end of siRNA sense strand, and RZ599001 conjugated with L96 at 3′ end of siRNA sense strand, on the target gene SOD1 in mice was evaluated.

6 to 8-week-old C57BL/6j mice were randomly divided into 5 groups according to their body weight, with 15 mice in each group. PBS solution and the siRNA conjugates in this example were respectively administered subcutaneously into the abdomen of mice of each group. Each mouse in the PBS control group was administered with a volume of 5 ml/kg, and each mouse in the siRNA conjugate test groups was administered with a dose of 3 mg/kg (calculated based on siRNA) and a volume of 5 ml/kg. The day of administration was noted as day 1 (D1). Five mice from each group were sacrificed on day 8 (D8), day 29 (D29), and 54 days (D54) after the administration, respectively.

Primer sequences used in this example are shown in Table 7 of example 2.

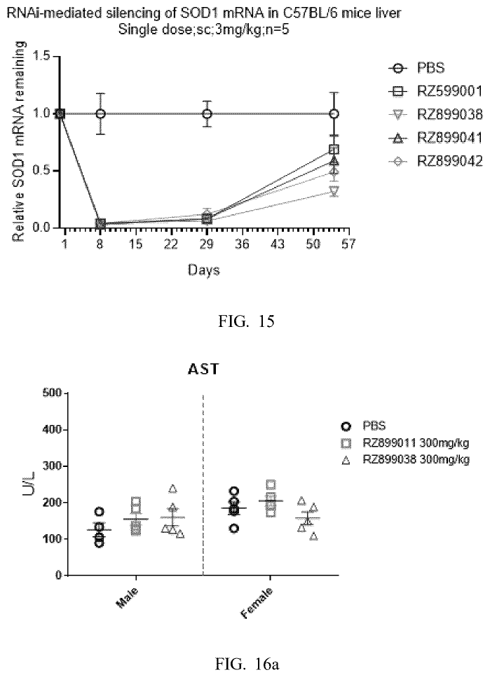

The results of example 15 showed that the siRNA conjugates RZ899038 conjugated with a dimeric CR01017 carrier at 3′ end of siRNA sense strand, RZ899041 conjugated with a monomeric CR01017 carrier at 3′ end of siRNA sense strand, and RZ899042 conjugated with monomeric CR01017 carriers at both the 5′ and 3′ ends of siRNA sense strand exhibited an inhibitory effect comparable to that of the conjugate RZ599001 conjugated with L96 on D8 and D29, and exhibited an inhibitory effect on target gene higher than that of the conjugate RZ599001 conjugated with L96 on D59, showing that CR01017 conjugates had longer duration of action (FIG. 15 and Table 22).

TABLE 22
Inhibition rate on target gene in mice after administration
of siRNA conjugate in this example

| | D8 | | D29 | | D54 | |
|---|---|---|---|---|---|---|
| | Average | | Average | | Average | |
| | inhibition | ±SD | inhibition | ±SD | inhibition | ±SD |
| Group | rate | value | rate | value | rate | value |
| PBS | 0.00% | 0.1788 | 0.00% | 0.1118 | 0.00% | 0.1850 |
| RZ599001 | 96.24% | 0.0073 | 91.87% | 0.0144 | 31.07% | 0.1161 |
| RZ899038 | 95.83% | 0.0133 | 93.54% | 0.0111 | 67.79% | 0.0420 |
| RZ899041 | 96.64% | 0.0049 | 91.65% | 0.0305 | 40.49% | 0.1130 |
| RZ899042 | 95.68% | 0.0128 | 87.69% | 0.0538 | 50.73% | 0.0801 |

Example 16: In the Present Disclosure, the Sequences RZ899011 (siRNA Targeting SOD1 Conjugated with Dimeric CR01022) and RZ899038 (siRNA Targeting SOD1 Conjugated With Dimeric CR01017) were Selected for In Vivo Toxicological Evaluation in Mice.

In this example, the siRNA sequences RZ899011 conjugated with dimeric CR01022 and RZ899038 conjugated with dimeric CR01017 carrier were administered via subcutaneous injection, and the nature and severity of toxic response to the two siRNAs in ICR mice were evaluated.

6 to 8-week-old ICR mice were randomly divided into 3 groups according to their body weight, with 10 mice in each group (half male and half female). PBS solution (without containing siRNA conjugate) and the siRNA conjugates in this example were respectively administered subcutaneously into the abdomen of mice of each group. Each mouse in the PBS control group was administered with a volume of 10 ml/kg body weight of mouse, and each mouse in the siRNA conjugate administration groups was administered with a dose of 300 mg/kg body weight of mouse (calculated based on siRNA) and a volume of 10 ml/kg.

The day of administration was noted as day 1 (D1). Clinical observations were performed at least once daily after the first administration during the study period. On day 15 of the study (noted as D15), blood samples were collected from all animals for hematological analysis and blood biochemistry analysis, where mice were fasted for at least 12 h (with free access to water) before sampling. For routine blood test, blood was collected using EDTA-K2 as anticoagulant and a routine blood test was carried out by using an automated hematology analyzer (Sysmex XN-10B1). Blood was collected using sodium citrate as anticoagulant and centrifuged at approximately 2,000 g at room temperature for 10 min. The plasma was separated and the blood coagulation indexes were detected using an automated coagulation analyzer (Mindray C3510). Blood samples were collected without using anticoagulant and left to stand at room temperature for approximately 30 min. After coagulated, blood was centrifuged at 2,000 g at 4 °C for 10 min. The blood biochemistry analysis was carried out using an automated biochemistry analyzer (Sysmex BX-4000). On D15 of the study (noted as D15), all animals were dissected. Mice to be dissected were fasted (with free access to water) for at least 12 h before dissection. Animals were anesthetized with Zoletil 50 (8G4VA, Virbac, France) for blood collection. After the blood was collected, mice were euthanized by exsanguination via abdominal aorta and subjected to gross dissection observation. The livers collected from all animals were subjected to histopathological examination by hematoxylin-eosin staining. If necessary, additional organs showing apparent abnormalities in size, shape, color, or texture were also subjected to histopathological examination.

Primer sequences used in this example are shown in Table 7 of example 2.

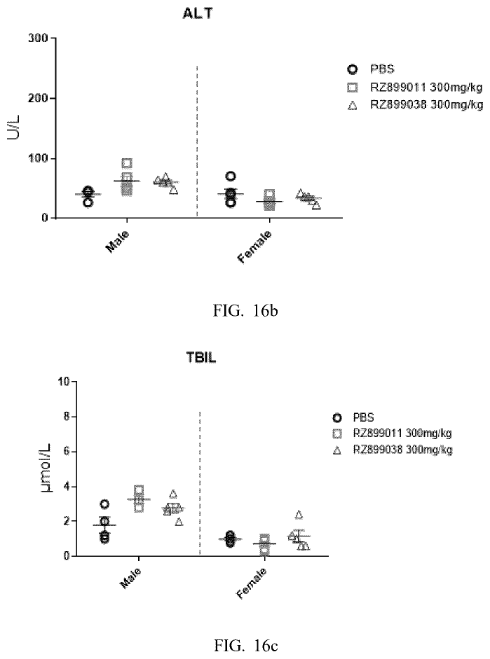

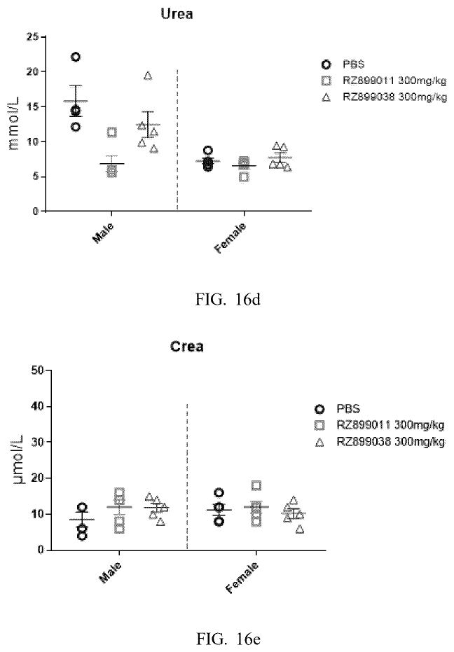

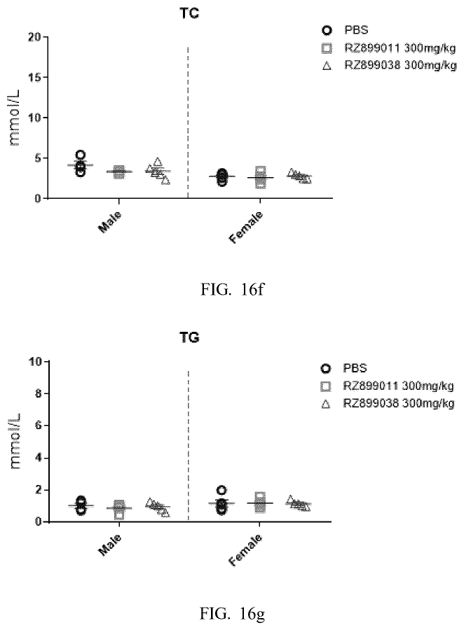

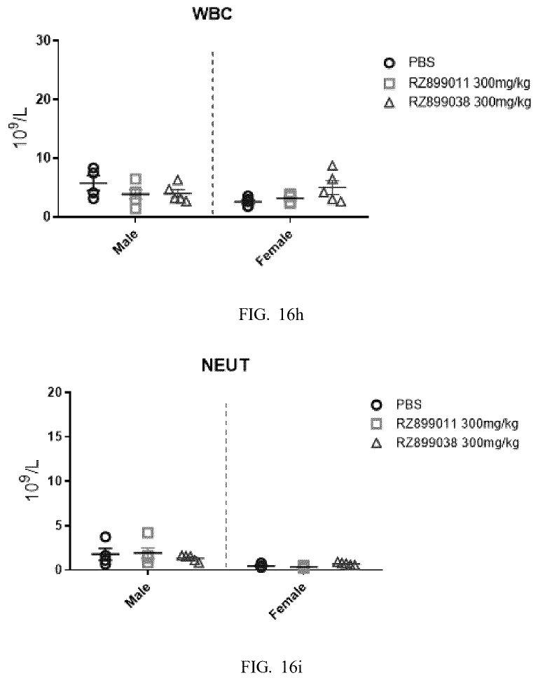

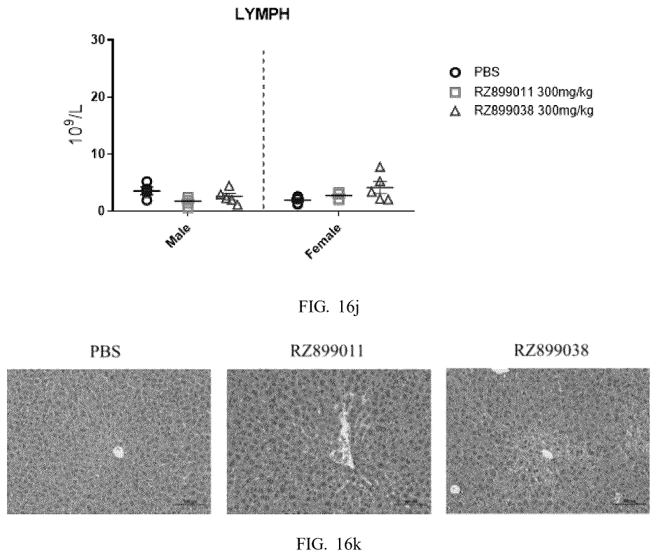

The results of example 16 showed that after administration of the siRNA conjugates in this example, no clinical abnormalities were observed in the PBS group, the RZ899011 administration group and the RZ899038 administration group during the study period. At the endpoint of the study (D15), no obvious abnormalities were observed in gross dissection observation. There were no abnormalities in hematological and blood biochemical analyses of animals in each group (FIG. 16a-j and Table 23), and no abnormalities in histopathological staining of liver tissues of animals in each group (FIG. 16k). These results show that the siRNA conjugates and their carrier molecules of the present disclosure have favorable safety profiles and low toxicity at animal level.

TABLE 23
Results of hematological and blood biochemical analyses in mice after
administration of siRNA conjugates in this example

| | PBS | | | | RZ899011 | | | | RZ899038 | | | |
|---|---|---|---|---|---|---|---|---|---|---|---|---|
| | Male | | Female | | Male | | Female | | Male | | Female | |
| Detection | Average | | Average | | Average | | Average | | Average | | Average | |
| index | value | ±SD | value | ±SD | value | ±SD | value | ±SD | value | ±SD | value | ±SD |
| Aspartate | 126.5 | 37.7 | 185.4 | 37.6 | 155.2 | 36.4 | 205.4 | 29.0 | 160.2 | 53.0 | 157.6 | 40.0 |
| aminotransferase | | | | | | | | | | | | |
| (AST) | | | | | | | | | | | | |
| Alanine | 40.0 | 9.4 | 40.6 | 18.0 | 62.0 | 18.7 | 28.0 | 7.2 | 60.2 | 7.8 | 33.2 | 7.6 |
| aminotransferase | | | | | | | | | | | | |
| (ALT) | | | | | | | | | | | | |
| Total | 1.8 | 0.9 | 1.0 | 0.2 | 3.3 | 0.5 | 0.7 | 0.3 | 2.8 | 0.6 | 1.2 | 0.7 |
| bilirubin | | | | | | | | | | | | |
| (TBIL) | | | | | | | | | | | | |
| Urea | 15.8 | 4.4 | 7.2 | 0.9 | 6.9 | 2.5 | 6.5 | 0.9 | 12.4 | 4.2 | 7.7 | 1.5 |
| Creatinine | 8.5 | 4.1 | 11.2 | 3.3 | 12.0 | 4.7 | 12.0 | 3.7 | 11.8 | 2.9 | 10.2 | 3.0 |
| (Crea) | | | | | | | | | | | | |
| Total | 4.2 | 0.9 | 2.8 | 0.4 | 3.4 | 0.2 | 2.6 | 0.5 | 3.4 | 0.9 | 2.8 | 0.3 |
| cholesterol | | | | | | | | | | | | |
| (TC) | | | | | | | | | | | | |
| Triglyceride | 1.0 | 0.3 | 1.2 | 0.5 | 0.9 | 0.2 | 1.2 | 0.2 | 0.9 | 0.3 | 1.1 | 0.2 |
| (TG) | | | | | | | | | | | | |
| White blood | 5.8 | 2.5 | 2.6 | 0.8 | 3.9 | 1.9 | 3.2 | 0.7 | 4.0 | 1.5 | 5.0 | 2.6 |
| cell (WBC) | | | | | | | | | | | | |
| count | | | | | | | | | | | | |
| Neutrophil | 1.8 | 1.4 | 0.5 | 0.2 | 1.9 | 1.3 | 0.3 | 0.1 | 1.3 | 0.3 | 0.7 | 0.2 |
| (NEUT) | | | | | | | | | | | | |
| count | | | | | | | | | | | | |
| Lymphocyte | 3.6 | 1.4 | 2.0 | 0.6 | 1.8 | 0.8 | 2.7 | 0.6 | 2.6 | 1.2 | 4.1 | 2.4 |
| (LYMPH) | | | | | | | | | | | | |
| count | | | | | | | | | | | | |

The above provides a detailed description of the compounds, conjugates, compositions, and use thereof provided by the present disclosure. The principle and embodiments of the present disclosure are illustrated by specific examples herein. The above descriptions of the examples are only used to facilitate understanding of the method and the core idea of the present disclosure. It should be noted that, those skilled in the art can make several changes and modifications to the present disclosure without departing from the principle of the present disclosure, and the changes and modifications shall fall within the protection scope of the claims.

1. A compound having a structure represented by formula (Ia), or a stereoisomer, a pharmaceutically acceptable salt or a prodrug thereof, 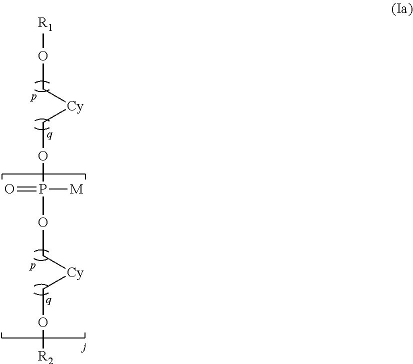
in formula (Ia), R₁ is selected from the group consisting of H, a hydroxyl protecting group and 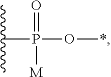
wherein * represents a site for linking an active pharmaceutical molecule;
R₂ is selected from the group consisting of H, a reactive phosphorus group and 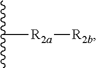
wherein R_{2b} is a solid phase support containing an amino functional group, and R₂ₐ is a covalent linking group linked to the amino functional group;
j is selected from the group consisting of 0, 1, 2 and 3;
each M is independently selected from the group consisting of hydroxyl and mercapto;
each p is independently selected from the group consisting of 0, 1, 2, 3 and 4;
each q is independently selected from the group consisting of 0, 1, 2, 3 and 4;
each Cy is independently 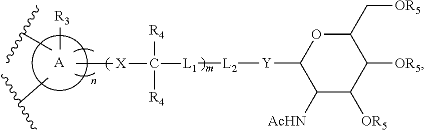
wherein A is a 5-6 membered oxygen-containing heterocycle, R₃ is selected from the group consisting of H and C₁-C₆ alkoxy, n is selected from the group consisting of 1 and 2, each X is independently selected from the group consisting of O, S and NH, each R₄ is independently selected from the group consisting of H, C₁-C₆ alkyl, and C₁-C₆ alkoxy, each L₁ is independently selected from the group consisting of a substituted or unsubstituted triazolyl and a substituted or unsubstituted amido, m is selected from the group consisting of 1 and 2, and each L₂ is independently selected from the group consisting of a substituted or unsubstituted C₁-C₃₀ alkylidene and 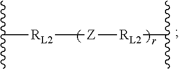
wherein each r is independently selected from the group consisting of 1, 2, 3, 4, 5, 6, 7, 8, 9, and 10, each Z is independently selected from the group consisting of O, S and NH, each R_{L2} is independently a substituted or unsubstituted C₁-C₁₀ alkylidene, each Y is independently selected from the group consisting of O, S and NH, and each R₅ is independently selected from the group consisting of H, a substituted or unsubstituted C₁-C₄ alkyacyl and a substituted or unsubstituted C₆-C₁₂ arylacyl.

2. The compound, or the stereoisomer, the pharmaceutically acceptable salt or the prodrug thereof according to claim 1, wherein A is a 5 to 6-membered saturated oxygen-containing heterocycle;
optionally, A is a 5 to 6-membered saturated heterocycle containing one oxygen atom;
optionally, m is 1;
optionally, X is O;
optionally, each R₄ is H;
optionally, each L₁ is independently selected from the group consisting of: 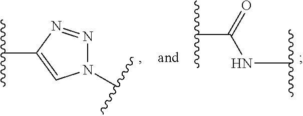 [SMILES: *c1cn(*)nn1 ; *NC(*)=O]
optionally, each L₁ is 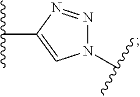 [SMILES: *c1cn(*)nn1]
optionally, each L₁ is 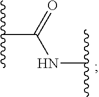 [SMILES: *NC(*)=O]
optionally, R₃ is selected from the group consisting of H and C₁-C₃ alkoxy;
optionally, R₃ is selected from the group consisting of H and methoxy;
optionally, each L₂ is independently 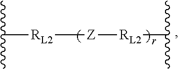
wherein each r is independently selected from the group consisting of 1, 2, 3, 4, 5, 6, 7, 8, 9 and 10, each Z is independently selected from the group consisting of O, S and NH, and each R_{L2} is independently a substituted or unsubstituted C₁-C₁₀ alkylidene;
optionally, each L_{L2} is independently a substituted or unsubstituted C₁-C₅ alkylidene;
optionally, each L_{L2} is independently a substituted or unsubstituted C₁-C₃ alkylidene;
optionally, each L_{L2} is independently a substituted or unsubstituted C₂ alkylidene;
optionally, each r is independently selected from the group consisting of 2 and 3;
optionally, each L₂ is independently 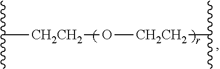
wherein r is selected from the group consisting of 1, 2, 3, 4, 5, 6, 7, 8, 9 and 10;
optionally, each L₂ is independently 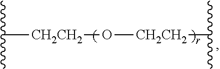
wherein r is selected from the group consisting of 2 and 3;
optionally, Y is O;
optionally, each R₅ is independently selected from the group consisting of H, 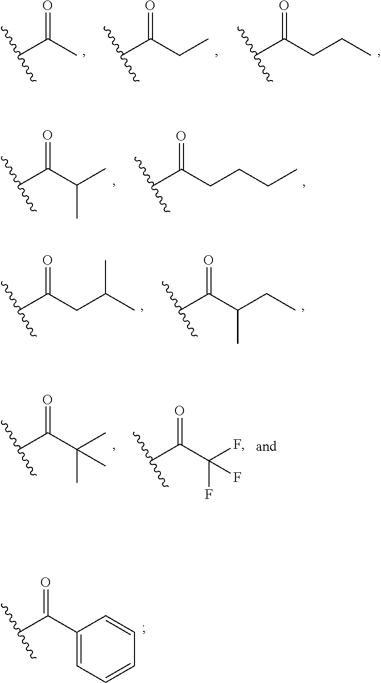
optionally, each R₅ is 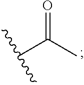 [SMILES: *C(C)=O]
optionally, each R₅ is H;
optionally, p and q are each independently selected from the group consisting of 0 and 1;
optionally, p=1 and q=1, or p=1 and q=0, or p=0 and q=1, or p=0 and q=0;
optionally, p=1 and q=0;
optionally, the hydroxyl protecting group is selected from the group consisting of trityl, 4-methoxytrityl, 4,4′-dimethoxytrityl and 4,4′,4″-trimethoxytrityl;
optionally, the hydroxyl protecting group is 4,4′-dimethoxytrityl;
optionally, the reactive phosphorus group is selected from the group consisting of 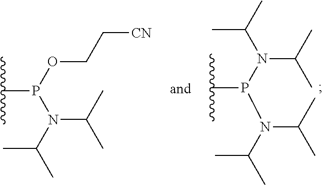 [SMILES: *P(OCCC#N)N(C(C)C)C(C)C ; *P(N(C(C)C)C(C)C)N(C(C)C)C(C)C]
optionally, the reactive phosphorus group is 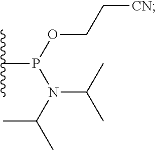 [SMILES: *P(OCCC#N)N(C(C)C)C(C)C]
optionally, the active pharmaceutical molecule is selected from the group consisting of a small molecule drug, an antibody, and an oligonucleotide;
optionally, the oligonucleotide is selected from the group consisting of a single-stranded oligonucleotide and a double-stranded oligonucleotide;
optionally, the active pharmaceutical molecule is a double-stranded oligonucleotide;
optionally, the double-stranded oligonucleotide is siRNA;
optionally, the solid phase support is selected from the group consisting of resin containing an amino functional group and controlled pore glass bead containing an amino functional group;
optionally, R_{2b} is 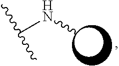
wherein [symbol] represents resin or controlled pore glass bead;
optionally, R₂ₐ is 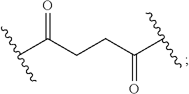 [SMILES: *C(=O)CCC(*)=O]
optionally, 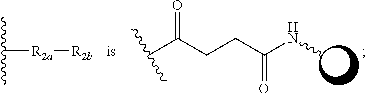
optionally, j is selected from the group consisting of 0, 1 and 2;
optionally, j is 0;
optionally, j is 1; and
optionally, j is 2.

3. The compound, or the stereoisomer, the pharmaceutically acceptable salt or the prodrug thereof according to claim 1, wherein each Cy independently has a structure selected from the group consisting of:

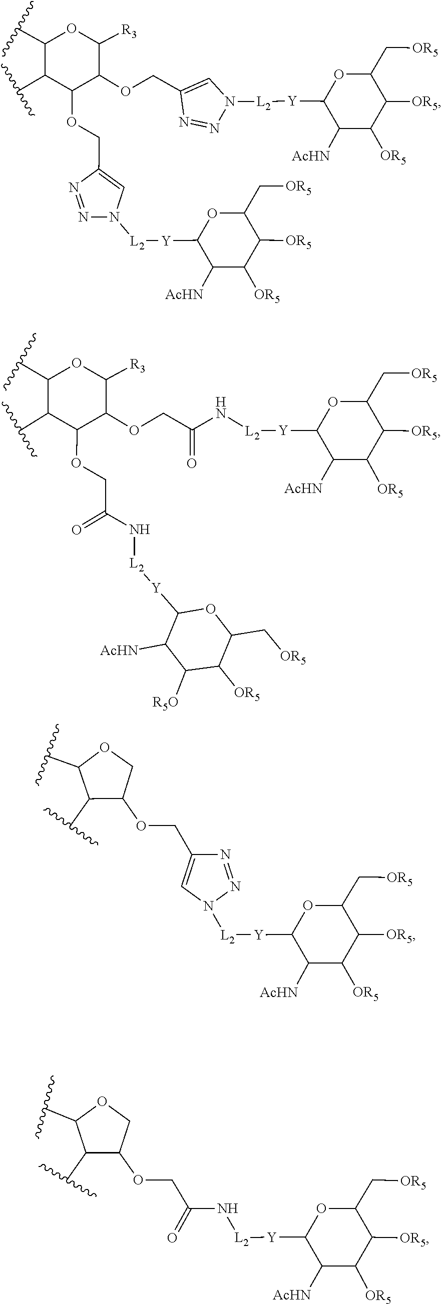

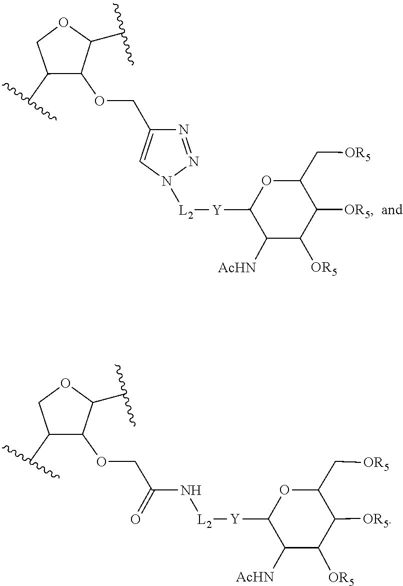

4. The compound, or the stereoisomer, the pharmaceutically acceptable salt or the prodrug thereof according to claim 1, wherein the compound has a structure selected from the group consisting of:

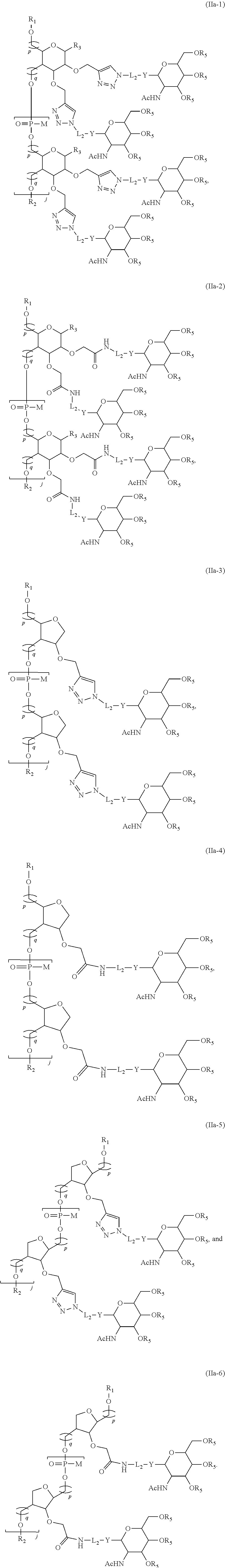

5. The compound, or the stereoisomer, the pharmaceutically acceptable salt or the prodrug thereof according to claim 1, wherein the compound is selected from the group consisting of:

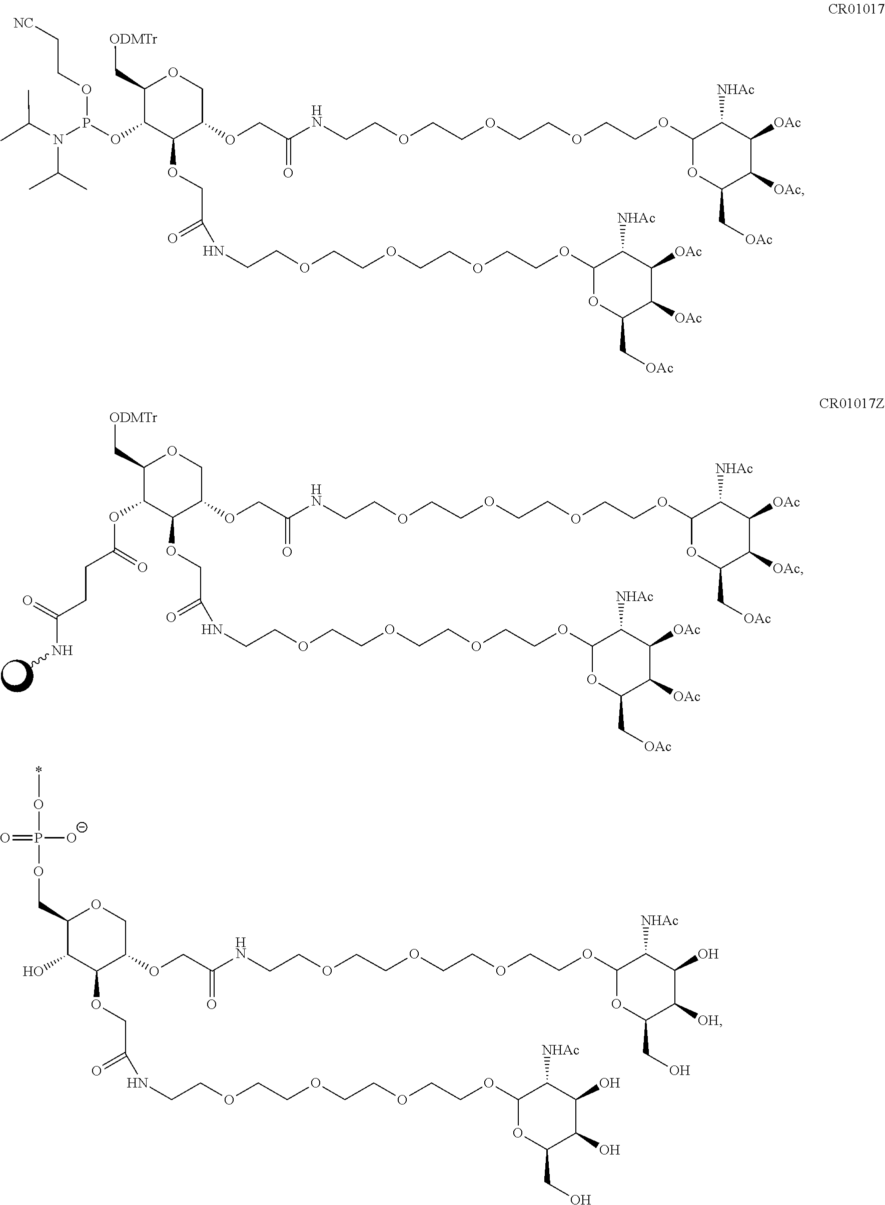

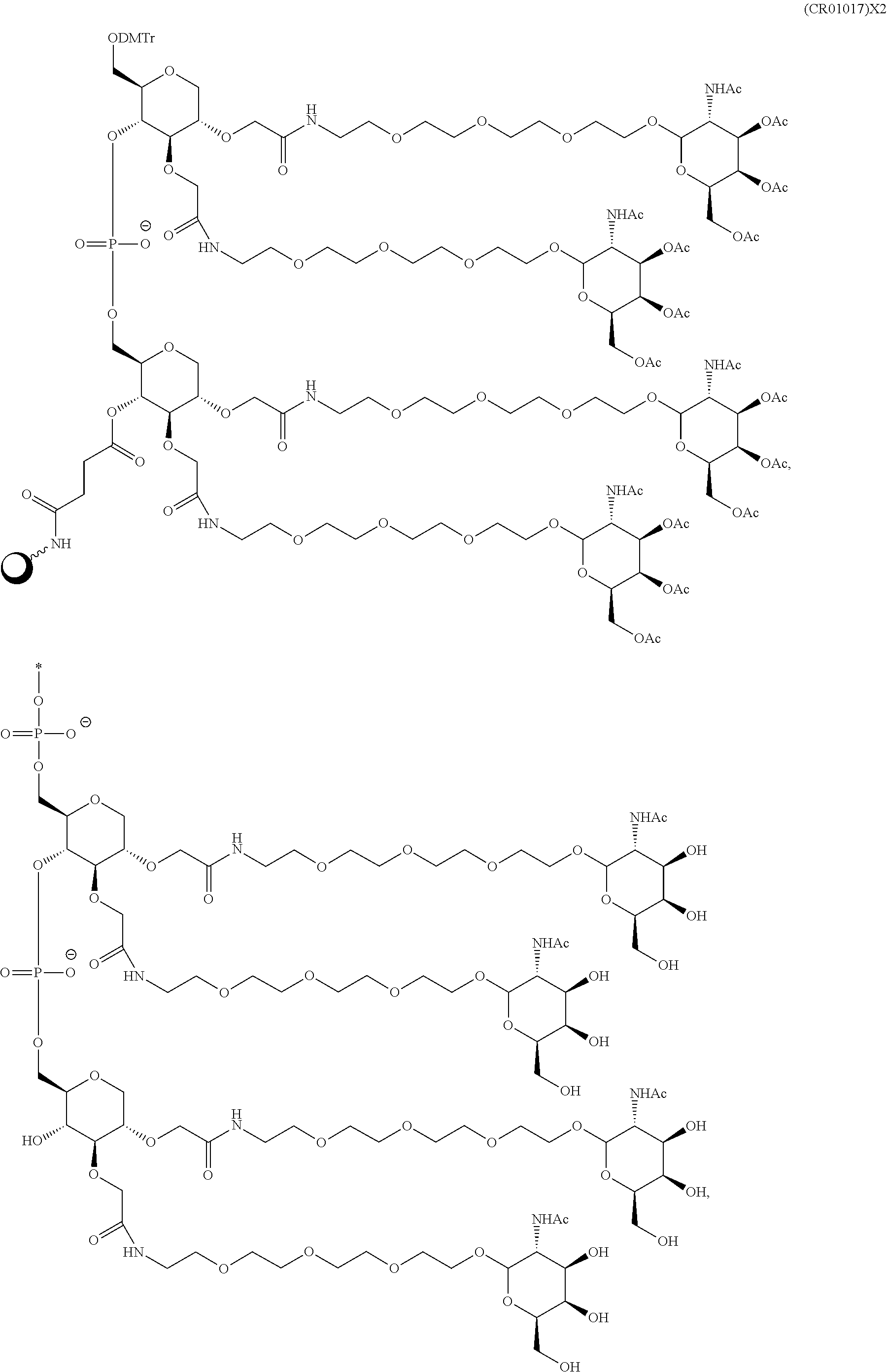

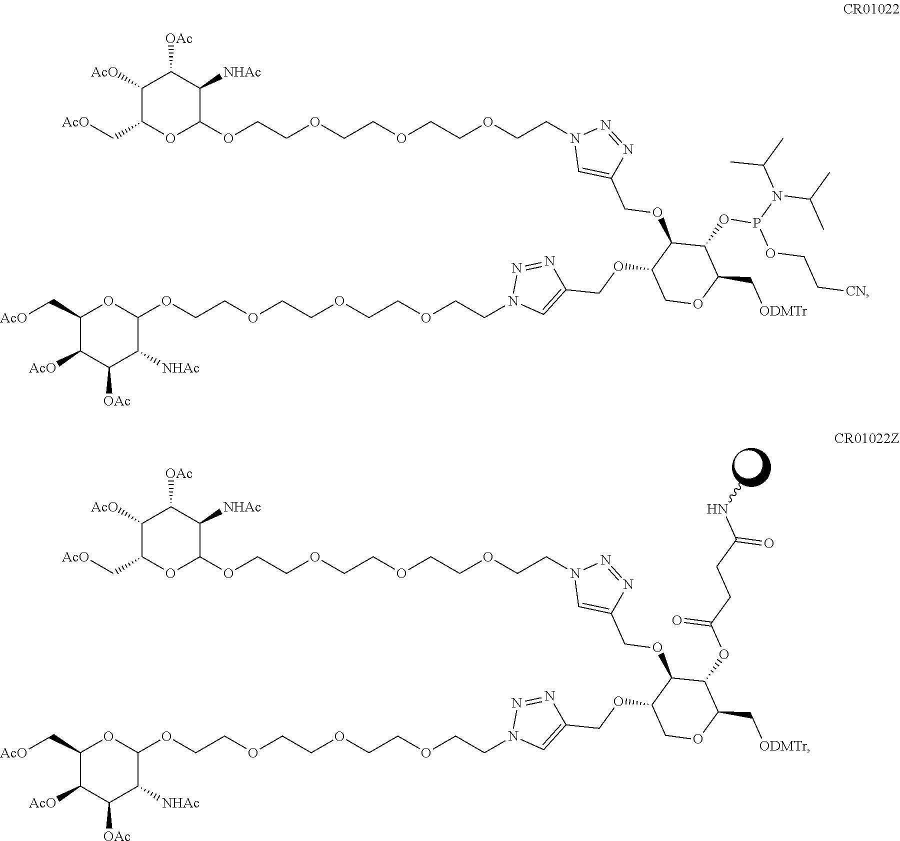

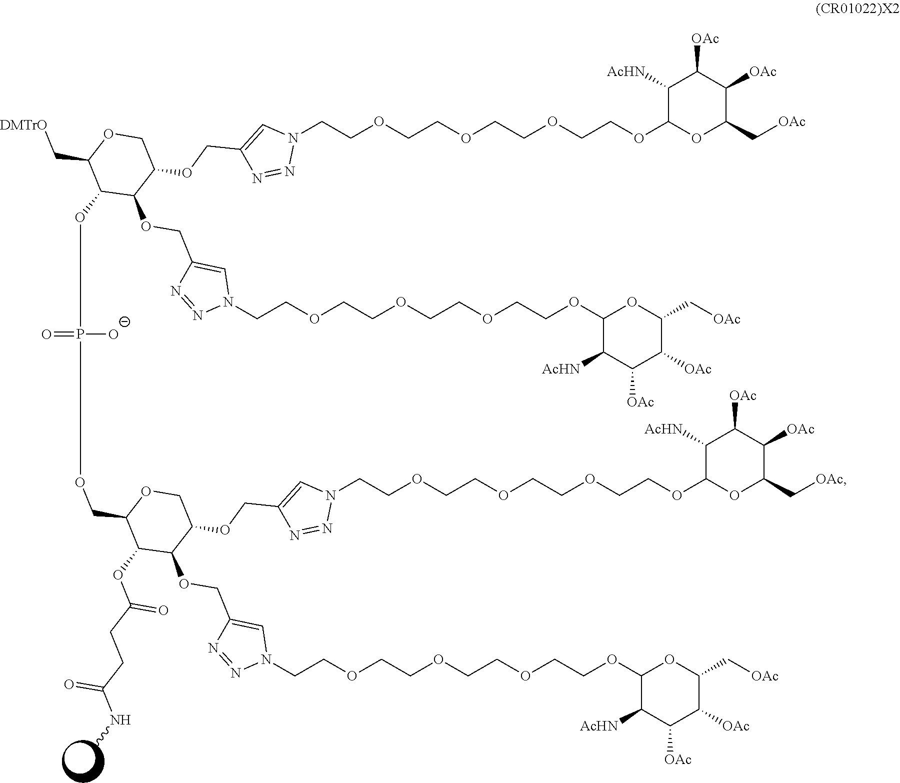

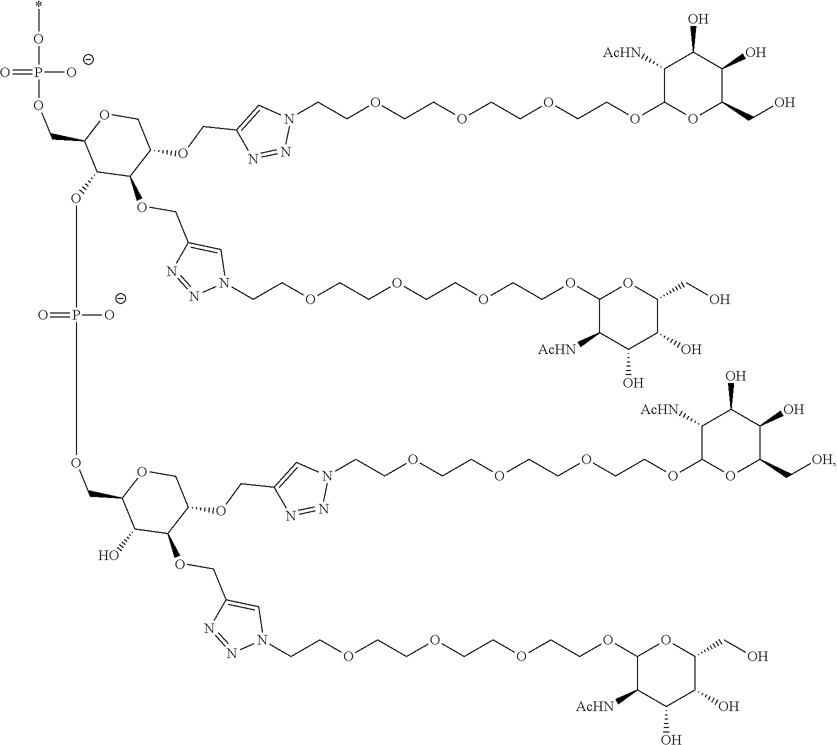 [SMILES: *OP(=O)([O-])OC[C@H]1OC[C@H](OCc2cn(CCOCCOCCOCCOC3O[C@H](CO)[C@H](O)[C@H](O)[C@H]3NC(C)=O)nn2)[C@@H](OCc2cn(CCOCCOCCOCCOC3O[C@H](CO)[C@H](O)[C@H](O)[C@H]3NC(C)=O)nn2)[C@@H]1OP(=O)([O-])OC[C@H]1OC[C@H](OCc2cn(CCOCCOCCOCCOC3O[C@H](CO)[C@H](O)[C@H](O)[C@H]3NC(C)=O)nn2)[C@@H](OCc2cn(CCOCCOCCOCCOC3O[C@H](CO)[C@H](O)[C@H](O)[C@H]3NC(C)=O)nn2)[C@@H]1O]

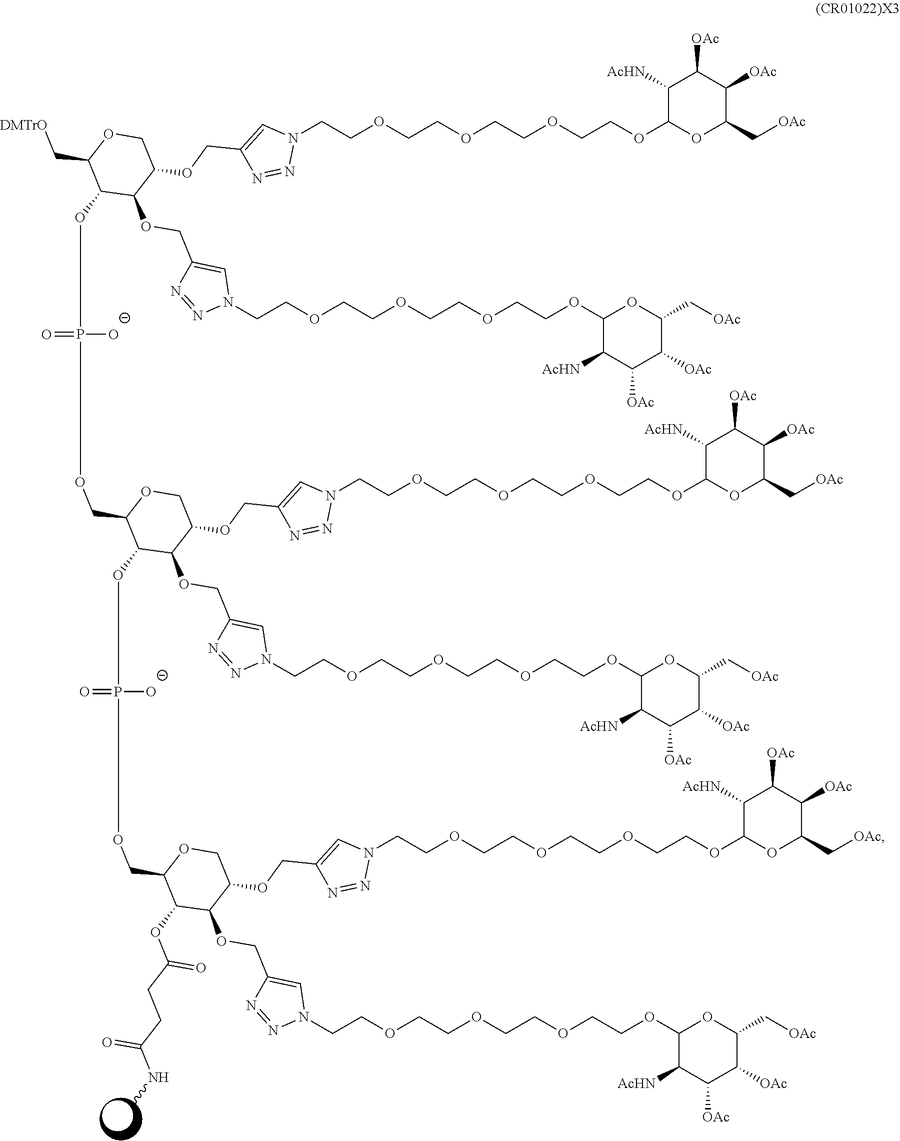

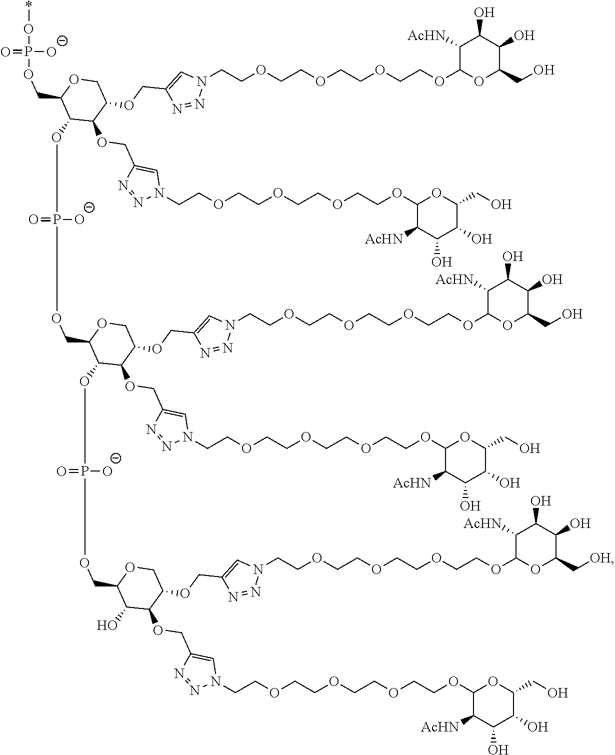

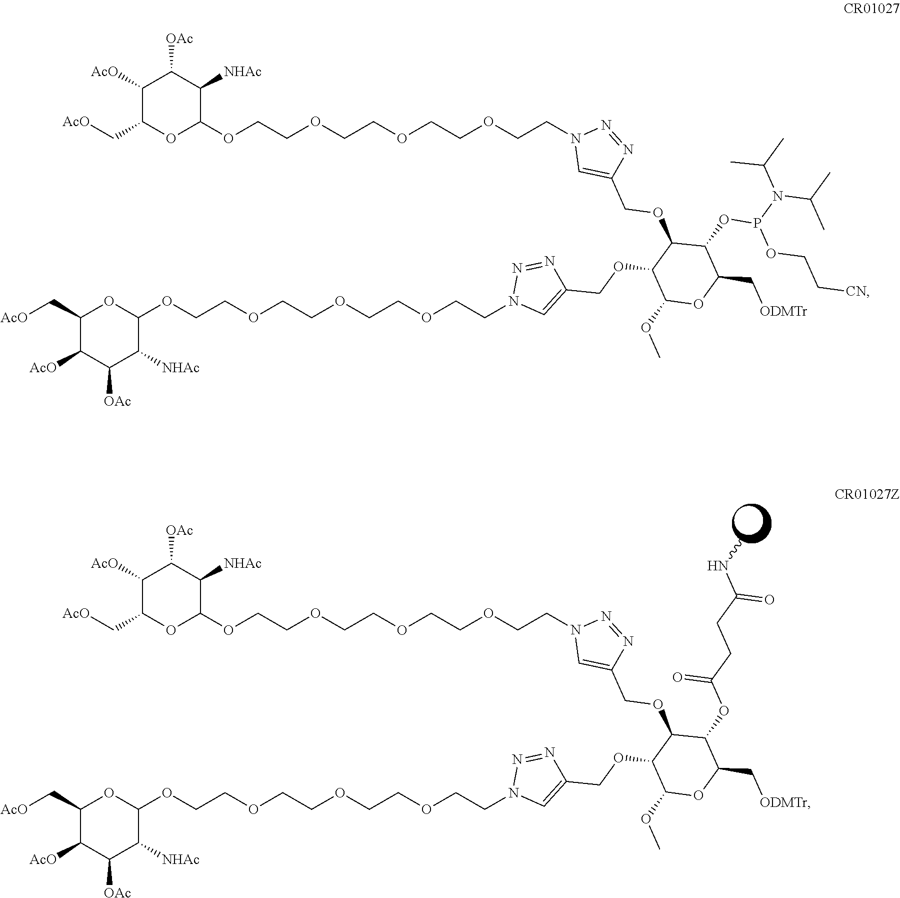

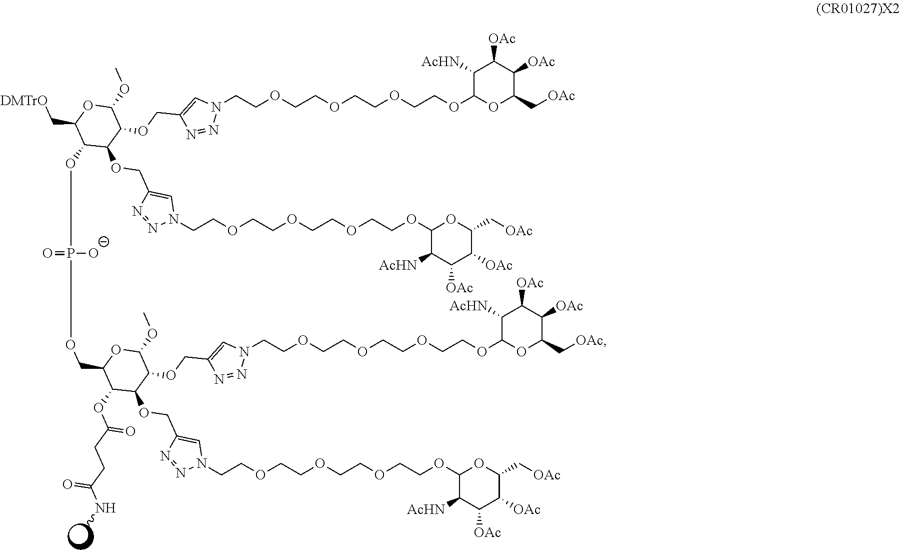

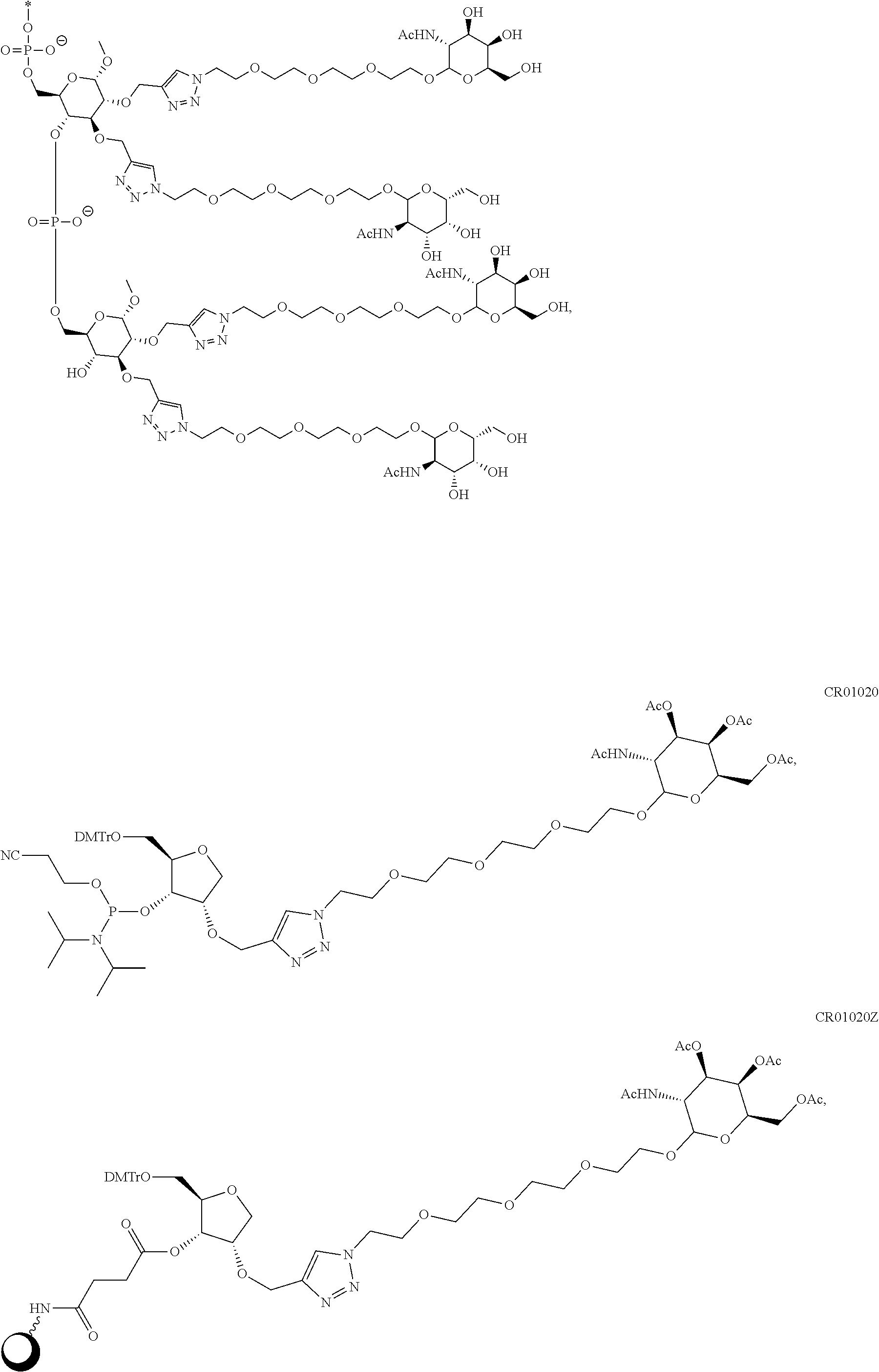

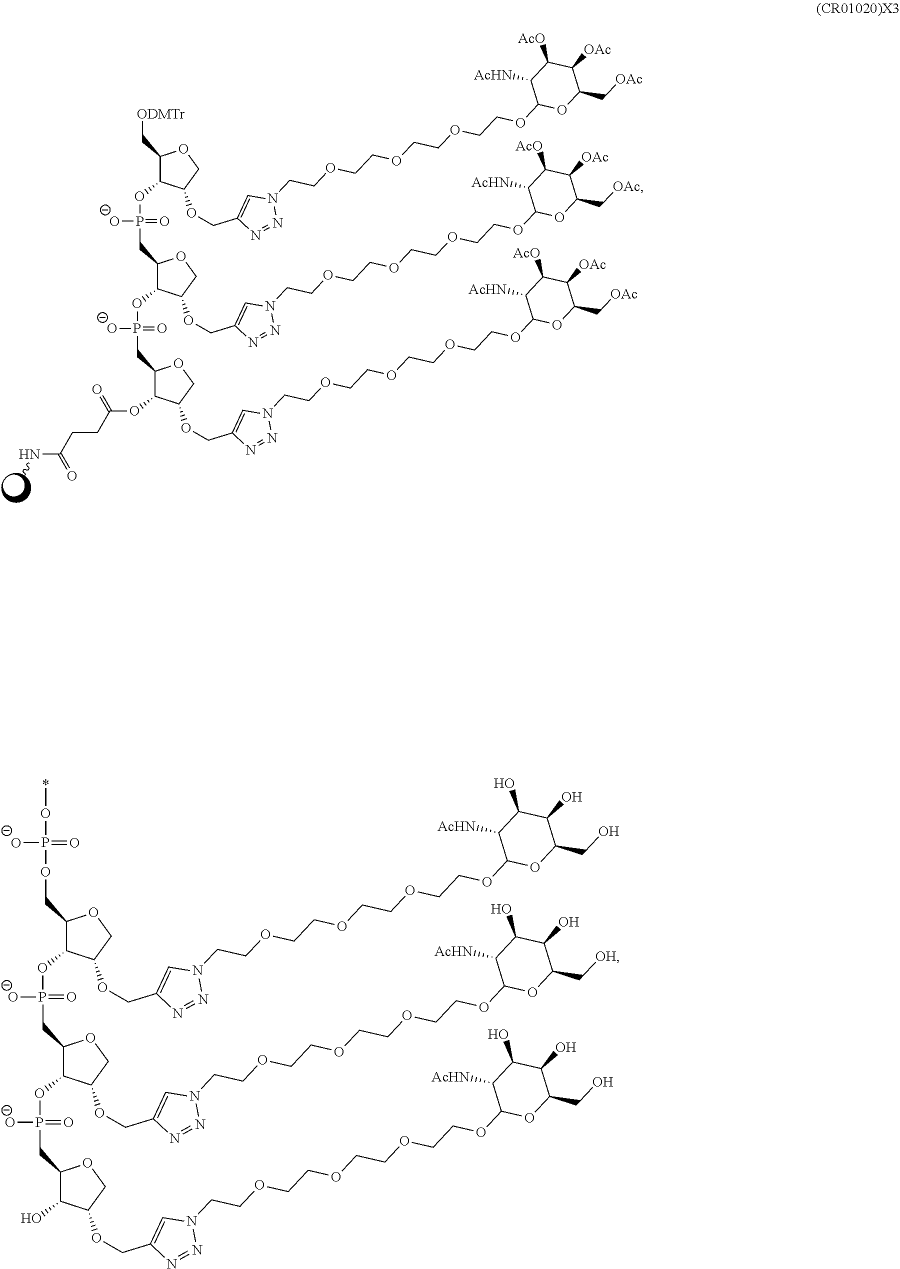

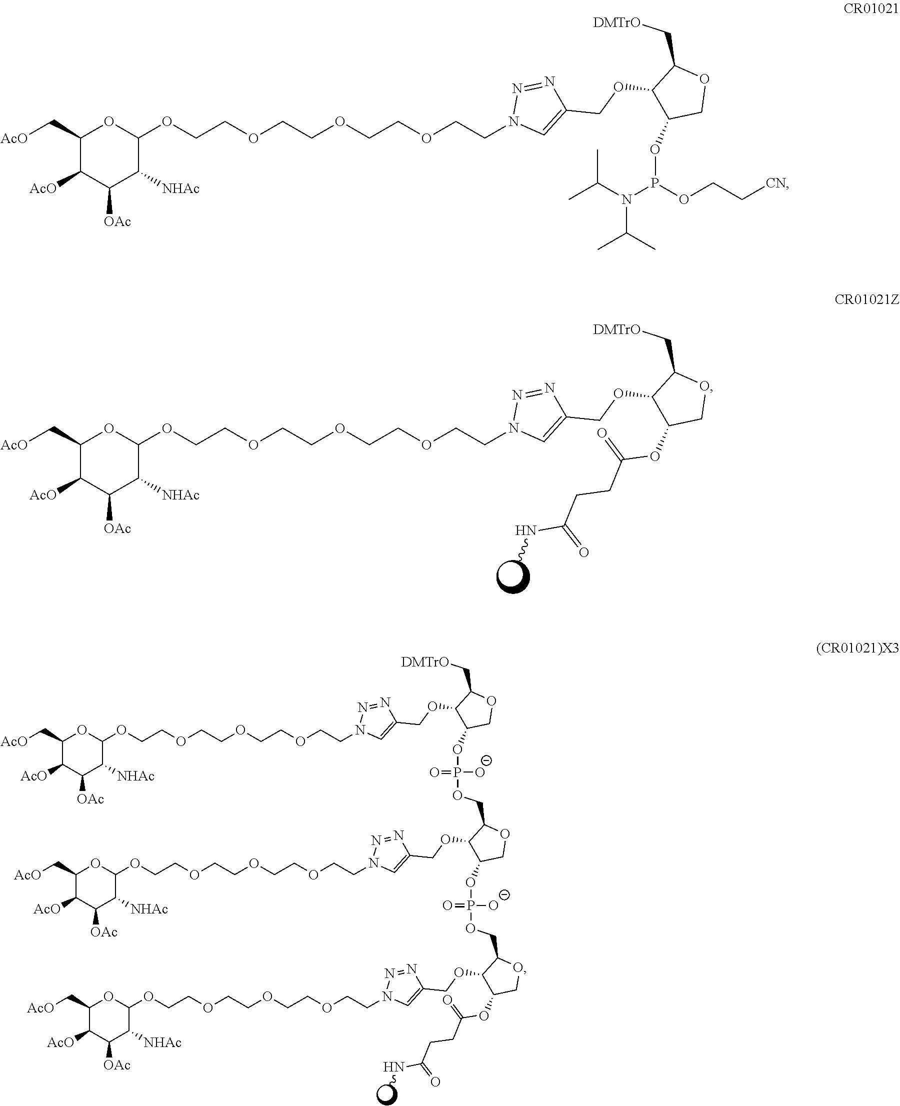

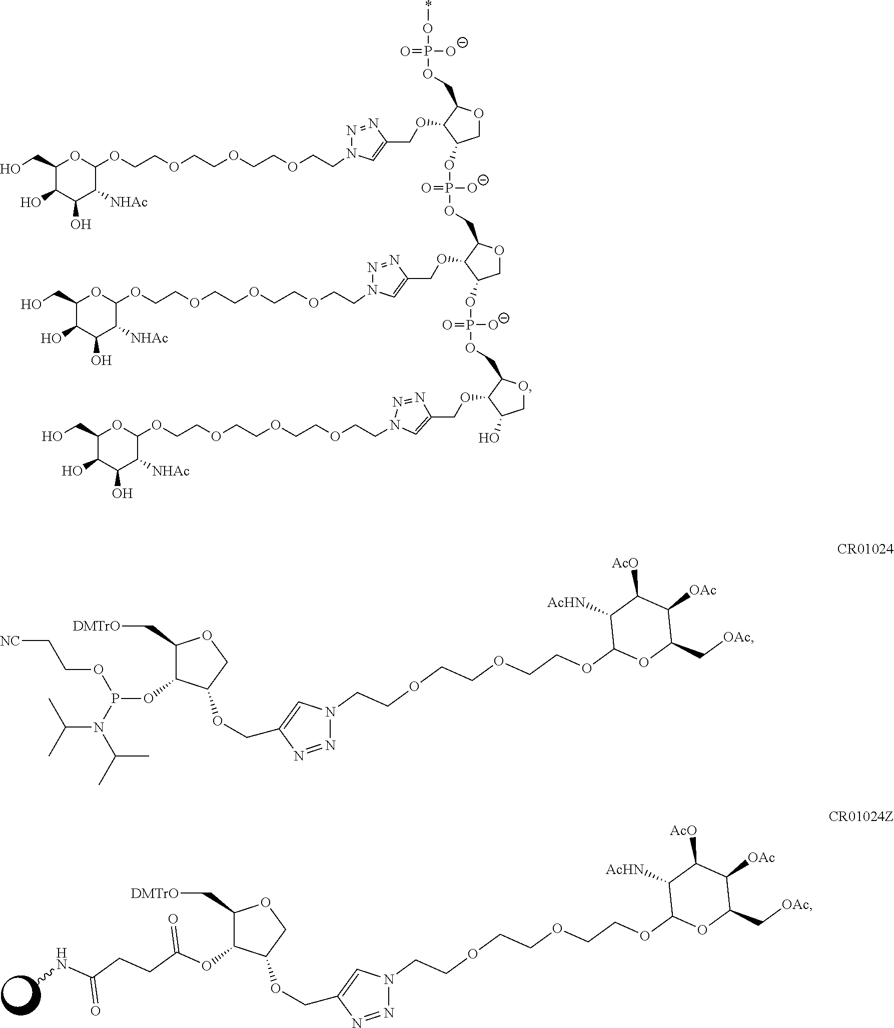

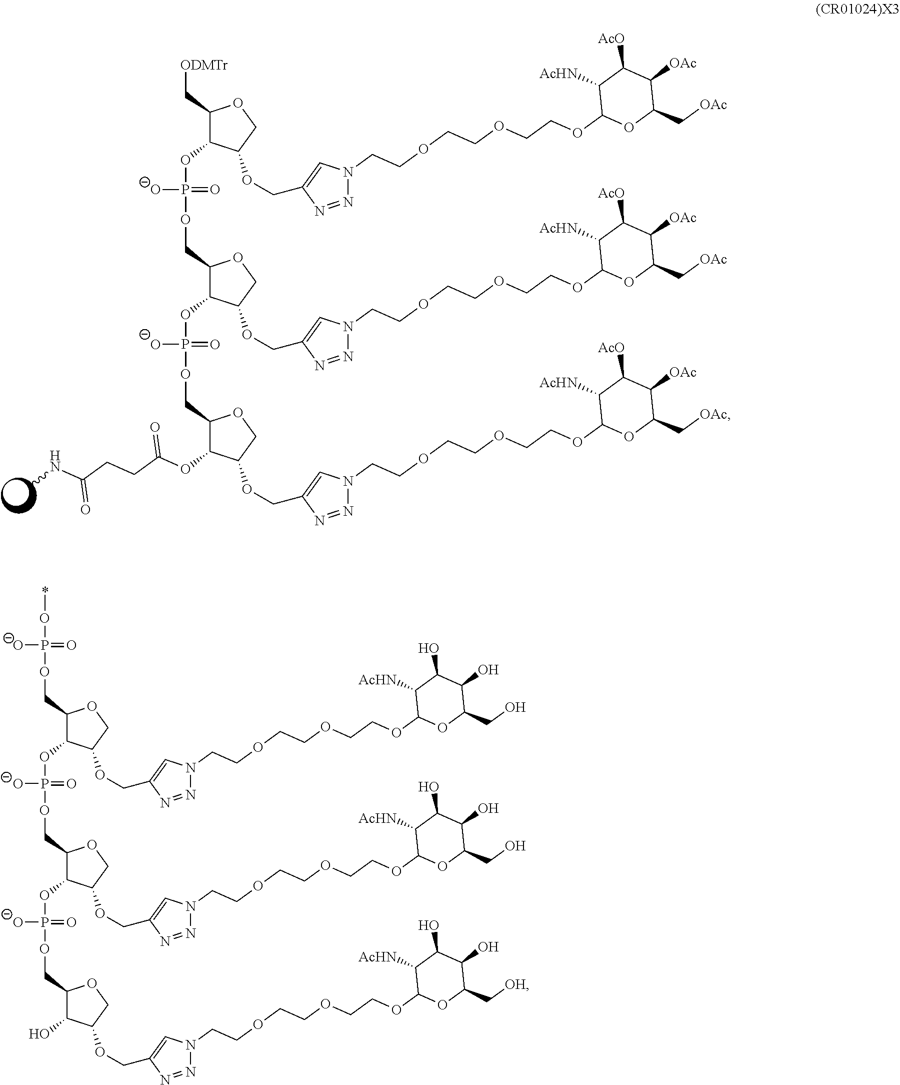

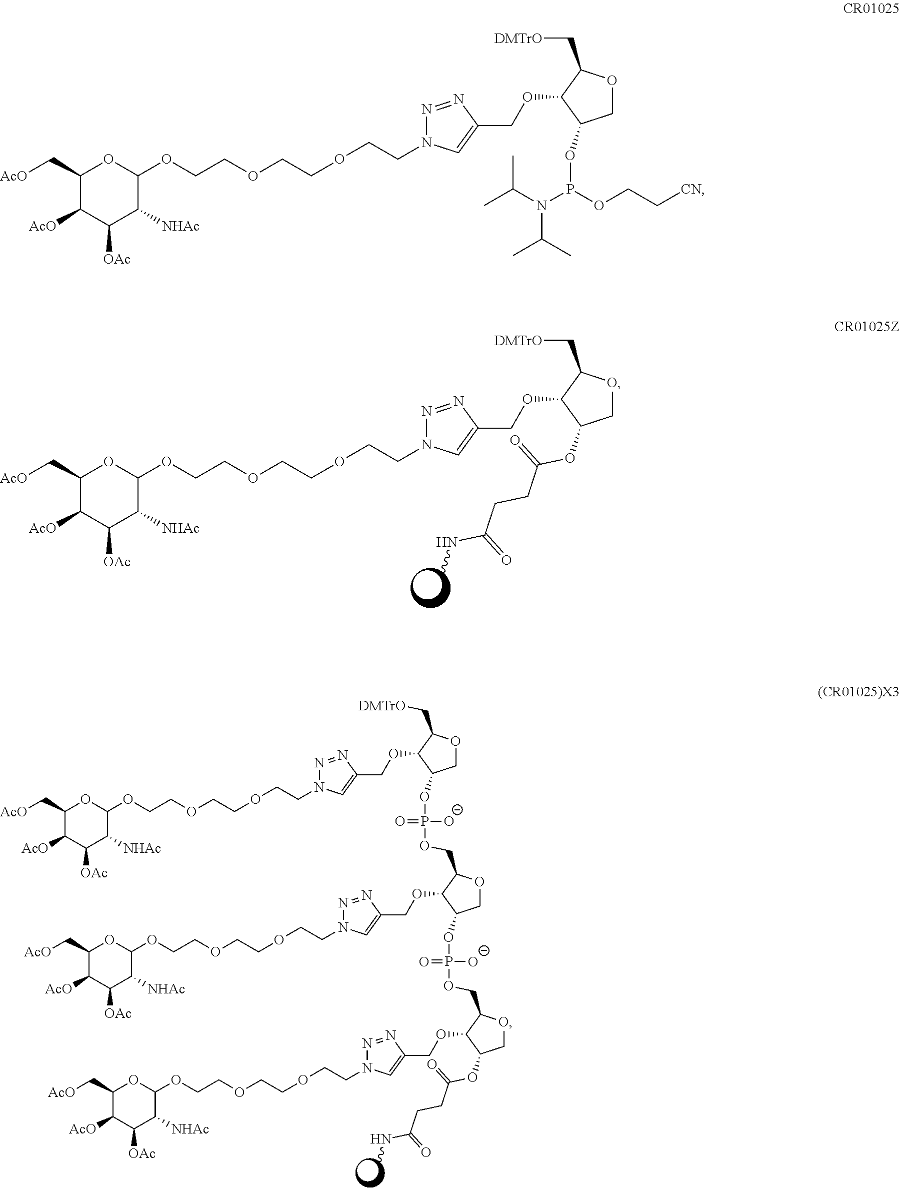

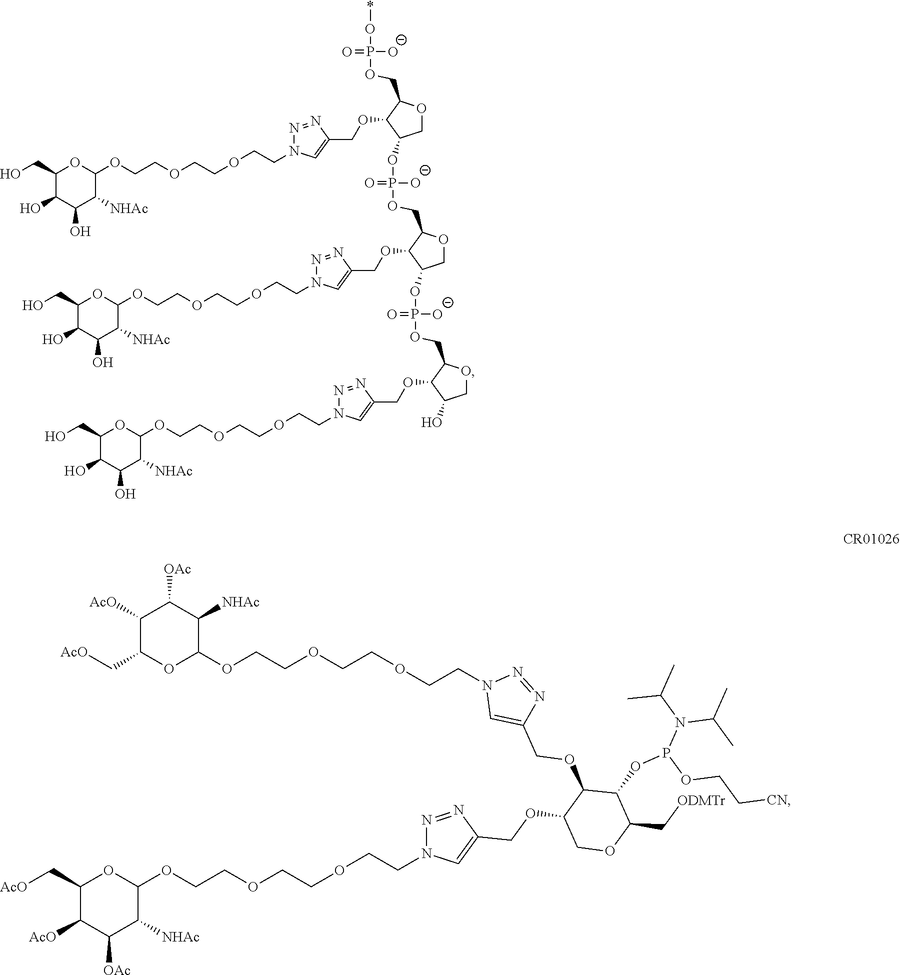

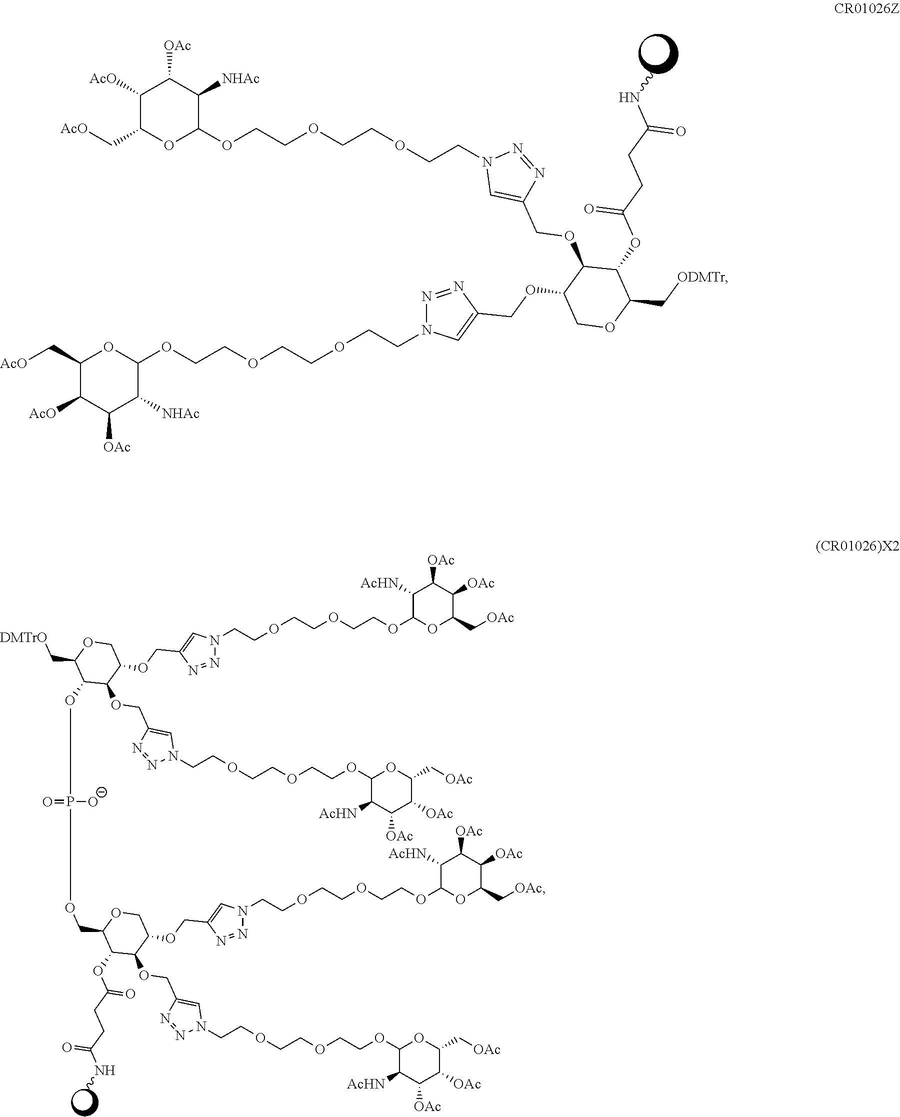

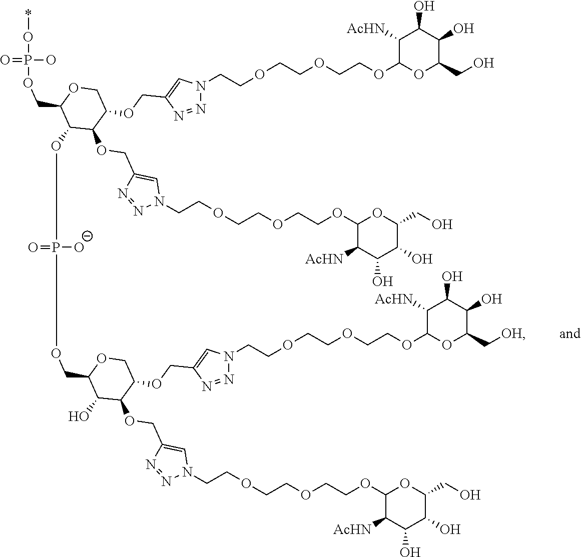 [SMILES: *OP(=O)(O)OC[C@H]1OC[C@H](OCc2cn(CCOCCOCCOC3O[C@H](CO)[C@H](O)[C@H](O)[C@H]3NC(C)=O)nn2)[C@@H](OCc2cn(CCOCCOCCOC3O[C@H](CO)[C@H](O)[C@H](O)[C@H]3NC(C)=O)nn2)[C@@H]1OP(=O)([O-])OC[C@H]1OC[C@H](OCc2cn(CCOCCOCCOC3O[C@H](CO)[C@H](O)[C@H](O)[C@H]3NC(C)=O)nn2)[C@@H](OCc2cn(CCOCCOCCOC3O[C@H](CO)[C@H](O)[C@H](O)[C@H]3NC(C)=O)nn2)[C@@H]1O]

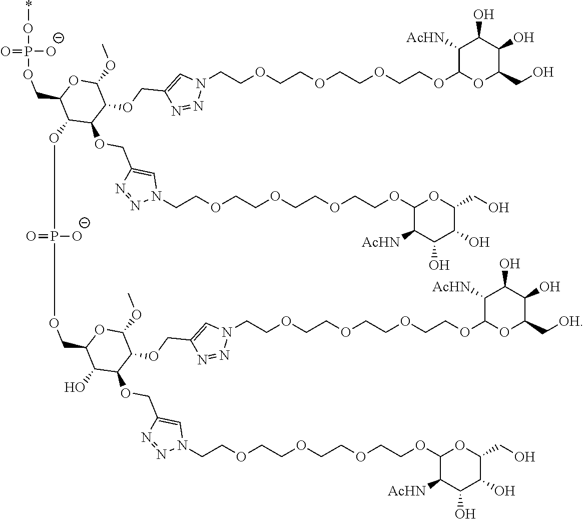 [SMILES: *OP(=O)([O-])OC[C@H]1O[C@H](OC)[C@H](OCc2cn(CCOCCOCCOCCOC3O[C@H](CO)[C@H](O)[C@H](O)[C@H]3NC(C)=O)nn2)[C@@H](OCc2cn(CCOCCOCCOCCOC3O[C@H](CO)[C@H](O)[C@H](O)[C@H]3NC(C)=O)nn2)[C@@H]1OP(=O)([O-])OC[C@H]1O[C@H](OC)[C@H](OCc2cn(CCOCCOCCOCCOC3O[C@H](CO)[C@H](O)[C@H](O)[C@H]3NC(C)=O)nn2)[C@@H](OCc2cn(CCOCCOCCOCCOC3O[C@H](CO)[C@H](O)[C@H](O)[C@H]3NC(C)=O)nn2)[C@@H]1O]

6. A conjugate having a structure represented by formula (Ib), or a stereoisomer, a pharmaceutically acceptable salt or a prodrug thereof, 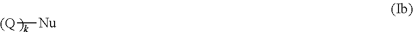
in formula (Ib), Nu represents an oligonucleotide;
k is selected from the group consisting of 1 and 2;
Q has a structure of 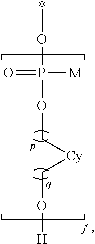
wherein j′ is selected from the group consisting of 1, 2, 3 and 4, and *, M, p, q and Cy are as defined in claim 1.

7. The conjugate according to claim 6, wherein j′ is selected from the group consisting of 1, 2 and 3;
optionally, j′ is 1;
optionally, j′ is 2;
optionally, j′ is 3;
optionally, k is 1;
optionally, k is 2;
optionally, the oligonucleotide is selected from the group consisting of a single-stranded oligonucleotide and a double-stranded oligonucleotide;
optionally, Nu represents a double-stranded oligonucleotide;
optionally, the double-stranded oligonucleotide is siRNA;
optionally, k is 1, and one Q is conjugated to the 3′ end of the siRNA sense strand; and
optionally, k is 2, and two Q are respectively conjugated to the 3′ end and 5′ end of the siRNA sense strand.

8. The conjugate, or the stereoisomer, the pharmaceutically acceptable salt or the prodrug thereof according to claim 6, wherein the conjugate has a structure selected from the group consisting of:

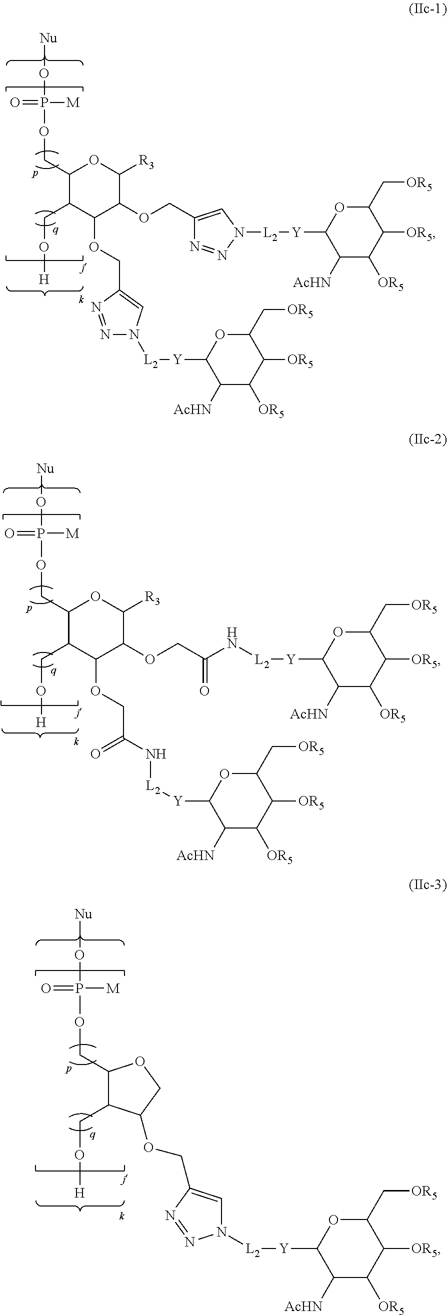

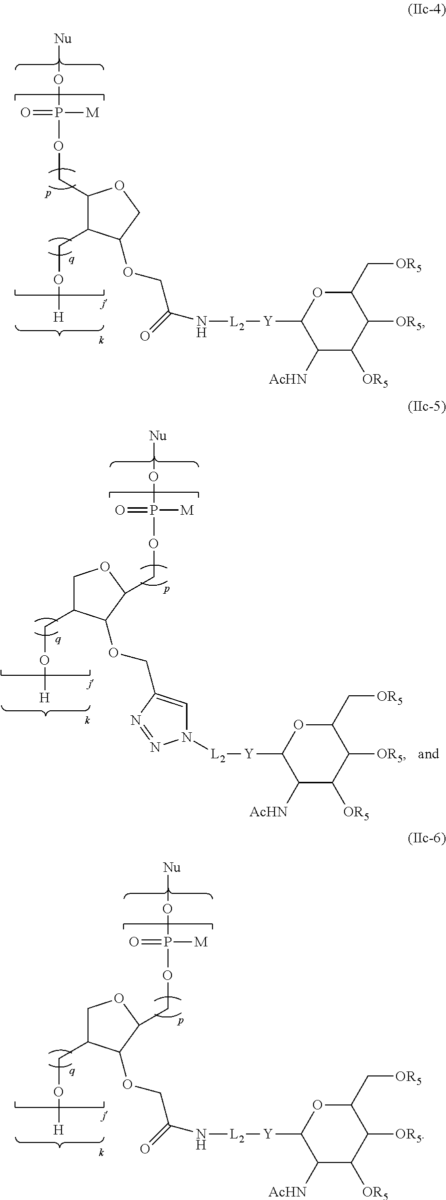

9. The conjugate, or the stereoisomer, the pharmaceutically acceptable salt or the prodrug thereof according to claim 6, wherein the conjugate is selected from the group consisting of:

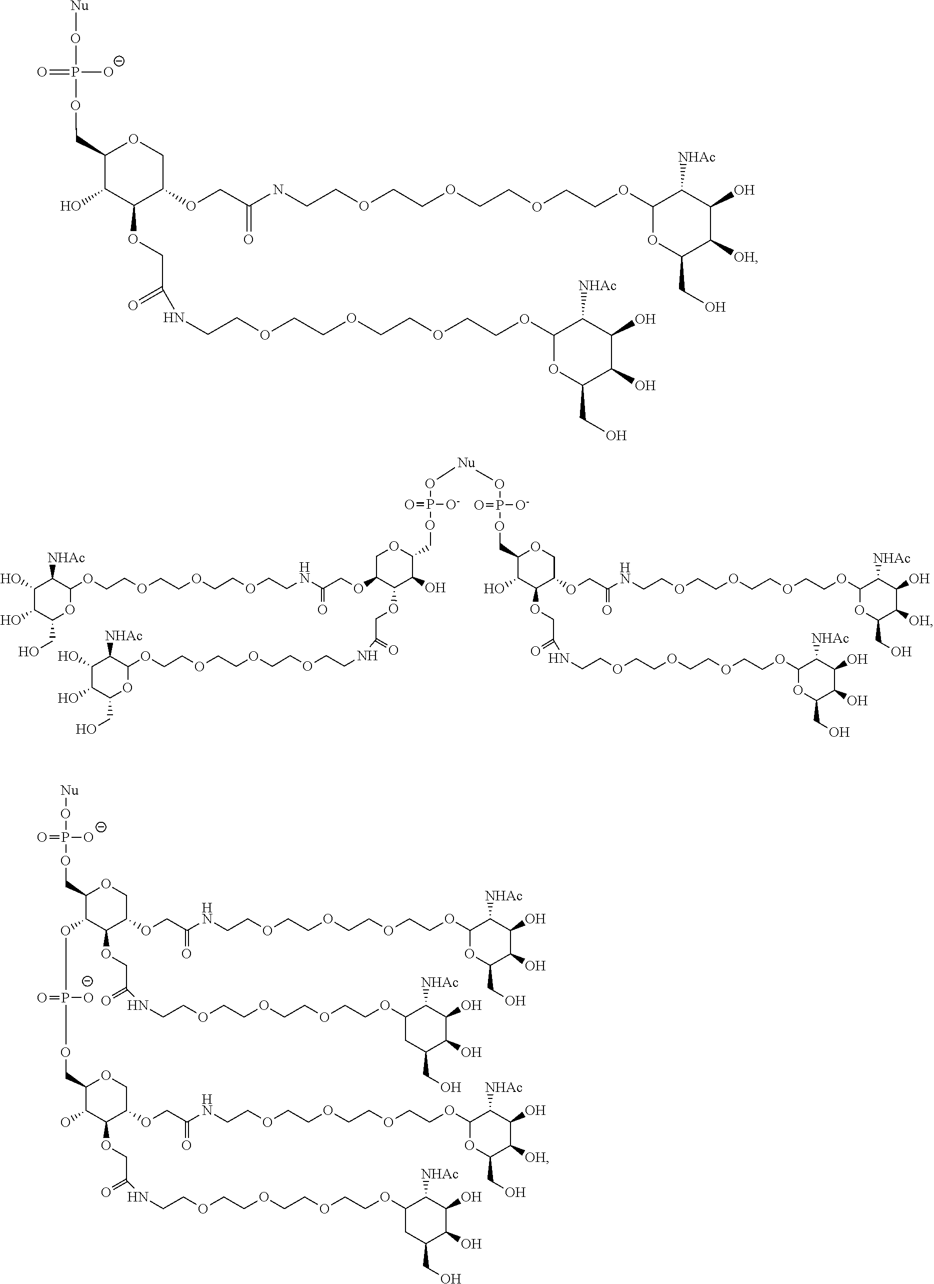

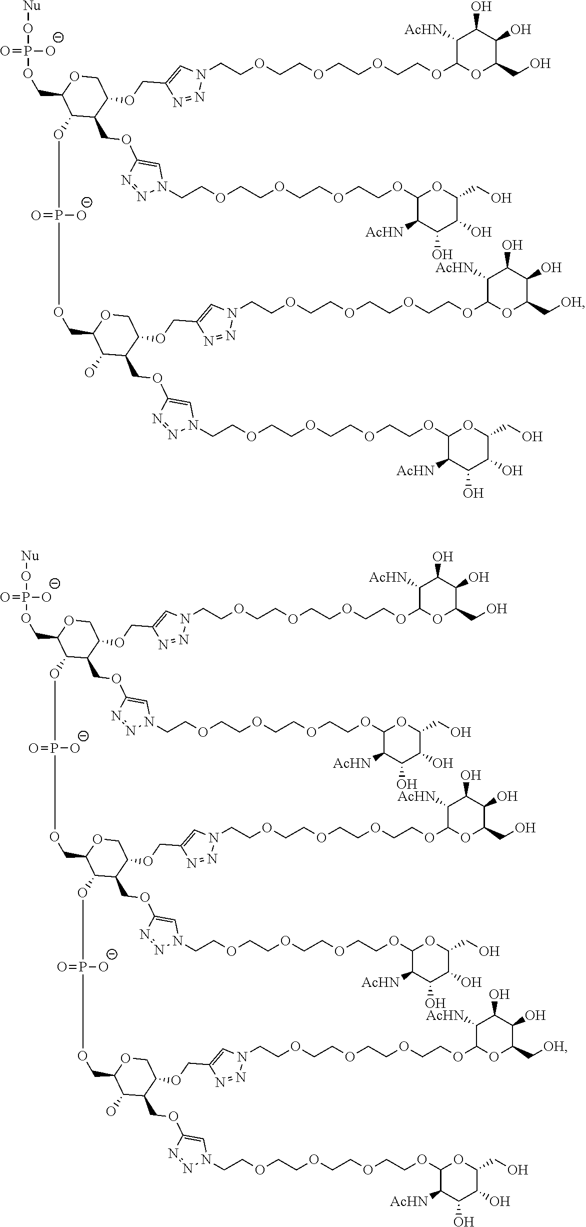

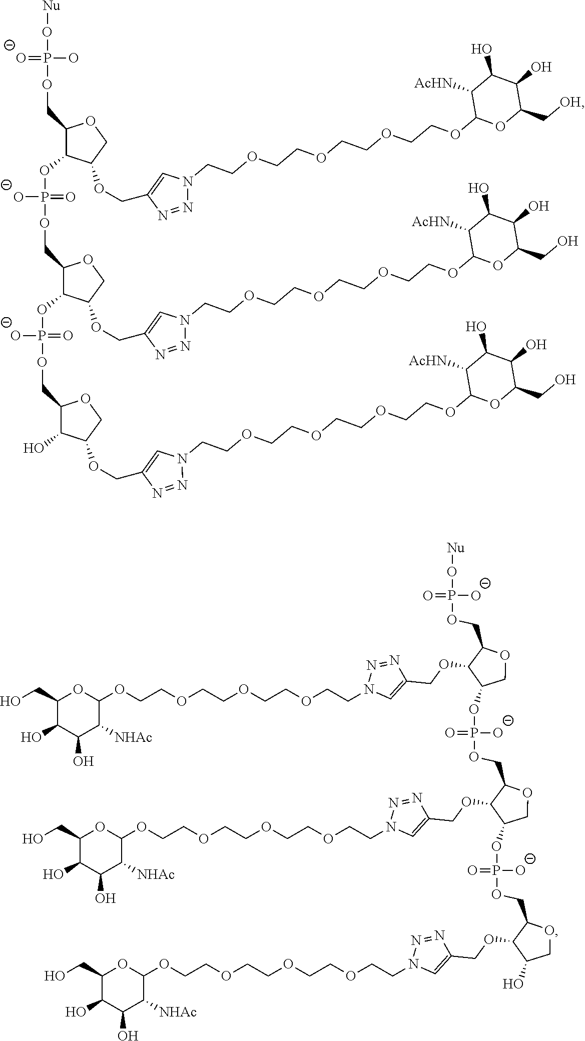

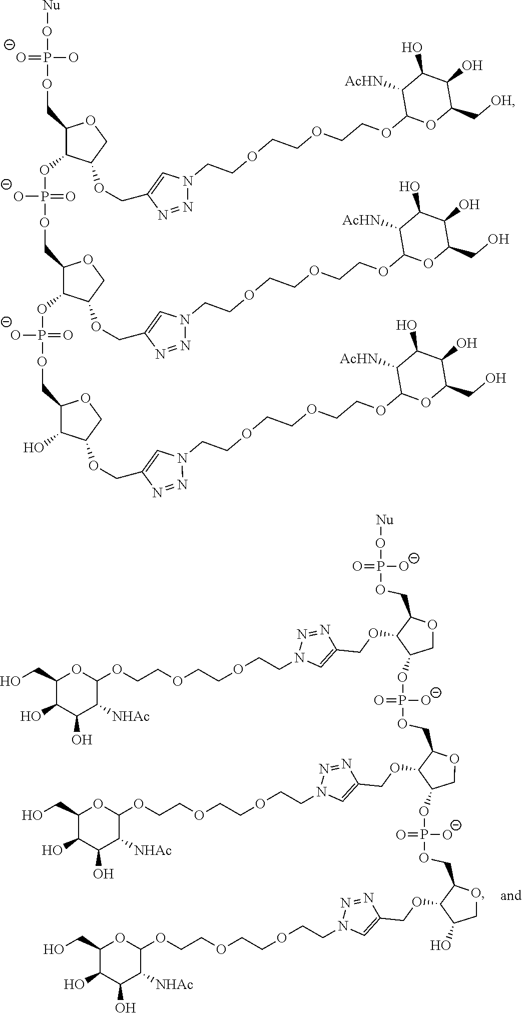

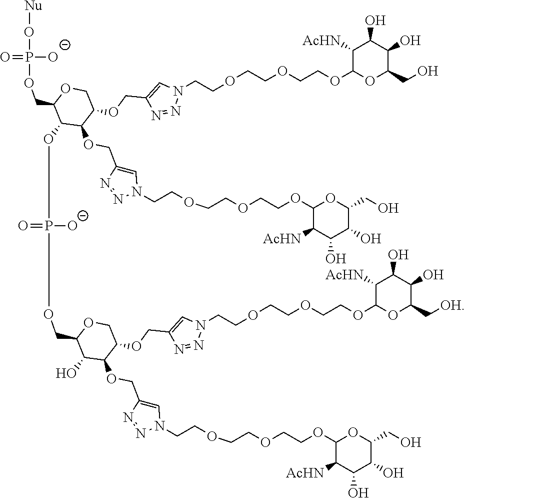

10. A composition comprising the conjugate, or the stereoisomer, the pharmaceutically acceptable salt or the prodrug thereof according to claim 6,
optionally, the composition further comprises optionally one or more pharmaceutically acceptable carriers or excipients.

11. A method for preventing and/or treating a disease in a subject in need thereof, comprising administering to the subject
the compound, or the stereoisomer, the pharmaceutically acceptable salt or the prodrug thereof according to
claim 1
optionally, wherein the disease is a hepatogenic disease.

12. A method for reducing expression or activity of a target gene in a cell, comprising contacting the cell with
the compound, or the stereoisomer, the pharmaceutically acceptable salt or the prodrug thereof according to
claim 1.

13. A medicament comprising the conjugate, or the stereoisomer, the pharmaceutically acceptable salt or the prodrug thereof according to claim 6, and a pharmaceutically acceptable excipient or adjuvant.

14. A pharmaceutical combination comprising the medicament according to claim 13 and any additional active ingredient.

15. A method for treating and/or preventing a disease in a subject in need thereof, comprising administering to the subject a pharmaceutically acceptable amount of
the conjugate, or the stereoisomer, the pharmaceutically acceptable salt or the prodrug thereof according to
claim 6.

16. A method for reducing expression or activity of a target gene, comprising contacting the conjugate, or the stereoisomer, the pharmaceutically acceptable salt or the prodrug thereof according to claim 6 with a cell.

17. A compound represented by formula (Ic), or a stereoisomer thereof: 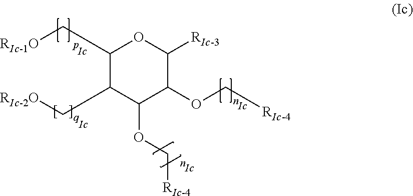
in formula (Ic), p_{Ic} and q_{Ic} are each independently selected from the group consisting of 0, 1, 2 and 3;
each n_{Ic} is independently selected from the group consisting of 1, 2, 3, 4 and 5;
R_{Ic-1} is selected from the group consisting of R_{Ic-1}′, and R_{Ic-1}′ is a hydroxyl protecting group;
R_{Ic-2} is selected from the group consisting of R_{Ic-2}′, and R_{Ic-2}′ is 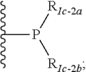
wherein R_{Ic-2a} is a secondary amino group, and R_{Ic-2b} is a secondary amino group or a C₁-C₃ alkoxy substituted with a cyano;
R_{Ic-3} is selected from the group consisting of H and C₁-C₃ alkoxy; and
each R_{Ic-4} is independently selected from the group consisting of

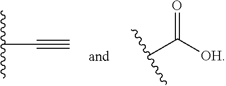

18. The compound or the stereoisomer thereof according to claim 17, wherein each R_{Ic-4} is

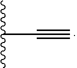 [SMILES: *C#C]

19. The compound or the stereoisomer thereof according to claim 17, wherein each R_{Ic-4} is

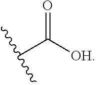 [SMILES: *C(=O)O]

20. The compound or the stereoisomer thereof according to claim 17, wherein p_{Ic} and q_{Ic} are each independently selected from the group consisting of 0 and 1;
optionally, p_{Ic}=1 and q_{Ic}=0;
optionally, each n_{Ic} is 1;
optionally, the hydroxyl protecting group has a structure of 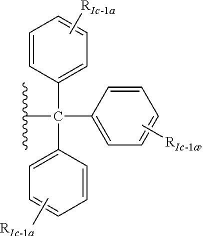
wherein each R_{Ic-a} is independently selected from the group consisting of H, C₁-C₃ alkyl and C₁-C₃ alkoxy;
optionally, each R_{Ic-1a} is independently selected from the group consisting of H and methoxy;
optionally, the hydroxyl protecting group is selected from the group consisting of trityl (Tr), 4-methoxytrityl (MMTr), 4,4′-dimethoxytrityl (DMTr) and 4,4′,4″-trimethoxytrityl (TMTr);
optionally, the hydroxyl protecting group is 4,4′-dimethoxytrityl (DMTr);
optionally, R_{Ic-2a} is 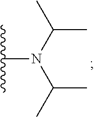 [SMILES: *N(C(C)C)C(C)C]
optionally, R_{Ic-2b} is selected from the group consisting of 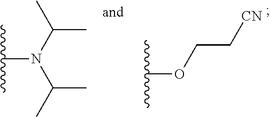
optionally, 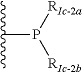
is selected from the group consisting of 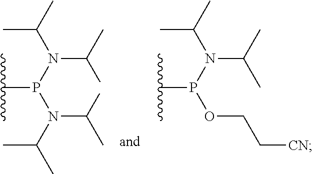 [SMILES: *P(N(C(C)C)C(C)C)N(C(C)C)C(C)C ; *P(OCCC#N)N(C(C)C)C(C)C]
and
optionally,

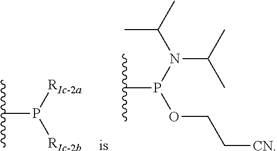

21. The compound or the stereoisomer thereof according to claim 17, wherein the compound has a structure selected from the group consisting of:

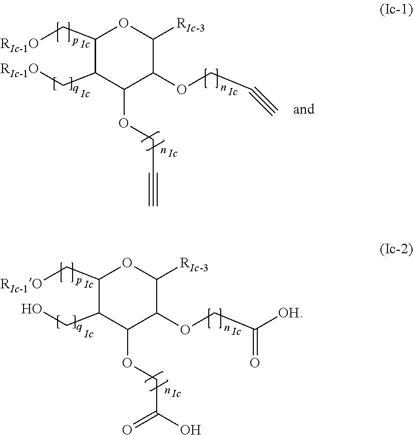

22. The compound or the stereoisomer thereof according to claim 17, wherein the compound has a structure selected from the group consisting of:

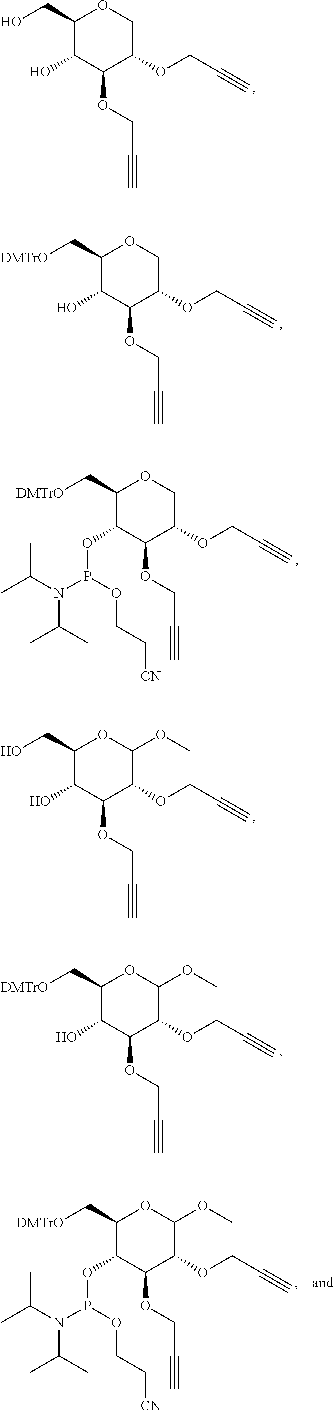

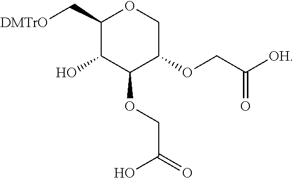

23. (canceled)

24. A method for reducing expression or activity of a target gene in a target cell, comprising contacting the compound or the stereoisomer thereof according to claim 17 with the target cell,
optionally, the target cell is a hepatocyte;
optionally, the medicament is a RNAi drug; and
optionally, the medicament is a siRNA drug.


SEQUENCE LISTING (15 sequences)

SEQ ID NO: 1 - RNA, 19 nt, synthetic construct
  source 1..19: mol_type = other RNA
ttttaatcctcactctaaa

SEQ ID NO: 2 - RNA, 21 nt, synthetic construct
  source 1..21: mol_type = other RNA
tttagagtgaggattaaaatg

SEQ ID NO: 3 - RNA, 18 nt, synthetic construct
  source 1..18: mol_type = other RNA
cagacagacaagaccatc

SEQ ID NO: 4 - RNA, 21 nt, synthetic construct
  source 1..21: mol_type = other RNA
agatggtcttgtctgtctgga

SEQ ID NO: 5 - RNA, 19 nt, synthetic construct
  source 1..19: mol_type = other RNA
cagacagacaagaccatct

SEQ ID NO: 6 - RNA, 19 nt, synthetic construct
  source 1..19: mol_type = other RNA
ccaagagcaccaagaacta

SEQ ID NO: 7 - RNA, 21 nt, synthetic construct
  source 1..21: mol_type = other RNA
tagttcttggtgctcttggtt

SEQ ID NO: 8 - DNA, 20 bp, synthetic construct
  source 1..20: mol_type = other DNA
ccagctccccattagctctg

SEQ ID NO: 9 - DNA, 22 bp, synthetic construct
  source 1..22: mol_type = other DNA
gcacttgcctctttaggaagtc

SEQ ID NO: 10 - DNA, 19 bp, synthetic construct
  source 1..19: mol_type = other DNA
tgcaccaccaactgcttag

SEQ ID NO: 11 - DNA, 19 bp, synthetic construct
  source 1..19: mol_type = other DNA
ggatgcagggatgatgttc

SEQ ID NO: 12 - DNA, 20 bp, synthetic construct
  source 1..20: mol_type = other DNA
gggttccacgtccatcagta

SEQ ID NO: 13 - DNA, 20 bp, synthetic construct
  source 1..20: mol_type = other DNA
acaccgtcctttccagcagt

SEQ ID NO: 14 - DNA, 23 bp, synthetic construct
  source 1..23: mol_type = other DNA
gaggagcagctaaccaacttaat

SEQ ID NO: 15 - DNA, 23 bp, synthetic construct
  source 1..23: mol_type = other DNA
tctgcatgtgctgttgacttaat